(19) United States
(12) Patent Application Publication
(10) Pub. No.: US 2025/0295749 A1
(43) Pub. Date: Sep. 25, 2025

(54) INACTIVATED STAPHYLOCOCCUS COMPOSITIONS AND METHODS OF MAKING AND USING THE SAME

(71) Applicant: Biological Mimetics, Inc., Frederick, MD (US)

(72) Inventors: Stephen J. Dollery, Herndon, VA (US); Gregory J. Tobin, Frederick, MD (US); Taralyn J. Wiggins, Sharpsburg, MD (US); Ruth V. Bushnell, Frederick, MD (US); John K. Tobin, Middletown, MD (US); Naomi J.P.E.R. Tasker, Frederick, MD (US); David A. MacLeod, Pepper Pike, OH (US)

(21) Appl. No.: 18/863,030

(22) PCT Filed: May 5, 2023
(86) PCT No.: PCT/US2023/066663

§ 371 Date: Nov. 5, 2024

Related U.S. Application Data
(60) Provisional application No. 63/339,195, filed on May 6, 2022.

(51) Int. Cl. A61K 39/085; A61K 39/00; A61P 37/04; C12N 1/20

(52) U.S. Cl. CPC A61K 39/085; A61P 37/04; C12N 1/20; A61K 2039/521

(57) ABSTRACT

Presented herein are inactivated Staphylococcal bacterial immunogens. Also described herein are compositions including Staphylococcal immunogens. Methods for preparing and using the same are also described. Immunogens may enable a host immune response that can protect the host from infection and/or disease. Differential analysis of antigens that stimulate protective (immunogenic) and non-protective immunity can be used to identify correlates of protection that can be developed as subunit vaccine candidates.

### STATEMENT OF PRIORITY

This application claims the benefit, under 35 U.S.C. § 119(e), of U.S. Provisional Application No. 63/339,195, filed May 6, 2022, the entire contents of which is incorporated by reference herein in its entirety.

### STATEMENT OF GOVERNMENT SUPPORT

This invention was made with government support under Grant Number A1145457, awarded by the National Institutes of Health. The United States government has certain rights in the invention.

### STATEMENT REGARDING ELECTRONIC FILING OF A SEQUENCE LISTING

A Sequence Listing in XML format, entitled 1472-6WO_ST26.xml, 503,540 bytes in size, generated on May 5, 2023, and filed herewith, is hereby incorporated by reference in its entirety for its disclosures.

### FIELD

The present invention relates to inactivated Staphylococcus aureus (S. aureus) compositions and methods for preparing and using the same, and to subunit correlates of immune protection.

### BACKGROUND

The clinical treatment of infections from antibiotic-resistant bacteria is complex, expensive, and often ineffective. The continuous evolution of antibiotic resistance complicates the development of medical countermeasures. The availability of safe and effective vaccines against these types of pathogens would be of high value in preventing or mitigating infections and reducing the evolution of additional resistance.

Many Staphylococcal isolates are resistant to antibiotic treatment and no vaccines are currently available to prevent their infection. In recent years, infections from multi-drug resistant bacteria have increased throughout the world causing world health authorities to call for increased efforts to develop new countermeasures.

In one example, Methicillin-resistant Staphylococcus aureus (MRSA) is a Gram-positive, round-shaped Firmicutes bacterium. MRSA is largely an opportunistic human pathogen that causes a range of disease in humans. MRSA resistance to penicillin is mediated by blaZ, a gene that encodes a B-lactamase enzyme that hydrolyzes the B-lactam ring of penicillin-type antibiotics. In addition to blaZ, MRSA strains can encode a variety of additional antibiotic resistance factors including a penicillin-binding protein (PBP2a), the plasmid-encoded vanA gene (for vancomycin resistance), and others.

MRSA diseases are commonly associated with community-acquired (CA-MRSA) and hospital-acquired (HA-MRSA) infections. As many as 33% of people in the US may be chronically infected yet do not show signs of disease. These people can act as carriers to spread the bacteria to others.

The CDC reports that significant progress was made to reduce MRSA bloodstream infections in healthcare settings from 2005 to 2012 where rates of infection decreased by about 17% per year due largely to more effective cleaning and other preventive procedures.

The World Health Organization has identified antimicrobial resistance as one of the most serious health threats worldwide. Because of the difficulty in treating multiple drug-resistant MRSA, prevention by cleaning and awareness have played major roles in reducing hospital acquired infections.

The present invention overcomes shortcomings in the art by providing staphylococcal immunogenic compositions, as well as methods of making and methods of using the same.

### SUMMARY OF THE INVENTION

The invention relates, in part, to novel whole-cell immunogenic compositions of staphylococcus which may have enhanced and/or novel immunogenicity. A staphylococcal immunogen composition of interest can serve as an immunogenic preparation and be used to produce antibodies, stimulate protective immunity from infection or disease, and/or to identify correlates of protective immunity.

Examples in this invention include compositions containing irradiation-inactivated MRSA that stimulate an immune response for protection from disease and/or production of antibodies.

Embodiments of the present invention may produce compositions containing irradiation-inactivated (such as by gamma ray, x-ray, and/or UV (e.g., UVC)) staphylococcus, which may improve the current practice of vaccine development by reducing damage to protective epitopes caused by chemical inactivation methods and thereby produce more immunogenic preparations.

In some embodiments, a protective antioxidant complex is used to reduce damage to protective epitopes during irradiation and the optimization of growth conditions that lead to the expression of protective antigens.

In some embodiments, inclusion of antioxidants such as manganese-peptide-orthophosphate (MDP) complexes may protect exterior macromolecules from damage during the radiation-inactivation process. Alternative antioxidants such as Vitamin C, superoxide dismutase, manganese-porphyrin complexes, others known to the art may substitute for MDP.

Embodiments of the present invention can be used to stimulate protective immunity. Such protective immunity can be analyzed by methods known in the art to identify subunits of the bacteria that can be developed as subunit vaccines.

In some embodiments, the present invention provides a method by which novel immunogens of MRSA are designed and produced. The present invention may utilize a manganese-decapeptide-orthophosphate (MDP) complex to protect staphylococcal immunogens during supralethal irradiation thereby uncoupling cell death due to DNA damage from epitope destruction. The MDP complex may protect enzymatic proteins within bacteria from oxidative damage caused by reactive oxygen species (ROS) that are formed during gamma and x-ray irradiation. Once protected, the enzymes may be able to repair DNA that has been damaged by both photons and/or ROS and this method has been hypothesized as the mechanism of radioresistance.

In other embodiments, the use of inactivated whole-cell immunogenic compositions is used to identify bacterial proteins and other subunits that are present in higher concentrations in protective immunogenic compositions than in non-protective immunogenic compositions. These correlates of protective immunity can be developed as second-generation subunit immunogens, immunogenic compositions, or vaccine candidates. It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1. (Top image) Coomassie analysis of MRSA proteins cultured using varying conditions. FIG. 1, Table 1 summarizes culture conditions (all were cultured under atmospheric gas).

FIG. 2. UVC irradiation kills MRSA and MDP protects epitopes. FIG. 2 panel A) MDP has minimal impact on MRSA survival following UVC exposure: 2×108 MRSA were exposed to UVC for varying times and 1% plated. FIG. 2 panel B) Quantitation of A. FIG. 2 panel C) 5 mins UVC exposure reduces 4×108 CFU to 0 CFU (6 experiments). FIG. 2 panel D) MDP protects proteins from oxidation. Planktonic MRSA were irradiated with UVC for 5 min in PBS, Mn+ buffer, or with MDP. Coomassie stain was used to control for concentration (left) and a western blot used to detect derivatized carbonyl groups (DNP) (right). FIG. 2 panels E, F, G) MDP protects epitopes detected by the immune system. Planktonic, Synovial fluid, or titanium drip cultured MRSA were irradiated for 5 mins with MDP or buffer, lysed, and epitopes probed with anti-MRSA mouse sera. The major band oxidized in the MDP-protected sample migrates at a molecular weight consistent with Protein A.

FIG. 3. Infected-bone-implant model. FIG. 3 panel A) Mice were vaccinated on day 0 and boosted on day 21. Mice were challenged on day 42 and observed for 7 days post challenge. CFU in the tibia following implant were determined per mg of bone. FIG. 3 panel B) Protection of mice vaccinated with different whole-cell preparations and challenged. Mice with a greater than 1 log reduction in CFU per mg of bone were judged protected. **=P 0.003, students T-test calculated using raw CFU values, 8 mice per group. FIG. 3 panel C) Western blot of MRSA (planktonic) probed with sera from mice in A (pre-challenge/post-boost). In Lanes 3-6 sera were from single mice that were protected. FIG. 3 panel D) A second, confirmatory study showing clearance of bacteria from mice vaccinated with new preparations of the inactivated cultures.

### DETAILED DESCRIPTION OF THE INVENTION

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, 5%, 1%, 0.5%, or even ±0.1% of X. A range provided herein for a measurable value may include any other range and/or individual value therein.

“Pharmaceutically acceptable” as used herein means that the compound, anion, cation, or composition is suitable for administration to a subject to achieve a treatment, such as one described herein, without unduly deleterious side effects in light of the severity of the disease and necessity of the treatment.

As used herein, the terms “increase,” “increases,” “increased,” “increasing,” “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “antigen” refers to a molecule capable of inducing the production of immunoglobulins (e.g., antibodies). As used herein, the term “immunogen” refers to when a molecule is capable of inducing a multi-faceted humoral and/or cellular-mediated immune response. In some embodiments, an antigen may be referred to as an immunogen, e.g., under conditions when the antigen is capable of inducing a multi-faceted humoral and/or cellular-mediated immune response. A molecule and/or composition (e.g., including but not limited to a nucleic acid, protein, polysaccharide, ribonucleoprotein (RNP), whole bacterium, and/or composition comprising the same) that is capable of antibody may be referred to as “antigenic” and/or that is capable of immune response stimulation may be referred to as “immunogenic,” and can be said to have the ability of antigenicity and/or immunogenicity, respectively. The binding site for an antibody within an antigen and/or immunogen may be referred to as an epitope (e.g., an antigenic epitope). The term “vaccine antigen,” “vaccine immunogen” or a composition comprising the same (e.g., an immunogenic composition, e.g., a subunit vaccine, e.g., a whole cell vaccine) as used herein refers to such an antigen and/or immunogen as used in a vaccine, e.g., a prophylactic, preventative, and/or therapeutic vaccine.

An “immunogenic amount” is an amount of a composition and/or immunogen of this invention that is sufficient to elicit, induce and/or enhance an immune response in a subject to which the composition is administered or delivered.

A vaccine is an immunogen or immunogenic composition that is used to generate an immunoprotective response, e.g., by priming the immune system such that upon further exposure to an antigen (e.g., an immunogen and/or antigen of an infectious entity such as, e.g., an infectious bacterium), the immune response is more protective to the host (e.g., vaccine recipient, e.g., the subject) as compared to the immune response against exposure to the antigen without prior vaccination. For example, an induced antibody can be provided by a vaccine that reduces the negative impact of the immunogen found on an infectious bacterium, or entity expressing same, in a host. The dosage for a vaccine may be derived, extrapolated, and/or determined from preclinical and clinical studies, as known to those of skill in the art. Multiple doses of a vaccine may be administered as known in the art and/or may be administered as needed to ensure a prolonged prophylactic and/or anamnestic (memory) state (e.g., a primed state). In some embodiments, the successful endpoint of the utility of a vaccine for the purpose of this invention is the resulting presence of an induced immune response (e.g., humoral and/or cell-mediated) resulting, for example, in the production of serum antibody or antibodies made by the host which recognizes the intended antigen. Such antibodies can be measured as is known in the art by a variety of assays such as, e.g., neutralization assays of serum sampled from animals or humans immunized with said vaccine, immunogen, and/or immunogenic composition. The design of vaccines against bacteria generally fall into two categories: (1) subunit vaccines and (2) whole-cell vaccines. Subunit vaccines, such as those for pertussis, pneumococcal, and meningococcal bacteria can be effective and their administration generally causes mild adverse reactions. However, the use of subunit vaccines generally requires many years or decades of research to identify the antigens of a bacterium that stimulate protective immunity. In addition, the manufacturing process by which recombinant proteins are expressed and purified requires considerable development to ensure that the proteins are produced in native form to stimulate protective immunity. For the majority of bacterial pathogens, including MRSA, a subunit-based vaccine that stimulates protective immunity has not been identified and validated. Whole-cell vaccines, such as those for pertussis and anthrax often stimulate immunity with improved durability, but can cause more significant adverse reactions, especially at the site of immunization. Multiple strategies exist for the development of whole-cell bacterial vaccines including chemical inactivation, physical disruption, and irradiation. All three methods may produce a safe vaccine but may also induce suboptimal immunity due to the disruption of or damage to antigenic epitopes during the inactivation process. Due to its relative rapidity of development, a whole-cell immunogenic composition may be developed as a first-generation vaccine for use in at-risk populations such as healthcare workers, military personnel, and patients awaiting planned surgeries. The whole-cell first-generation vaccine may also be suitable as a treatment option for patients struggling with chronic infections. A second-generation vaccine may be developed later after identification of immunogens that correlate with protection. Analysis of whole-cell immunogenic compositions that stimulate protective immunity can be compared to those that do not stimulate protective immunity to identify correlates of protection. These correlates can be developed as subunit immunogens in a second-generation vaccine.

The present invention relates to species, strains, and isolated of Staphylococcal bacteria, including but not limited to S. aureus. In some embodiments, the S. aureus species of the invention may be a drug-resistant S. aureus such as but not limited to methicillin-resistant SA (MSRA), multidrug-resistant SA (also referred to as MRSA), hospital-acquired MRSA (HA-MRSA), and/or community-acquired MRSA (CA-MSRA). MRSA is defined herein as strains of the Gram-positive firmicutes (Staphylococcus aureus) which infect humans and other animals, sometimes leading to hospitalization and or death. Multiple strains of MRSA are associated with antibiotic resistance and are difficult to treat with antibiotic therapies. There is no licensed vaccine against MRSA and therapeutic countermeasures to treat human infections are limited in both effectiveness and variety.

Staphylococci bacterial cells are propagated in a variety of methods to produce progeny cells that express varying protein profiles. Cells propagated in liquid and collected from liquid are normally termed “planktonic” bacterial forms while those propagated on solid substrate are normally termed “biofilm” forms. A variety of growth media is used including minimal nutrient and/or rich nutrient broths and/or agars. Biofilm forms are grown on the surface of agar nutrient plates, on the inside surfaces of plastic tubing and/or other bioreactors, on the surface of plates of various materials underneath growth media, and/or using other methods known to the art. Many growth platforms are adapted to aerobic and anaerobic growth conditions and a range of biologically suitable growth temperatures are also employed. Cells grown using a variety of methods are characterized by growth morphology, protein profiles, or other methods known to the art.

In order to propagate bacterial cultures for protein/proteomic analysis, immunogen screening and vaccine testing, many methods are applied and combined.

Bacteria are grown in a variety of rich media, limiting media and variations thereof including but not limited to M9, TSA, TSB, LB, CY, TB and TYB to yield unique protein expression profiles (immunogens).

Bacteria are grown in media at varying concentrations to induce virulence factors and other factors that generate unique protein expression profiles including but not limited to media concentrations of 1×, 0.5×, 0.2×, and 0.05×.

Media is supplemented with materials of animal or human origin, including but not limited to sera, blood, synovial fluid, plasma, brain extract to yield unique protein expression profiles. This is particularly relevant when microbes prefer proteins as a nutritional source or form biofilms in response to elements present in biological materials.

Bacteria are grown at different temperatures to induce virulence factors and other regulatory events that alter protein expression profiles including but not limited to temperatures of 72, 43, 40, 37, 32, 30, 28, 25, 23, 20, 17, 15, and 12° C. or any value or range therein.

Bacteria are grown in the presence of varying concentrations of gasses including low oxygen and high carbon dioxide concentrations. Oxygen is varied to a range of concentrations including but not limited to 0% to 20%. CO2 is varied to a range of concentrations including but not limited to 0% to 5%. Non-atmospheric gas concentrations are achieved in a variable atmospheric incubator or by total or near total displacement of atmospheric gasses with heavier inert gasses.

Bacteria are grown and harvested at several time points yielding unique protein expression profiles. Time points are designed to harvest bacteria from different growth phases ranging from lag, exponential, stationery (stable) and death phases of culture. Time points include but are not limited to 30 mins, 1 h, 2 h, 4 h, 6 h, 12 h, 18 h, 24 h, 48 h, 96 h, 192, and 240 hr, or any value or range therein.

Bacteria are cultured using a variety of platforms to generate planktonic and biofilm forms with unique protein profiles. Platforms include but are not limited to, continuous flow cultures such as tubing reactors, drip reactors, CDC tube reactors, inline reactors, annular reactors, and solid media plates (e.g., agar), shaking aqueous culture and static (motionless) aqueous cultures.

Composite materials of bioreactor growth surfaces are substituted to generate cultures with unique protein profiles including but not limited to, silicone, silicone-rubber, stainless steel, carbon steel, glass, polycarbonate, polypropylene, PVC, HDPE, polyurethane, nylon, rubber, titanium, iron, brass, bronze, nickel, concrete, hydroxyapatite and glass.

Cultures are harvested and chilled to less than 10° C. to limit further growth and alteration of protein expression. Cultures are pelleted via centrifugation, and resuspended in phosphate buffered saline an optimal number of times (e.g., 2 times) to enhance the neutralizing effects of radiation but preserve integrity of the sample.

Mass spectroscopy-high-performance liquid chromatography (HPLC) is applied to whole-cell lysates to analyze proteins expressed in growth conditions.

Mass spectroscopy is used on whole-cell lysates to correlate proteins expressed in growth condition with protection in animal studies (Tables 2 and 3)

Fractionation of membranes cam be used to downsize the number of candidate proteins identified as protective immunogens. Fractions include but are not limited to cell surface membrane fractions.

To down-select and define which proteins in a protein profile are protective (e.g., may be immunogens), several biochemical approaches are used in combination with mass spectroscopy.

Sera from vaccinated mice is harvested and stored. The survival of animals following challenge is noted as it indicates which animals have a protective antibody response and which sera samples contain protective and non-protective antibodies.

Sera is also generated by vaccinating animals with test antigens already known to be non-protective or protective (immunogens).

Serum antibodies and protein A and or protein G are used to bind and purify antigens which are then identified with methods including but not limited to mass spectroscopy.

Similarly, immunoprecipitation combined with mass spectroscopy analysis is performed to identify proteins that are uniquely immunogenic from a specific growth condition.

In addition to analyses such as mass spectroscopy (MS), other analyses by those skilled in the art are performed to characterize the differences in protein profiles among samples of bacteria grown in varying conditions including high-performance liquid chromatography (HPLC). Such characterizations are useful in identifying proteins that correlate with protective immunity and, thereby, identify bacterial proteins that are targets for subunit vaccine development.

Bacteria, both viable and radiation-inactivated cells, are analyzed for stability by plating diluted samples for CFU counts on agar growth plates or by microscopic counting of cells in a hemocytometer.

UVC rays, x-rays, and other ionizing radiations are used in the sterilization of medical supplies and equipment. In cells, UVC- and gamma-radiation causes direct damage through photons that indiscriminately introduce nicks into DNA, lesions into proteins, as well as lipid damage. However, the vast majority of radiation damage in aqueous conditions is indirect in nature and a result of ROS formed from the radiolysis of water. Superoxide (O2·−) is particularly dangerous to proteins because of its selective reactivity with certain amino acids and more so with Fe2+ bound to proteins. Dismutation of O2·− by Mn2+-peptide antioxidants produces hydrogen peroxide (H₂O₂) that are known to escape cells through membranes, unlike O2·−

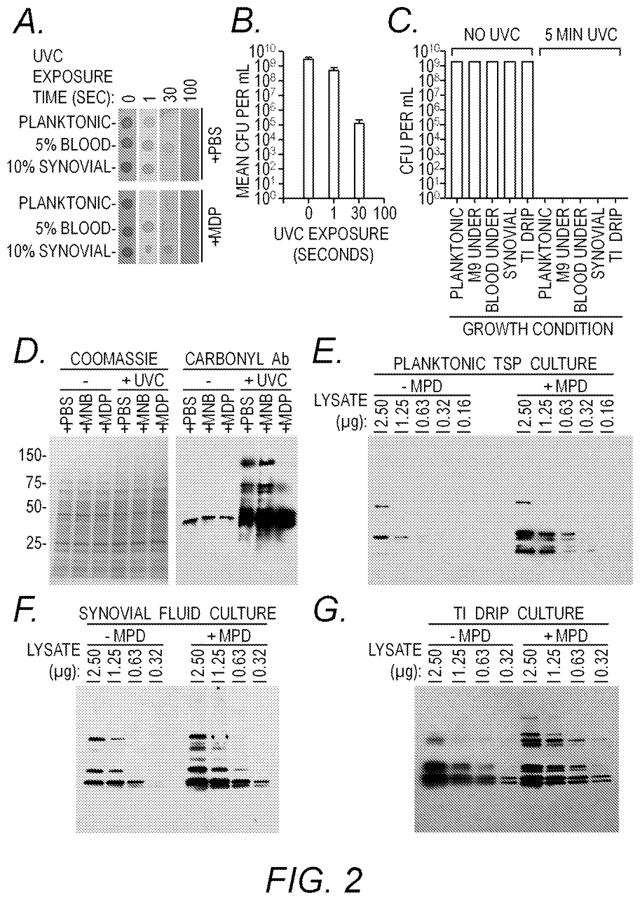

The data in FIG. 2 demonstrate a typical UVC kill-curve with a starting CFU count of approximately 5×109 bacteria/mL. Exposure to about 4 mW/cm2 of UVC for 100 seconds reduce CFU to zero sterilizing the samples. Exposure to Gamma-irradiation can also be used to inactivate the replicative capability of MRSA.

According to embodiments of the present invention, bacterial cells can be propagated in various ways to produce progeny cells that express varying protein profiles (e.g., protein antigens and immunogens). Cells propagated in liquid (e.g., liquid growth media) and collected from liquid are normally termed “planktonic” bacterial forms, while those propagated on solid substrate are normally termed “biofilm” forms. In some embodiments, a variety of growth media can be used including minimal nutrient and/or rich nutrient broths and/or agars. Biofilm forms can be grown on (e.g., above and/or underneath) surfaces of media (e.g., solid and/or liquid growth media). For example, biofilm forms can be grown on the surface of agar nutrient plates, on the inside surfaces of plastic tubing, on the surface of plastic plates underneath growth media, and/or using other methods known to the art. Cells grown using a variety of methods can be characterized by growth morphology, protein profiles, and/or other methods known to the art. Embodiments of the invention produce and/or provide a whole-cell bacterial vaccine and/or include a method for propagating bacteria such that the bacteria expresses proteins and/or other antigens that stimulate immune protection from later infection. For example, planktonic forms can be grown for about 2 to about 6 hours (e.g., about 2 hours, about 3 hours, about 4 hours, about 5 hours, about 6 hours or any value or range therein) in TSB media to exhibit logarithmic growth characteristics and/or for about 16 to about 36 hours (e.g., about 16 hours, about 17 hours, about 18 hours, about 19 hours, about 20 hours, about 21 hours, about 22 hours, about 23 hours, about 36 hours, or any value or range therein) in TSB to exhibit stationary growth phase characteristics. In further examples, biofilm forms can be grown for about 2 days to about 10 days (e.g., about 2 days, 3 days, 4 days, 5 days, 6 days, 7 days, 8 days, 9 days, 10 days, or any value or range therein) on plastic surfaces underneath liquid media, such as but not limited to M9 minimal media; for about 1 day to about 10 days (e.g., about 1 days, about 2 days, about 3 days, about 4 days, about 5 days, about 6 days, about 7 days, about 8 days, about 9 days, about 10 days, or any value or range therein) on the surface of agar plates using M9 media; for about 2 days to about 10 days (e.g., about 2 days, about 3 days, about 4 days, about 5 days, about 6 days, about 7 days, about 8 days, about 9 days, about 10 days, or any value or range therein) on plastic surfaces under rich media, such as but not limited to TSB; for about 2 days to about 10 days (e.g., about 2 days, about 3 days, about 4 days, about 5 days, about 6 days, about 7 days, about 8 days, about 9 days, about 10 days, or any value or range therein) inside plastic tubing using slowly flowing minimal media such as but not limited to M9 or 0.1×TSB; or other methods known to the art.

Growing bacteria under a variety of conditions may induce differential expression of virulence factors and/or bacterial antigens that stimulate a protective immune response.

Media used in embodiments of the present invention can be supplemented with materials of animal and/or human origin, including but not limited to sera, blood, synovial fluid, plasma, and/or brain extract, which may yield unique protein expression profiles (e.g., immunogens). Media supplements may be particularly relevant when microbes prefer proteins as a nutritional source or form biofilms in response to nutrients or scaffolds present in biological materials.

Bacteria can be grown at different temperatures, different atmospheric oxygen, and/or different CO2 concentrations, which may induce virulence factors and/or other regulatory events that may alter protein expression profiles (e.g., immunogens) including but not limited to temperatures of 72, 43, 40, 37, 32, 30, 28, 25, 23, 20, 17, 15, and 12° C., or any value or range therein.

Bacteria can be grown in the presence of varying concentrations of gasses including low oxygen and/or high carbon dioxide concentrations. Oxygen can be varied to a range of concentrations including but not limited to about 0% to about 20% (e.g., about 0%, about 0.5%, about 1%, about 5%, about 10%, about 15%, about 17.5%, about 20%, or any value or range therein). CO2 can be varied to a range of concentrations including but not limited to about 0% to about 15% (e.g., about 0%, about 1%, about 2%, about 5%, about 10%, about 12.5%, about 15%, or any value or range therein). Non-atmospheric gas concentrations can be achieved in a variable atmospheric incubator or by total or near total displacement of atmospheric gasses with heavier inert gasses.

Bacteria can be grown and harvested at one or more time point(s) which may yield unique protein expression profiles. Time points may be designed to harvest bacteria from different growth phases ranging from lag, exponential, stationery (stable) and/or death phases of culture. Time points can include but are not limited to 30 minutes, 1 hour, 2 hours, 4 hours, 6 hours, 12 hours, 18 hours, 24 hours, 48 hours, 96 hours, 192 hours, 240 hours, or any value or range therein.

Bacteria can be cultured using a variety of platforms to generate planktonic and/or biofilm forms, optionally with unique protein profiles (e.g., antigens and immunogens). Platforms include but are not limited to, continuous flow cultures such as tubing reactors, drip reactors, CDC tube reactors, inline reactors, annular reactors, solid media plates (e.g., agar), shaking aqueous culture and/or stationary aqueous cultures.

Composite materials of bioreactor growth surfaces can be substituted to generate cultures with unique protein profiles (e.g., antigens and immunogens) including but not limited to, silicone, silicone-rubber, stainless steel, carbon steel, glass, polycarbonate, polypropylene, PVC, HDPE, polyurethane, nylon, rubber, titanium, iron, brass, bronze, nickel, concrete, hydroxyapatite and glass.

Cultures may be harvested and chilled to 4° C. to limit further growth and alteration of protein expression.

Cultures may be pelleted via centrifugation, and washed and resuspended in phosphate buffered saline (PBS) an optimal number of times (e.g., 2 times). This may serve to stabilize potential protective effects of an antioxidant (e.g., MDP) comprised in the immunogenic composition upon irradiation (e.g., gamma and/or UVC) needed to preserve the integrity of the sample epitopes while removing nutrients to inhibit additional growth.

Methods known to the art for propagating bacterial cultures can be varied to promote the differential expression of proteins such that the inactivated bacteria stimulate protective immunity. Coomassie-stained polyacrylamide gels, Western blots, 2-dimensional electrophoresis, and other methods can be used to analyze the cultures to identify unique patterns of protein expression.

Additional analyses such as high-performance liquid chromatography (HPLC) and/or mass spectroscopy (MS), and/or other analyses known in the art may be performed to characterize the differences in protein profiles among samples of bacteria grown in varying conditions. Such characterizations may be useful for assessing the consistency of a whole-cell immunogen and in identifying proteins that correlate with protective immunity and, thereby, identifying bacterial proteins that may be targeted for subunit vaccine development.

Examples of bacterial proteins, e.g., antigens and immunogens, that may be expressed by bacteria grown under conditions as described in the present invention include, but are not limited to, virulence factors such as Panton-Valentine Leukocidin and alpha-hemolysin, AAA family ATPases, GlcNAc Transferases and other protein modifying enzymes, sodium/glutamate symporter molecules, amino acid permeases, ABC transporters and other importer and exporters of metabolites including iron response proteins. Yycl like proteins and other regulatory proteins including kinases, stress response proteins, biofilm associated proteins, planktonic associated proteins, and hypothetical and uncharacterized proteins and/or any protein shown in Table 2. Other bacterial proteins identified by analysis of differential expression between protective and non-protective whole-cell immunogens includes but is not limited tomaltose-binding periplasmic proteins, FAD-dependent oxidoreductase proteins, stress family proteins, cytochrome D subunit proteins, RecX regulatory protein, sulfate-binding proteins, amino acid carrier proteins, ABC transporter proteins, periplasmic molybdate-binding proteins, drug resistance MFS transporter proteins, protein K, type II secretions system proteins, NMT1/TH15-like protein, tRNA methyltransferase proteins, hydratase PaaB, coproporphyrinogen III oxidase, ribosomal protein L36, amidohydrolases, YedL, N-acetyltransferases, OmpW family proteins, U32 family peptidases, alanine racemase, surface or membrane-bound, and/or any protein shown in Tables 2 and 3.

Bacteria (e.g., viable bacterial cells and/or radiation-inactivated bacterial cells) may be analyzed for stability, for example, by plating diluted samples for colony-forming units (CFU) counts on agar growth plates and/or by microscopic counting of cells, for example, in a hemocytometer.

Gamma (γ) rays, x-rays, and/or other types of radiations may be used in the sterilization of medical supplies and equipment. In cells, gamma- and x-radiation may cause direct damage by photons that indiscriminately introduce nicks into DNA, lesions into proteins, as well as lipid damage. However, the vast majority of radiation damage in aqueous conditions is indirect in nature and a result of reactive oxygen species (ROS) formed from the radiolysis of water. For example, the ROS superoxide (O2·−) is particularly dangerous to proteins because of its selective reactivity with certain amino acids and with Fe2+ bound to proteins. Dismutation of O2·− by Mn2+-peptide antioxidants may produce hydrogen peroxide (H₂O₂) that can escape cells through membranes, unlike O2·−.

The MDP complex acts as an antioxidant by preventing the accumulation of superoxide, which limits the propagation of ROS. Manganese antioxidants like MDP are unique among redox active metal complexes accumulated in cells: Mn2+ ions are innocuous under conditions where other biologically active transition metals (e.g., Fe2+) tend to promote ROS, so many cells tolerate millimolar concentrations of Mn2+ within the cytoplasm. Moreover, Mn redox-cycling favors O2·−-scavenging without the release of extremely reactive hydroxy (HO*) radicals. In contrast, the redox-cycling of Fe, Cr and Cu gives rise to HO radicals by Fenton-type reactions. Thus, without Mn antioxidants, O₂^{·-} radicals become a significant source of HO· radicals, and hence a significant factor in the biochemical mechanism of epitope damage during the preparation of irradiated vaccines.

In the presence of MDP, UVC irradiation of bacteria causes far fewer oxidative lesions to proteins than when MDP is absent. MDP, however, does not protect nucleic acids (DNA or RNA). When bacteria are complexed with MDP, the immunogenic epitopes on the exterior of the bacteria (its surface molecules) are protected from ROS while the DNA inside the cell is fragmented and oxidized.

The MDP-bacteria complexes are produced by compounding a known quantity of bacteria (e.g., 105, 106, 107, 108, 109, 1010, 1011, or any value or range therein and/or other amounts) with stocks of manganese chloride, a peptide, and phosphate buffer. The final concentration of manganese chloride is 0.5 to 10 mM or higher with optimal concentrations in the range of 1 to 5 mM. The final concentration of the peptide is 0.5 to 10 mM with the optimal concentration in the range of 2 to 5 mM. The final concentration of phosphate buffer is 5 to 500 mM with optimal concentrations in the range of 25 to 200 mM.

The peptide can be a decapeptide with the amino acid composition of Asp-Glu-His-Gly-Thr-Ala-Val-Met-Leu-Lys (DEHGTAVMLK; SEQ ID NO:387). The peptide can be truncated to an amino acid composition of Asp-Glu-His-Gly-Thr-Ala-Val-Met (positions 1-8 of SEQ ID NO:387) or Asp-Glu-His-Met (positions 1-3 and 8 of SEQ ID NO:387) or similar, or rearrangements thereof. The composition of the peptide is more important than the sequence of the peptide. However, peptides composed of the above amino acids are preferred. The peptide can be manufactured synthetically in either the L- or D-configuration. The peptide can be produced by enzymatic or chemical degradation of polypeptides such as casein, ovalbumin, whey, or other abundant and relatively inexpensive proteins.

The peptide can be assessed for suitability by performing a functional assay. In one example of a functional assay, the peptide is formulated with manganese chloride, phosphate buffer, and a target enzyme, such as the restriction enzyme, BamHI. The concentrations of each component are varied. The mixture is exposed to Gamma-irradiation at doses of 0, 5, 10, 15, 20, 25, 30, 35, and 40 kGy on ice (or a range of UVC doses). After exposure the irradiated enzyme samples are mixed with a plasmid, such as pUC19 or pGEM1, containing the BamHI restriction site in duplicate samples. One set of duplicate samples are incubated for 15 minutes at 37° C. and the other set for 60 minutes at 37° C. At the end of the incubations, the samples are electrophoresed on a standard agarose gel known in the art. The residual activity of the enzyme is assessed by the percentage of DNA which has been linearized by enzymatic activity and these data are used to compare the mixtures of each component in the complex. With practice, the assay can be simplified by exposing the enzyme at fewer doses of Gamma (or UVC) irradiation, such as 0, 10, and 30 kGy to determine the optimal concentrations and compositions of the complex more rapidly.

The ortho-phosphate buffer in the MDP complex can be composed of sodium phosphate or potassium phosphate at pH values between 6.0 and 8.5. The optimal buffer for use with MRSA is potassium phosphate pH 7.4.

Closed test tubes containing the bacteria-MDP complex are placed on ice and introduced into chilled irradiation chamber or directly atop a UVC light-emitting wand. The dose of UVC, x-ray, UVC or other irradiation required to inactivate the replicative ability of the bacteria is assessed by spreading the bacteria onto agar plates containing nutrient media, incubating the plates at 37° C. for 16-36 hours, and counting the colonies. The colony counts are expressed as colony forming units (CFU) per unit of bacteria (e.g., 1010 bacterial cells). A kill curve is performed using doses ranging from 0 seconds to 600 seconds exposure to about 4 mW/cm2 of UVC. The minimum dose of irradiation required to kill 100% of the bacteria can be determined in a small number of experiments. In many cases, exposure to about 4 mW/cm2 of UVC for 10, 30, 60, or 100 s kills (sterilizes) 100% of the bacteria in samples of 108, 109, 1010 or 1011 cells.

A method of the present invention may use an antioxidant and/or antioxidant composition to protect antigenic epitope(s) on the surface of a bacterium, optionally while leaving the nucleic acid inside the bacterium subject to damage and/or destruction from ionizing radiation (e.g., gamma and/or x-ray radiation) and/or UV radiation (e.g., UVC radiation).

In some embodiments, a method of the present invention comprises providing an antioxidant composition (e.g., a composition comprising a peptide such as, e.g., a manganese-decapeptide-phosphate (MDP) composition) comprising a complex, which may protect bacterial epitopes during irradiation. An antioxidant composition of the present invention may comprise a divalent cation (e.g., Mn2+), a peptide, and a buffer system. In some embodiments, the antioxidant composition comprises manganese chloride (MnCl2), a decapeptide, and a phosphate buffer. In some embodiments, the antioxidant composition comprises manganese chloride (MnCl2), a decapeptide, and a Tris buffer. In some embodiments, the antioxidant composition comprises manganese chloride (MnCl2), a decapeptide, and a 2-(N-morpholino)ethanesulfonic acid (MES) buffer. In some embodiments, an antioxidant composition of the present invention comprises a manganese-decapeptide-phosphate (MDP) complex.

The MDP complex may act as an antioxidant by preventing the accumulation of superoxide, which may limit the propagation of ROS. Manganese antioxidants like MDP are unique among redox active metal complexes accumulated in cells. Mn2+ ions are innocuous under conditions where other biologically active transition metals (e.g., Fe2+) tend to promote ROS; therefore, many cells can tolerate millimolar concentrations of Mn2+ within the cytoplasm. Moreover, Mn redox-cycling favors O2·−-scavenging without the release of extremely reactive hydroxy (HO·) radicals. In contrast, the redox-cycling of Fe, Cr and Cu gives rise to HO·radicals by Fenton-type reactions. Thus, without Mn antioxidants, O2·− radicals can become a significant source of HO·radicals, and hence a significant factor in the biochemical mechanism of epitope damage during the preparation of irradiated immunogens and/or vaccines.

While not wishing to be bound by theory, in the presence of MDP, irradiation (e.g., gamma and/or x-ray irradiation) of a bacterium (e.g., MRSA) may cause fewer oxidative lesions to proteins than when MDP is absent. MDP, however, may not protect nucleic acids (e.g., DNA and/or RNA) leading to abolition of replicative capabilities (i.e., lack of colony-forming activity). When a bacterium is complexed with MDP, one or more immunogenic epitope(s) on the exterior of the bacteria (e.g., surface molecules) may be protected from ROS, optionally while the DNA inside the cell may be fragmented and/or oxidized. The end result may be an immunogenic cell that lacks replicative ability.

Compositions comprising MDP and bacteria of the present invention (e.g., MDP-MRSA complexes) may be produced by combining and/or contacting (e.g., “complexed”) an amount of bacteria (e.g., 105, 106, 107, 108, 109, 1010, 1011, or other amounts of bacteria) with an amount of a divalent cation (e.g., manganese chloride), an amount of a peptide, and an amount of a buffer (e.g., a phosphate buffer).

In some embodiments, an antioxidant composition of the present invention may comprise a divalent cation, such as, e.g., manganous Mn2+. In some embodiments, the divalent cation may be provided as a salt, e.g., MnCl2. In some embodiments, an antioxidant composition of the present invention may comprise a divalent cation (e.g., Mn2+) in a concentration of about 0.5 mM to about 10 mM, e.g., about 0.5, 1, 1.5, 2, 2.5, 3, 3.5, 4, 4.5, 5, 5.5, 6, 6.5, 7, 7.5, 8, 8.5, 9, 9.5, or 10 mM, or any value or range therein. In some embodiments, a composition of the present invention may comprise a divalent cation in the range of about 2 mM to about 5 mM (e.g., about 2, 2.5, 3, 3.5, 4, 4.5, or 5 mM or any value or range therein). For example, in some embodiments, an antioxidant composition of the present invention may comprise Mn2+ in an amount of about 1.4 mM to about 5.3 mM, about 2 mM to about 7 mM, or about 1 mM to about 9.8 mM. In some embodiments, an antioxidant composition of the present invention may comprise about 3 mM Mn2+. In some embodiments, an antioxidant composition of the present invention may comprise about 3 mM MnCl2.

In some embodiments, an antioxidant composition of the present invention may comprise a peptide in a concentration of about 0.5 mM to about 10 mM (e.g., about 0.5, 1, 1.5, 2, 2.5, 3, 3.5, 4, 4.5, 5, 5.5, 6, 6.5, 7, 7.5, 8, 8.5, 9, 9.5, 10 mM or any value or range therein). In some embodiments, an antioxidant composition for the present invention may comprise a peptide in a concentration in the range of about 2 mM to about 5 mM (e.g., about 2, 2.5, 3, 3.5, 4, 4.5, or 5 mM or any value or range therein). Thus, in some embodiments, the concentration of peptide (e.g., decapeptide) may be, for example, about 0.5 mM to about 5 mM, about 2 mM to about 7.5 mM, about 1.5 mM to about 8.5 mM, or about 2 mM, about 3.5 mM, about 3 mM, or about 5 mM in the composition.

In some embodiments, an antioxidant composition of the present invention may comprise a buffer, e.g., a phosphate buffer, a Tris buffer, an MES buffer, a HEPES buffer, and/or the like. The buffer may have a concentration of about 5 mM to about 500 mM (e.g., about 5, 10, 20, 30, 40, 50, 60, 70, 80, 90, 100, 150, 200, 250, 300, 350, 400, 450, 460, 470, 480, 490, 495, or 500 mM or any value or range therein). In some embodiments, the buffer may have a concentration in the range of about 25 mM to about 200 mM (e.g., about 25, 50, 75, 100, 125, 150, 175, or 200 mM or any value or range therein). Thus, in some embodiments, the concentration of buffer may be, for example, about 5 mM to about 450 mM, about 20 mM to about 500 mM, about 15 mM to about 350 mM, or about 25 mM, about 75 mM, about 200 mM or about 150 mM. In some embodiments, the buffer and/or antioxidant composition may have a pH of about 5 to about 9, or any value or range therein, e.g., about 6 to about 8.5, about 5 to about 7.8, about 6.5 to about 8, or about 6, about 6.8, about 7.4, or about 8.5. In some embodiments, an antioxidant composition of the present invention and/or method of their use may comprise a composition and/or method as described in PCT/US2008/073479; PCT/US2011/034484; and/or PCT/US2012/062998, the disclosures of which are incorporated herein by reference.).

A peptide of the present invention may comprise 2 or more amino acids (e.g., 2, 3, 4, 5, 6, 7, 8, 9, 10, 11, 12, 13, 14, 15, 16, 17, 18, 19, 20, or more), optionally wherein the peptide comprises two or more amino acids residues from the sequence DEHGTAVMLK (SEQ ID NO:387) in any order and/or length. The exact sequence and/or length of the peptide may vary and the peptide may contribute to antioxidant activities and/or function as an antioxidant in a composition of the present invention. For example, in some embodiments the peptide may be a tetrapeptide (4mer), a pentapeptide (5mer), a hexapeptide (6mer), a heptapeptide (7mer), an octapeptide (8mer), a nonapeptide (9mer), and/or a decapeptide (10mer). The peptide may be manufactured synthetically in either the L- or D-configuration. In some embodiments, a peptide (e.g., a decapeptide) of the present invention may comprise the amino acids DEHGTAVMLK (SEQ ID NO:387) in any order and/or length, e.g., the peptide may comprise the sequence of amino acids HMLK (SEQ ID NO:385), a scrambled sequence of the amino acids HMLK (SEQ ID NO:386), the sequence of amino acids HMHMHM (SEQ ID NO:386), a scrambled sequence of the amino acids HMHMHM (SEQ ID NO:386), the sequence of amino acid DEHGTAVMLK (SEQ ID NO:387), and/or a scrambled sequence of the amino acids DEHGTAVMLK (SEQ ID NO:387). In some embodiments, a peptide may comprise an amino acid sequence having at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence DEHGTAVMLK (SEQ ID NO:387). In some embodiments, a peptide may comprise an amino acid sequence having at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence HMLK (SEQ ID NO:385). In some embodiments, a peptide may comprise an amino acid sequence having at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence HMHMHM (SEQ ID NO:386).

The peptide may be a decapeptide with the amino acid composition of Asp-Glu-His-Gly-Thr-Ala-Val-Met-Leu-Lys (DEHGTAVMLK; SEQ ID NO:387). The peptide can be truncated to an amino acid composition of Asp-Glu-His-Gly-Thr-Ala-Val-Met or Asp-Glu-His-Met or similar, or rearrangements thereof. In some embodiments, the peptide may comprise an aspartic acid residue, a glutamic acid residue, a histidine residue, a glycine residue, a threonine residue, an alanine residue, a valine residue, a methionine residue, a leucine residue, and/or a lysine residue. In some embodiments, the peptide may comprise 1, 2, or more amino acid residues having a negatively charged side chain (e.g., aspartic acid and/or glutamic acid residues); 1, 2, or more amino acid residues having a positively charged side chain (e.g., histidine, lysine, and/or arginine residues); 1, 2, or more amino acid residues having a polar, uncharged side chain (e.g., threonine and/or serine residues); 1, 2, or more glycine residues; and/or 1, 2, 3, 4, or more amino acid residues having a hydrophobic side chain group (e.g., alanine, valine, methionine, leucine, and/or isoleucine residues). In some embodiments, the composition of amino acid residues in the peptide may be more important than the sequence of the amino acids.

A peptide of the present invention can be produced by enzymatic and/or chemical degradation of polypeptides such as casein, ovalbumin, whey, or other abundant and relatively inexpensive proteins. In some embodiments, the peptide contained within the MDP complex is not immunogenic and its inclusion with irradiated cells during injection into animals does not result in detected anti-peptide antibody production.

In some embodiments, an antioxidant composition of the present invention comprises MnCl2 in a concentration of about 0.5 mM to about 10 mM, a decapeptide (e.g., DEHGTAVMLK [SEQ ID NO:387]) in a concentration of about 0.5 mM to about 10 mM, and a phosphate buffer in a concentration of about 5 mM to about 500 mM. In some embodiments, an antioxidant composition of the present invention comprises about 3 mM MnCl2, about 3 mM decapeptide (e.g., DEHGTAVMLK [SEQ ID NO:387]), and about 200 mM phosphate buffer. However, concentrations of the components in the antioxidant composition may be varied as long as there is little degradation of effectiveness. An antioxidant composition may further comprise one or more excipient(s) such as, e.g., sorbitol, trehalose, etc., and/or one or more peptide(s) such as, e.g., HMHMHM (SEQ ID NO:386), HMLK (SEQ ID NO:385), and/or the like.

A method of the present invention may comprise growing and/or culturing a bacterium (e.g., planktonic and/or biofilm culture). The method may further comprise exposing a bacterium, optionally in the presence or absence of an antioxidant composition, to radiation (e.g., ionizing (e.g., gamma and/or x-ray) radiation and/or ultraviolet (e.g., UVC) radiation), which may result in protection of one or more epitopes (e.g., bacterial antigens) while leaving the bacterial genome open to damage and/or destruction from the radiation. In some embodiments, bacteria are exposed to ionizing radiation (e.g., gamma rays and/or x-rays) in an amount of at least about 0.5, 1, 0.5, 2, 2.5, 3, 3.5, 4, 4.5, 5, 6, 7, 8, 9, 10, 15, 20, 25, 30, 35, 40, 45, 50, 55, or 60 kGy, or any value or range therein. For example, in some embodiments, a bacterium is exposed to ionizing radiation (e.g., gamma radiation and/or x-rays) in an amount of at least about 0.5 to about 15 kGy, about 4 to about 9 kGy, about 1.5 to about 15 kGy, about 2 kGy to about 15 kGy, about 5 kGy to about 10 kGy, or about 1.5 kGy, about 4 kGy, about 7 kGy, about 8 kGy, or about 10 kGy or more. In some embodiments, a bacterium is exposed to UV (e.g., UVC) radiation in an amount of about 0.01, 0.5, or 0.1 kJ/m2 to about 5, 10, or 15 kJ/m2 (e.g., about 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15 kJ/m2), or an equivalent derived exposure time, surface area and/or light source wavelength and/or wattage. In some embodiments, a bacterium is exposed to UV (e.g., UVC) radiation in an amount of about 0.01, 0.5, or 0.1 kJ/m2 to about 0.5, 1, 1.5, 2, 2.5, 3, 3.5, 4, 4.5, 5, 5.5, or 6 kJ/m2.

In some embodiments, a bacterium may be exposed for about 60 minutes to a UVC light source emitting about 4 mW/cm2, optionally in an opaque plastic tube to destroy replicative activity. In some embodiments, a bacterium is exposed for about 1, 5, 10, or 30 seconds to about 1, 1.5, or 2 minutes to a UVC light source emitting about 4 mW/cm2 when the bacterium is contained in a UV-transparent vessel or tube also to destroy replicative activity. Thus, the transmissibility of the plastic tube affects the time required to inactivate replicative activity. In some embodiments, bacteria may be exposed to a UV source (e.g., a UVC light source) having an intensity and/or for a period of time sufficient to inactivate the infectivity as determined experimentally.

In some embodiments, a method of the present invention replaces air and/or dioxygen in contact with a composition of the present invention with argon. For example, the air in tubes comprising the bacterium and optional antioxidant composition may be at least partially replaced with argon. In some embodiments, a method of the present invention reduces the concentration and/or removes metals such as, e.g., iron, from compositions comprising the bacterium and/or antioxidant composition. For example, the amount of trace iron contamination in phosphate buffers and other reagents may lead to increased oxidative damage of protein epitopes. Thus, in some embodiments, iron and/or other metals may be removed from buffers and water using methods known to those of skill in the art such as, e.g., by passage through a chelating chromatographic column (Chelex column, BioRad). In some embodiments, iron and/or other metals may be present in a concentration less than about 100 mM.

Provided, according to some embodiments of the present invention, is increased protection of bacterial epitopes (e.g., surface protein epitopes) from damage during the irradiation process (e.g., ionizing and/or ultraviolet irradiation). Increased protection of bacterial epitopes may be compared to a control, e.g., increased protection of epitopes during gamma and/or UVC irradiation inactivation as compared to formalin/formaldehyde inactivation. Increased protection may be accomplished by at least partially replacing ambient air with a non-reactive gas (e.g., argon) in containers (e.g., tubes) containing the bacteria and/or removing and/or decreasing the amount of iron in compositions comprising the pre-inactivated bacteria. In some embodiments, air may be at least partially replaced with a non-reactive gas such that the content of oxygen is reduced by about 50% or more such as, e.g., by about 50%, 55%, 60%, 65%, 70%, 75%, 80%, 85%, 90%, 95%, 96%, 97%, 98%, 99% or more compared to the content of oxygen in the atmosphere and/or prior to the at least partial replacement.

Ultraviolet light may be used to inactivate MRSA with minimal to no damage to epitopes that stimulate protective immunity. Ultraviolet light can be divided into categories based on wavelength. UVA is 315-400 nm, UVB is 280-315 nm, and UVC is 100-280 nm. The infectivity of MRSA bacteria may be completely inactivated when exposed to a UVC (e.g., comprising a wavelength of about 220 to about 280 nm) light source emitting about 1 mW/cm2 for about 60 minutes, e.g., in a partially opaque plastic tube, or a UVC light source emitting about 5 mW/cm2 for about 1, 5, 10, or 30 seconds to about 1, 1.5, or 2 minutes if the bacterium is contained in a UV-transparent vessel or tube.

As described herein, a method of the present invention may comprise exposing a bacterium to radiation while the bacterium is in a vessel (e.g., a tube or container). As one of skill in the art would understand, the exposure conditions (e.g., intensity of radiation and/or time of exposure) may vary depending on the type and intensity of irradiation and the type and/or properties of the vessel. Ionizing radiation, such as gamma rays, are not easily blocked and the type of plastic or glass used in the vessel may not be critical. UVC radiation is more readily blocked by various types of plastic or glass. For example, in some embodiments, a vessel and/or tube may be clear and/or transparent, or may be opaque and/or frosted. In some embodiments, a vessel or tube may have a thickness of about 1 mm or more (e.g., about 1, 1.25, 1.5, 1.75, 2, 2.25, 2.5, 2.75, or 3 mm or more) (e.g., a “thick-walled” tube or vessel). In some embodiments, a vessel or a tube may have a thickness of about less than 1 mm (e.g., about 0.05, 0.1, 0.15, 0.2, 0.25, 0.30, 0.35, 0.40, 0.45, 0.50, 0.55, 0.60, 0.65, 0.70, 0.75, 0.80, 0.85, 0.90, or 0.95 mm) (e.g., a “thin-walled” tube or vessel). In some embodiments, a method of the present invention may comprise exposing an immunogen of the present invention to radiation while the immunogen is flowing in and/or being transported through a vessel and/or tube (e.g., a flow cell).

Thus, in some embodiments, a method of the present invention may expose a bacterium, optionally in a UV-transparent vessel or tube, to ultraviolet light (e.g., UVC) in an amount sufficient to at least partially inactivate the infectivity of the bacterium. In some embodiments, an amount sufficient to inactivate the bacterium may be a wavelength of about 220 to about 280, e.g., about 220, 225, 230, 235, 240, 245, 250, 255, 260, 265, 270, 275, or 280, or any range or value therein. In some embodiments, an amount sufficient to inactivate the bacterium may be a UVC light source emitting about 0.5 mW/cm2 to about 10 or 20 mW/cm2 or more, e.g., about 0.5, 0.75, 1, 1.25, 1.5, 1.75, 2, 3, 4, 5, 6, 7, 8, 9, 10, 11, 12, 13, 14, 15, 16, 17, 18, 19, or 20 mW/cm2 or any value or range therein. In some embodiments, an amount sufficient to inactivate the bacterium may be a UVC light source emitting about 10 mW/cm2 or higher, e.g., wherein the bacterium is highly concentrated, e.g., highly concentrated bacterial samples. While not wishing to be bound to theory, high-concentrated bacterial samples may require more UVC to inactivate potentially due to partial shielding of the light by the cells. In some embodiments, an amount sufficient to inactivate the bacterium may be a UVC light source exposure for about 10 seconds to about 75 minutes, e.g., about 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, or 75 minutes, or any value or range therein. For example, in some embodiments, a method of the present invention may comprise exposing a bacterium to UVC in an amount sufficient to completely inactivate the bacterium, e.g., about 30 minutes of exposure to a UVC light source at wavelength of about 254 emitting about 0.7 mW/cm2, or about 10 seconds to 5 minutes of exposure to a UVC light source at wavelength of about 254 emitting about 5 mW/cm2 e.g., in a UVC-transparent tube or vessel. In some embodiments, when complexed with a MDP complex during UVC-inactivation, epitopes are protected from damage as evidenced by stimulation of antibacterial antibodies and/or protective immune responses in animals or humans.

In some embodiments, a method of the present invention comprises exposing a bacterium and optionally a MDP composition to ultraviolet (UV) light (e.g., UVC) and then to ionizing (e.g., gamma) radiation.

The sterilizing effects of x-rays and/or gamma-rays in vaccine production are a result of direct damage to proteins and nucleic acids by photons and, more significantly (by far), indirect damage caused by reactive oxygen species (ROS) generated from the radiolysis of water molecules.

Some embodiments of the present invention result in protection of all or at least a portion (e.g., 10% or more, e.g., about 10%, about 20%, about 30%, about 40%, about 50%, about 60%, about 70%, about 80%, about 90%, about 95%, about 96%, about 97%, about 98%, or about 99% or more) of the exterior proteins that form the epitopes of the bacterium while leaving the RNA genome susceptible to destruction.

The potency of a bacterial vaccine, immunogen, or immunogenic composition can be measured by analysis of the antibacterial immune activity in immunized humans or selected test animals, wherein higher quantification of antibacterial immune activity (e.g., IgA, IgM and/or IgG, e.g., IgG1, IgG2, IgG3, and/or IgG4) activity in immunized subjects (e.g., humans and/or test animals) correlates with improved immunoprotective responses in subsequent exposure to bacteria. The potency of a bacterial vaccine or immunogen can be measured by analysis of the protective immunity raised in an immunized human or selected test animal that is challenged by either natural or experimental exposure to the bacterial pathogen. The quantitation of potency is measured using analyses known to the art which may include but are not limited to reduced bacterial burden in tissues, reduced disease parameters (e.g., reduced weight loss or behavioral signs), or reduced morbidity and/or mortality

The irradiation-inactivated bacterial immunogens and immunogenic compositions may be formulated in a simple solution such as water, a standard buffer, a standard saline solution, and/or the like. In some embodiments, an adjuvant may be included in a composition of the present invention, which may augment the magnitude and/or extend the duration of the immune response.

An immunogen and/or immunogenic composition of the present invention may be provided and/or packaged in any suitable package and/or container. In some embodiments, an immunogen and/or immunogenic composition of the present invention may be provided in a package suitable for administering the immunogen and/or composition to a subject. In some embodiments, glass vials, ampules, or other containers known to those of skill in the art may comprise an immunogen and/or composition of the present invention, optionally in single or multiple doses.

The amount of an immunogen and/or immunogenic composition administered to a subject and/or present in composition of the present invention is typically an amount sufficient to induce the desired immune response in the target host. Generally, the dosage employed may be about 0.1 microgram to about 100 micrograms of protein per dose (e.g., about 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, or 100 micrograms of protein per dose, or any value or range therein).

The immunogen and/or immunogenic composition of the present invention may be used to stimulate protective immunity in a subject (e.g., a human). The immunogen and/or immunogenic composition may be injected intramuscularly, intradermally, subcutaneously, and/or the like, into animals and/or humans, optionally using a standard syringe. In some embodiments, an immunogen and/or immunogenic composition of the present invention may be introduced into animals or humans using microneedles, patches designed to allow immunogens to penetrate the skin surface, and/or other methods known to the art.

In some embodiments, a manufacturing process for an immunogen and/or immunogenic composition of the present invention may include a procedure in which the immunogen and/or immunogenic composition is dried (e.g., desiccated by lyophilization, spray-drying, and/or the like). In some embodiments, the drying may increase the thermostability (e.g., by at least 10%, 15%, 20%, 25%, 30%, 35%, 40%, 45%, 50%, 55%, 60%, or more) of the immunogen and/or immunogenic composition and/or the drying may extend the shelf-life of the immunogen and/or immunogenic composition as measured (e.g., by at least 10%, 15%, 20%, 25%, 30%, 35%, 40%, 45%, 50%, 55%, 60%, or more), optionally by maintaining the immunogenic nature of the composition. The drying process may include compounding an immunogen and/or immunogenic composition of the present invention with one or more stabilizing excipient(s) known to those of skill in the art such as, but not limited to, sorbitol, trehalose, sucrose, polyethylene glycol, amino acids, and/or other additives. The drying procedure may utilize freeze-drying such as, e.g., lyophilization, spray-drying, and/or other methods known in the art.

In some embodiments, an adjuvant may be present in a vaccine of the present invention and the adjuvant may optionally stimulate an improved immune response. Example adjuvants include, but are not limited to, alum, aluminum hydroxide, aluminum phosphate, monophosphoryl Lipid A, saponin derivatives (e.g., QS-21), nucleic acids including oligonucleotides such as CpG, lipopolysaccharides, oil-and-water emulsions, squalene, saponin, and/or other adjuvanting substance(s) (e.g., flagellin).

The present invention is explained in greater detail in the following non-limiting examples.

### EXAMPLES

The following data, examples, and drawings are provided to exemplify various aspects of the instant invention and are in no way to be interpreted as limiting the scope of the invention of interest.

### Example 1: Data, Figures, and Tables

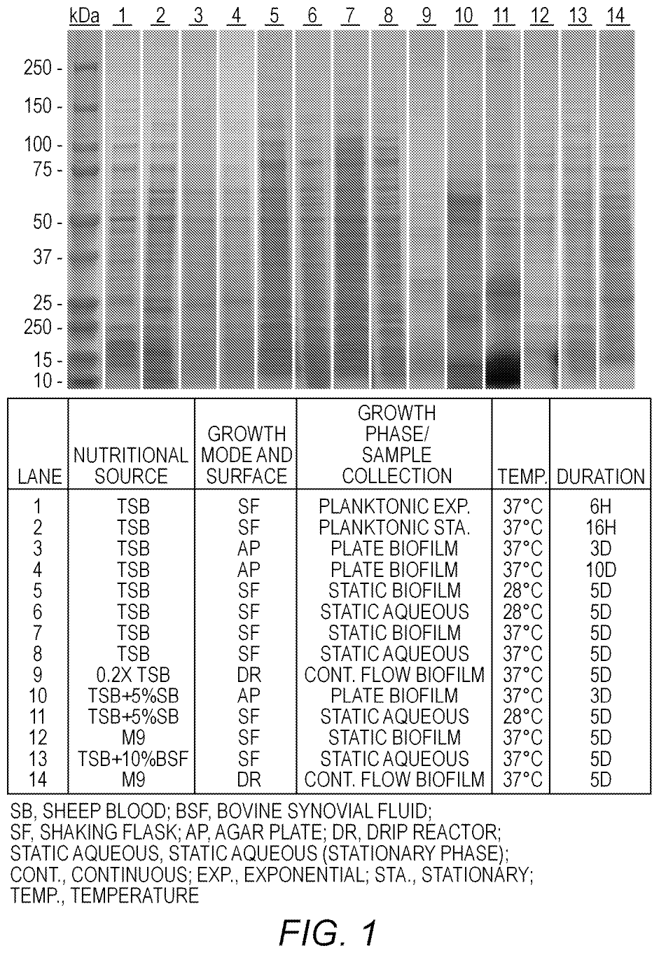

FIG. 1: Differential protein expression analysis of MRSA propagated under various growth conditions. Bacteria grown as either planktonic or biofilm forms were denatured and electrophoresed in an SDS-PAGE gel. Growth conditions are summarized in Table 1. Lane 1: cells grown at 37° C. in TSB as a planktonic culture while agitated (shaker), cells were harvested at 6 hrs during exponential phase. Lane 2: cells grown at 37° C. in TSB as a planktonic culture while agitated (shaker), cells were harvested at 16 hrs during stationary phase. Lane 3: cells grown at 37° C. as a biofilm on an TSB agar plate for 3 days. Lane 4: cells grown at 37° C. as a biofilm on an TSB agar plate for 10 days. Lane 5: cells grown as an aqueous biofilm by culturing in a motionless flask while submerged in TSB at 28° C. for 5 days. Adherent cells are harvested. Lane 6: cells grown to static phase in an aqueous motionless suspension, by culturing in a stationary flask containing TSB at 28° C. for 5 days. Non-adherent cells from the suspension were harvested. Lane 7: cells grown as an aqueous biofilm by culturing in a stationary flask submerged in TSB at 37° C. for 5 days. Adherent cells were harvested. Lane 8: cells grown to static phase in an aqueous motionless suspension, by culturing in a flask containing TSB at 37° C. for 5 days. Non-adherent cells from the suspension were harvested. Lane 9: cells grown as a biofilm in a continuous flow drip reactor. Cells were incubated at 37° C. and cultured with 0.2 g/L TSB, 0.2 g/L D-glucose at a flow rate of 240 μL per minute. Lane 10: cells grown at 37° C. as a biofilm on an TSB agar plate supplemented with 5% sheep's blood for 3 days. Lane 11: cells grown to static phase in an aqueous motionless suspension, by culturing in a stationary flask containing TSB supplemented with 5% sheep's blood at 28° C. for 5 days. Non-adherent cells from the suspension were harvested. Lane 12: cells grown to static phase in an aqueous motionless suspension, by culturing in a stationary flask containing M9 at 37° C. for 5 days. Adherent cells were harvested. Lane 13: cells grown to static phase in an aqueous motionless suspension, by culturing in a stationary flask containing TSB supplemented with 10% bovine synovial fluid at 37° C. for 5 days. Non-adherent cells from the suspension were harvested. Lane 14: cells grown as a biofilm in a continuous flow drip reactor. Cells were incubated at 37° C. and cultured with 0.2 g/L TSB, 0.2 g/L D-glucose at a flow rate of approximately 300 μL per minute.

FIG. 2. UVC irradiation kills MRSA and MDP protects epitopes. A) MDP has minimal impact on MRSA survival following UVC exposure. 2×108 MRSA were exposed to UVC for varying times and 1% were plated on agar growth media to show the rapid inactivation of MRSA by 30 seconds of UVC exposure in most samples B) Quantitation of A. C) 5 mins UVC exposure reduces 4×108 CFU to 0 CFU for all of the indicated conditions (6 experiments). D) MDP protects proteins from oxidation. Planktonic MRSA were irradiated with UVC for 5 min in PBS, Mn+ buffer, or with MDP. Coomassie staining was used to control for concentration (left) and a western blot used to detect derivatized carbonyl groups (DNP) (right). The proteins identified in the right panel have oxidized amino acid side chains which correlate with the lack of MDP during UVC inactivation. E, F, and, G) MDP protects epitopes detected by the immune system. MRSA were irradiated for 5 mins with MDP or buffer, lysed, and epitopes probed with anti-MRSA mouse sera. Bacteria inactivated in the presence of MDP exhibit stronger/darker signals than those inactivated without MDP indicating that MDP protects amino acids in the epitopes recognized by the immune sera. The major band shown in the MDP-protected sample migrates at a molecular weight consistent with Protein A which binds the Fc domains of antibodies in an antigen-independent manner.

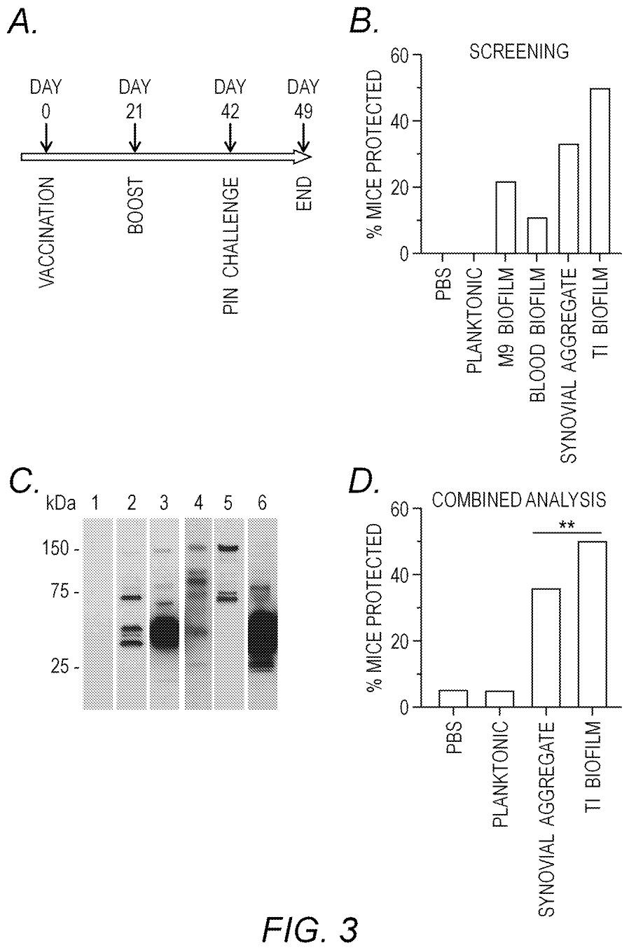

FIG. 3. Animal studies (infected-bone-implant model) show differential protection between groups. FIG. 3 panel A) Mice were vaccinated with either PBS (mock) or UVC inactivated preparations of MRSA. The following are the culture conditions of the samples shown in FIG. 3 Panels B, C, and D prior to UVC inactivation: Planktonic are cells grown at 37° C. in TSB as a planktonic culture while agitated (shaker). Cells were harvested at 16 hrs during stationary phase. M9 biofilm are cells grown to static phase in an aqueous motionless suspension by culturing in a stationary flask containing M9 at 37° C. for 5 days. Adherent cells are harvested. Blood biofilm are cells grown to static phase in an aqueous motionless suspension, by culturing in a stationary flask containing TSB supplemented with 5% sheep's blood at 28° C. for 5 days. Non-adherent cells from the suspension were harvested. Synovial aggregate are cells grown to static phase in an aqueous motionless suspension, by culturing in a stationary flask containing TSB supplemented with 10% bovine synovial fluid at 37° C. for 5 days. Non-adherent cells from the suspension are harvested. Ti biofilm are cells grown as a biofilm in a continuous flow drip reactor on a titanium plate. Cells were incubated at 37° C. and cultured with 0.2 g/L TSB, 0.2 g/L D-glucose at a flow rate of 240 μL per minute. FIG. 3 panel B) Protection of mice vaccinated with different whole-cell preparations and challenged. FIG. 3 panel C) Western blot of MRSA (planktonic) probed with sera from mice in B (pre-challenge/post-vaccination). In Lanes 3-6 sera were from single mice that was protected and identifies correlates of immunity in each sample. FIG. 3 panel D) To test the reproducibility of protection, the challenge study shown in FIG. 3 Panel B was replicated with select immunogens. In this study, an even greater degree of protection was observed, with clearance of bacteria to undetectable levels seen in 50% of the mice for the immunogen prepared from the bacteria grown on the titanium drip culture. The pattern of protection seen in FIG. 3 panel D was consistent with that seen in FIG. 3 panel B. This approach can be used to identify differential immune responses and measure different levels of protection in animals.

Table 2: Identification of corelates of immunity via protein analysis. The culture conditions used to make the inactivated whole-cell preparations that are protective are expected to express protective antigens. Conversely, protective antigens are not expected to be expressed in culture conditions that did not produce protective inactivate whole-cell immunogenic compositions. To identify protective proteins in the pin-implant infection model (FIG. 3), we compared the proteomes (without immunoprecipitation) of the protective synovial and Ti-plate biofilms to the nonprotective stationary-phase planktonic culture (same cultures as in FIG. 3 panel A). A combined average of 2 individual titanium cultures and 2 individual synovial fluid cultures identified 207 proteins increased by 1.5-fold and 97 proteins increased 2-fold vs. planktonic bacteria. Among these are well-studied virulence factors and vaccine candidates such as Leukocidin, alpha hemolysin, beta soluble modulin, SpA and as well as novel candidates as summarized. In Table 2, the GenBank® accession number, the descriptive name of the protein, and the fold increase in expression compared to non-protective planktonic proteins is shown.

TABLE 2
Table of protein immunogen candidates identified via non-IP method.

| | | | | Fold |
|---|---|---|---|---|
| No. | Accession No. | Description | SEQ ID NO: | Increase |
| 4-1 | Q4L7L3.1 | RecName: | 1 | 11.2 |
| | | Full = Aspartyl/glutamyl- | | |
| | | tRNA(Asn/Gln) amidotransferase | | |
| | | subunit B; Short = Asp/Glu-ADT | | |
| | | subunit B | | |
| 4-2 | A8Z414.1 | RecName: Full = Extracellular | 2 | 9.9 |
| | | matrix-binding protein ebh; | | |
| | | AltName: Full = ECM-binding | | |
| | | protein homolog; Flags: | | |
| | | Precursor | | |
| 4-3 | Q49YD9.1 | RecName: Full = Alanine | 3 | 7.4 |
| | | dehydrogenase | | |
| 4-4 | WP_000527296.1 | MULTISPECIES: hypothetical | 4 | 6.1 |
| | | protein [Bacilli] | | |
| 4-5 | WP_000437967.1 | MULTISPECIES: GAF domain- | 5 | 5.7 |
| | | containing sensor histidine | | |
| | | kinase [Staphylococcus] | | |
| 4-6 | WP_000911657.1 | MULTISPECIES: pyruvate | 6 | 4.7 |
| | | formate lyase-activating protein | | |
| | | [Staphylococcus] | | |
| 4-7 | WP_000959276.1 | MULTISPECIES: competence | 7 | 4.6 |
| | | protein CoiA [Staphylococcus] | | |
| 4-8 | B9DM35.1 | RecName: Full = 50S ribosomal | 8 | 4.3 |
| | | protein L5 | | |
| 4-9 | Q49VN8.1 | RecName: Full = Malate | 9 | 4.3 |
| | | dehydrogenase | | |
| 4-10 | P38507.1 | RecName: Full = Immunoglobulin | 10 | 4.2 |
| | | G-binding protein A; Short = IgG- | | |
| | | binding protein A; AltName: | | |
| | | Full = Staphylococcal protein A; | | |
| | | Short = SpA; Flags: Precursor | | |
| 4-11 | Q8NWQ3.1 | RecName: Full = Alanine | 11 | 4.1 |
| | | dehydrogenase 1 | | |
| 4-12 | Q49X39.1 | RecName: Full = ATP-dependent | 12 | 3.8 |
| | | protease ATPase subunit HsIU; | | |
| | | AltName: Full = Unfoldase HsIU | | |
| 4-13 | Q49XD1.1 | RecName: Full = 30S ribosomal | 13 | 3.8 |
| | | protein S14 | | |
| 4-14 | WP_000398672.1 | MULTISPECIES: beta-class | 14 | 3.6 |
| | | phenol-soluble modulin [Bacteria] | | |
| 4-15 | WP_094669328.1 | YfcC family protein | 15 | 3.5 |
| | | [Staphylococcus aureus] | | |
| 4-16 | Q08854.3 | RecName: Full = 60 kDa | 16 | 3.5 |
| | | chaperonin; AltName: | | |
| | | Full = GroEL protein; AltName: | | |
| | | Full = Heat shock protein 60; | | |
| | | AltName: Full = Protein Cpn60 | | |
| 4-17 | WP_000606724.1 | MULTISPECIES: hypothetical | 17 | 3.5 |
| | | protein [Staphylococcus] | | |
| 4-18 | WP_011058338.1 | MULTISPECIES: cysteine-rich | 18 | 3.4 |
| | | KTR domain-containing protein | | |
| | | [Bacteria] | | |
| 4-19 | WP_000660054.1 | MULTISPECIES: | 19 | 3.4 |
| | | argininosuccinate synthase | | |
| | | [Staphylococcus] | | |
| 4-20 | Q8NXF3.1 | RecName: | 20 | 3.3 |
| | | Full = Argininosuccinate lyase; | | |
| | | Short = ASAL; AltName: | | |
| | | Full = Arginosuccinase | | |
| 4-21 | WP_017466774.1 | AAA family ATPase | 21 | 3.3 |
| | | [Staphylococcus aureus] | | |
| 4-22 | Q6G6F1.1 | RecName: Full = D-lactate | 22 | 3.2 |
| | | dehydrogenase; Short = D-LDH; | | |
| | | AltName: Full = D-specific 2- | | |
| | | hydroxyacid dehydrogenase | | |
| 4-23 | WP_001144055.1 | MULTISPECIES: amino acid | 23 | 3.2 |
| | | ABC transporter ATP-binding | | |
| | | protein [Staphylococcus] | | |
| 4-24 | WP_001074353.1 | MULTISPECIES: carbamate | 24 | 3.1 |
| | | kinase [Staphylococcus] | | |
| 4-25 | WP_000719183.1 | MULTISPECIES: | 25 | 3.1 |
| | | glycosyltransferase | | |
| | | [Staphylococcus] | | |
| 4-26 | WP_000410718.1 | MULTISPECIES: hypothetical | 26 | 3.1 |
| | | protein [Staphylococcus] | | |
| 4-27 | WP_000448227.1 | MULTISPECIES: nitrite | 27 | 3.1 |
| | | reductase small subunit NirD | | |
| | | [Staphylococcus] | | |
| 4-28 | WP_000149686.1 | MULTISPECIES: cysteine | 28 | 3.1 |
| | | hydrolase [Staphylococcus] | | |
| 4-29 | WP_000138475.1 | MULTISPECIES: recombinase | 29 | 3 |
| | | RecT [Staphylococcus] | | |
| 4-30 | Q2YWH4.2 | RecName: Full = Carbamate | 30 | 3 |
| | | kinase 2 | | |
| 4-31 | WP_000959426.1 | MULTISPECIES: alanine | 31 | 3 |
| | | dehydrogenase [Staphylococcus] | | |
| 4-32 | B9DN51.1 | RecName: Full = L-lactate | 32 | 3 |
| | | dehydrogenase; Short = L-LDH | | |
| 4-33 | Q2YZB8.1 | RecName: Full = tRNA | 33 | 3 |
| | | modification GTPase MnmE | | |
| 4-34 | Q8CQ56.1 | RecName: Full = Alcohol | 34 | 2.9 |
| | | dehydrogenase; Short = ADH | | |
| 4-35 | WP_000720821.1 | MULTISPECIES: peptide ABC | 35 | 2.9 |
| | | transporter substrate-binding | | |
| | | protein [Staphylococcus] | | |
| 4-36 | P47729.2 | RecName: Full = DNA polymerase | 36 | 2.8 |
| | | III PolC-type; Short = Pollll | | |
| 4-37 | WP_000939496.1 | MULTISPECIES: hypothetical | 37 | 2.8 |
| | | protein [Bacteria] | | |
| 4-38 | WP_000960862.1 | MULTISPECIES: L-lactate | 38 | 2.8 |
| | | permease [Staphylococcus] | | |
| 4-39 | WP_000735554.1 | MULTISPECIES: hypothetical | 39 | 2.8 |
| | | protein [Staphylococcus] | | |
| 4-40 | WP_000943842 | MULTISPECIES: YSIRK domain- | 40 | 2.8 |
| | | containing triacylglycerol lipase | | |
| | | Lip2/Geh [Staphylococcus] | | |
| 4-41 | Q8NWQ4.1 | RecName: Full = L-threonine | 41 | 2.8 |
| | | dehydratase catabolic TdcB; | | |
| | | AltName: Full = Threonine | | |
| | | deaminase | | |
| 4-42 | Q2YSL2.1 | RecName: Full = Lipoteichoic acid | 42 | 2.8 |
| | | synthase; Contains: RecName: | | |
| | | Full = Glycerol phosphate | | |
| | | lipoteichoic acid synthase; | | |
| | | Short = LTA synthase; AltName: | | |
| | | Full = Polyglycerol phosphate | | |
| | | synthase; Contains: RecName: | | |
| | | Full = Processed glycerol | | |
| | | phosphate lipoteichoic acid | | |
| | | synthase | | |
| 4-43 | Q5HKH9.1 | RecName: Full = Formate | 43 | 2.8 |
| | | acetyltransferase; AltName: | | |
| | | Full = Pyruvate formate-lyase | | |
| 4-44 | WP_000739205.1 | MULTISPECIES: fibrinogen- | 44 | 2.7 |
| | | binding protein [Staphylococcus] | | |
| 4-45 | WP_001031880.1 | MULTISPECIES: L-lactate | 45 | 2.6 |
| | | dehydrogenase [Staphylococcus] | | |
| 4-46 | WP_001071721.1 | MULTISPECIES: anaerobic | 46 | 2.6 |
| | | ribonucleoside-triphosphate | | |
| | | reductase [Staphylococcus] | | |
| 4-47 | WP_000955797.1 | MULTISPECIES: bifunctional | 47 | 2.6 |
| | | acetaldehyde-CoA/alcohol | | |
| | | dehydrogenase [Staphylococcus] | | |
| 4-48 | WP_001200748.1 | MULTISPECIES: alcohol | 48 | 2.6 |
| | | dehydrogenase AdhP | | |
| | | [Staphylococcus] | | |
| 4-49 | B9DPI8.1 | RecName: Full = 50S ribosomal | 49 | 2.6 |
| | | protein L19 | | |
| 4-50 | WP_000649898.1 | MULTISPECIES: ABC | 50 | 2.5 |
| | | transporter permease subunit | | |
| | | [Staphylococcus] | | |
| 4-51 | Q5HEG6.3 | RecName: Full = Delta-hemolysin; | 51 | 2.5 |
| | | Short = Delta-lysin; AltName: | | |
| | | Full = Delta-toxin | | |
| 4-52 | WP_001010115.1 | MULTISPECIES: glycerol-3- | 52 | 2.5 |
| | | phosphate transporter | | |
| | | [Staphylococcus] | | |
| 4-53 | Q5HGR3.1 | RecName: Full = Ornithine | 53 | 2.5 |
| | | carbamoyltransferase; | | |
| | | Short = OTCase | | |
| 4-54 | A6QKC0.1 | RecName: Full = HTH-type | 54 | 2.5 |
| | | transcriptional regulator ArcR | | |
| 4-55 | WP_000161541.1 | MULTISPECIES: D-lactate | 55 | 2.4 |
| | | dehydrogenase [Staphylococcus] | | |
| 4-56 | WP_000546609.1 | MULTISPECIES: potassium- | 56 | 2.4 |
| | | transporting ATPase subunit | | |
| | | KdpB [Staphylococcus] | | |
| 4-57 | Q2YYS9.1 | RecName: Full = Urease | 57 | 2.4 |
| | | accessory protein UreF | | |
| 4-58 | WP_000660023.1 | MULTISPECIES: carbamate | 58 | 2.4 |
| | | kinase [Staphylococcus] | | |
| 4-59 | WP_000857479.1 | MULTISPECIES: alpha- | 59 | 2.4 |
| | | hemolysin [Staphylococcus] | | |
| 4-60 | WP_000136159.1 | MULTISPECIES: ornithine | 60 | 2.4 |
| | | carbamoyltransferase | | |
| | | [Staphylococcus] | | |
| 4-61 | Q4L3K1.1 | RecName: Full = 50S ribosomal | 61 | 2.4 |
| | | protein L7/L12 | | |
| 4-62 | WP_000129413.1 | MULTISPECIES: arginine | 62 | 2.4 |
| | | deiminase [Staphylococcus] | | |
| 4-63 | Q49XF1.1 | RecName: Full = Protein GlcT | 63 | 2.4 |
| 4-64 | WP_000894660.1 | MULTISPECIES: formate C- | 64 | 2.4 |
| | | acetyltransferase | | |
| | | [Staphylococcus] | | |
| 4-65 | Q4L3G8.1 | RecName: Full = ATP-dependent | 65 | 2.3 |
| | | zinc metalloprotease FtsH | | |
| 4-66 | WP_017466734.1 | YjiH family protein | 66 | 2.3 |
| | | [Staphylococcus aureus] | | |
| 4-67 | Q4L941.1 | RecName: Full = L-lactate | 67 | 2.3 |
| | | dehydrogenase; Short = L-LDH | | |
| 4-68 | P60890.1 | RecName: Full = Glutamine | 68 | 2.2 |
| | | synthetase; Short = GS; AltName: | | |
| | | Full = Glutamate--ammonia ligase; | | |
| | | AltName: Full = Glutamine | | |
| | | synthetase I alpha; Short = GSI | | |
| | | alpha | | |
| 4-69 | P13977.1 | RecName: Full = DegV domain- | 69 | 2.2 |
| | | containing 15.5 kDa protein | | |
| 4-70 | WP_000617348.1 | MULTISPECIES: N- | 70 | 2.2 |
| | | acetyltransferase | | |
| | | [Staphylococcus] | | |
| 4-71 | P35138.2 | RecName: Full = 50S ribosomal | 71 | 2.1 |
| | | protein L15 | | |
| 4-72 | WP_001066444.1 | MULTISPECIES: dUTP | 72 | 2.1 |
| | | diphosphatase [Staphylococcus] | | |
| 4-73 | Q6GFT9.1 | RecName: Full = HTH-type | 73 | 2.1 |
| | | transcriptional regulator rot; | | |
| | | AltName: Full = Repressor of | | |
| | | toxins | | |
| 4-74 | Q5HQW9.1 | RecName: Full = UvrABC system | 74 | 2.1 |
| | | protein A; Short = UvrA protein; | | |
| | | AltName: Full = Excinuclease ABC | | |
| | | subunit A | | |
| 4-75 | P00807.1 | RecName: Full = Beta-lactamase; | 75 | 2.1 |
| | | AltName: Full = Penicillinase; | | |
| | | Flags: Precursor | | |
| 4-76 | WP_001063330.1 | MULTISPECIES: ATP- | 76 | 2.1 |
| | | dependent Clp protease ATP- | | |
| | | binding subunit ClpL | | |
| | | [Staphylococcus] | | |
| 4-77 | P02751.5 | RecName: Full = Fibronectin; | 77 | 2.1 |
| | | Short = FN; AltName: Full = Cold- | | |
| | | insoluble globulin; Short = CIG; | | |
| | | Contains: RecName: | | |
| | | Full = Anastellin; Contains: | | |
| | | RecName: Full = Ugl-Y1; | | |
| | | Contains: RecName: Full = Ugl- | | |
| | | Y2; Contains: RecName: | | |
| | | Full = Ugl-Y3; Flags: Precursor | | |
| 4-78 | B9DN29.1 | RecName: Full = 6,7-dimethyl-8- | 78 | 2.1 |
| | | ribityllumazine synthase; | | |
| | | Short = DMRL synthase; | | |
| | | Short = LS; Short = Lumazine | | |
| | | synthase | | |
| 4-79 | Q4L530.1 | RecName: Full = Serine protease | 79 | 2.1 |
| | | HtrA-like | | |
| 4-80 | WP_001574605.1 | MULTISPECIES: DeoR/GlpR | 80 | 2.1 |
| | | transcriptional regulator | | |
| | | [Staphylococcus] | | |
| 4-81 | Q4L9J9.1 | RecName: Full = Arginine | 81 | 2.1 |
| | | deiminase; Short = ADI; AltName: | | |
| | | Full = Arginine dihydrolase; | | |
| | | Short = AD | | |
| 4-82 | WP_001140238.1 | MULTISPECIES: L-serine | 82 | 2.1 |
| | | ammonia-lyase, iron-sulfur- | | |
| | | dependent, subunit beta | | |
| | | [Staphylococcus] | | |
| 4-83 | WP_000154555.1 | MULTISPECIES: phage major | 83 | 2.1 |
| | | capsid protein [Staphylococcus] | | |
| 4-84 | B9DPQ9.1 | RecName: Full = Ribosomal RNA | 84 | 2.1 |
| | | small subunit methyltransferase | | |
| | | H; AltName: Full = 16S rRNA | | |
| | | m(4)C1402 methyltransferase; | | |
| | | AltName: Full = rRNA (cytosine- | | |
| | | N(4)-)-methyltransferase RsmH | | |
| 4-85 | WP_000803176.1 | MULTISPECIES: SMC family | 85 | 2.1 |
| | | ATPase [Staphylococcus] | | |
| 4-86 | Q6GAE1.1 | RecName: | 86 | 2.1 |
| | | Full = Phosphoribosylglycinamide | | |
| | | formyltransferase; AltName: | | |
| | | Full = 5′- | | |
| | | phosphoribosylglycinamide | | |
| | | transformylase; AltName: | | |
| | | Full = GAR transformylase; | | |
| | | Short = GART | | |
| 4-87 | WP_000666808.1 | MULTISPECIES: | 87 | 2.1 |
| | | phosphoribosylformylglycinamidine | | |
| | | synthase I [Staphylococcus] | | |
| 4-88 | Q49V12.1 | RecName: Full = Transcription- | 88 | 2.1 |
| | | repair-coupling factor; | | |
| | | Short = TRCF | | |
| 4-89 | B9DPF5.1 | RecName: Full = Translation | 89 | 2 |
| | | initiation factor IF-2 | | |
| 4-90 | A7X111.1 | RecName: Full = Divalent metal | 90 | 2 |
| | | cation transporter MntH | | |
| 4-91 | Q5HQC3.1 | RecName: Full = 1,4-dihydroxy-2- | 91 | 2 |
| | | naphthoyl-CoA synthase; | | |
| | | Short = DHNA-CoA synthase | | |
| 4-92 | B9DN19.1 | RecName: Full = S- | 92 | 2 |
| | | adenosylmethionine synthase; | | |
| | | Short = AdoMet synthase; | | |
| | | AltName: Full = MAT; AltName: | | |
| | | Full = Methionine | | |
| | | adenosyltransferase | | |
| 4-93 | WP_000291835.1 | MULTISPECIES: oleate | 93 | 2 |
| | | hydratase [Staphylococcus] | | |
| 4-94 | WP_001126664.1 | MULTISPECIES: Veg family | 94 | 2 |
| | | protein [Bacteria] | | |
| 4-95 | WP_000186117.1 | MULTISPECIES: acetolactate | 95 | 2 |
| | | decarboxylase [Staphylococcus] | | |
| 4-96 | WP_017466594.1 | extracellular adherence protein | 96 | 1.9 |
| | | Eap/Map, partial [Staphylococcus | | |
| | | aureus] | | |
| 4-97 | Q5HQA2.1 | RecName: | 97 | 1.9 |
| | | Full = Phosphoribosylformylglycinamidine | | |
| | | synthase subunit PurQ; | | |
| | | Short = FGAM synthase; AltName: | | |
| | | Full = Formylglycinamide | | |
| | | ribonucleotide amidotransferase | | |
| | | subunit I; Short = FGAR | | |
| | | amidotransferase I; | | |
| | | Short = FGAR-AT I; AltName: | | |
| | | Full = Glutaminase PurQ; | | |
| | | AltName: | | |
| | | Full = Phosphoribosylformylglycinamidine | | |
| | | synthase subunit I | | |
| 4-98 | WP_000622698.1 | MULTISPECIES: low specificity | 98 | 1.9 |
| | | L-threonine aldolase | | |
| | | [Staphylococcus] | | |
| 4-99 | WP_000074001.1 | MULTISPECIES: MerR family | 99 | 1.9 |
| | | transcriptional regulator | | |
| | | [Staphylococcus] | | |
| 4-100 | Q8CPQ1.1 | RecName: Full = Bifunctional | 100 | 1.9 |
| | | autolysin; Includes: RecName: | | |
| | | Full = N-acetylmuramoyl-L-alanine | | |
| | | amidase; Includes: RecName: | | |
| | | Full = Mannosyl-glycoprotein | | |
| | | endo-beta-N- | | |
| | | acetylglucosaminidase; Flags: | | |
| | | Precursor | | |
| 4-101 | Q8CRH6.1 | RecName: Full = 50S ribosomal | 101 | 1.9 |
| | | protein L18 | | |
| 4-102 | Q5HEP0.1 | RecName: Full = Response | 102 | 1.9 |
| | | regulator protein VraR | | |
| 4-103 | WP_001118708.1 | MULTISPECIES: NAD(P)/FAD- | 103 | 1.9 |
| | | dependent oxidoreductase | | |
| | | [Staphylococcus] | | |
| 4-104 | WP_000174050.1 | MULTISPECIES: | 104 | 1.9 |
| | | phosphoribosylaminoimidazolesuccinocarboxamide | | |
| | | synthase | | |
| | | [Staphylococcus] | | |
| 4-105 | WP_000289131.1 | GTP pyrophosphokinase family | 105 | 1.9 |
| | | protein [Staphylococcus aureus] | | |
| 4-106 | Q53599.1 | RecName: Full = Protein map; | 106 | 1.9 |
| | | AltName: Full = MHC class II | | |
| | | analog protein; Flags: Precursor | | |
| 4-107 | WP_001045133.1 | MULTISPECIES: response | 107 | 1.9 |
| | | regulator transcription factor | | |
| | | [Staphylococcus] | | |
| 4-108 | Q6G8X2.1 | RecName: Full = Shikimate | 108 | 1.9 |
| | | dehydrogenase (NADP(+)); | | |
| | | Short = SDH | | |
| 4-109 | Q2FXP7.1 | RecName: Full = Threonine--tRNA | 109 | 1.9 |
| | | ligase; AltName: Full = Threonyl- | | |
| | | tRNA synthetase; Short = ThrRS | | |
| 4-110 | Q8CS75.1 | RecName: Full = Translation | 110 | 1.8 |
| | | initiation factor IF-3 | | |
| 4-111 | B9DLM7.1 | RecName: Full = GMP synthase | 111 | 1.8 |
| | | [glutamine-hydrolyzing]; | | |
| | | AltName: Full = GMP synthetase; | | |
| | | AltName: Full = Glutamine | | |
| | | amidotransferase | | |
| 4-112 | P69905.2 | RecName: Full = Hemoglobin | 112 | 1.8 |
| | | subunit alpha; AltName: | | |
| | | Full = Alpha-globin; AltName: | | |
| | | Full = Hemoglobin alpha chain | | |
| 4-113 | WP_000149064.1 | MULTISPECIES: cell wall-active | 113 | 1.8 |
| | | antibiotics response protein | | |
| | | [Staphylococcus] | | |
| 4-114 | Q4L9E7.1 | RecName: Full = Sulfate | 114 | 1.8 |
| | | adenylyltransferase; AltName: | | |
| | | Full = ATP-sulfurylase; AltName: | | |
| | | Full = Sulfate adenylate | | |
| | | transferase; Short = SAT | | |
| 4-115 | Q5HF07.1 | RecName: Full = Riboflavin | 115 | 1.8 |
| | | biosynthesis protein RibBA; | | |
| | | Includes: RecName: Full = 3,4- | | |
| | | dihydroxy-2-butanone 4- | | |
| | | phosphate synthase; | | |
| | | Short = DHBP synthase; Includes: | | |
| | | RecName: Full = GTP | | |
| | | cyclohydrolase-2; AltName: | | |
| | | Full = GTP cyclohydrolase II | | |
| 4-116 | WP_000460421.1 | MULTISPECIES: L-serine | 116 | 1.8 |
| | | ammonia-lyase, iron-sulfur- | | |
| | | dependent, subunit alpha | | |
| | | [Staphylococcus] | | |
| 4-117 | Q8GN53.1 | RecName: Full = Holliday junction | 117 | 1.8 |
| | | resolvase RecU; AltName: | | |
| | | Full = Penicillin-binding protein- | | |
| | | related factor A homolog; | | |
| | | Short = PBP-related factor A | | |
| | | homolog; AltName: | | |
| | | Full = Recombination protein U | | |
| | | homolog | | |
| 4-118 | WP_000383804.1 | MULTISPECIES: hypothetical | 118 | 1.8 |
| | | protein [Bacilli] | | |
| 4-119 | Q6GIB2.1 | RecName: Full = Chaperone | 119 | 1.8 |
| | | protein ClpB | | |
| 4-120 | Q6GD13.1 | RecName: Full = HTH-type | 120 | 1.8 |
| | | transcriptional regulator SarS; | | |
| | | AltName: Full = Staphylococcal | | |
| | | accessory regulator S | | |
| 4-121 | WP_001246011.1 | MULTISPECIES: helix-turn-helix | 121 | 1.8 |
| | | transcriptional regulator | | |
| | | [Bacteria] | | |
| 4-122 | WP_001104171.1 | MULTISPECIES: two-component | 122 | 1.8 |
| | | system regulatory protein Yycl | | |
| | | [Staphylococcus] | | |
| 4-123 | WP_000201875.1 | MULTISPECIES: lipoyl synthase | 123 | 1.7 |
| | | [Staphylococcus] | | |
| 4-124 | Q49X24.1 | RecName: Full = 30S ribosomal | 124 | 1.7 |
| | | protein S16 | | |
| 4-125 | WP_000064778.1 | MULTISPECIES: amino acid | 125 | 1.7 |
| | | permease [Staphylococcus] | | |
| 4-126 | B9DM19.1 | RecName: Full = 50S ribosomal | 126 | 1.7 |
| | | protein L3 | | |
| 4-127 | WP_000608835.1 | MULTISPECIES: iron-sulfur | 127 | 1.7 |
| | | cluster repair di-iron protein ScdA | | |
| | | [Staphylococcus] | | |
| 4-128 | P03864.2 | RecName: Full = Plasmid | 128 | 1.7 |
| | | recombination enzyme type 3; | | |
| | | AltName: Full = Mobilization | | |
| | | protein; AltName: Full = Plasmid | | |
| | | recombinase | | |
| 4-129 | WP_001266540.1 | MULTISPECIES: PTS sugar | 129 | 1.7 |
| | | transporter subunit IIA | | |
| | | [Staphylococcus] | | |
| 4-130 | Q8CQK5.1 | RecName: Full = DNA replication | 130 | 1.7 |
| | | and repair protein RecF | | |
| 4-131 | Q8CMQ2.1 | RecName: Full = Alkyl | 131 | 1.7 |
| | | hydroperoxide reductase C; | | |
| | | AltName: Full = Peroxiredoxin; | | |
| | | AltName: Full = Thioredoxin | | |
| | | peroxidase | | |
| 4-132 | WP_000179061.1 | MULTISPECIES: acetolactate | 132 | 1.7 |
| | | decarboxylase [Staphylococcus] | | |
| 4-133 | Q8CSP9.2 | RecName: Full = 50S ribosomal | 133 | 1.7 |
| | | protein L33 2 | | |
| 4-134 | WP_000394700.1 | MULTISPECIES: hypothetical | 134 | 1.7 |
| | | protein [Staphylococcus] | | |
| 4-135 | Q2YU83.1 | RecName: Full = Uncharacterized | 135 | 1.7 |
| | | leukocidin-like protein 2; Flags: | | |
| | | Precursor | | |
| 4-136 | WP_000517908.1 | MULTISPECIES: 50S ribosomal | 136 | 1.7 |
| | | protein L28 [Bacteria] | | |
| 4-137 | Q2YXW0.1 | RecName: Full = Protein GlcT | 137 | 1.7 |
| 4-138 | WP_000072295.1 | MULTISPECIES: sensor histidine | 138 | 1.7 |
| | | kinase KdpD [Staphylococcus] | | |
| 4-139 | WP_000128656.1 | MULTISPECIES: hypothetical | 139 | 1.7 |
| | | protein [Bacilli] | | |
| 4-140 | WP_017466787.1 | carboxypeptidase regulatory-like | 140 | 1.6 |
| | | domain-containing protein, partial | | |
| | | [Staphylococcus aureus] | | |
| 4-141 | WP_001101911.1 | phosphoribosylamine--glycine | 141 | 1.6 |
| | | ligase [Staphylococcus aureus] | | |
| 4-142 | Q8CQ88.1 | RecName: Full = ATP-dependent | 142 | 1.6 |
| | | Clp protease ATP-binding | | |
| | | subunit ClpC | | |
| 4-143 | B9DM98.1 | RecName: | 143 | 1.6 |
| | | Full = Phosphoglucosamine | | |
| | | mutase | | |
| 4-144 | Q4L7Z4.1 | RecName: Full = Serine | 144 | 1.6 |
| | | hydroxymethyltransferase; | | |
| | | Short = SHMT; Short = Serine | | |
| | | methylase | | |
| 4-145 | WP_000186931.1 | MULTISPECIES: GAF domain- | 145 | 1.6 |
| | | containing protein | | |
| | | [Staphylococcus] | | |
| 4-146 | WP_001819878.1 | MULTISPECIES: hypothetical | 146 | 1.6 |
| | | protein [Staphylococcus] | | |
| 4-147 | WP_001187612.1 | MULTISPECIES: HD domain- | 147 | 1.6 |
| | | containing protein | | |
| | | [Staphylococcus] | | |
| 4-148 | P18357.1 | RecName: Full = Regulatory | 148 | 1.6 |
| | | protein BlaR1 | | |
| 4-149 | Q6GI14.1 | RecName: | 149 | 1.6 |
| | | Full = Amidophosphoribosyltransferase; | | |
| | | Short = ATase; AltName: | | |
| | | Full = Glutamine | | |
| | | phosphoribosylpyrophosphate | | |
| | | amidotransferase; | | |
| | | Short = GPATase; Flags: | | |
| | | Precursor | | |
| 4-150 | WP_000922328.1 | MULTISPECIES: GntR family | 150 | 1.6 |
| | | transcriptional regulator | | |
| | | [Staphylococcus] | | |
| 4-151 | WP_000709288.1 | MULTISPECIES: bifunctional | 151 | 1.6 |
| | | phosphoribosylaminoimidazolecarboxamide | | |
| | | formyltransferase/IMP | | |
| | | cyclohydrolase [Staphylococcus] | | |
| 4-152 | Q49ZF5.1 | RecName: Full = 30S ribosomal | 152 | 1.6 |
| | | protein S14 type Z | | |
| 4-153 | WP_000633782.1 | MULTISPECIES: flavodoxin | 153 | 1.6 |
| | | family protein [Staphylococcus | | |
| 4-154 | WP_000375864.1 | MULTISPECIES: helix-turn-helix | 154 | 1.6 |
| | | transcriptional regulator | | |
| | | [Staphylococcus] | | |
| 4-155 | WP_001196351.1 | MULTISPECIES: proline | 155 | 1.6 |
| | | dehydrogenase [Staphylococcus] | | |
| 4-156 | B9DKT3.1 | RecName: Full = Uracil-DNA | 156 | 1.6 |
| | | glycosylase; Short = UDG | | |
| 4-157 | WP_000735863.1 | MULTISPECIES: homoserine | 157 | 1.6 |
| | | dehydrogenase [Staphylococcus] | | |
| 4-158 | WP_001033875.1 | MULTISPECIES: 5′- | 158 | 1.6 |
| | | nucleotidase, lipoprotein e(P4) | | |
| | | family [Staphylococcus] | | |
| 4-159 | WP_001255453.1 | MULTISPECIES: threonine | 159 | 1.6 |
| | | synthase [Staphylococcus] | | |
| 4-160 | WP_000333457.1 | MULTISPECIES: DUF3885 | 160 | 1.6 |
| | | domain-containing protein | | |
| | | [Staphylococcus] | | |
| 4-161 | WP_000545928.1 | MULTISPECIES: urease subunit | 161 | 1.6 |
| | | gamma [Bacteria] | | |
| 4-162 | WP_000675909.1 | MULTISPECIES: NAD- | 162 | 1.6 |
| | | dependent malic enzyme | | |
| | | [Staphylococcus] | | |
| 4-163 | WP_001216937.1 | MULTISPECIES: hypothetical | 163 | 1.6 |
| | | protein [Staphylococcus] | | |
| 4-164 | A8Z0F5.2 | RecName: Full = PTS system | 164 | 1.6 |
| | | glucose-specific EIICBA | | |
| | | component; AltName: | | |
| | | Full = EIICBA-Glc; Short = EII-Glc; | | |
| 4-165 | WP_001229250.1 | MULTISPECIES: | 165 | 1.6 |
| | | magnesium/cobalt transporter | | |
| | | CorA [Staphylococcus] | | |
| 4-166 | WP_001005516.1 | MULTISPECIES: fructosamine | 166 | 1.6 |
| | | kinase family protein | | |
| | | [Staphylococcus] | | |
| 4-167 | WP_000640738.1 | MULTISPECIES: 1- | 167 | 1.6 |
| | | phosphofructokinase | | |
| | | [Staphylococcus] | | |
| 4-168 | WP_017466647.1 | MobA/MobL family protein, | 168 | 1.6 |
| | | partial [Staphylococcus aureus] | | |
| 4-169 | WP_000691584.1 | MULTISPECIES: GntR family | 169 | 1.6 |
| | | transcriptional regulator | | |
| | | [Staphylococcus] | | |
| 4-170 | Q49WJ5.1 | RecName: | 170 | 1.5 |
| | | Full = Phosphoribosylformylglycin | | |
| | | amidine cyclo-ligase; AltName: | | |
| | | Full = AIR synthase; AltName: | | |
| | | Full = AIRS; AltName: | | |
| | | Full = Phosphoribosyl- | | |
| | | aminoimidazole synthetase | | |
| 4-171 | WP_000759233.1 | MULTISPECIES: phosphate | 171 | 1.5 |
| | | ABC transporter substrate- | | |
| | | binding protein PstS | | |
| | | [Staphylococcus] | | |
| 4-172 | Q2YX52.1 | RecName: | 172 | 1.5 |
| | | Full = Phosphoribosylformylglycinamidine | | |
| | | cyclo-ligase; AltName: | | |
| | | Full = AIR synthase; AltName: | | |
| | | Full = AIRS; AltName: | | |
| | | Full = Phosphoribosyl- | | |
| | | aminoimidazole synthetase | | |
| 4-173 | Q5HDJ4.1 | RecName: Full = Heme response | 173 | 1.5 |
| | | regulator HssR | | |
| 4-174 | WP_017466558.1 | teichoic acid D-Ala esterase | 174 | 1.5 |
| | | FmtA [Staphylococcus aureus] | | |
| 4-175 | Q6GH10.1 | RecName: Full = Uncharacterized | 175 | 1.5 |
| | | hydrolase SAR1410 | | |
| 4-176 | WP_000802949.1 | MULTISPECIES: NAD(P)- | 176 | 1.5 |
| | | dependent oxidoreductase | | |
| | | [Staphylococcus] | | |
| 4-177 | WP_000566670.1 | MULTISPECIES: DUF3578 | 177 | 1.5 |
| | | domain-containing protein | | |
| | | [Staphylococcus] | | |
| 4-178 | WP_000008670.1 | MULTISPECIES: urease subunit | 178 | 1.5 |
| | | alpha [Staphylococcus] | | |
| 4-179 | WP_000848351.1 | MULTISPECIES: | 179 | 1.5 |
| | | phosphoribosylformylglycinamidine | | |
| | | synthase subunit PurS | | |
| | | [Staphylococcus] | | |
| 4-180 | Q7A3X3.1 | RecName: Full = Putative hemin | 180 | 1.5 |
| | | import ATP-binding protein HrtA | | |
| 4-181 | Q5HPB5.1 | RecName: Full = PTS system | 181 | 1.5 |
| | | glucose-specific EIIA component; | | |
| | | AltName: Full = EIIA-Glc; | | |
| | | AltName: Full = EIII-Glc; AltName: | | |
| | | Full = Glucose-specific | | |
| | | phosphotransferase enzyme IIA | | |
| | | component | | |
| 4-182 | Q8NVF6.2 | RecName: Full = Pyrimidine- | 182 | 1.5 |
| | | nucleoside phosphorylase; | | |
| | | Short = PYNP; Short = Py-NPase | | |
| 4-183 | Q5HNW6.1 | RecName: Full = Chaperone | 183 | 1.5 |
| | | protein Dnak; AltName: | | |
| | | Full = HSP70; AltName: Full = Heat | | |
| | | shock 70 kDa protein; AltName: | | |
| | | Full = Heat shock protein 70 | | |
| 4-184 | Q2YV56.1 | RecName: Full = Transcriptional | 184 | 1.5 |
| | | regulatory protein HptR | | |
| 4-185 | Q49XU5.1 | RecName: Full = Tyrosine | 185 | 1.5 |
| | | recombinase XerD | | |
| 4-186 | WP_001005833.1 | MULTISPECIES: arginine- | 186 | 1.5 |
| | | ornithine antiporter | | |
| | | [Staphylococcus] | | |
| 4-187 | B9DKX1.1 | RecName: Full = 50S ribosomal | 187 | 1.5 |
| | | protein L1 | | |
| 4-188 | WP_001281140.1 | MULTISPECIES: alanine | 188 | 1.5 |
| | | racemase [Staphylococcus] | | |
| 4-189 | Q7A462.1 | RecName: Full = 30S ribosomal | 189 | 1.5 |
| | | protein S17 | | |
| 4-190 | WP_000634691.1 | MULTISPECIES: galactose | 190 | 1.5 |
| | | mutarotase [Staphylococcus] | | |
| 4-191 | WP_001242307.1 | MULTISPECIES: pyrimidine- | 191 | 1.5 |
| | | nucleoside phosphorylase | | |
| | | [Staphylococcus] | | |
| 4-192 | WP_001273859.1 | MULTISPECIES: recombinase | 192 | 1.5 |
| | | family protein [Bacilli] | | |
| 4-193 | WP_000704122.1 | MULTISPECIES: Rrf2 family | 193 | 1.5 |
| | | transcriptional regulator | | |
| | | [Staphylococcus] | | |
| 4-194 | WP_000897633.1 | MULTISPECIES: nitric oxide | 194 | 1.5 |
| | | synthase oxygenase | | |
| | | [Staphylococcus] | | |
| 4-195 | WP_000154162.1 | MULTISPECIES: peptide | 195 | 1.5 |
| | | resistance ABC transporter ATP- | | |
| | | binding subunit VraD | | |
| | | [Staphylococcus] | | |
| 4-196 | Q00990.1 | RecName: Full = 50S ribosomal | 196 | 1.5 |
| | | protein L13 | | |
| 4-197 | WP_000524902.1 | MULTISPECIES: YqeG family | 197 | 1.5 |
| | | HAD IIIA-type phosphatase | | |
| | | [Staphylococcus] | | |
| 4-198 | P04635.1 | RecName: Full = Lipase; AltName: | 198 | 1.5 |
| | | Full = Phospholipase A1; | | |
| | | AltName: Full = Triacylglycerol | | |
| | | lipase; Contains: RecName: | | |
| | | Full = Lipase 86 kDa form; | | |
| | | Contains: RecName: Full = Lipase | | |
| | | 46 kDa form; Flags: Precursor | | |
| 4-199 | P23533.2 | RecName: | 199 | 1.5 |
| | | Full = Phosphoenolpyruvate- | | |
| | | protein phosphotransferase; | | |
| | | AltName: | | |
| | | Full = Phosphotransferase | | |
| | | system, enzyme I | | |
| 4-200 | WP_000073195.1 | MULTISPECIES: MurR/RpiR | 200 | 1.5 |
| | | family transcriptional regulator | | |
| | | [Staphylococcus] | | |
| 4-201 | WP_001012237.1 | MULTISPECIES: FemA/FemB | 201 | 1.5 |
| | | family glycyltransferase FmhA | | |
| | | [Staphylococcus] | | |
| 4-202 | WP_000700901.1 | carboxylesterase/lipase family | 202 | 1.5 |
| | | protein [Staphylococcus aureus] | | |
| 4-203 | WP_001213908.1 | MULTISPECIES: metal ABC | 203 | 1.5 |
| | | transporter ATP-binding protein | | |
| | | [Staphylococcus] | | |
| 4-204 | B9DM42.1 | RecName: Full = 50S ribosomal | 204 | 1.5 |
| | | protein L22 | | |
| 4-205 | Q9RGS5.1 | RecName: Full = Thiamine- | 205 | 1.5 |
| | | phosphate synthase; Short = TP | | |
| | | synthase; Short = TPS; AltName: | | |
| | | Full = Thiamine-phosphate | | |
| | | pyrophosphorylase; Short = TMP | | |
| | | pyrophosphorylase; Short = TMP- | | |
| | | PPase | | |
| 4-206 | Q6GHP6.1 | RecName: Full = Cell division | 206 | 1.5 |
| | | protein SepF | | |
| 4-207 | WP_001108122.1 | MULTISPECIES: PTS sucrose | 207 | 1.5 |
| | | transporter subunit IIBC | | |
| | | [Staphylococcus] | | |

Table 3. Identification of subunit proteins recognized by sera from protected mice. Sera from protected and non-protected mice, prior to challenge, were used to immunoprecipitate (IP) proteins from their corresponding lysates—e.g., planktonic sera were used to IP planktonic lysate- and the immunoprecipitates were subjected to LC/MS/MS. A total of 136 proteins were identified as being unique or eliciting a greater antibody response in protected mice (comparing 10 non-protected and 11 protected mice). A subset of these proteins is shown. Interestingly, the proteins identified by methods #1 (non-IP) and #2 (IP) are different except for α-hemolysin, this approach may be used to identify a protein or set of proteins that are protective. This adds a multi-sample differential component to reverse vaccinology. Table 3 shows the GenBank® accession number, the description, and the fold increase in expression of the protein in protective immunogenic compositions compared to non-protective compositions. A fold increase of “infinity” indicates that the non-protective compositions did not express this protein to detectable levels.

TABLE 3
Table of protein immunogen candidates identified via IP method.

| | | | SEQ | fold |
|---|---|---|---|---|
| | Accession | | ID | in- |
| No. | No. | Description | NO: | crease |
| 5-1 | WP_ | MULTISPECIES: YSIRK domain- | 40 | infinity |
| | 000943842.1 | containing triacylglycerol lipase | | |
| | | Lip2/Geh [Staphylococcus] | | |
| 5-2 | Q8NYC2.1 | RecName: Full = Lipase 2; | 208 | infinity |
| | | AltName: Full = Glycerol ester | | |
| | | hydrolase 2; Flags: Precursor | | |
| 5-3 | Q2YWH4.2 | RecName: Full = Carbamate kinase | 30 | infinity |
| | | 2 | | |
| 5-4 | Q6G7F8.3 | RecName: Full = Glutamine-- | 209 | infinity |
| | | fructose-6-phosphate | | |
| | | aminotransferase [isomerizing]; | | |
| | | AltName: Full = D-fructose-6- | | |
| | | phosphate amidotransferase; | | |
| | | AltName: Full = GFAT; AltName: | | |
| | | Full = Glucosamine-6-phosphate | | |
| | | synthase; AltName: | | |
| | | Full = Hexosephosphate | | |
| | | aminotransferase; AltName: | | |
| | | Full = L-glutamine--D-fructose-6- | | |
| | | phosphate amidotransferase | | |
| 5-5 | Q2G155.1 | RecName: Full = Lipase 2; | 210 | infinity |
| | | AltName: Full = Glycerol ester | | |
| | | hydrolase 2; Flags: Precursor | | |
| 5-6 | P02751.5 | RecName: Full = Fibronectin; | 77 | infinity |
| | | Short = FN; AltName: Full = Cold- | | |
| | | insoluble globulin; Short = CIG; | | |
| | | Contains: RecName: | | |
| | | Full = Anastellin; Contains: | | |
| | | RecName: Full = Ugl-Y1; Contains: | | |
| | | RecName: Full = Ugl-Y2; Contains: | | |
| | | RecName: Full = Ugl-Y3; Flags: | | |
| | | Precursor | | |
| 5-7 | Q2FWD6.1 | RecName: Full = Putative aldehyde | 211 | infinity |
| | | dehydrogenase | | |
| 5-8 | Q5HPU4.1 | RecName: Full = Succinate--CoA | 212 | infinity |
| | | ligase [ADP-forming] subunit | | |
| | | alpha; AltName: Full = Succinyl- | | |
| | | CoA synthetase subunit alpha; | | |
| | | Short = SCS-alpha | | |
| 5-9 | Q8CS69.1 | RecName: Full = Pyruvate kinase; | 213 | infinity |
| | | Short = PK | | |
| 5- | WP_ | MULTISPECIES: | 214 | infinity |
| 10 | 000160914.1 | undecaprenyldiphospho- | | |
| | | muramoylpentapeptide beta-N- | | |
| | | acetylglucosaminyltransferase | | |
| | | [Staphylococcus] | | |
| 5- | B9DM33.1 | RecName: Full = 30S ribosomal | 215 | infinity |
| 11 | | protein S8 | | |
| 5- | WP_ | MULTISPECIES: class I SAM- | 216 | infinity |
| 12 | 000487150.1 | dependent RNA methyltransferase | | |
| | | [Staphylococcus] | | |
| 5- | Q02413.2 | RecName: Full = Desmoglein-1; | 217 | infinity |
| 13 | | AltName: Full = Cadherin family | | |
| | | member 4; AltName: | | |
| | | Full = Desmosomal glycoprotein 1; | | |
| | | Short = DG1; Short = | | |
| | | DGI; AltName: | | |
| | | Full = Pemphigus foliaceus antigen; | | |
| | | Flags: Precursor | | |
| 5- | Q4LAK8.1 | RecName: Full = Serine--tRNA | 218 | infinity |
| 14 | | ligase; AltName: Full = Seryl-tRNA | | |
| | | synthetase; Short = SerRS; | | |
| | | AltName: Full = Seryl- | | |
| | | RNA(Ser/Sec) synthetase | | |
| 5- | Q6G9C6.1 | RecName: Full = Quinolone | 219 | infinity |
| 15 | | resistance protein NorB | | |
| 5- | Q8CPN3.1 | RecName: Full = Pyruvate | 220 | infinity |
| 16 | | dehydrogenase E1 component | | |
| | | subunit alpha | | |
| 5- | Q8CRI5.1 | RecName: Full = 50S ribosomal | 221 | infinity |
| 17 | | protein L17 | | |
| 5- | Q5HFM3.1 | RecName: Full = Probable glycine | 222 | infinity |
| 18 | | dehydrogenase (decarboxylating) | | |
| | | subunit 1; AltName: Full = Glycine | | |
| | | cleavage system P-protein subunit | | |
| | | 1; AltName: Full = Glycine | | |
| | | decarboxylase subunit 1; | | |
| | | AltName: Full = Glycine | | |
| | | dehydrogenase (aminomethyl- | | |
| | | transferring) subunit 1 | | |
| 5- | WP_ | MULTISPECIES: acetolactate | 132 | infinity |
| 19 | 000179061.1 | decarboxylase [Staphylococcus] | | |
| 5- | WP_ | MULTISPECIES: phenol-soluble | 223 | infinity |
| 20 | 000763048.1 | modulin export ABC transporter | | |
| | | ATP-binding protein PmtC | | |
| | | [Staphylococcus] | | |
| 5- | Q99U09.3 | RecName: Full = Elastin-binding | 224 | infinity |
| 21 | | protein EbpS | | |
| 5- | WP_ | MULTISPECIES: DivIVA domain- | 225 | infinity |
| 22 | 001117066.1 | containing protein | | |
| | | [Staphylococcus] | | |
| 5- | WP_ | MULTISPECIES: alpha-keto acid | 226 | infinity |
| 23 | 000813536.1 | decarboxylase family protein | | |
| | | [Staphylococcus] | | |
| 5- | Q6GG73.1 | RecName: Full = Aspartate--tRNA | 227 | infinity |
| 24 | | ligase; AltName: Full = Aspartyl- | | |
| | | tRNA synthetase; Short = AspRS | | |
| 5- | WP_ | MULTISPECIES: Asp23/Gls24 | 228 | infinity |
| 25 | 000171418.1 | family envelope stress response | | |
| | | protein [Bacteria] | | |
| 5- | Q5HRL0.1 | RecName: Full = DNA-directed | 229 | infinity |
| 26 | | RNA polymerase subunit beta; | | |
| | | Short = RNAP subunit beta; | | |
| | | AltName: Full = RNA polymerase | | |
| | | subunit beta; AltName: | | |
| | | Full = Transcriptase subunit beta | | |
| 5- | Q8NX33.1 | RecName: Full = Cell division | 230 | infinity |
| 27 | | protein FtsA | | |
| 5- | WP_ | MULTISPECIES: replication | 231 | infinity |
| 28 | 000043161.1 | initiator protein A [Staphylococcus] | | |
| 5- | Q5HF24.1 | RecName: Full = D-alanine | 232 | infinity |
| 29 | | aminotransferase; AltName: | | |
| | | Full = D-amino acid | | |
| | | aminotransferase; AltName: | | |
| | | Full = D-amino acid transaminase; | | |
| | | Short = DAAT; AltName: Full = D- | | |
| | | aspartate aminotransferase | | |
| 5- | Q8NX29.1 | RecName: Full = Isoleucine--tRNA | 233 | infinity |
| 30 | | ligase; AltName: Full = Isoleucyl- | | |
| | | tRNA synthetase; Short = IIeRS | | |
| 5- | Q2YSC4.3 | RecName: Full = 50S ribosomal | 234 | infinity |
| 31 | | protein L11 | | |
| 5- | Q5HF42.2 | RecName: Full = Formate-- | 235 | infinity |
| 32 | | tetrahydrofolate ligase; AltName: | | |
| | | Full = Formyltetrahydrofolate | | |
| | | synthetase; Short = FHS; | | |
| | | Short = FTHFS | | |
| 5- | Q8NX12.1 | RecName: Full = Uncharacterized | 236 | infinity |
| 33 | | protein MW1109 | | |
| 5- | Q8CU95.1 | RecName: Full = Serine--tRNA | 237 | infinity |
| 34 | | ligase; AltName: Full = Seryl-tRNA | | |
| | | synthetase; Short = SerRS; | | |
| | | AltName: Full = Seryl- | | |
| | | tRNA(Ser/Sec) synthetase | | |
| 5- | WP_ | MULTISPECIES: class 1b | 238 | infinity |
| 35 | 000562498.1 | ribonucleoside-diphosphate | | |
| | | reductase subunit beta [Bacteria] | | |
| 5- | WP_ | MULTISPECIES: hypothetical | 239 | infinity |
| 36 | 000495684.1 | protein [Staphylococcus] | | |
| 5- | A6QKC0.1 | RecName: Full = HTH-type | 54 | infinity |
| 37 | | transcriptional regulator ArcR | | |
| 5- | POC1S7.1 | RecName: Full = DNA | 240 | infinity |
| 38 | | topoisomerase 4 subunit B; | | |
| | | AltName: Full = Topoisomerase IV | | |
| | | subunit B | | |
| 5- | Q6GJ80.1 | RecName: Full = Phosphate | 24′ | infinity |
| 39 | | acetyltransferase; AltName: | | |
| | | Full = Phosphotransacetylase | | |
| 5- | WP_ | MULTISPECIES: molecular | 242 | infinity |
| 40 | 000034716.1 | chaperone Dnak [Staphylococcus] | | |
| 5- | WP_ | MULTISPECIES: type B 50S | 243 | infinity |
| 41 | 000808968.1 | ribosomal protein L31 [Bacteria] | | |
| 5- | WP_ | Glu/Leu/Phe/Val dehydrogenase | 244 | infinity |
| 42 | 025175252.1 | [Staphylococcus aureus] | | |
| 5- | WP_ | MULTISPECIES: penicillin-binding | 245 | infinity |
| 43 | 000138342.1 | protein [Staphylococcus] | | |
| 5- | WP_ | MULTISPECIES: RluA family | 246 | infinity |
| 44 | 000451297.1 | pseudouridine synthase | | |
| | | [Staphylococcus] | | |
| 5- | WP_ | MULTISPECIES: YtxH domain- | 247 | infinity |
| 45 | 000648118.1 | containing protein | | |
| | | [Staphylococcus] | | |
| 5- | Q8CPQ1.1 | RecName: Full = Bifunctional | 100 | infinity |
| 46 | | autolysin; Includes: RecName: | | |
| | | Full = N-acetylmuramoyl-L-alanine | | |
| | | amidase; Includes: RecName: | | |
| | | Full = Mannosyl-glycoprotein endo- | | |
| | | beta-N-acetylglucosaminidase; | | |
| | | Flags: Precursor | | |
| 5- | Q8CRH5.1 | RecName: Full = 50S ribosomal | 248 | infinity |
| 47 | | protein L6 | | |
| 5- | WP_ | MULTISPECIES: DNA | 249 | infinity |
| 48 | 001038301.1 | polymerase \| [Staphylococcus] | | |
| 5- | Q49WI3.1 | RecName: Full = Probable quinol | 250 | infinity |
| 49 | | oxidase subunit 1; AltName: | | |
| | | Full = Quinol oxidase polypeptide I | | |
| 5- | Q5HM47.1 | RecName: Full = Alkaline shock | 251 | infinity |
| 50 | | protein 23 | | |
| 5- | Q5HNK6.1 | RecName: Full = ATP-dependent 6- | 252 | infinity |
| 51 | | phosphofructokinase; Short = ATP- | | |
| | | PFK; Short = Phosphofructokinase; | | |
| | | AltName: | | |
| | | Full = Phosphohexokinase | | |
| 5- | Q49V34.1 | RecName: Full = ATP-dependent | 253 | infinity |
| 52 | | Clp protease ATP-binding subunit | | |
| | | ClpC | | |
| 5- | Q49Y83.1 | RecName: Full = 50S ribosomal | 254 | infinity |
| 53 | | protein L27 | | |
| 5- | B9DM32.1 | RecName: Full = 50S ribosomal | 255 | infinity |
| 54 | | protein L6 | | |
| 5- | Q5HRP1.1 | RecName: Full = Cysteine | 256 | infinity |
| 55 | | synthase; Short = CSase; AltName: | | |
| | | Full = O-acetylserine (thiol)-lyase; | | |
| | | Short = OAS-TL; | | |
| | | AltName: Full = O- | | |
| | | acetylserine sulfhydrylase | | |
| 5- | Q6GD13.1 | RecName: Full = HTH-type | 120 | infinity |
| 56 | | transcriptional regulator SarS; | | |
| | | AltName: Full = Staphylococcal | | |
| | | accessory regulator S | | |
| 5- | Q8NVS9.1 | RecName: Full = Staphopain A; | 257 | infinity |
| 57 | | AltName: Full = Staphylococcal | | |
| | | cysteine proteinase A; AltName: | | |
| | | Full = Staphylopain A; Flags: | | |
| | | Precursor | | |
| 5- | WP_ | MULTISPECIES: ribosomal | 258 | infinity |
| 58 | 000372755.1 | protein S18-alanine N- | | |
| | | acetyltransferase | | |
| | | [Staphylococcus] | | |
| 5- | WP_ | MULTISPECIES: 5′-3′ | 259 | infinity |
| 59 | 001133021.1 | exonuclease [Staphylococcus] | | |
| 5- | A6TY44.1 | RecName: Full = Iron-sulfur cluster | 260 | infinity |
| 60 | | repair protein ScdA | | |
| 5- | Q2FG29.1 | RecName: Full = Alanine | 261 | infinity |
| 61 | | dehydrogenase 2 | | |
| 5- | Q2YZB9.1 | RecName: Full = tRNA uridine 5- | 262 | infinity |
| 62 | | carboxymethylaminomethyl | | |
| | | modification enzyme MnmG; | | |
| | | AltName: Full = Glucose-inhibited | | |
| | | division protein A | | |
| 5- | Q8NW75.1 | RecName: Full = Glutamate-1- | 263 | infinity |
| 63 | | semialdehyde 2, 1-aminomutase 1; | | |
| | | Short = GSA 1; AltName: | | |
| | | Full = Glutamate-1-semialdehyde | | |
| | | aminotransferase 1; Short = GSA- | | |
| | | AT 1 | | |
| 5- | A5IVJ8.1 | RecName: Full = Immunoglobulin- | 264 | infinity |
| 64 | | binding protein Sbi; Flags: | | |
| | | Precursor | | |
| 5- | A6U4E6.1 | RecName: Full = 2,3- | 265 | infinity |
| 65 | | bisphosphoglycerate-dependent | | |
| | | phosphoglycerate mutase; | | |
| | | Short = BPG-dependent PGAM; | | |
| | | Short = PGAM; | | |
| | | Short = Phosphoglyceromutase; | | |
| | | Short = dPGM | | |
| 5- | Q2YTB5.1 | RecName: Full = ATP-dependent | 266 | infinity |
| 66 | | Clp protease ATP-binding subunit | | |
| | | ClpX | | |
| 5- | Q5HF67.1 | RecName: Full = Uncharacterized | 267 | infinity |
| 67 | | peptidase SACOL1756 | | |
| 5- | Q6G6D6.1 | RecName: Full = PTS system | 268 | infinity |
| 68 | | glucoside-specific EIICBA | | |
| | | component; Includes: RecName: | | |
| | | Full = Glucoside permease IIC | | |
| | | component; AltName: Full = PTS | | |
| | | system glucoside-specific EIIC | | |
| | | component; Includes: RecName: | | |
| | | Full = Glucoside-specific | | |
| | | phosphotransferase enzyme IIB | | |
| | | component; AltName: Full = PTS | | |
| | | system glucoside-specific EIIB | | |
| | | component; Includes: RecName: | | |
| | | Full = Glucoside-specific | | |
| | | phosphotransferase enzyme IIA | | |
| | | component; AltName: Full = PTS | | |
| | | system glucoside-specific EIIA | | |
| | | component | | |
| 5- | Q931T1.1 | RecName: Full = Ribonuclease 3; | 269 | infinity |
| 69 | | AltName: Full = Ribonuclease III; | | |
| | | Short = RNase III | | |
| 5- | WP_ | MULTISPECIES: protein arginine | 270 | infinity |
| 70 | 000149503.1 | kinase [Staphylococcus] | | |
| 5- | WP_ | MULTISPECIES: bifunctional | 271 | infinity |
| 71 | 001185462.1 | hydroxymethylpyrimidine | | |
| | | kinase/phosphomethylpyrimidine | | |
| | | kinase [Staphylococcus] | | |
| 5- | WP_ | MULTISPECIES: oxidoreductase | 272 | infinity |
| 72 | 000140783.1 | [Staphylococcus] | | |
| 5- | WP_ | MULTISPECIES: purine- | 273 | infinity |
| 73 | 000160304.1 | nucleoside phosphorylase | | |
| | | [Staphylococcus] | | |
| 5- | WP_ | MULTISPECIES: cysteine | 274 | infinity |
| 74 | 000409167.1 | desulfurase [Staphylococcus] | | |
| 5- | WP_ | MULTISPECIES: polysaccharide | 275 | infinity |
| 75 | 000459062.1 | biosynthesis protein | | |
| | | [Staphylococcus] | | |
| 5- | WP_ | MULTISPECIES: ABC transporter | 276 | infinity |
| 76 | 000569116.1 | permease [Staphylococcus] | | |
| 5- | WP_ | MULTISPECIES: GNAT family N- | 277 | infinity |
| 77 | 000569884.1 | acetyltransferase | | |
| | | [Staphylococcus] | | |
| 5- | WP_ | MULTISPECIES: PG:teichoic acid | 278 | infinity |
| 78 | 000613541.1 | D-alanyltransferase DIltB | | |
| | | [Staphylococcus] | | |
| 5- | WP_ | MULTISPECIES: | 279 | infinity |
| 79 | 000710549.1 | phosphatidylinositol-specific | | |
| | | phospholipase C [Staphylococcus] | | |
| 5- | WP_ | MULTISPECIES: GTP | 280 | infinity |
| 80 | 001077683.1 | pyrophosphokinase family protein | | |
| | | [Staphylococcus] | | |
| 5- | WP_ | class I SAM-dependent rRNA | 281 | infinity |
| 81 | 017466559.1 | methyltransferase | | |
| | | [Staphylococcus aureus] | | |
| 5- | B9DKV7.1 | RecName: Full = Elongation factor | 282 | infinity |
| 82 | | G; Short = EF-G | | |
| 5- | B9DN77.1 | RecName: Full = Formate-- | 283 | infinity |
| 83 | | tetrahydrofolate ligase; AltName: | | |
| | | Full = Formyltetrahydrofolate | | |
| | | synthetase; Short = FHS; | | |
| | | Short = FTHFS | | |
| 5- | B9DPT1.1 | RecName: Full = UvrABC system | 284 | infinity |
| 84 | | protein C; Short = Protein UvrC; | | |
| | | AltName: Full = Excinuclease ABC | | |
| | | subunit C | | |
| 5- | O31211.1 | RecName: Full = UDP-N- | 285 | infinity |
| 85 | | acetylmuramate--L-alanine ligase; | | |
| | | AltName: Full = UDP-N- | | |
| | | acetylmuramoyl-L-alanine | | |
| | | synthetase | | |
| 5- | P13978.1 | RecName: Full = rRNA adenine N- | 286 | infinity |
| 86 | | 6-methyltransferase; AltName: | | |
| | | Full = Erythromycin resistance | | |
| | | protein; AltName: Full = Macrolide- | | |
| | | lincosamide-streptogramin B | | |
| | | resistance protein | | |
| 5- | P63513.1 | RecName: Full = Probable | 287 | infinity |
| 87 | | branched-chain-amino-acid | | |
| | | aminotransferase; Short = BCAT | | |
| 5- | Q05207.2 | RecName: Full = Protein | 288 | infinity |
| 88 | | translocase subunit SecY | | |
| 5- | Q2FDV8.1 | RecName: Full = ATP-dependent | 289 | infinity |
| 89 | | Clp protease ATP-binding subunit | | |
| | | ClpL | | |
| 5- | Q2YU46.1 | RecName: Full = Low molecular | 290 | infinity |
| 90 | | weight protein-tyrosine- | | |
| | | phosphatase PtpA; AltName: | | |
| | | Full = Phosphotyrosine | | |
| | | phosphatase A; Short = PTPase A | | |
| 5- | Q49X73.1 | RecName: Full = Protein RecA; | 291 | infinity |
| 91 | | AltName: Full = Recombinase A | | |
| 5- | Q49ZE5.1 | RecName: Full = 50S ribosomal | 292 | infinity |
| 92 | | protein L36 | | |
| 5- | Q4L607.1 | RecName: Full = Glycerol kinase; | 293 | infinity |
| 93 | | AltName: Full = ATP:glycerol 3- | | |
| | | phosphotransferase; AltName: | | |
| | | Full = Glycerokinase; Short = GK | | |
| 5- | Q5HFI1.1 | RecName: Full = Chaperone | 294 | infinity |
| 94 | | protein DnaJ | | |
| 5- | Q5HJI8.1 | RecName: Full = Ornithine | 295 | infinity |
| 95 | | aminotransferase 1; | | |
| | | Short = OAT 1; | | |
| | | AltName: Full = Ornithine-- | | |
| | | oxo-acid aminotransferase 1 | | |
| 5- | Q5HM69.3 | RecName: Full = Glutamine-- | 296 | infinity |
| 96 | | fructose-6-phosphate | | |
| | | aminotransferase [isomerizing]; | | |
| | | AltName: Full = D-fructose-6- | | |
| | | phosphate amidotransferase; | | |
| | | AltName: Full = GFAT; AltName: | | |
| | | Full = Glucosamine-6-phosphate | | |
| | | synthase; AltName: | | |
| | | Full = Hexosephosphate | | |
| | | aminotransferase; AltName: | | |
| | | Full = L-glutamine--D-fructose-6- | | |
| | | phosphate amidotransferase | | |
| 5- | Q5HQK0.1 | RecName: Full = Argininosuccinate | 297 | infinity |
| 97 | | synthase; AltName: | | |
| | | Full = Citrulline-- | | |
| | | aspartate ligase | | |
| 5- | Q6G931.1 | RecName: Full = Probable glycine | 298 | infinity |
| 98 | | dehydrogenase (decarboxylating) | | |
| | | subunit 2; AltName: Full = Glycine | | |
| | | cleavage system P-protein subunit | | |
| | | 2; AltName: Full = Glycine | | |
| | | decarboxylase subunit 2; | | |
| | | AltName: Full = Glycine | | |
| | | dehydrogenase (aminomethyl- | | |
| | | transferring) subunit 2 | | |
| 5- | Q6GDE0.1 | RecName: Full = Capsular | 299 | infinity |
| 99 | | polysaccharide biosynthesis | | |
| | | protein CapA | | |
| 5- | Q6GEY5.1 | RecName: Full = Membrane protein | 300 | infinity |
| 100 | | insertase YidC; AltName: | | |
| | | Full = Foldase YidC; AltName: | | |
| | | Full = Membrane integrase YidC; | | |
| | | AltName: Full = Membrane protein | | |
| | | YidC; Flags: Precursor | | |
| 5- | Q6GIH0. 1 | RecName: Full = UPF0051 protein | 301 | infinity |
| 101 | | SAR0880 | | |
| 5- | Q8CS60.1 | RecName: Full = Acetate kinase; | 302 | infinity |
| 102 | | AltName: Full = Acetokinase | | |
| 5- | Q8CSE3.1 | RecName: Full = 50S ribosomal | 303 | infinity |
| 103 | | protein L33 1 | | |
| 5- | Q8NWN1.1 | RecName: Full = Nucleoside | 304 | infinity |
| 104 | | diphosphate kinase; Short = NDK; | | |
| | | Short = NDP kinase; AltName: | | |
| | | Full = Nucleoside-2-P kinase | | |
| 5- | Q99TT5.1 | RecName: Full = RNA polymerase | 305 | infinity |
| 105 | | sigma factor SigA | | |
| 5- | Q8NXF2.1 | RecName: Full = Argininosuccinate | 306 | 17.3 |
| 106 | | synthase; AltName: | | |
| | | Full = Citrulline-- | | |
| | | aspartate ligase | | |
| 5- | WP_ | MULTISPECIES: PTS sugar | 129 | 9.8 |
| 107 | 001266540.1 | transporter subunit IIA | | |
| | | [Staphylococcus] | | |
| 5- | Q5HQV3.1 | RecName: Full = Phosphoglycerate | 307 | 9.8 |
| 108 | | kinase | | |
| 5- | Q4L7Y4.1 | RecName: Full = ATP synthase | 308 | 6.5 |
| 109 | | subunit beta; AltName: Full = ATP | | |
| | | synthase F1 sector subunit beta; | | |
| | | AltName: Full = F-ATPase subunit | | |
| | | beta | | |
| 5- | POA075.1 | RecName: Full = Gamma- | 309 | 6.5 |
| 110 | | hemolysin component B; AltName: | | |
| | | Full = H-gamma-1; AltName: | | |
| | | Full = H-gamma-l; Flags: Precursor | | |
| 5- | WP_ | MULTISPECIES: HTH-type | 310 | 6.1 |
| 111 | 000036076.1 | transcriptional regulator SarR | | |
| | | [Bacteria] | | |
| 5- | WP_ | MULTISPECIES: HTH-type | 311 | 6.1 |
| 112 | 000066900.1 | transcriptional regulator SarV | | |
| | | [Staphylococcus] | | |
| 5- | WP_ | MULTISPECIES: transcriptional | 312 | 5.4 |
| 113 | 001018677.1 | regulator [Bacteria] | | |
| 5- | Q57071.1 | RecName: Full = PTS system | 313 | 5.4 |
| 114 | | glucose-specific EIICBA | | |
| | | component; AltName: | | |
| | | Full = EIICBA-Glc; | | |
| | | Short = EII-Glc; | | |
| | | AltName: Full = EIICBA-Glc 1; | | |
| | | Includes: RecName: Full = Glucose | | |
| | | permease IIC component; | | |
| | | AltName: Full = PTS system | | |
| | | glucose-specific EIIC component; | | |
| | | Includes: RecName: | | |
| | | Full = Glucose-specific | | |
| | | phosphotransferase enzyme IIB | | |
| | | component; AltName: Full = PTS | | |
| | | system glucose-specific EIIB | | |
| | | component; Includes: RecName: | | |
| | | Full = Glucose-specific | | |
| | | phosphotransferase enzyme IIA | | |
| | | component; AltName: Full = PTS | | |
| | | system glucose-specific EIIA | | |
| | | component | | |
| 5- | Q2YX14.1 | RecName: Full = Probable quinol | 314 | 5.4 |
| 115 | | oxidase subunit 2; AltName: | | |
| | | Full = Quinol oxidase | | |
| | | polypeptide II; | | |
| | | Flags: Precursor | | |
| 5- | Q2YYV2.1 | RecName: Full = Urease accessory | 315 | 4.9 |
| 116 | | protein UreG | | |
| 5- | A5IW62.1 | RecName: Full = Arginine | 316 | 4.3 |
| 117 | | deiminase; Short = ADI; AltName: | | |
| | | Full = Arginine dihydrolase; | | |
| | | Short = AD | | |
| 5- | Q2YWS3.1 | RecName: Full = Glucose-6- | 317 | 4.3 |
| 118 | | phosphate isomerase; Short = GPI; | | |
| | | AltName: Full = Phosphoglucose | | |
| | | isomerase; Short = PGI; AltName: | | |
| | | Full = Phosphohexose isomerase; | | |
| | | Short = PHI | | |
| 5- | Q4L739.1 | RecName: Full = Pyruvate kinase; | 318 | 4.3 |
| 119 | | Short = PK | | |
| 5- | Q5HQ06.1 | RecName: Full = Cell division | 319 | 4.3 |
| 120 | | protein FtsZ | | |
| 5- | WP_ | MULTISPECIES: AAA family | 320 | 4.3 |
| 121 | 000584639.1 | ATPase [Staphylococcus] | | |
| 5- | Q8NXF3.1 | RecName: Full = Argininosuccinate | 20 | 4.3 |
| 122 | | lyase; Short = ASAL; AltName: | | |
| | | Full = Arginosuccinase | | |
| 5- | A6U4Y6.1 | RecName: Full = Fructose- | 321 | 4.3 |
| 123 | | bisphosphate aldolase class 1; | | |
| | | AltName: Full = Fructose- | | |
| | | bisphosphate aldolase class I; | | |
| | | Short = FBP aldolase | | |
| 5- | Q5HNW6.1 | RecName: Full = Chaperone | 183 | 3.8 |
| 124 | | protein Dnak; AltName: | | |
| | | Full = HSP70; | | |
| | | AltName: Full = Heat | | |
| | | shock 70 kDa protein; AltName: | | |
| | | Full = Heat shock protein 70 | | |
| 5- | Q8NUM4.1 | RecName: Full = Probable | 322 | 3.3 |
| 125 | | malate:quinone oxidoreductase 2; | | |
| | | AltName: Full = MQO 2; AltName: | | |
| | | Full = Malate dehydrogenase | | |
| | | [quinone] 2 | | |
| 5- | A8Z414.1 | RecName: Full = Extracellular | 2 | 3.3 |
| 126 | | matrix-binding protein ebh; | | |
| | | AltName: Full = ECM-binding | | |
| | | protein homolog; Flags: Precursor | | |
| 5- | A8Z3U3.1 | RecName: Full = Proline--tRNA | 323 | 3.3 |
| 127 | | ligase; AltName: | | |
| | | Full = Prolyl-tRNA | | |
| | | synthetase; Short = ProRS | | |
| 5- | WP_ | MULTISPECIES: nitrate reductase | 324 | 3.3 |
| 128 | 000514375.1 | subunit alpha [Staphylococcus] | | |
| 5- | WP_ | MULTISPECIES: ATP-binding | 325 | 3.3 |
| 129 | 000942303.1 | cassette domain-containing | | |
| | | protein [Staphylococcus] | | |
| 5- | Q2YX35.1 | RecName: Full = Ribonuclease J 1; | 326 | 3.3 |
| 130 | | Short = RNase J1 | | |
| 5- | Q49UQ2.1 | RecName: Full = 30S ribosomal | 327 | 3.3 |
| 131 | | protein S18 | | |
| 5- | Q5HKJ3.1 | RecName: Full = Carbamate kinase | 328 | 3.3 |
| 132 | | | | |
| 5- | B9DM20.1 | RecName: Full = 30S ribosomal | 329 | 3.3 |
| 133 | | protein S10 | | |
| 5- | WP_ | MULTISPECIES: 50S ribosomal | 330 | 3.0 |
| 134 | 000024830.1 | protein L4 [Staphylococcus] | | |
| 5- | WP_ | MULTISPECIES: ornithine | 60 | 3.0 |
| 135 | 000136159.1 | carbamoyltransferase | | |
| | | [Staphylococcus] | | |
| 5- | WP_ | MULTISPECIES: amino acid ABC | 23 | 2.7 |
| 136 | 001144055.1 | transporter ATP-binding protein | | |
| | | [Staphylococcus] | | |
| 5- | WP_ | MULTISPECIES: manganese- | 331 | 2.7 |
| 137 | 001140871.1 | dependent inorganic | | |
| | | pyrophosphatase | | |
| | | [Staphylococcus] | | |
| 5- | WP_ | MULTISPECIES: histidine | 332 | 2.6 |
| 138 | 016187137.1 | phosphatase family protein | | |
| | | [Staphylococcus] | | |
| 5- | Q14U76.1 | RecName: Full = Bone sialoprotein- | 333 | 2.5 |
| 139 | | binding protein; Short = BSP- | | |
| | | binding protein; Flags: Precursor | | |
| 5- | P00807.1 | RecName: Full = Beta-lactamase; | 75 | 2.4 |
| 140 | | AltName: Full = Penicillinase; | | |
| | | Flags: Precursor | | |
| 5- | Q8CPC2.1 | RecName: Full = Aconitate | 334 | 2.3 |
| 141 | | hydratase A; Short = ACN; | | |
| | | Short = Aconitase; AltName: | | |
| | | Full = (2R,3S)-2-methylisocitrate | | |
| | | dehydratase; AltName: | | |
| | | Full = (2S,3R)-3-hydroxybutane- | | |
| | | 1,2,3-tricarboxylate dehydratase; | | |
| | | AltName: Full = Iron-responsive | | |
| | | protein-like; Short = IRP-like; | | |
| | | AltName: Full = Probable 2-methyl- | | |
| | | cis-aconitate hydratase; AltName: | | |
| | | Full = RNA-binding protein | | |
| 5- | Q6GJD0.1 | RecName: Full = 50S ribosomal | 335 | 2.3 |
| 142 | | protein L1 | | |
| 5- | Q2YXM1.1 | RecName: Full = Succinate--CoA | 336 | 2.2 |
| 143 | | ligase [ADP-forming] subunit beta; | | |
| | | AltName: Full = Succinyl-CoA | | |
| | | synthetase subunit beta; | | |
| | | Short = SCS-beta | | |
| 5- | WP_ | DNA-directed RNA polymerase | 337 | 2.2 |
| 144 | 017466657.1 | subunit beta [Staphylococcus | | |
| | | aureus] | | |
| 5- | A5IRE8.1 | RecName: Full = Coenzyme A | 338 | 2.2 |
| 145 | | disulfide reductase; Short = CoA- | | |
| | | disulfide reductase; Short = CoADR | | |
| 5- | A6QGF4.1 | RecName: Full = ATP-dependent | 339 | 2.2 |
| 146 | | protease ATPase subunit HsIU; | | |
| | | AltName: Full = Unfoldase HsIU | | |
| 5- | Q2YUU9.1 | RecName: Full = Superoxide | 340 | 2.2 |
| 147 | | dismutase [Mn/Fe] 2 | | |
| 5- | WP_ | MULTISPECIES: flavodoxin family | 153 | 2.2 |
| 148 | 000633782.1 | protein [Staphylococcus] | | |
| 5- | Q4L3G8.1 | RecName: Full = ATP-dependent | 65 | 2.2 |
| 149 | | zinc metalloprotease FtsH | | |
| 5- | Q2FJ80.1 | RecName: Full = FMN-dependent | 341 | 2.2 |
| 150 | | NADPH-azoreductase; AltName: | | |
| | | Full = NADPH-dependent flavo- | | |
| | | azoreductase; AltName: | | |
| | | Full = NADPH-flavin azoreductase | | |
| 5- | WP_ | MULTISPECIES: NAD-dependent | 342 | 2.2 |
| 151 | 001793810.1 | epimerase/dehydratase family | | |
| | | protein [Staphylococcus] | | |
| 5- | P07944.1 | RecName: Full = Beta-lactam- | 343 | 2.2 |
| 152 | | inducible penicillin-binding protein | | |
| 5- | WP_ | MULTISPECIES: cyclic-di-AMP | 344 | 2.2 |
| 153 | 001081643.1 | phosphodiesterase GdpP | | |
| | | [Staphylococcus] | | |
| 5- | Q49ZF8.1 | RecName: Full = 50S ribosomal | 345 | 2.2 |
| 154 | | protein L14 | | |
| 5- | Q4L4H7.1 | RecName: Full = Ribosome | 346 | 2.2 |
| 155 | | hibernation promotion factor; | | |
| | | Short = HPF | | |
| 5- | WP_ | MULTISPECIES: D-lactate | 55 | 2.2 |
| 156 | 000161541.1 | dehydrogenase [Staphylococcus] | | |
| 5- | WP_ | MULTISPECIES: single-stranded | 347 | 2.2 |
| 157 | 000608970.1 | DNA-binding protein [Bacilli] | | |
| 5- | WP_ | MULTISPECIES: hypothetical | 348 | 2.2 |
| 158 | 000011827.1 | protein [Staphylococcus] | | |
| 5- | Q2FKQ1.3 | RecName: Full = DNA gyrase | 349 | 2.2 |
| 159 | | subunit B | | |
| 5- | Q49VN8.1 | RecName: Full = Malate | 9 | 2.2 |
| 160 | | dehydrogenase | | |
| 5- | A6U2T7.1 | RecName: Full = Uroporphyrinogen | 350 | 2.2 |
| 161 | | decarboxylase; Short = UPD; | | |
| | | Short = URO-D | | |
| 5- | WP_ | MULTISPECIES: oleate hydratase | 93 | 2.2 |
| 162 | 000291835.1 | [Staphylococcus] | | |
| 5- | Q49XS9.1 | RecName: Full = GTPase Der; | 351 | 2.2 |
| 163 | | AltName: Full = GTP-binding | | |
| | | protein EngA | | |
| 5- | Q99XF3.2 | RecName: Full = Transcriptional | 352 | 2.2 |
| 164 | | regulatory protein WalR | | |
| 5- | WP_ | MULTISPECIES: DUF47 domain- | 353 | 2.2 |
| 165 | 000491755.1 | containing protein | | |
| | | [Staphylococcus] | | |
| 5- | P65258.1 | RecName: Full = L-lactate | 354 | 2.1 |
| 166 | | dehydrogenase 2; Short = L-LDH 2 | | |
| 5- | WP_ | MULTISPECIES: pyruvate | 355 | 2.0 |
| 167 | 001062662.1 | oxidase [Staphylococcus] | | |
| 5- | Q7A5N4.1 | RecName: | 356 | 2.0 |
| 168 | | Full = Dihydrolipoyllysine-residue | | |
| | | succinyltransferase component of | | |
| | | 2-oxoglutarate dehydrogenase | | |
| | | complex; AltName: Full = 2- | | |
| | | oxoglutarate dehydrogenase | | |
| | | complex component E2; | | |
| | | Short = OGDC-E2; AltName: | | |
| | | Full = Dihydrolipoamide | | |
| | | succinyltransferase component of | | |
| | | 2-oxoglutarate dehydrogenase | | |
| | | complex | | |
| 5- | Q6GE14.1 | RecName: Full = Gamma- | 357 | 1.9 |
| 169 | | hemolysin component A; AltName: | | |
| | | Full = H-gamma-2; AltName: | | |
| | | Full = H-gamma-II; | | |
| | | Flags: Precursor | | |
| 5- | P50307.1 | RecName: Full = S- | 358 | 1.9 |
| 170 | | adenosylmethionine synthase; | | |
| | | Short = AdoMet | | |
| | | synthase; AltName: | | |
| | | Full = MAT; AltName: | | |
| | | Full = Methionine | | |
| | | adenosyltransferase | | |
| 5- | P64269.1 | RecName: Full = 2,3- | 359 | 1.9 |
| 171 | | bisphosphoglycerate-independent | | |
| | | phosphoglycerate mutase; | | |
| | | Short = BPG-independent PGAM; | | |
| | | Short = Phosphoglyceromutase; | | |
| | | Short = iPGM | | |
| 5- | WP_ | phosphopyruvate hydratase | 360 | 1.9 |
| 172 | 017466665.1 | [Staphylococcus aureus] | | |
| 5- | Q8NWQ4.1 | RecName: Full = L-threonine | 41 | 1.8 |
| 173 | | dehydratase catabolic TdcB; | | |
| | | AltName: Full = Threonine | | |
| | | deaminase | | |
| 5- | WP_ | MULTISPECIES: | 361 | 1.8 |
| 174 | 000082722.1 | oligoendopeptidase F | | |
| | | [Staphylococcus] | | |
| 5- | Q8CMY4.1 | RecName: Full = Probable | 362 | 1.8 |
| 175 | | malate:quinone oxidoreductase 4; | | |
| | | AltName: Full = MQO 4; AltName: | | |
| | | Full = Malate dehydrogenase | | |
| | | [quinone] 4 | | |
| 5- | WP_ | MULTISPECIES: triose-phosphate | 363 | 1.8 |
| 176 | 001260089.1 | isomerase [Bacteria] | | |
| 5- | Q6GI31.1 | RecName: Full = Bifunctional | 364 | 1.8 |
| 177 | | autolysin; Includes: RecName: | | |
| | | Full = N-acetylmuramoyl-L-alanine | | |
| | | amidase; Includes: RecName: | | |
| | | Full = Mannosyl-glycoprotein endo- | | |
| | | beta-N-acetylglucosaminidase; | | |
| | | Flags: Precursor | | |
| 5- | WP_ | MULTISPECIES: elongation factor | 365 | 1.8 |
| 178 | 000090315.1 | G [Staphylococcus] | | |
| 5- | Q7A559.1 | RecName: Full = Pyruvate kinase; | 366 | 1.8 |
| 179 | | Short = PK | | |
| 5- | WP_ | MULTISPECIES: DNA | 367 | 1.7 |
| 180 | 000969811.1 | polymerase III subunit beta | | |
| | | [Staphylococcus] | | |
| 5- | Q2FK11.1 | RecName: Full = Iron-sulfur cluster | 368 | 1.6 |
| 181 | | repair protein ScdA | | |
| 5- | Q4A0C4.2 | RecName: Full = PTS system | 369 | 1.6 |
| 182 | | glucose-specific EIICBA | | |
| | | component; AltName: | | |
| | | Full = EIICBA-Glc; | | |
| | | Short = EII-Glc; | | |
| | | AltName: Full = EIICBA-Glc 1; | | |
| | | Includes: RecName: Full = Glucose | | |
| | | permease IIC component; | | |
| | | AltName: Full = PTS system | | |
| | | glucose-specific EIIC component; | | |
| | | Includes: RecName: | | |
| | | Full = Glucose-specific | | |
| | | phosphotransferase enzyme IIB | | |
| | | component; AltName: Full = PTS | | |
| | | system glucose-specific EIIB | | |
| | | component; Includes: RecName: | | |
| | | Full = Glucose-specific | | |
| | | phosphotransferase enzyme IIA | | |
| | | component; AltName: Full = PTS | | |
| | | system glucose-specific EIIA | | |
| | | component | | |
| 5- | Q4L7Y6.1 | RecName: Full = ATP synthase | 370 | 1.6 |
| 183 | | subunit alpha; AltName: Full = ATP | | |
| | | synthase F1 sector subunit alpha; | | |
| | | AltName: Full = F-ATPase subunit | | |
| | | alpha | | |
| 5- | Q6G640.1 | RecName: Full = Ornithine | 371 | 1.6 |
| 184 | | carbamoyltransferase, catabolic; | | |
| | | Short = OTCase | | |
| 5- | Q8CSD1.1 | RecName: Full = UPF0365 protein | 372 | 1.6 |
| 185 | | SE 1260 | | |
| 5- | Q2FWD1.1 | RecName: Full = CTP synthase; | 373 | 1.6 |
| 186 | | AltName: Full = Cytidine 5′- | | |
| | | triphosphate synthase; AltName: | | |
| | | Full = Cytidine triphosphate | | |
| | | synthetase; Short = CTP | | |
| | | synthetase; Short = CTPS; | | |
| | | AltName: Full = UTP--ammonia | | |
| | | ligase | | |
| 5- | Q6GAV1.1 | RecName: Full = Chaperone | 374 | 1.6 |
| 187 | | protein ClpB | | |
| 5- | A5ISH0.1 | RecName: Full = Ribonuclease Y; | 375 | 1.6 |
| 188 | | Short = RNase Y; AltName: | | |
| | | Full = Conserved virulence factor A | | |
| 5- | Q6GC92.1 | RecName: Full = Alkyl | 376 | 1.6 |
| 189 | | hydroperoxide reductase subunit | | |
| | | F | | |
| 5- | P63940.1 | RecName: Full = D-lactate | 377 | 1.6 |
| 190 | | dehydrogenase; Short = D-LDH; | | |
| | | AltName: Full = D-specific 2- | | |
| | | hydroxyacid dehydrogenase | | |
| 5- | Q2FFJ5.1 | RecName: Full = Glutamyl- | 378 | 1.6 |
| 191 | | tRNA(GIn) amidotransferase | | |
| | | subunit A; Short = Glu-ADT | | |
| | | subunit A | | |
| 5- | WP_ | FOF1 ATP synthase subunit alpha | 379 | 1.6 |
| 192 | 017466629.1 | [Staphylococcus aureus] | | |
| 5- | Q2YXR6.1 | RecName: Full = Glycerol kinase; | 380 | 1.5 |
| 193 | | AltName: Full = ATP:glycerol 3- | | |
| | | phosphotransferase; AltName: | | |
| | | Full = Glycerokinase; Short = GK | | |
| 5- | Q2FVK2.1 | RecName: Full = Gamma- | 381 | 1.5 |
| 194 | | hemolysin component C; | | |
| | | AltName: Full = Leukocidin s | | |
| | | subunit; Flags: Precursor | | |
| 5- | Q2YUH3.1 | RecName: Full = DEAD-box ATP- | 382 | 1.5 |
| 195 | | dependent RNA helicase CshA | | |
| 5- | Q2YUM9.1 | RecName: Full = UDP-N- | 383 | 1.5 |
| 196 | | acetylglucosamine 1- | | |
| | | carboxyvinyltransferase 2; | | |
| | | AltName: Full = Enoylpyruvate | | |
| | | transferase 2; AltName: | | |
| | | Full = UDP-N-acetylglucosamine | | |
| | | enolpyruvyl transferase 2; | | |
| | | Short = EPT 2 | | |
| 5- | Q8NVK6.3 | RecName: Full = Delta-hemolysin; | 384 | 1.5 |
| 197 | | Short = Delta-lysin; AltName: | | |
| | | Full = Delta-toxin | | |

A series of computational analyses can be performed based upon the amino acid sequences of the protein shown in Tables 2 and 3 to further identify correlates of immunity that could be developed as subunit vaccines. For example, if subunit vaccine proteins are expected to be located on the surface of the bacteria so as to be exposed to antibody immunity, then the subsets of proteins can be selected for (1) the presence of a signal sequence directing the proteins to the outer membrane or the presence of a hydrophobic transmembrane domain (Tommassen 2010) and/or (2) annotations related to “outer membrane proteins”, “resistance-nodulator-division (RND) transporter”, and the like (from KEGG protein functional database search, Kanehisha et al., 2016).

### Example 2: Growth of MRSA in Varying Culture Conditions Leads to the Expression of Different Proteins Depending on Culture Condition Used

MRSA was propagated in planktonic and biofilm forms as shown in FIG. 1.

For Planktonic Culture #1, 10 ml of TSB was inoculated with approximately 10 ul of MRSA-M2 from a freshly streaked plate and incubated at 37° C., and shaken at 200 rpm overnight. The following day the OD600 of the overnight culture was measured at 1.25. 1 ml of culture was added to 100 ml of TSB in a 250 ml Erlenmeyer flask and again incubated overnight at 200 rpm. The next day at 16 hours post inoculation (day 3), the culture was collected at an OD of 1.25. Cells were centrifuged at 5500 rcf for 15 mins at 4° C. and washed in cold PBS (4° C.). Centrifugation and washing in cold PBS were repeated. Cells were resuspended to a volume of 35 ml of cold PBS and stored at 4° C. for titration, protein analysis, and irradiation.

For the Titanium plate drip culture, 1×10⁸ CFU of MRSA was inoculated into 15 ml of 1×TSB and placed into each chamber of a drip reactor under sterile conditions with the effluent tubing clamped. The inoculum was placed over an 18.75 cm² medical grade titanium coupon (harvestable growth surface). The drip reactor was sealed and incubated at 37° C. for 16 hours. Following incubation, the effluent tubing was unclamped and the reactor was placed at a 10° angle. The culture was fed by continuous flow media consisting of 2 g/L TSB and 2 g/L D-glucose dripped through an inlet port at the high end of the chamber through 22-gauge syringe. Gravity allowed the drips to drain over the coupon to feed the culture. The flow rate was set at 0.3 ml per minute by peristaltic pump. The culture was allowed to grow for 5 days at 37° C. before harvesting under sterile conditions. Cells were centrifuged at 5500×g for 15 mins at 4° C. and washed in cold PBS (4° C.). Centrifugation and washing in cold PBS were repeated. Cells were resuspended to a volume of 10 ml of cold PBS and stored at 4° C. for titration, protein analysis, and irradiation.

For “M9 under” (submerged biofilm), 65 ml of M9 media was inoculated with around 10 ml of MRSA-M2 from a freshly streaked plate and incubated at 37° C. for 18 h while shaking at 200 rpm. The following day, the OD600 was measured from the overnight culture at 1.25 optical absorbance units. 6 ml volumes of overnight culture were added to 225 cm² flasks containing 95 ml of M9 media. Media and culture were mixed and placed in a 37° C. incubator without shaking. Media was replaced with fresh M9 media the following day (day 3). On day 4, media was removed from the flask and replenished with 25 ml of 4° C. cold PBS. Cells were scraped into the cold PBS using a cell scraper. Cells were centrifuged at 5500×g for 15 mins at 4° C. and washed in cold PBS. Centrifugation and washing in cold PBS were repeated. Cells were resuspended to a volume of 35 ml of cold PBS and stored at 4° C. for titration, protein analysis, and irradiation.

For static biofilms cultured under aqueous conditions, 65 ml of TSB media was inoculated with around 10 ul of MRSA-M2 from a freshly streaked plate and incubated at 37° C., and shaken at 200 rpm overnight. The following day, the OD600 was measured from the overnight culture at 1.25 ABS. 6 ml of overnight culture were added to 225 cm² flasks containing 95 ml of TSB supplemented with 10% bovine synovial fluid. Media and culture were mixed and placed in a 37° C. incubator without shaking. Media was replenished with fresh TSB supplemented with 10% bovine synovial fluid the following day (day 3); 20 ml of media was removed being careful not to disturb clusters if possible and the culture was replenished with 22 ml of fresh media. On day 4, cells and media were collected from the flask. Cells were scraped into the cold PBS using a cell scraper. Cells were centrifuged at 5500×g for 15 mins at 4° C. and washed in cold PBS. Centrifugation and washing in cold PBS were repeated. Cells were resuspended to a volume of 35 ml of cold PBS and stored at 4° C. for titration, protein analysis, and irradiation. Shown in FIG. 1 and table 1 lanes from Coomassie-stained SDS-PAGE gels, and their corresponding culture conditions for 14 distinctly grown samples of MRSA. Throughout the samples, it appears that no two samples have entirely the same banding pattern indicating that each growth condition results in a different proteomic profile. Note, in some instances changes in the relative ratio of bands may be just as important as the apparent presence and absence of bands. We find similar results for every bacterium we have tested to date. Alternative methods can be used for protein analysis such as 2D gel and mass spectroscopy.

### Example 3: UVC-Irradiation Inactivation of MRSA Leads to Partially Protective Immunogens

Sterilization of pathogens with gamma and UVC irradiation can be used to inactivate the replicative capability of MRSA for the generation of whole-organism vaccines (Moore, 1936). FIG. 2 panel A shows 2×10\hat{ }8 CFU per ml of MRSA samples exposure to a UVC lamp (4.8 mW/cm²) for the indicated times and plated on LB agar plates. Cells were propagated using a variety of methods and complexed with MDP using 3 mM MnCl2, 3 mM DP1 (DEHGTAVMLK; SEQ ID NO:387) decapeptide, and 25 mM potassium phosphate buffer (pH 7.4). A 100-second exposure to a UVC lamp (4.8 mW/cm²) completely killed bacteria grown under multiple conditions, planktonically or as biofilm, whether or not MDP was present (FIG. 2 panel A). The presence of the MDP complex reduces the oxidation of proteins generated by MDP. Samples were cultured and washed twice in PBS. Samples were then lysed with lysostaphin treatment and boiled in SDS loading dye with beta-mercaptoethanol. Oxidized groups were derivatized to dinitrophenyl groups by reaction with dinitrophenylhydrazine followed by neutralization of dinitrophenylhydrazine. Samples were then resolved by SDS-PAGE followed by western analysis for detection of dinitrophenyl groups with an anti-Dinitrophenyl (DPN) antibody. In FIG. 3 panel D there is lower detection of oxidized groups in the samples that were either protected by MDP or not irradiated. In FIG. 2 panels E, F and G we show that epitopes detected by the immune system are damaged during irradiation, and that damage is protected by MDP. The addition of MDP when irradiating samples can be used generate better quality, more protective immunogens. In the example, planktonic MRSA were irradiated for 5 mins with MDP or buffer only, samples were lysed, and epitopes were probed with anti-MRSA mouse sera. In the +MDP samples, epitopes are clearly better detected by mouse antibody than in the sample prepared in buffer alone.

### Example 4. Efficacy of Irradiated Whole-Cell S. aureus Vaccines in a Prosthetic Implant Model of Infection

The efficacy of the whole cell irradiated vaccines can be measured in animal models of infection. In this example five UVC-MDP-inactivated whole-cell S. aureus preparations, which were grown under different conditions-planktonic, M9-biofilm, blood, synovial, and titanium-plate (Ti) biofilm-were tested for protection in a bone-implant challenge model. The UVC-MDP-inactivated bacteria (2.5×10⁷ CFUs) were emulsified in Alum (for boosting the Th2 response) and injected intramuscularly on days 0 and 21. On day 42, the mice were anesthetized and sterile stainless-steel pins were implanted transcortically into the tibiae, trimmed flush with the bone surface, and inoculated with 3,000 CFUs of S. aureus M2 (Prabhakara, 2011). One week later, the mice were euthanized and bacterial burden in the infected tibiae were enumerated (FIG. 3). Mice with >1 log 10 reduction in CFU/mg of bone were considered protected; the average reduction in such protected mice was 3 logs. Protection was least potent with the planktonic vaccines, while protection was greatest with the synovial and Ti-plate immunogens. The efficacy of the Ti-plate immunogen, protecting 50% of mice, may be due to “like” epitopes protecting against “like” infection. In a second experiment complete clearance of infection was observed in 50% of mice. Complete protection may require both planktonic and biofilm proteins. This approach can be used to find whole-cell vaccines, or the approach can be used in combination with Examples 5 and 6 to discover subunits.

### Example 5. Subunit Identification by Differential Mass Spectroscopy of Protective Vs. Non-Protective Culture Conditions

The culture conditions used to make the inactivated whole-cell preparations that are protective are thought to express protective antigens. Conversely, protective antigens are not expected to be expressed in culture conditions that did not produce inactivate whole-cell immunogens that protected mice from challenge. To identify protective proteins in the pin-implant infection model (FIG. 3), differential analysis of protective and non-protective samples via mass spectroscopy can be performed. We compared the proteomes (without immunoprecipitation) of the protective synovial and Ti-plate biofilms to the proteomes of the nonprotective stationary-phase planktonic culture (same cultures as in FIG. 4 panel A) via LC/MS/MS. This analysis identified 53 proteins increased by 2-fold in the synovial fluid culture and 92 proteins increased 2-fold in the Ti-plate culture vs. planktonic bacteria. Among these are well-studied virulence factors and vaccine candidates such as α-hemolysin and as well as novel candidates as summarized in Table 3.

### Example 6. Identification of Subunit Proteins Recognized by Sera from Protected Mice

Following vaccination with multiple whole cell vaccines, immune correlates from each sample can be compared to reveal the most protective epitopes. In this example, serum samples containing antibodies from protected and non-protected mice were obtained after vaccination but prior to challenge and used to immunoprecipitated (IP) proteins from their corresponding lysates—e.g., planktonic sera were used to IP planktonic lysate- and the immunoprecipitates were subjected to LC/MS/MS. These were then computationally ranked for each sample, and then each ranked by order of its correlation with protection for every sample. A total of 136 proteins were identified as being unique or eliciting a greater antibody response in protected mice (comparing 10 non-protected and 11 protected mice). A subset of these proteins is shown in Table 3. Interestingly, the proteins identified by Examples 5 and 6 have numerous differences, which may be expected since no single whole-cell vaccine protected all mice.

Examples 5 and 6 can be used to identify novel immunogens that can be used as single subunit or multivalent subunit vaccines.

1-12. (canceled)

13. A staphylococcal immunogen, wherein the staphylococcal immunogen is and/or is derived from an irradiation-inactivated bacteria, optionally grown under conditions such that one or more different bacterial immunogens that stimulate protective immunity are present as compared to control, e.g., as compared to a staphylococcal immunogen that is not irradiation-inactivated and/or is not derived from an irradiation/inactivated bacteria.

14. (canceled)

15. The staphylococcal immunogen of claim 13, wherein the staphylococcal immunogen has been inactivated using ionizing radiation and/or using ultraviolet light irradiation.

16. (canceled)

17. The staphylococcal immunogen of claim 13, wherein the irradiation-inactivated bacteria are complexed with an antioxidant.

18. The staphylococcal immunogen of claim 13, wherein the staphylococcal immunogen is not complexed with an antioxidant and/or has been irradiation-inactivated in the absence of an antioxidant.

19. The staphylococcal immunogen of claim 13, wherein the immunogen has been isolated from a planktonic culture or a biofilm culture of irradiation-inactivated bacteria.

20. (canceled)

21. A composition comprising the staphylococcal immunogen of claim 13, and a pharmaceutically acceptable carrier, diluent and/or adjuvant.

22-57. (canceled)

58. An immunogenic composition comprising one or more staphylococcal immunogen, wherein the immunogenic composition does not comprise a whole cell bacterium.

59-60. (canceled)

61. The immunogenic composition of claim 58, wherein the immunogenic composition comprises one or more immunogen(s) and wherein the one or more immunogen(s) comprises at least one immunogen comprising the amino acid sequence of any one of SEQ ID NOs: 1-384 or an amino acid sequence having at least 90% sequence identity thereto.

62-91. (canceled)

92. A method of making an immunogenic composition, comprising:
a) culturing a live staphylococcal bacterium to produce a bacterial population, optionally wherein the bacterial population comprises one or more immunogen(s), and
b) exposing the bacterial population produced in step (a) to irradiation to produce an inactivated bacterial population, and/or
c) isolating the one or more immunogen(s),
thereby producing the immunogenic composition.

93. The method of claim 92, wherein step a) of culturing the live staphylococcal bacterium comprises:
culturing the live staphylococcal bacterium under planktonic culture conditions to produce a non-adherent bacterial population, and
obtaining the non-adherent bacterial population during the exponential growth phase or the stationary growth phase of the population, to provide an obtained population.

94. (canceled)

95. The method of claim 92, wherein step a) of culturing the live staphylococcal bacterium comprises:
culturing the live staphylococcal bacterium under plate biofilm culture conditions or aqueous biofilm culture conditions to produce an adherent bacterial population and optionally a non-adherent bacterial population, and
obtaining the adherent bacterial population and optionally the non-adherent bacterial population, to provide an obtained population.

96-98. (canceled)

99. The method of claim 95, wherein the plate biofilm culture conditions comprise growing the live staphylococcal bacterium in and/or on a medium on a solid plate, optionally a solidified medium agar plate and/or a plate drip reactor system.

100. The method of claim 95, wherein the plate biofilm culture conditions comprise growing the live staphylococcal bacterium in a static (motionless) aqueous medium.

101-102. (canceled)

103. The method of claim 93, wherein the adherent and/or non-adherent bacterial population comprises one or more immunogen(s) comprising the amino acid sequence of any one of SEQ ID NOs: 1-384 or an amino acid sequence having at least 90% sequence identity thereto, in any combination.

104-105. (canceled)

106. The method of claim 92, wherein exposing the bacterial population to irradiation comprises exposing the bacterial population to said irradiation for about 30 seconds to about 10 minutes.

107-123. (canceled)

124. A method of producing an immune response to a staphylococcal bacterium in a subject, comprising administering to the subject an effective amount of the immunogenic composition of claim 58, thereby producing an immune response to a staphylococcal bacterium in the subject.

125. A method of treating a staphylococcal infection in a subject in need thereof, comprising administering to the subject an effective amount of the immunogenic composition of claim 58, thereby treating a staphylococcal infection in the subject.

126. A method of reducing the risk of a disease or disorder associated with or caused by a staphylococcal infection in a subject, comprising administering to the subject an effective amount of the immunogenic composition of claim 58, thereby preventing or reducing the risk of a disease or disorder associated with or caused by a staphylococcal infection in the subject.

127. A method of protecting a subject from the effects of a staphylococcal infection, comprising administering to the subject an effective amount of the immunogenic composition of claim 58, thereby protecting the subject from the effects of a staphylococcal infection.

128-137. (canceled)


SEQUENCE LISTING (387 sequences)

SEQ ID NO: 1 - protein, 475 aa, Staphylococcus haemolyticus
MHFETVIGLEVHVELKTDSKMFSPSPAHFGAEPNSNTNVIDLAYPGVLPVVNRRAVDWAMRASMALNMDIATNSKFDRKNYFYPDNPKAYQISQFDQPIGENGYIDIEVDGETKRIGITRLHMEEDAGKSTHKDGYSLVDLNRQGTPLIEIVSEPDIRSPKEAYAYLEKLRSIIQYTGVSDCKMEEGSLRCDANISLRPYGQEEFGTKTELKNLNSFNYVRKGLEYEEKRQEEELLNGGTIGQETRRFDESTGKTILMRVKEGSDDYRYFPEPDIVPLYVDEEWKECVRQTIPELPDERKAKYVNDLGLPEYDAHVLTLTKEMSDFFEGAIEKGADVKLTSNWLMGGVNEYLNKNQVELQDTKLTPENLAGMIKLIEDGTMSSKIAKKVFPELAENGGDAKQIMEDKGLVQISDEATLTKFVTEALDNNPQSVEDYKNGKGKAMGFLVGQIMKASKGQANPQKVNQILKQELDKR

SEQ ID NO: 2 - protein, 10421 aa, Staphylococcus aureus
MNYRDKIQKFSIRKYTVGTFSTVIATLVFLGFNTSQAHAAETNQPASVVKQKQQSNNEQTENRESQVQNSQNSQNGQSLSATHENEQPNISQANLVDQKVAQSSTTNDEQPASQNVNTKKDSATAATTQPDKEQSKHKQNESQSANKNGNDNRAAHVENHEANVVTASDSSDNGNVQHDRNELQAFFDANYHDYRFIDRENADSGTFNYVKGIFDKINTLLGSNDPINNKDLQLAYKELEQAVALIRTMPQRQQTSRRSNRIQTRSVESRAAEPRSVSDYQNANSSYYVENANDGSGYPVGTYINASSKGAPYNLPTTPWNTLKASDSKEIALMTAKQTGDGYQWVIKFNKGHAPHQNMIFWFALPADQVPVGRTDFVTVNSDGTNVQWSHGAGAGANKPLQQMWEYGVNDPHRSHDFKIRNRSGQVIYDWPTVHIYSLEDLSRASDYFSEAGATPATKAFGRQNFEYINGQKPAESPGVPKVYTFIGQGDASYTISFKTQGPTVNKLYYAAGGRALEYNQLFMYSQLYVESTQDHQQRLNGLRQVVNRTYRIGTTKRVEVSQGNVQTKKVLESTNLNIDDFVDDPLSYVKTPSNKVLGFYSNNANTNAFRPGGAQQLNEYQLSQLFTDQKLQEAARTRNPIRLMIGFDYPDAYGNSETLVPVNLTVLPEIQHNIKFFKNDDTQNIAEKPFSKQAGHPVFYVYAGNQGNASVNLGGSVTSIQPLRINLTSNENFTDKDWQITGIPRTLHIENSTNRPNNARERNIELVGNLLPGDYFGTIRFGRKEQLFEIRVKPHTPTITTTAEQLRGTALQKVPVNISGIPLDPSALVYLVAPTNQTTNGGSEADQIPSGYTILATGTPDGVHNTITIRPQDYVVFIPPVGKQIRAVVYYNKVVASNMSNAVTILPDDIPPTINNPVGINAKYYRGDEVNFTMGVSDRHSGIKNTTITTLPNGWTSNLTKADKNNGSLSITGRVSMNQAFNSDITFKVSATDNVNNTTNDSQSKHVSIHVGKISEDAHPIVLGNTEKVVVVNPTAVSNDEKQSIITAFMNKNQNIRGYLASTDPVTVDNNGNVTLHYRDGSSTTLDATNVMTYEPVVKPEYQTVNAAKTATVTIAKGQSFSIGDIKQYFTLSNGQPIPSGTFTNITSDRTIPTAQEVSQMNAGTQLYHITATNAYHKDSEDFYISLKIIDVKQPEGDQRVYRTSTYDLTTDEISKVKQAFINANRDVITLAEGDISVTNTPNGANVSTITVNINKGRLTKSFASNLANMNFLRWVNFPQDYTVTWTNAKIANRPTDGGLSWSDDHKSLIYRYDATLGTQITTNDILTMLKATTTVPGLRNNITGNEKSQAEAGGRPNFRTTGYSQSNATTDGQRQFTLNGQVIQVLDIINPSNGYGGQPVTNSNTRANHSNSTVVNVNEPAANGAGAFTIDHVVKSNSTHNASDAVYKAQLYLTPYGPKQYVEHLNQNTGNTTDAINIYFVPSDLVNPTISVGNYTNHQVFSGETFTNTITANDNFGVQSVTVPNTSQITGTVDNNHQHVSATAPNVTSATNKTINLLATDTSGNTATTSFNVTVKPLRDKYRVGTSSTAANPVRIANISNNATVSQADQTTIINSLTFTETVPNRSYARASANEITSKTVSNVSRTGNNANVTVTVTYQDGTTSTVTVPVKHVIPEIVAHSHYTVQGQDFPAGNGSSASDYFKLSNGSDIADATITWVSGQAPNKDNTRIGEDITVTAHILIDGETTPITKTATYKVVRTVPKHVFETARGVLYPGVSDMYDAKQYVKPVNNSWSTNAQHMNFQFVGTYGPNKDVVGISTRLIRVTYDNRQTEDLTILSKVKPDPPRIDANSVTYKAGLTNQEIKVNNVLNNSSVKLFKADNTPLNVTNITHGSGFSSVVTVSDALPNGGIKAKSSISMNNVTYTTQDEHGQVVTVTRNESVDSNDSATVTVTPQLQATTEGAVFIKGGDGFDFGHVERFIQNPPHGATVAWHDSPDTWKNTVGNTHKTAVVTLPNGQGTRNVEVPVKVYPVANAKAPSRDVKGQNLTNGTDAMNYITFDPNTNTNGITAAWANRQQPNNQQAGVQHLNVDVTYPGISAAKRVPVTVNVYQFEFPQTTYTTTVGGTLASGTQASGYAHMQNATGLPTDGFTYKWNRDTTGTNDANWSAMNKPNVAKVVNAKYDVIYNGHTFATSLPAKFVVKDVQPAKPTVTETAAGAITIAPGANQTVNTHAGNVTTYADKLVIKRNGNVVTTFTRRNNTSPWVKEASAATVAGIAGTNNGITVAAGTFNPADTIQVVATQGSGETVSDEQRSDDFTVVAPQPNQATTKIWQNGHIDITPNNPSGHLINPTQAMDIAYTEKVGNGAEHSKTINVVRGQNNQWTIANKPDYVTLDAQTGKVTFNANTIKPNSSITITPKAGTGHSVSSNPSTLTAPAAHTVNTTEIVKDYGSNVTAAEINNAVQVANKRTATIKNGTAMPTNLAGGSTTTIPVTVTYNDGSTEEVQESIFTKADKRELITAKNHLDDPVSTEGKKPGTITQYNNAMHNAQQQINTAKTEAQQVINNERATPQQVSDALTKVRAAQTKIDQAKALLQNKEDNSQLVTSKNNLQSSVNQVPSTAGMTQQSIDNYNAKKREAETEITAAQRVIDNGDATAQQISDEKHRVDNALTALNQAKHDLTADTHALEQAVQQLNRTGTTTGKKPASITAYNNSIRALQSDLTSAKNSANAIIQKPIRTVQEVQSALTNVNRVNERLTQAINQLVPLADNSALKTAKTKLDEEINKSVTTDGMTQSSIQAYENAKRAGQTESTNAQNVINNGDATDQQIAAEKTKVEEKYNSLKQAIAGLTPDLAPLQTAKTQLQNDIDQPTSTTGMTSASIAAFNEKLSAARTKIQEIDRVLASHPDVATIRQNVTAANAAKSALDQARNGLTVDKAPLENAKNQLQHSIDTQTSTTGMTQDSINAYNAKLTAARNKIQQINQVLAGSPTVEQINTNTSTANQAKSDLDHARQALTPDKAPLQTAKTQLEQSINQPTDTTGMTTASLNAYNQKLQAARQKLTEINQVLNGNPTVQNINDKVTEANQAKDQLNTARQGLTLDRQPALTTLHGASNLNQAQQNNFTQQINAAQNHAALETIKSNITALNTAMTKLKDSVADNNTIKSDQNYTDATPANKQAYDNAVNAAKGVIGETTNPTMDVNTVNQKAASVKSTKDALDGQQNLQRAKTEATNAITHASDLNQAQKNALTQQVNSAQNVQAVNDIKQTTQSLNTAMTGLKRGVANHNQVVQSDNYVNADTNKKNDYNNAYNHANDIINGNAQHPVITPSDVNNALSNVTSKEHALNGEAKLNAAKQEANTALGHLNNLNNAQRQNLQSQINGAHQIDAVNTIKQNATNLNSAMGNLRQAVADKDQVKRTEDYADADTAKQNAYNSAVSSAETIINQTTNPTMSVDDVNRATSAVTSNKNALNGYEKLAQSKTDAARAIDALPHLNNAQKADVKSKINAASNIAGVNTVKQQGTDLNTAMGNLQGAINDEQTTLNSQNYQDATPSKKTAYTNAVQAAKDILNKSNGQNKTKDQVTEAMNQVNSAKNNLDGTRLLDQAKQTAKQQLNNMTHLTTAQKTNLTNQINSGTTVAGVQTVQSNANTLDQAMNTLRQSIANKDATKASEDYVDANNDKQTAYNNAVAAAETIINANSNPEMNPSTITQKAEQVNSSKTALNGDENLAAAKQNAKTYLNTLTSITDAQKNNLISQITSATRVSGVDTVKQNAQHLDQAMASLQNGINNESQVKSSEKYRDADTNKQQEYDNAITAAKAILNKSTGPNTAQNAVEAALQRVNNAKDALNGDAKLIAAQNAAKQHLGTLTHITTAQRNDLTNQISQATNLAGVESVKQNANSLDGAMGNLQTAINDKSGTLASQNFLDADEQKRNAYNQAVSAAETILNKQTGPNTAKTAVEQALNNVNNAKHALNGTQNLNNAKQAAITAINGASDLNQKQKDALKAQANGAQRVSNAQDVQHNATELNTAMGTLKHAIADKTNTLASSKYVNADSTKQNAYTTKVTNAEHIISGTPTVVTTPSEVTAAANQVNSAKQELNGDERLREAKQNANTAIDALTQLNTPQKAKLKEQVGQANRLEDVQTVQTNGQALNNAMKGLRDSIANETTVKTSQNYTDASPNNQSTYNSAVSNAKGIINQTNNPTMDTSAITQATTQVNNAKNGLNGAENLRNAQNTAKQNLNTLSHLTNNQKSAISSQIDRAGHVSEVTATKNAATELNTQMGNLEQAIHDQNTVKQSVKFTDADKAKRDAYTNAVSRAEAILNKTQGANTSKQDVEAAIQNVSSAKNALNGDQNVTNAKNAAKNALNNLTSINNAQKRDLTTKIDQATTVAGVEAVSNTSTQLNTAMANLQNGINDKTNTLASENYHDADSDKKTAYTQAVTNAENILNKNSGSNLDKTAVENALSQVANAKGALNGNHNLEQAKSNANTTINGLQHLTTAQKDKLKQQVQQAQNVAGVDTVKSSANTLNGAMGTLRNSIQDNTATKNGQNYLDATERNKTNYNNAVDSANGVINATSNPNMDANAINQIATQVTSTKNALDGTHNLTQAKQTATNAIDGATNLNKAQKDALKAQVTSAQRVANVTSIQQTANELNTAMGQLQHGIDDENATKQTQKYRDAEQSKKTAYDQAVAAAKAILNKQTGSNSDKAAVDRALQQVTSTKDALNGDAKLAEAKAAAKQNLGTLNHITNAQRTDLEGQINQATTVDGVNTVKTNANTLDGAMNSLQGSINDKDATLRNQNYLDADESKRNAYTQAVTAAEGILNKQTGGNTSKADVDNALNAVTRAKAALNGADNLRNAKTSATNTIDGLPNLTQLQKDNLKHQVEQAQNVAGVNGVKDKGNTLNTAMGALRTSIQNDNTTKTSQNYLDASDSNKNNYNTAVNNANGVINATNNPNMDANAINGMANQVNTTKAALNGAQNLAQAKTNATNTINNAHDLNQKQKDALKTQVNNAQRVSDANNVQHTATELNSAMTALKAAIADKERTKASGNYVNADQEKRQAYDSKVTNAENIISGTPNATLTVNDVNSAASQVNAAKTALNGDNNLRVAKEHANNTIDGLAQLNNAQKAKLKEQVQSATTLDGVQTVKNSSQTLNTAMKGLRDSIANEATIKAGQNYTDASPNNRNEYDSAVTAAKAIINQTSNPTMEPNTITQVTSQVTTKEQALNGARNLAQAKTTAKNNLNNLTSINNAQKDALTRSIDGATTVAGVNQETAKATELNNAMHSLQNGINDETQTKQTQKYLDAEPSKKSAYDQAVNAAKAILTKASGQNVDKAAVEQALQNVNSTKTALNGDAKLNEAKAAAKQTLGTLTHINNAQRTALDNEITQATNVEGVNTVKAKAQQLDGAMGQLETSIRDKDTTLQSQNYQDADDAKRTAYSQAVNAAATILNKTAGGNTPKADVERAMQAVTQANTALNGIQNLDRAKQAANTAITNASDLNTKQKEALKAQVTSAGRVSAANGVEHTATELNTAMTALKRAIADKAETKASGNYVNADANKRQAYDEKVTAAENIVSGTPTPTLTPADVTNAATQVTNAKTQLNGNHNLEVAKQNANTAIDGLTSLNGPQKAKLKEQVGQATTLPNVQTVRDNAQTLNTAMKGLRDSIANEATIKAGQNYTDASQNKQTDYNSAVTAAKAIIGQTTSPSMNAQEINQAKDQVTAKQQALNGQENLRTAQTNAKQHLNGLSDLTDAQKDAVKRQIEGATHVNEVTQAQNNADALNTAMTNLKNGIQDQNTIKQGVNFTDADEAKRNAYTNAVTQAEQILNKAQGPNTSKDGVETALENVQRAKNELNGNQNVANAKTTAKNALNNLTSINNAQKEALKSQIEGATTVAGVNQVSTTASELNTAMSNLQNGINDEAATKAAQKYTDADREKQTAYNDAVTAAKTLLDKTAGSNDNKAAVEQALQRVNTAKTALNGDERLNEAKNTAKQQVATMSHLTDAQKANLTSQIESGTTVAGVQGIQANAGTLDQAMNQLRQSIASKDATKSSEDYQDANADLQNAYNDAVTNAEGIISATNNPEMNPDTINQKASQVNSAKSALNGDEKLAAAKQTAKSDIGRLTDLNNAQRTAANAEVDQAPNLAAVTAAKNKATSLNTAMGNLKHALAEKDNTKRSVNYTDADQPKQQAYDTAVTQAEAITNANGSNANETQVQAALNQLNQAKNDLNGDNKVAQAKESAKRALASYSNLNNAQSTAAISQIDNATTVAGVTAAQNTANELNTAMGQLQNGINDQNTVKQQVNFTDADQGKKDAYTNAVTNAQGILDKAHGQNMTKAQVEAALNQVTTAKNALNGDANVRQAKSDAKANLGTLTHLNNAQKQDLTSQIEGATTVNGVNGVKTKAQDLDGAMQRLQSAIANKDQTKASENYIDADPTKKTAFDNAITQAESYLNKDHGANKDKQAVEQAIQSVTSTENALNGDANLQRAKTEAIQAIDNLTHLNTPQKTALKQQVNAAQRVSGVTDLKNSATSLNNAMDQLKQAIADHDTIVASGNYTNASPDKQGAYTDAYNAAKNIVNGSPNVITNAADVTAATQRVNNAETGLNGDTNLATAKQQAKDALRQMTHLSDAQKQSITGQIDSATQVTGVQSVKDNATNLDNAMNQLRNSIANKDDVKASQPYVDADRDKQNAYNTAVTNAENIINATSQPTLDPSAVTQAANQVSTNKTALNGAQNLANKKQETTANINQLSHLNNAQKQDLNTQVTNAPNISTVNQVKTKAEQLDQAMERLINGIQDKDQVKQSVNFTDADPEKQTAYNNAVTAAENIINQANGTNANQSQVEAALSTVTTTKQALNGDRKVTDAKNNANQTLSTLDNLNNAQKGAVTGNINQAHTVAEVTQAIQTAQELNTAMGNLKNSLNDKDTTLGSQNFADADPEKKNAYNEAVHNAENILNKSTGTNVPKDQVEAAMNQVNATKAALNGTQNLEKAKQHANTAIDGLSHLTNAQKEALKQLVQQSTTVAEAQGNEQKANNVDAAMDKLRQSIADNATTKQNQNYTDASQNKKDAYNNAVTTAQGIIDQTTSPTLDPTVINQAAGQVSTTKNALNGNENLEAAKQQASQSLGSLDNLNNAQKQTVTDQINGAHTVDEANQIKQNAQNLNTAMGNLKQAIADKDATKATVNFTDADQAKQQAYNTAVTNAENIISKANGGNATQAEVEQAIKQVNAAKQALNGNANVQHAKDEATALINSSNDLNQAQKDALKQQVQNATTVAGVNNVKQTAQELNNAMTQLKQGIADKEQTKADGNFVNADPDKQNAYNQAVAKAEALISATPDVVVTPSEITAALNKVTQAKNDLNGNTNLATAKQNVQHAIDQLPNLNQAQRDEYSKQITQATLVPNVNAIQQAATTLNDAMTQLKQGIANKAQIKGSENYHDADTDKQTAYDNAVTKAEELLKQTTNPTMDPNTIQQALTKVNDTNQALNGNQKLADAKQDAKTTLGTLDHLNDAQKQALTTQVEQAPDIATVNNVKQNAQNLNNAMTNLNNALQDKTETLNSINFTDADQAKKDAYTNAVSHAEGILSKANGSNASQTEVEQAMQRVNEAKQALNGNDNVQRAKDAAKQVITNANDLNQAQKDALKQQVDAAQTVANVNTIKQTAQDLNQAMTQLKQGIADKDQTKANGNFVNADTDKQNAYNNAVAHAEQIISGTPNANVDPQQVAQALQQVNQAKGDLNGNHNLQVAKDNANTAIDQLPNLNQPQKTALKDQVSHAELVTGVNAIKQNADALNNAMGTLKQQIQANSQVPQSVDFTQADQDKQQAYNNAANQAQQIANGIPTPVLTPDTVTQAVTTMNQAKDALNGDEKLAQAKQEALANLDTLRDLNQPQRDALRNQINQAQALATVEQTKQNAQNVNTAMSNLKQGIANKDTVKASENYHDADADKQTAYTNAVSQAEGIINQTTNPTLNPDEITRALTQVTDAKNGLNGEAKLATEKQNAKDAVSGMTHLNDAQKQALKGQIDQSPEIATVNQVKQTATSLDQAMDQLSQAINDKAQTLADGNYLNADPDKQNAYKQAVAKAEALLNKQSGTNEVQAQVESITNEVNAAKQALNGNDNLANAKQQAKQQLANLTHLNDAQKQSFESQITQAPLVTDVTTINQKAQTLDHAMELLRNSVADNQTTLASEDYHDATAQRQNDYNQAVTAANNIINQTTSPTMNPDDVNGATTQVNNTKVALDGDENLAAAKQQANNRLDQLDHLNNAQKQQLQSQITQSSDIAAVNGHKQTAESLNTAMGNLINAIADHQAVEQRGNFINADTDKQTAYNTAVNEAAAMINKQTGQNANQTEVEQAITKVQTTLQALNGDHNLQVAKTNATQAIDALTSLNDPQKTALKDQVTAATLVTAVHQIEQNANTLNQAMHGLRQSIQDNAATKANSKYINEDQPEQQNYDQAVQAANNIINEQTATLDNNAINQAATTVNTTKAALHGDVKLQNDKDHAKQTVSQLAHLNNAQKHMEDTLIDSETTRTAVKQDLTEAQALDQLMDALQQSIADKDATRASSAYVNAEPNKKQSYDEAVQNAESIIAGLNNPTINKGNVSSATQAVISSKNALDGVERLAQDKQTAGNSLNHLDQLTPAQQQALENQINNATTRGEVAQKLTEAQALNQAMEALRNSIQDQQQTEAGSKFINEDKPQKDAYQAAVQNAKDLINQTNNPTLDKAQVEQLTQAVNQAKDNLHGDQKLADDKQHAVTDLNQLNGLNNPQRQALESQINNAATRGEVAQKLAEAKALDQAMQALRNSIQDQQQTESGSKFINEDKPQKDAYQAAVQNAKDLINQTGNPTLDKSQVEQLTQAVTTAKDNLHGDQKLARDQQQAVTTVNALPNLNHAQQQALTDAINAAPTRTEVAQHVQTATELDHAMETLKNKVDQVNTDKAQPNYTEASTDKKEAVDQALQAAESITDPTNGSNANKDAVDQVLTKLQEKENELNGNERVAEAKTQAKQTIDQLTHLNADQIATAKQNIDQATKLQPIAELVDQATQLNQSMDQLQQAVNEHANVEQTVDYTQADSDKQNAYKQAIADAENVLKQNANKQQVDQALQNILNAKQALNGDERVALAKTNGKHDIDQLNALNNAQQDGFKGRIDQSNDLNQIQQIVDEAKALNRAMDQLSQEITDNEGRTKGSTNYVNADTQVKQVYDETVDKAKQALDKSTGQNLTAKQVIKLNDAVTAAKKALNGEERLNNRKAEALQRLDQLTHLNNAQRQLAIQQINNAETLNKASRAINRATKLDNAMGAVQQYIDEQHLGVISSTNYINADDNLKANYDNAIANAAHELDKVQGNAIAKAEAEQLKQNIIDAQNALNGDQNLANAKDKANAFVNSLNGLNQQQQDLAHKAINNADTVSDVTDIVNNQIDLNDAMETLKHLVDNEIPNAEQTVNYQNADDNAKTNFDDAKRLANTLLNSDNTNVNDINGAIQAVNDAIHNLNGDQRLQDAKDKAIQSINQALANKLKEIEASNATDQDKLIAKNKAEELANSIINNINKATSNQAVSQVQTAGNHAIEQVHANEIPKAKIDANKDVDKQVQALIDEIDRNPNLTDKEKQALKDRINQILQQGHNGINNAMTKEEIEQAKAQLAQALQDIKDLVKAKEDAKQDVDKQVQALIDEIDQNPNLTDKEKQALKYRINQILQQGHNDINNALTKEEIEQAKAQLAQALQDIKDLVKAKEDAKNAIKALANAKRDQINSNPDLTPEQKAKALKEIDEAEKRALQNVENAQTIDQLNRGLNLGLDDIRNTHVWEVDEQPAVNEIFEATPEQILVNGELIVHRDDIITEQDILAHINLIDQLSAEVIDTPSTATISDSLTAKVEVTLLDGSKVIVNVPVKVVEKELSVVKQQAIESIENAAQQKINEINNSVTLTLEQKEAAIAEVNKLKQQAIDHVNNAPDVHSVEEIQQQEQAHIEQFNPEQFTIEQAKSNAIKSIEDAIQHMIDEIKARTDLTDKEKQEAIAKLNQLKEQAIQAIQRAQSIDEISEQLEQFKAQMKAANPTAKELAKRKQEAISRIKDFSNEKINSIRNSEIGTADEKQAAMNQINEIVLETIRDINNAHTLQQVEAALNNGIARISAVQIVTSDRAKQSSSTGNESNSHLTIGYGTANHPFNSSTIGHKKKLDEDDDIDPLHMRHFSNNFGNVIKNAIGVVGISGLLASFWFFIAKRRRKEDEEEELEIRDNNKDSIKETLDDTKHLPLLFAKRRRKEDEEDVTVEEKDSLNNGESLDKVKHTPFFLPKRRRKEDEEDVEVTNENTDEKVLKDNEHSPLLFAKRRKDKEEDVETTTSIESKDEDVPLLLAKKKNQKDNQSKDKKSASKNTSKKVAAKKKKKKAKKNKK

SEQ ID NO: 3 - protein, 371 aa, Staphylococcus saprophyticus
MIIGIPKEIKNNENRVSLSPSGVHALVEQGHTVIVEKSAGLGSYFEDVDYTEAGASIVNEQAEVWNVDMVMKVKEPLEEEFQYFKEGLILFTYLHLANEEKLTRALLENKVVGIAYETVQLPDRTLPLLTPMSEVAGRMSAQIGAEFLQKYKGGMGILLGGVPGVSKGRVSIIGGGQAGTNAAKIALGLGADVTILDVNPKRLQELEDLFDGRVHTIMSNPLNIEQCVKDSDLVIGAVLIPGAKAPNLVTEDMVKEMRDGAVIVDIAIDQGGIFETTDRISTHDDPTYKKHGVVHYAVANMPGAVPRTSTIALNNATLPYAQQLASKGYLKALQDNHALSLGLNTINGELTNKGVAEALNLSYTDIESALK

SEQ ID NO: 4 - protein, 204 aa, unidentified
  REGION 1..204: note = Bacilli sp.
MGNLDFDHEGYRKLLPLKDFKHFIKHSDSKTRNHFIGMAFNNKTDVFSRNFSNKMINIFPKNEFYIHIQQFPENDSPSYMVYNTDITAFIQFRLVGRSENKYLENIIRDYGVLIEVEALHSFKKGMGAMLVSQLEKLSDKLFIPIYLYDTNLKDELYYQDLGFFDTTKKGNHGEPLLVYKPKNLETNKKEKPIKSLFKKVFNIN

SEQ ID NO: 5 - protein, 370 aa, Staphylococcus sp.
MEQRTRLALLKEIAEFLNEETEMYSMTQGALKYLIEGSNFTTGWIFFINSVGEHELVSHVALPQSLTADHCHYIKDGSCWCVKAFNQRRLMKASNIVNCSRINLASKAFPSQNDNITHHATVPLKSGQEQFGILNVASPNTEIYSDEDLELLESVAFQLGSAIKRIYLTDREKEAAKINERNRLARDLHDSVNQMLFSVKLTAHAAYGMSNESIAKQAFKTIEETSQNAVNEMRALIWQLKPVGLEQGLIHALTAYSKLMHIQLNVNVEGLIDLSNEIEENIYRALQECINNVKKHADTNKMDLTLKQMNDILYIDVIDYGQGFEIDNVQIASSHGINNIKQRVKLLRGKVTFHSQPTKGTQIQFTIPIK

SEQ ID NO: 6 - protein, 251 aa, Staphylococcus sp.
MLKGHLHSVESLGTVDGPGLRYILFTQGCLLRCLYCHNPDTWKISEPSREVTVDEMVNEILPYKPYFDASGGGVTVSGGEPLLQMPFLEKLFAELKENGVHTCLDTSAGCANDTKAFQRHFEELQKHTDLILLDIKHIDNDKHIRLTGKPNTHILNFARKLSDMKQPVWIRHVLVPGYSDDKDDLIKLGEFINSLDNVEKFEILPYHQLGVHKWKTLGIAYELEDVEAPDDEAVKAAYRYVNFKGKIPVEL

SEQ ID NO: 7 - protein, 328 aa, Staphylococcus sp.
MLVALNEEKERVLATTALRKTQYFCPVCGKQVILKRGLKVISHFAHKHLAEQKCFNNESIKHYKSKLILAQMIQQQGCKVEIEPFLKEIKQIPDILINNKYVIELQYSPIPYKQILQRTEGLKKMGYKVSWLLNDVDYCHNKVKFNHFQSMFINPITRKLHTFNLEKKQIIMFQQIQYLGGHKYVAEKKNAKISELFNEAPCDYHAVYKLSKFAINQYIKYCRWQNSVLEPTLSAMYQLQLTDHEVVHNYGYIFPEQIYIENHPIEWQLQVDLWLKNGKSKLVSDNLNYFKLKKFIVALESKTAIIEKLINNYLNISSDRGNDVQILF

SEQ ID NO: 8 - protein, 179 aa, Staphylococcus carnosus
MSRLKEKFNTEVTENLMKEFNYSSVMEVPKIDKIVVNMGVGDAVQNSKVLDNAVEELELITGQKPLITKAKKSVATFRLREGMPIGAKVTLRGDRMYEFLDKLIAVSLPRVRDFHGVSKKAFDGRGNYTLGIKEQLIFPEIDYDKVSKVRGMDIVIVTTANTDEEGRELLKQFGMPFQK

SEQ ID NO: 9 - protein, 312 aa, Staphylococcus saprophyticus
MTKKKISIIGAGNTGATLAFIVAQHELADVVLIDRPDNEGQVKGKALDIFESSPVYGFDAKVTGSVNYADTADSDIVVITAGSPRKPGMSRDDLVQINEKVMFDVTKEIVKYSPDCKIIVLTNPVDAMTYSVLKASGFPKERVIGQSGVLDTARYQSFIAEALNVSIKDIRGLVLGGHGDTMVPLVNSTNVNGVPLHQLLNQTQIEQIVERTRKGGAEIVALLGNGSAYYAPASAVFEMIEAILKDQHRLLPSIALLEGEYGFSDICLGVPTVLSEKGIENIVELALSDNEQAQLRISADSVEEVKQALKNQ

SEQ ID NO: 10 - protein, 508 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNKKPGKEDGNKPGKEDNKKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 11 - protein, 372 aa, Staphylococcus aureus
MLVAVVKELKQGEGRVACTPENVRKLTDAGHKVIVEKNAGIGSGFSNDMYEKEGAKIVTHEQAWEADLVIKVKEPHESEYQYFKKNQIIWGFLHLASSKEIVEKMQEVGVTAISGETIIKNGKAELLAPMSAIAGQRSAIMGAYYSEAQHGGQGTLVTGVHENVDIPGSTYVIFGGGVAATNAANVALGLNAKVIIIELNDDRIKYLQDMYAEKDVTVVKSTPENLAEQIKKADVFISTILIPGAKPPKLVTREMVKSMKKGSVLIDIAIDQGGTIETIRPTTISDPVYEEEGVIHYGVPNQPGAVPRTSTMALAQGNIDYILEICDKGLEQAIKDNEALSTGVNIYQGQVTNQGLASSHDLDYKEILNVIE

SEQ ID NO: 12 - protein, 470 aa, Staphylococcus saprophyticus
METNGIKLTPRDIVSKLNEYIVGQDDAKRKVAIALRNRYRRSLLTEEEKQEVAPKNILMIGPTGVGKTEIARRMARLVGAPFIKVEATKFTEVGYVGRDVESMVRDLVDVAVRLVKDQKKALVQDEAQDKANEKLVKLLVPSMKKKANNNTNSNNPLESLFGGSIPNFGQNNDDEEETPTDEVKTKRSEIKQQLLNGQLEDEKVRLKVEQDPAAMGMLGTNQNQQMQDMMNQLMPKKKVEREVPVKTARKILTDEFADELIDQETANQEAIELAEQMGIIFIDEIDKVATNNQNSGQDVSRQGVQRDILPILEGSMVQTKYGTVNTEHMLFIGAGAFHVSKPSDLIPELQGRFPIRVELESLSVEDFVRILTEPKLSLIKQYEALLQTEQVTVKFTDEAIKRLAEIAFQVNQDTDNIGARRLHTILEKMLEDLSFEAPSMPNAVVDITPQYVDDKLKSISTNKDLSAFIL

SEQ ID NO: 13 - protein, 89 aa, Staphylococcus saprophyticus
MAKKSKIAKEQKREKLVAQYYELRKELKDKGDYEALRKLPRDSSPTRLTRRCKVTGRPRGVYRKFGMSRIALRDYAHKGQIPGVKKSSW

SEQ ID NO: 14 - protein, 44 aa, unidentified
  REGION 1..44: note = Eubacteria sp.
MEGLFNAIKDTVTAAINNDGAKLGTSIVSIVENGVGLLGKLFGF

SEQ ID NO: 15 - protein, 501 aa, Staphylococcus aureus
MPGAFMILFILTVVAVIATWVIPAGAYSKLSYEPSSQELKIVNPHNQVKKVPGTQQELDKMGVKIKIEQFKSGAINKPVSIPNTYERLKQHPAGPEQITSSMVEGTIEAVDIMVFILVLGGLIGVVQASGSFESGLLALTKKTKGHEFMLIVFVSILMIIGGTLCGIEEEAVAFYPILVPIFIALGYDSIVSVGAIFLASSVGSTFSTINPFSVVIASNAAGTTFTDGLYWRIGACIVGAIFVISYLYWYCKKIKKDPKASYSYEDKDAFEQQWSVLKDDDSAHFTLRKKIILTLFVLPFPIMVWGVMTQGWWFPVMASAFLIFTIIIMFIAGTGKSGLGEKGTVDAFVNGASSLVGVSLIIGLARGINLVLNEGMISDTILHFSSSLVQHMSGPLFIIVLLFIFFCLGFIVPSSSGLAVLSMPIFAPLADTVGMPRFVIVTTYQFGQYAMLFLAPTGLVMATLQMLNMRYSHWFRFVWPVVAFVLIFGGGVLITQVLIYS

SEQ ID NO: 16 - protein, 539 aa, Staphylococcus aureus
MVKQLKFSEDARQAMLRGVDQLANAVKVTIGPKGRNVVLDKEFTAPLITNDGVTIAKEIELEDPYENMGAKLVQEVANKTNEIAGDGTTTATVLAQAMIQEGLKNVTSGANPVGLRQGIDKAVKVAVEALHENSQKVENKNEIAQVGAISAADEEIGRYISEATEKVGNDGVITIITIEESNRLNTELELGMQFDRGYQSPYMVTDSDKMVAELERPYILVTDKKISSFQDILPLLEQVVQSNRPILIVADEVEGDALTNIVLNRMRGTFTAVAVKAPGFGDRRKAMLEDLAILTGAQVITDDLGLDLKDASIDMLGTASKVEVTKDNTTVVDGDGDENSIDARVSQLKSQIEETESDFDREKLQERLAKLAGGVAVIKVGAASETELKERKLRIEDALNSTRAAVEEGIVAGGGTALVNVYQKVSENEAEGDIETGVNIVLKALTAPVRQIAENAGLEGSVIVERLKNAEPGVGFNGATNEWVNMLRRGIVDPTKVTRSALQHAASVAAMFLTTEAVVASIPEKNNDQPNMGGMPGMM

SEQ ID NO: 17 - protein, 109 aa, Staphylococcus sp.
MINETKYMRQQITNLIQIIDTIKYNTLMTDWNIQTHIYKFNQIVTNELNKFKGFETSYIINENQVFYYEIITLLNKRPLRQVDYGNKIQYLNFYHTELSNALFAIKFAH

SEQ ID NO: 18 - protein, 61 aa, Lactobacillus iners
MNYKWILCPVCGNKTRLKIREDTELKKFPLYCPKCRQENLIEIKQFKVTVITEPDAKTQSR

SEQ ID NO: 19 - protein, 401 aa, Staphylococcus sp.
MKEKIVLAYSGGLDTSVAVQWLIDKGYDVVACCLDVGEGKDLDIVYKKALDMRAVECHIIDATKEFSDEYVSYAIKGNLMYENAYPLVSALSRPLIAKKLVEIAEKTNSVGIAHGCTGKGNDQVRFEVAIKALNPSLKAFAPVREWAWSREEEIDYAIKHNIPVSINHDSPYSIDQNLWGRANECGILEDPYAAPPEDAFDLTNALEETPDTADEIILTFDKGIPVQIDGKTYELDDLILTLNALAGKHGIGRIDHVENRLVGIKSREIYEAPAAEVILKAHKALETITLTKDVAHFKPIIEKQFAEQLYNGLWFSPLTDSLKLFIDSTQQYVSGDVRIKLFKGNAIVNGRKSPYTLYDEKLATYTKEDAFNQDAAVGFIDIYGLPTQVNAMLHGGYSNEQ

SEQ ID NO: 20 - protein, 459 aa, Staphylococcus aureus
MSNKAWGGRFEVQPEEWVDDFNASITFDQTLINQDIEGSIAHATMLANQGIISQQDSEQIIQGLKSIQHDYHQDQIQFSASLEDIHLNIEHELIKRIGDAGGKLHTGRSRNDQVATDMHLYTKKQVQDIIALIKSLQSVIVDIASNNVDTIMPGYTHLQRAQPISFAHHIMTYFWMLQRDQQRFEDSLKRIDINPLGAAALSGTTYPIDRHETTALLNFGSLYENSLDAVSDRDYIIETLHNISLTMVHLSRFAEEIIFWSTDEAKFITLSDAFSTGSSIMPQKKNPDMAELIRGKVGRTTGHLMSMLMTLKGLPLAYNKDMQEDKEGLFDAVHTIKGSLRIFEGMIQTMTINKERLNQTVKEDFSNATELADYLVTKNIPFRTAHEIVGKIVLECIQQGHYLLDVPLATYQQHHSSIDADIYDYLQPENCLKRRQSYGSTGQSSVKQQLDVAKQLLSQ

SEQ ID NO: 21 - protein, 647 aa, Staphylococcus aureus
MKINKLTISNFAGIKEEKFNFDGKDAKIYGNNATGKTTTATALQWLLFDKGLDGSTKSFNPVPLNEKNEENYELIPTVFAEFEIDGKITTFKKESHPKYTINQKTNRKEYSRSRTKKQYINDESIKVKDYKARIDELIDEDVFKLITNPQAFNLLDWKKRRSLLFEIAKPINDEDVIKTNDDFKELNNILGDHEIETKKKILTDKIKQINKDIKDIPIRINQTQQNKQDVPEFDNDRYAIIKQEIEQLENERIDIQNGAEEINLRNQLADKQSELKRIEDNNSASNENKIHTLTNELHVENGTVANLKTRLKQNKQQIAHEENRRNQLLENHKGLKSDLEKAENQKFEYLDDNVCSCCGQQLLAEQVNEAREKALQKFNASKSKELETIQVSINHIISEGKKIKPIIEKLVDDNNNLQIKINEAEERSARIQNKINKLKTTHVDVTQTDEYKAVMLEINEINQKRSNIRKTIKDNVSGIDDKISELTQEKSEIEVSRSIEKSNKHLDDVISELRNEEDRLLDEKEKYSHDLYILKEFTTTKVKMLTENINNEFEIAEFKLFNTLVNGELEETCSTTVNGVEYDSGLNNASRINVGLDIINTLSKHFKVTAPIFIDNAESVTELIKTESQQIQLIVNEQDKKLRMETI

SEQ ID NO: 22 - protein, 330 aa, Staphylococcus aureus
MTKIMFFGTRDYEKEMALNWGKKNNVEVTTSKELLSSATVDQLKDYDGVTTMQFGKLEDDVYPKLESYGIKQIAQRTAGFDMYDLDLAKKHNIVISNVPSYSPETIAEYSVSIALQLVRRFPDIERRVQAHDFTWQAEIMSKPVKNMTVAIIGTGRIGAATAKIYAGFGATITAYDAYPNKDLDFLTYKDSVKEAIKDADIISLHVPANKESYHLFDKAMFDHVKKGAILVNAARGAVINTPDLIAAVNDGTLLGAAIDTYENEAAYFTNDWTNKDIDDKTLLELIEHERILVTPHIAFFSDEAVQNLVEGGLNAALSVINTSTCETRLN

SEQ ID NO: 23 - protein, 242 aa, Staphylococcus sp.
MPVIKINNLNKVFGDNEVLKDINLEINQGEVVAIIGPSGSGKSTLLRCMNLLEVPTKGQVIFEGNDLTEKGTQVDKLRQKMGMVFQNFNLFPHKKVVDNIILAPKLLKKDNNDELHKEALSLLDKVGLKEKADVYPNQLSGGQKQRVAIARALAMHPDVILFDEPTSALDPEVVGDVLKVMKDLAKEGMTMVVVTHEMGFAKDVSDKVIFMADGVVVESGTPVEIFEQPQHERTQNFLARVL

SEQ ID NO: 24 - protein, 310 aa, Staphylococcus sp.
MAKIVVALGGNALGKSPQEQLKLVKNTAKSLVGLITKGHEIVISHGNGPQVGSINLGLNYAAEHNQGPAFPFAECGAMSQAYIGYQLQESLQNELHSIGMDKQVVTLVTQVEVDENDPAFNNPSKPIGLFYNKEEAEQIQKEKGFTFVEDAGRGYRRVVPSPQPISIIELESIKTLIKNDTLVIAAGGGGIPVIREQHDGFKGIDAVIDKDKTSALLGANIQCDQLIILTAIDYVYINFNTENQQPLKTTNVDELKRYIDENQFAKGSMLPKIEAAISFIENNPKGSVLITSLNELDAALEGKVGTVIKK

SEQ ID NO: 25 - protein, 493 aa, Staphylococcus sp.
MKKIFMMVHELDVNKGGMTSSMFNRSKEFYDADIPADIVTFDYKGNYDEIIKALKKQGKMDRRTKMYNVFEYFKQISNNKHFKSNKLLYKHISERLKNTIEIEESKGISRYFDITTGTYIAYIRKSKSEKVIDFFKDNKRIERFSFIDNKVHMKETFNVDNKVCYQVFYDEKGYPYISRNINANNGAVGKTYVLVNKKEFKNNLALCVYYLEKLIKDSKDSIMICDGPGSFPKMFNTNHKNAQKYGVIHVNHHENFDDTGAFKKSEKYIIENANKINGVIVLTEAQRLDILNQFDVENIFTISNFVKIHNAPKHFQTEKIVGHISRMVPTKRIDLLIEVAELVVKKDNAVKFHIYGEGSVKDKIAKMIEDKNLERNVFLKGYTTTPQKCLEDFKLVVSTSQYEGQGLSMIEAMISKRPVVAFDIKYGPSDFIEDNKNGYLIENHNINDMADKILKLVNNDVLAEEFGSKARENIIEKYSTESILEKWLNLFNS

SEQ ID NO: 26 - protein, 95 aa, Staphylococcus sp.
MEKGIFNYDNANVLKLDTNQLNENIKVIDDIFKNYEQIEPTIEIENGNTKLKLNGYFIASIISPLNLNKLNNLYVEEEFYHTYNELIVKYTEVKE

SEQ ID NO: 27 - protein, 104 aa, Staphylococcus sp.
METKEKIKVTTLDELTPLIGKKVIVKGKEIGLFLTESGTIHAIHNICPHKQGPLSEGTVSGEYVFCPLHDQKIDLNTGIVQEPDEGCVDVYEVEVTDGNVYICL

SEQ ID NO: 28 - protein, 186 aa, Staphylococcus sp.
MTHRALLVVDYSYDFIADDGLLTCGKPGQNIEDFIVSRINDFNYYQDHIFFLMDLHYLHDIHHPESKLFPPHNIVDTSGRELYGKVGKLYETIKAQPNVHFIDKTRYDSFFGTPLDSLLRERSINQVEIVGVCTDICVLHTAISAYNLGYKISVPAEGVASFNQKGHEWALAHFKNSLGAEVEQHV

SEQ ID NO: 29 - protein, 306 aa, Staphylococcus sp.
MTENNKLQTIEQQLVQEKNVSDNVLNKVRVLESQGNLELPNDYSPSNAMKQAWLQISQDNKLMSCNDTSKANALLDMVTQGLNPAKNQCYFIPYGNKMQLQRSYHGNVMMLKRDAGAQDVVAQVIYKGDTFKQEMGETGRIKAIKHEQDFFNIDKENIIGAYCTIVFNDGRDNYIEVMTIEQIKQAWMQSSMIKDEKALQNSKTHNNFKEEMAKKTVINRAAKRYINTSTDSNLFKYAQESEQRQRKEVLDAEVEENANQEQLDFEQPVLEEAQYTELENDKPIDVSDFEEIKEPATEKESEEEPF

SEQ ID NO: 30 - protein, 313 aa, Staphylococcus aureus
MKEKIVIALGGNAIQTKEATAEAQQTAIRRAMQNLKPLFDSPARIVISHGNGPQIGSLLIQQAKSNSDTTPAMPLDTCGAMSQGMIGYWLETEINRILTEMNSDRTVGTIVTRVEVDKNDPRFDNPTKPIGPFYTKEEVEELQKEQPDSVFKEDAGRGYRKVVASPLPQSILEHQLIRTLADGKNIVIACGGGGIPVIKKENTYEGVEAVIDKDFASEKLATLIEADTLMILTNVENVFINFNEPNQQQIDDIDVATLKKYVAQGKFAEGSMLPKIEAAIRFVESGENKKVIITNLEQAYEALIGNKGTHIHM

SEQ ID NO: 31 - protein, 372 aa, Staphylococcus sp.
MLVAVVKELKQGEGRVACTPENVRKLTDAGHKVIVEKNAGIGSGFSNDMYEKEGAKIVTHEQAWEADLVIKVKEPHESEYQYFKKNQIIWGFLHLASSKEIVEKMQEVGVTAISGETIIKNGKAELLAPMSAIAGQRSAIMGAYYSEAQHGGQGTLVTGVHENVDIPGSTYVIFGGGVAATNAANVALGLNAKVIIIELNDDRIKYLEDMYAEKDVTVVKSTPENLAEQIKKADVFISTILIPGAKPPKLVTREMVKSMKKGSVLIDIAIDQGGTIETIRPTTISDPVYEEEGVIHYGVPNQPGAVPRTSTMALAQGNIDYILEICDKGLEQAIKDNEALSTGVNIYQGQVTNQGLATSHDLDYKEILNVIE

SEQ ID NO: 32 - protein, 318 aa, Staphylococcus carnosus
MKNIKANKVVLVGDGAVGSSYAFAMVAQGVADEFVIVDIAVDKVKGDVLDLNHGMPYGESPSIIKAGSYEDCSDADLVVITAGAPQKPGETRLDLVEKNTKIFKSIVGQIMDSGFDGIFLIAANPVDVLTYVTKKVSGLPKERVIGSGTILDTARFKYELGAEFGIAPESVNASIIGEHGDSELAVWSQTSIAGQNLYDILKSNPEKEKRIEEIFLNTRDAAYDIIQAKGATYYGIAMGLLHISKAILRNQNLVLTVSSYLEGEYGNEDVYIGVPTLVNRAGAVKIYETSLNEKETKEFNHSVEVLKDITKSVDKLFQ

SEQ ID NO: 33 - protein, 459 aa, Staphylococcus aureus
MDLDTITSISTPMGEGAIGIVRLSGPQAVEIADKLYKGKHLLNDVPSHTINYGHIIDPESKEVVEEVMVSVLRAPKTFTREDIIEINCHGGILTINRVLELTMTYGARLAEPGEFTKRAFLNGRIDLSQAEAVMDFVRSKTDRASKVAMNQIEGRLSDLIKKQRQSILEILAQVEVNIDYPEYDDVEDATTEFLLEQSKEIKQEINRLLDTGAQGKIMREGLSTVIVGKPNVGKSSMLNNLIQDNKAIVTEVAGTTRDVLEEYVNVRGVPLRLVDTAGIRETEDIVEKIGVERSRKALSQADLILFVLNNNEALTQEDYTLYEVVKNEDVIVIVNKMDLEQNIDINEVKDMIGDTPLIQTSMLKQEGIDELEIQIRDLFFGGEVQNQDMTYVSNSRHISLLKQARQTIQDAIDAAESGVPMDMVQIDLTRTWEILGEIIGETASDELIDQLFSQFCLGK

SEQ ID NO: 34 - protein, 340 aa, Staphylococcus epidermidis
MKAAVVTKDHRVSIEEKRLRELRPGEALVKTEYCGVCHTDLHVKNADFGDVTGVTLGHEGIGRVIKIADNVDSLKVGDRVSIAWMYAACGNCEYCTTGRETLCRDVLNAGYTVDGAMAEEVIVDANYAVKVPENLDPAAASSITCAGVTTYKAVKVSGIEPGQWLGVFGVGGLGNLALQYAKNVMGAKVVAFDINDDKLNFAKELGADAIINSTNVDPIEEVNRLTNNKGLDATVITAVAKTPFNQAVDVVKAGARVVAVGLPVDKMDLDIPRLVLDGIEVVGSLVGTRQDLREAFQFAAENKVIPKIQLRQLSEINDIFDEMEKGTITGRMVIDMKSTH

SEQ ID NO: 35 - protein, 591 aa, Staphylococcus sp.
MKKIISIAIIVLALVLSGCGVPTKSEVAQKSSKVEVKGERPTIHFLGQASYENDMNIVKDQLENAGFNVKMNIQPDYGSYRTQRQAGNYDIQIDDWMTVFGDPNYAMTALFSSTGSNSLLKDKHVDQLLNKASTQNEADVKQTYKQIEDEVVFDKGYMAPLYGSKKNLVYDNKVLDKNSVGLPNSRALIWQQFDYNNSRERDTRPLVMTQQDGEIPTLDPIRSIAPSVYSINMNMYTRLLLLDENDHLTTKGSLSRDYAVNKDNKAFYFLLRDDDYFAKVVNGQARNTGERVSAEDVKFSLDRARDKKSVPNNNTYNMHKHINDIKILKDEDIDQLRKEKDKDDNSIYDKLIKAYNVKSLTTDGQKVNNKDGIYQIVKITTDQSMPREVNYLTHSSAGILSKKFVNQVNQEYPKGYGDSSTIPANSDGKNALYASGAYIMTQKNAYQATFQRNPGFNETEKGSYGPAKIKNITLKFNGDPNNALSELRNHSIDMLADVNQKHFDLIKSDKNLSIIRKNGRKSVFLMLNIKKGIFKTHPNLRQAVVNAIDQDQFIKFYRGDKFKIASPITPLVDTGNEQRQDLEKVEKAINQ

SEQ ID NO: 36 - protein, 1435 aa, Mycoplasmopsis pulmonis
MQESFLNFCQEINFDIPEYLKNTKIANPYHDEEKSFFSCEIDFLETPKFEDFKSFYLKTKNFLNQLVKEQKLLFKIENIFYEKSEIKKYLDWIAEHFFKNFDFKNTFRIDDLKVNLDGKIFLTAHSKHSFDLYKDFVEFANKKMQSFGFENFLLKLELNQIEITNNQPLVSSINSQVKTQASSENQSFKKSNFYNKKRHIQLSLKELIKTQEMFVSVVGMVFKKEIITTNSKTKIFKISITDFQEATRAQKFSYLEKDQEIFDSIQINDWVNVCGEIQLEKMSLKQFIKIEKIEKIRSPIKDRQDNEKEKRIELSFRSKMSTMDGILSPLDIVKQAKKFNHSSIAILDHNSVQAFPDFHNLTYKDKDFKVIYGMTLSVISKRNKIFVEPLKDKFFDYKILDQEYVVYDIETTGLSPMLNELIQFGASVIKNGRIIETHHFFIKPKSKLDSFTTKLTGITQEHLEKGYELQEALEKISSIFKARIMVAHNAAFDHNFLKQKFIDNNIEFEEMISIDTLNLAKVLNPIYRSYRLGEVASKLSVVYDPSIAHRADYDSSVLTNIFILQMSHLKEKGINLFKELNSLSSEKFYSKMFAKDISIIAKNQAGLKELFKINSDVLTKYYFANPKMFWEDIKRSKNLLIGSGGLNSPLIDKLLFSTQEDVLNEIDKYDYIEIPSPNNFSHFWTTKFSDQQIKIQLQKLIKWAKEKNKIIVAIGEPRYNEKWQKIIHEIYINSTGIENSLHPLFIIKKDMDTFYPEQIYKTTNEMKQEFSFLYDPELIEEIVVKNPNLISSQIEKIQVIKDKLYTPKFDDSHIKLKKLVYENAWKKYGKNLPEIIEQRIEKELTPILNYGFDVIYWISSILVQKSLSDGYLVGSRGSIGSSLVATLSGITEVNPLAPHYICSKCQYFELVKDPMTNSGFDLDDKKCPKCNSFLDKDGQTIPFETFLGFKADKVPDIDLNFSGEYQGKIHDEVKRLFGSKHTFRAGTISTVAEKTAYGYIKKYLETASLDYSDNFVDFLTEKSTGVKRTTGQHPGGIIIIPKEFDVEDFTPVNFPANDTESSWKTTHFDFHAIHDNVLKLDILGHDDPTALKMLEKLTGVNVKDIPKKDEKIISIFTSPKALGISSEEILGEKTGALGIPEFGTNFVRQMLSEAKPKTFADLVSISGLSHGTDVWINNAHYIIQSLGKTLDQVISCRDDIMVDLIKKGVPIDLSFTIMEQVRKGKGLSLEQKRKLIEHGIENWYIESMEKIKYMFPKAHATAYVLMAWRVAFYKVYYPLEYYATYFSTRTEFFDIEIMSKDKLTLESKIKELAYRENLRNDNQLTTKEKNTLPTLYIANEMKARGFNIQNINLKISLANDWIIDKNSKSLIPPFNVIDGLGETLAQKIVDSRNEKEFLSVEDFINRTGINSTLVEKFKSMGIFEQIPETNQIFLI

SEQ ID NO: 37 - protein, 46 aa, unidentified
  REGION 1..46: note = Eubacteria sp.
MLQKFRIAKEKNKLKLKLLKHASYCLERSNNPELLRAVAELLKKVN

SEQ ID NO: 38 - protein, 532 aa, Staphylococcus sp.
MLVNTFNPFDNLLLSSLIAAIPIVLFLLCLTVFKMKGIYAAITTLVVTLLIAIPFFKLPVGIASGAVVEGFFQGIIPIGYIVMMAVLLYKITVESGQFLTIQDSITNISQDQRIQVLLIGFAFNAFLEGAAGFGVPIAICALLLTQLGFNPLKAAMLCLVANAASGAFGAIGIPVGVVETLKLPGDVSVLGVSQSATLTLAIINFIIPFLLIFIIDGFRGVKETLPAILVVSITYTLTQGLLTVFSGPELADIIPPLLTMLALAVFSKKFQPKHIYRVNKDEEIEPAKAHSAKAVLHAWSPFIVLTVIVMIWSAPFFKNLFLPNGALSSLVFKFNLPGTVSEVTHKPLVLTLNIIGQTGTAILLTIIITILMSKKVNFKDAGRLFGVTFKELWLPVLTICFILAISKITTYGGLSAAMGQGIAKAGNVFPVLSPILGWIGVFMTGSVVNNNSLFAPIQASVAQQIGTSGSLLVSANTVGGVAAKLISPQSIAIATAAVKQVGKESELLKMTLKYSVCLLIFICIWTFILSLL

SEQ ID NO: 39 - protein, 168 aa, Staphylococcus sp.
MKKLLTASIIACSVVMGVGIANTSAEAASGNSIDTVKQLIKGDQSLENVKIGESIKDVLTKYKNPMYSYNEDGTEHYYEFHTKKGMLLVTTDGKKNNGKVTHISMMYNDANGPTYQAVKNYVGKAVTHTEYSKVAGNFGYIEKGKTTYQFASAPKDKNIKLYRIDLEK

SEQ ID NO: 40 - protein, 691 aa, Staphylococcus sp.
MLRGQEERKYSIRKYSIGVVSVLAATMFVVTSHEAQASEKIPTTNAAAQKETLNQPGEQGNAITSHQMQSGKQLDDMHKENGKSGTVTEGKDMLQSSKHQSTQNSKIIRTQNDNQVKQDSERQGSKQSHQNNATNKTERQNDQIQNTHHAERNGSQSTTSQSNDVDKSQPSIPAQKVIPNHDKAAPTSTTPPSNDKTAPKSTKEQDATTDKHPNQQDTHQPAHQIIDAKQDDNVHQSNQKPQVGDLSKHIDGQNSPEKPTDKNTDNKQLIKDALQAPKTRSTTNAAADAKKVRPLKANQVQPLNKYPVVFVHGFLGLVGDNAPALYPNYWGGNKYKVIEELRKQGYNVHQASVSAFGSNYDRAVELYYYIKGGRVDYGAAHAAKYGHERYGKTYKGIMPNWEPGKKVHLVGHSMGGQTIRLMEEFLRNGNKEEIAYHKAHGGEISPLFTGGHNNMVASITTLATPHNGSQAADKFGNTEAVRKIMFALNRFMGNKYSNIDLGLTQWGFKQLPNESYIDYIKRVSKSKIWTSDDNAAYDLTLNGSAKLNNMTSMNPNITYTTYTGVSSHTGPLGYENPDLGTFFLMDTTSRIIGHDAREEWRKNDGVVPVISSLHPSNQPFVNVTNDEPATRRGIWQVKPIIQGWDHVDFIGVDFLDFKRKGAELANFYTGIINDLLRVEATESKGTQLKAS

SEQ ID NO: 41 - protein, 346 aa, Staphylococcus aureus
MTTNTVTLQTAHIVSLGDIEEAKASIKPFIRRTPLIKSMYLSQSITKGNVFLKLENMQFTGSFKFRGASNKINHLTDEQKEKGIIAASAGNHAQGVALTAKLLGIDATIVMPETAPQAKQQATKGYGAKVILKGKNFNETRLYMEELAKENGMTIVHPYDDKFVMAGQGTIGLEILDDIWNVNTVIVPVGGGGLIAGIATALKSFNPSIHIIGVQSENVHGMAESFYKRDLTEHRVDSTIADGCDVKVPGEQTYEVVKHLVDEFILVTEEEIEHAMKDLMQRAKIITEGAGALPTAAILSGKINNKWLEDKNVVALVSGGNVDLTRVSGVIEHGLNIADTSKGVVG

SEQ ID NO: 42 - protein, 646 aa, Staphylococcus aureus
MSSQKKKISLFAFFLLTVITITLKTYFSYYVDFSLGVKGLVQNLILLMNPYSLVALVLSVFLFFKGKKAFWFMFIGGFLLTFLLYANVVYFRFFSDFLTFSTLNQVGNVESMGGAVSASFKWYDFVYFIDTLVYLFILIFKTKWLDTKAFSKKFVPVVMAASVALFFLNLAFAETDRPELLTRTFDHKYLVKYLGPYNFTVYDGVKTIENNQQKALASEDDLTKVLNYTKQRQTEPNPEYYGVAKKKNIIKIHLESFQTFLINKKVNGKEVTPFLNKLSSGKQQFTYFPNFFHQTGQGKTSDSEFTMDNSLYGLPQGSAFSLKGDNTYQSLPAILDQKQGYKSDVMHGDYKTFWNRDQVYKHFGIDKFYDATYYDMSDKNVVNLGLKDKIFFKDSANYQAKMKSPFYSHLITLTNHYPFTLDEKDATIEKSNTGDATVDGYIQTARYLDEALEEYINDLKKKGLYDNSVIMIYGDHYGISENHNNAMEKLLGEKITPAKFTDLNRTGFWIKIPGKSGGINNEYAGQVDVMPTILHLAGIDTKNYLMFGTDLFSKGHNQVVPFRNGDFITKDYKYVNGKIYSNKNNELITTQPADFEKNKKQVEKDLEMSDNVLNGDLFRFYKNPDFKKVNPSKYKYETGPKANSKK

SEQ ID NO: 43 - protein, 748 aa, Staphylococcus epidermidis
MLETNNHTNAWQGFKTGRWNKNIDVREFIQLNYSLYEGDDEFLEGPTKATETLWDQVMQLSKEERERGGMWDMDTKVASTITSHDAGYLDKDLEKVVGVQTEKPFKRSMQPFGGIRMAKAACEAYGYELDPETEKIFTEYRKTHNQGVFDAYSREMLNCRKAGIITGLPDAYGRGRIIGDYRRVALYGVDFLMEQKLKDFNTMSTEMSEDVIRLREELSEQYRSLQDLKELGQKYGFDISRPATNFKEAVQWLYLAYLAAIKEQNGAAMSLGRTSTFLDIYAERDLQNGDITEQEVQEIIDHFIMKLRIVKFARTPEYNELFSGDPTWVTESIGGVGIDGRPMVTKNSFRFLHSLDNLGPAPEPNLTVLWSTRLPENFKIYCAKMSIKTSSIQYENDDLMRESYGDDYGIACCVSAMKIGKQMQFFGARANLAKALLYAINGGKDEKSGKQVGPSYEGIKSDVLDYDEVFERYEKMMDWLAGVYINSLNIIHYMHDKYSYERLEMALHDTEIIRTMATGIAGLSVAADSLSAIKYAQVKPIRNEEGLVTDFEIEGDFPKYGNNDSRVDEIAVDLVERFMTKLRSHKTYRNSEHTMSVLTITSNVVYGKKTGNTPDGRKAGEPFAPGANPMHGRDQKGALSSLSSVAKIPYDCCKDGISNTFSIVPKSLGKEEVDQNKNLTSMLDGYAMQHGHHLNINVFNRETLIDAMEHPEEYPQLTIRVSGYAVNFIKLTREQQLDVISRTFHESM

SEQ ID NO: 44 - protein, 105 aa, Staphylococcus sp.
MKKNFIGKSILSIAAISLTVSTFAGESHAQTKAEKYNEYQTNFKKQVNKKVVDAQKAVNLFKRTRTVATHRKAQRAVNLIHFQHSYEKKKLQRQIDLVLKYNTLK

SEQ ID NO: 45 - protein, 317 aa, Staphylococcus sp.
MNKFKGNKVVLIGNGAVGSSYAFSLVNQSIVDELVIIDLDTEKVRGDVMDLKHATPYSPTTVRVKAGEYSDCHDADLVVICAGAAQKPGETRLDLVSKNLKIFKSIVGEVMASKFDGIFLVATNPVDILAYATWKFSGLPKERVIGSGTILDSARFRLLLSEAFDVAPRSVDAQIIGEHGDTELPVWSHANIAGQPLKTLLEQRPEGKAQIEQIFVQTRDAAYDIIQAKGATYYGVAMGLARITEAIFRNEDAVLTVSALLEGEYDEEDVYIGVPAVINRNGIRNVVEIPLNDEEQSKFAHSAKTLKDIMAEAEELK

SEQ ID NO: 46 - protein, 616 aa, Staphylococcus sp.
MNQIEEALTGLISKDPAIVNENANKDSDTFSTMRDLTAGIVSKSYALNHLLPKHVADAHQRGDIHFHDLDYHPFQPLTNCCLIDAKNMLHNGFEIGNANVTSPKSIQTASAQLVQIIANVSSSQYGGCTVDRVDELLSTYARHNEEQHRNIAKQFVKESEIDRYVDQQVTKDINDAIESLEYEINTLYTSNGQTPFVTLGFGLGTDHLSRKIQQAILNTRIKGLGKDRTTAIFPKLVFSIKKGTNFSPQDPNYDIKQLALKCSTKRMYPDILNYDKLVEILGDFKAPMGCRSFLPSWKDAEGHFENNGRCNLGVVTLNLPRMALESAGNMTKFWEIFYERIDVLHDALLYRINRLKDAVPNNAPILYKSGAFNYKLKETDDVAELFKNKRATISMGYIGLYETATVFYGPNWETSQEAKAFTLEILKEMKRYQTKWTELYDIWFSIYSTPSESLTDRFCRLDQERFGDIKDITDKGYYQNSFHYDVRKDVTPFEKLDFEKDYPYYTSGGFIHYCEYPKLQHNLKALEAVWDYSYDKVGYLGTNIPIDHCYECDYDGDFEATEKGFKCPNCGNDNPKTVDVVKRTCGYLGNPVQRPVIKGRHKEICARVKHMKAPKE

SEQ ID NO: 47 - protein, 869 aa, Staphylococcus sp.
MLTIPEKENRGSKEQEVAIMIDALADKGKKALEALSKKSQEEINHIVHQMSLAAVDQHMVLAKLAHEETGRGIYEDKAIKNLYASEYIWNSIKDNKTVGIIGEDKEKGLTYVAEPIGVICGVTPTTNPTSTTIFKAMIAIKTGNPIIFAFHPSAQESSKRAAEVVLEAAMKAGAPKDIIQWIEVPSIEATKQLMNHKGIALVLATGGSGMVKSAYSTGKPALGVGPGNVPSYIEKTAHIKRAVNDIIGSKTFDNGMICASEQVVVIDKEIYKDVTNEFKAHQAYFVKKDELQRLENAIMNEQKTSIKPDIVGKSAVEIAELAGIPVPENTKLIIAEISGVGSDYPLSREKLSPVLALVKAQSTKQAFQICEDTLHFGGLGHTAVIHTEDETLQKDFGLRMKACRVLVNTPSAVGGIGDMYNELIPSLTLGCGSYGRNSISHNVSATDLLNIKTIAKRRNNTQIFKVPAQIYFEENAIMSLTTMDKIEKVMIVCDPGMVEFGYTKTVENVLRQRTEQPQIKIFSEVEPNPSTNTVYKGLEMMVDFQPDTIIALGGGSAMDAAKAMWMFFEHPETSFFGAKQKFLDIGKRTYKIGMPENATFICIPTTSGTGSEVTPFAVITDSETNVKYPLADFALTPDVAIIDPQFVMSVPKSVTADTGMDVLTHAMESYVSVMASDYTRGLSLQAIKLTFEYLKSSVEKGDKVSREKMHNASTLAGMAFANAFLGIAHSIAHKIGGEYGIPHGRANAILLPHIIRYNAKDPQKHALFPKYEFFRADTDYADIAKFLGLKGNTTEALVESLAKAVYELGQSVGIEMNLKSQGVSEEELNESIDRMAELAFEDQCTTANPKEALISEIKDIIQTSYDYKQ

SEQ ID NO: 48 - protein, 336 aa, Staphylococcus sp.
MRAAVVTKDHKVSIEDKKLRALKPGEALVQTEYCGVCHTDLHVKNADFGDVTGVTLGHEGIGKVIEVAEDVESLKIGDRVSIAWMFESCGRCEYCTTGRETLCRSVKNAGYTVDGAMAEQVIVTADYAVKVPEKLDPAAASSITCAGVTTYKAVKVSNVKPGQWLGVFGIGGLGNLALQYAKNVMGAKIVAFDINDDKLAFAKELGADAIINSKDVDPVAEVMKLTDNKGLDATVVTSVAKTPFNQAVDVVKAGARVVAVGLPVDKMNLDIPRLVLDGIEVVGSLVGTRQDLREAFEFAAENKVTPKVQLRKLEEINDIFEEMENGTITGRMVIKF

SEQ ID NO: 49 - protein, 116 aa, Staphylococcus carnosus
MSNHKLIEAVTKSQLRTDLPTFRPGDTVRVHVRIVEGTRERIQVFEGVVIKRHGGGISETFTVRKISSGVGVERTFPLHTPKIEKIEVKRRGKVRRAKLYYLRNLRGKAARIKEIR

SEQ ID NO: 50 - protein, 485 aa, Staphylococcus sp.
MKCLIRFILVLGLLISSAMVYINPAAYAEQDQKWEKIKERGELRVGLSADYAPMEFEHTVNGKTEYAGVDIDLAKKIAKDNNLKLKIVNMSFDSLLGALKTGKIDIIISGMTSTPERKKQVDFSDSYMMTKNIMLVKKDKVNDYKDIKDFNNKKVGAQKGTEQEKIAQTEIENASITSLSRLPDVILALKSGKVEGVVVEKPVAEAYLKQNPKLGISNVKFNEEEKDTVIAVPKDSPKLLSQINKTIKEVKDKGLIDKYMTNAANAMNDDSGFISKYGSFFLKGIKITILISLIGVALGSILGAFVALMKLSKIKIISWIASIYIEILRGTPMLVQVFIVFFGITAALGLDISALVCGTIALVINSSAYIAEIIRAGINAVDKGQMEAARSLGLNYRQTMKSVIMPQAIKNILPALGNEFVTLIKESSIVSTIGVGEIMFNAQVVQGISFDPFTPLLVAAALYFVLTFVLTRIMNMIEGRLNASD

SEQ ID NO: 51 - protein, 26 aa, Staphylococcus aureus
MAQDIISTIGDLVKWIIDTVNKFTKK

SEQ ID NO: 52 - protein, 452 aa, Staphylococcus sp.
MNFLKPAKHIKPLPENQIDDTYKRLRLQVFLGIFIGYAGYYLLRKNFSLAMPALQEQGFTKAELGFALSAVSIAYGFSKFFMGTVSDRSNARIFLVLGLVLTAIVNLLMGFVPFFTSGIGIMFVLLFLNGWFQGMGWPPSGRVLVHWFSVSERGSKTALWNVAHNVGGGIMAPIAAWGITTTAFINFGYLKGFEGVFIYPALLALIIAAISYILIRDTPQSQGLPPIEIYKNDFATSDKKTLETELTTKEILFKYVLNNKWVWAIAFANIFVYFVRYGVLDWAPVYLSEEKHFDLKASGWAYFLYEWAGIPGTLLCGYISDKLFKGRRGPAGFFFMLGVTVFVLIYWLNPPGNAWLDNVSLIAIGFLIYGPVMLIGLQALDYVPKKAAGTAAGLTGLFGYLFGAVMANIVLGAVVDKFGWDVGFILLTAISVFAMLSFILTWNKVGQETVHH

SEQ ID NO: 53 - protein, 333 aa, Staphylococcus aureus
MKNLRNRSFLTLLDFSRQEVEFLLTLSEDLKRAKYIGTEKPMLKNKNIALLFEKDSTRTRCAFEVAAHDQGANVTYLGPTGSQMGKKETTKDTARVLGGMYDGIEYRGFSQRTVETLAEYSGVPVWNGLTDEDHPTQVLADFLTAKEVLKKDYADINFTYVGDGRNNVANALMQGAAIMGMNFHLVCPKELNPTDELLNRCKNIAAENGGNILITDDIDQGVKGSDVIYTDVWVSMGEPDEVWKERLELLKPYQVNKEIMDKTGNPNVIFEHCLPSFHNADTKIGQQIFEKYGIREMEVTDEVFESKASVVFQEAENRMHTIKAVMVATLGEF

SEQ ID NO: 54 - protein, 234 aa, Staphylococcus aureus
MTENFILGRNNKLEHELKALADYINIPYSILQPYQSECFVRHYTKGQVIYFSPQESSNIYFLIEGNIIREHYNQNGDVYRYFNKEQVLFPISNLFHPKEVNELCTALTDCTVLGLPRELMAFLCKANDDIFLTLFALINDNEQQHMNYNMALTSKFAKDRIIKLICHLCQTVGYDQDEFYEIKQFLTIQLMSDMAGISRETAGHIIHELKDEKLVVKDHKNWLVSKHLFNDVCV

SEQ ID NO: 55 - protein, 330 aa, Staphylococcus sp.
MTKIMFFGTRDYEKEMALNWGKKNNVEVTTSKELLSSATVDQLKDYDGVTTMQFGKLENDVYPKLESYGIKQIAQRTAGFDMYDLDLAKKHNIVISNVPSYSPETIAEYSVSIALQLVRRFPDIERRVQAHDFTWQAEIMSKPVKNMTVAIIGTGRIGAATAKIYAGFGATITAYDAYPNKDLDFLTYKDSVKEAIKDADIISLHVPANKESYHLFDKTMFDHVKKGAILVNAARGAVINTPDLIDAVNDGTLLGAAIDTYENEAAYFTNDWTNKDIDDKTLLELIEHERILVTPHIAFFSDEAVQNLVEGGLNAALSVINTGTCETRLN

SEQ ID NO: 56 - protein, 675 aa, Staphylococcus sp.
MHHVNKYFNQTMVIEALKMSFYKLNPKQLIKNPIMFVVEVGMLLTLILICFPDIFGTSYLSRGYLITIFIILLITILFANFSEAFAEGRGKAQADSLRQAQSNLTARLIEENGAYRIVNATELKAGQNIRVENGETIPADGVVINGLATVDESAITGESAPVIKESGGDFDGVIGGTLVTSDWLEIRVESEAGTSFLDKMIALVEGAERNKTPNEIALFTLLTTLTIIFLVVIVTLYPIASYLHLILPIAMLIALTVCLIPTTIGGLLSAIGIAGMDRVTQFNVLAKSGRAVEVCGDVDVMILDKTGTITYGNRIASEFLPVNQQMMEKLIVAAYMSSIYDDTPEGKSIVRLAKQMYINELPKDIDGTYKPFTAETRMSGIITNEISVFKGAPNSMINLVKQQQGNIPLNIESICMDVSSKGGTPLIVIENNVMLGVIYLKDVIKDGLVERFAELRKMGIETVMCTGDNALTAATIAKEAGVDRFVAECKPEDKIKVIKDEQAKGHIVAMTGDGTNDAPALAQANIGLAMNSGTISAKEAANLIDLDSNPTKLIEVVKIGKQLLMTRGALTTFSLANDVAKYFAILPALMMSTIPEMTSLNIMHLSSPKSAIISALIFNALIIVALIPIAMKGVKVKGYSIDRIFINNMLIYGLGGLIVPFLGIKLIDMIVQFFV

SEQ ID NO: 57 - protein, 229 aa, Staphylococcus aureus
MIDHTHLRLFQFCDSQFPTGAFSHSFGLETYIQRNIIHDDHTFIAWLKMFLQEQLTYSDGLAMRLVYDALENDDTQKVLHIDKLMFVQNLPKETRVGAKQMGTRIVKLALELYNSPWIAWYHQQMQDKKAKLNPAICFTMLGHYLGVDIETIIDYYLYQNVSSLTQNAVRAIPLGQTAGQKIVTHMIPYIEETRKQIFELKEADFGMTAPGLELNQMAHENVNVRIFIS

SEQ ID NO: 58 - protein, 313 aa, Staphylococcus sp.
MKEKIVIALGGNAIQTKEATAEAQQTAIRRAMQNLKPLFDSPARIVISHGNGPQIGGLLIQQAKSNSDTTPAMPLDTCGAMSQGMIGYWLETEINRILTEMNSDRTVGTIVTRVEVDKDDPRFDNPTKPIGPFYTKEEVEELQKEQPGSVFKEDAGRGYRKVVASPLPQSILEHQLIRTLADGKNIVIACGGGGIPVIKKENTYEGVEAVIDKDFASEKLATLIEADTLMILTNVENVFINFNEPNQQQIDDIDVATLKKYAAQGKFAEGSMLPKIEAAIRFVESGENKKVIITNLEQAYEALIGNKGTHIHM

SEQ ID NO: 59 - protein, 319 aa, Staphylococcus sp.
MKTRIVSSVTTTLLLGSILMNPVANAADSDINIKTGTTDIGSNTTVKTGDLVTYDKENGMHKKVFYSFIDDKNHNKKILVIRTKGTIAGQYRVYSEEGANKSGLAWPSAFKVQLQLPDNEVAQISDYYPRNSIDTKEYMSTLTYGFNGNVTGDDSGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKATKQQTNIDVIYERVRDDYQLHWTSTNWKGTNTKDKWTDRSSERYKIDWEKEEMTN

SEQ ID NO: 60 - protein, 336 aa, Staphylococcus sp.
MTEIQKPYDLKGRSLLKESDFTKAEFEGLIDFAITLKEYKKNGIKHHYLSGKNIALLFEKNSTRTRAAFTVASIDLGAHPEFLGKNDIQLGKKESVEDTAKVLGRMFDGIEFRGFSQQAVEDLAKFSGVPVWNGLTDDWHPTQMLADFMTIKENFGYLEGINLTYVGDGRNNIAHSLMVAGAMLGVNVRICTPKSLNPKDAYVDIAKEKASQYGGSVMITDNIAEAVENTDAIYTDVWVSMGEESEFEQRINLLKDYQVNQQLFDLTGKDSTIFLHCLPAFHDTNTLYGQEIYEKYGLAEMEVTDQIFRSEHSKVFDQAENRMHTIKAVMAATLGS

SEQ ID NO: 61 - protein, 122 aa, Staphylococcus haemolyticus
MANQEQIIEAIKEMSVLELNDLVKAIEEEFGVTAAAPVAAAGAAGGGDAAAEKTEFDVELTSAGSSKIKVVKAVKEATGLGLKDAKELVDNAPKVIKEGVAKEEAEKLKEQLEEVGATVELK

SEQ ID NO: 62 - protein, 411 aa, Staphylococcus sp.
MTDGPIKVNSEIGALKTVLLKRPGKELENLVPDYLDGLLFDDIPYLEVAQKEHDHFAQVLREEGVEVLYLEKLAAESIENPQVRSEFIDDVLAESKKTILGHEEEIKALFATLSNQELVDKIMSGVRKEEINPKCTHLVEYMDDKYPFYLDPMPNLYFTRDPQASIGHGITINRMFWRARRRESIFIQYIVKHHPRFKDANIPIWLDRDCPFNIEGGDELVLSKEVLAIGVSERTSAQAIEKLARRIFENPQATFKKVVAIEIPTSRTFMHLDTVFTMIDYDKFTIHSAILKAEGNMNIFIIEYDDVNKDISIKQSSHLKDTLEDVLGIDDIQFIPTGNGDVIDGAREQWNDGSNTLCIRPGVVVTYDRNYVSNDLLRQKGIKVIEISGSELVRGRGGPRCMSQPLFREDI

SEQ ID NO: 63 - protein, 281 aa, Staphylococcus saprophyticus
MNDFLITKVLNNNVIICTKAQAEYVLIAKGIGFNKKSGMILQENQTIEKIYILDQKSQQDHYKTLMELANDTLIQAVIEAVNIITNSEMKVDNQKLVISLTDHIIFAYKRLKQNQLINNPFIVETKQLYSKEYKIAEKVIERLNHVLEVHFPEDEIGFIALHIASNTESLSMREMDLINELINKSVFILENDLKQTIDKDTIQYQRFIRHIQFLIKRLRNGESLKKTNAFEALLKEQYPLCFNIALKIMRLLQQELKIKVYEAEVIYLTLHVYHFMENHKS

SEQ ID NO: 64 - protein, 749 aa, Staphylococcus sp.
MLETNKNHATAWQGFKNGRWNRHVDVREFIQLNYTLYEGNDSFLAGPTEATSKLWEQVMQLSKEERERGGMWDMDTKVASTITSHDAGYLDKDLETIVGVQTEKPFKRSMQPFGGIRMAKAACEAYGYELDEETEKIFTDYRKTHNQGVFDAYSREMLNCRKAGVITGLPDAYGRGRIIGDYRRVALYGVDFLMEEKMHDFNTMSTEMSEDVIRLREELSEQYRALKELKELGQKYGFDLSRPAENFKEAVQWLYLAYLAAIKEQNGAAMSLGRTSTFLDIYAERDLKAGVITESEVQEIIDHFIMKLRIVKFARTPDYNELFSGDPTWVTESIGGVGIDGRPLVTKNSFRFLHSLDNLGPAPEPNLTVLWSVRLPDNFKTYCAKMSIKTSSIQYENDDIMRESYGDDYGIACCVSAMTIGKQMQFFGARANLAKTLLYAINGGKDEKSGAQVGPNFEGINSEVLEYDEVFKKFDQMMDWLAGVYINSLNVIHYMHDKYSYERIEMALHDTEIVRTMATGIAGLSVAADSLSAIKYAQVKPIRNEEGLVVDFEIEGDFPKYGNNDDRVDDIAVDLVERFMTKLRSHKTYRDSEHTMSVLTITSNVVYGKKTGNTPDGRKAGEPFAPGANPMHGRDQKGALSSLSSVAKIPYDCCKDGISNTFSIVPKSLGKEPEDQNRNLTSMLDGYAMQCGHHLNINVFNRETLIDAMEHPEEYPQLTIRVSGYAVNFIKLTREQQLDVISRTFHESM

SEQ ID NO: 65 - protein, 727 aa, Staphylococcus haemolyticus
MQKAFRNVLVIAIIGVIIFGVFSYINGNGNTPKQLSYSQFVEKLDKGDLKTLEIQPEQNVYLVSGKTKNDEDYSSTILYNNEKDLQKITNEAKSQKGLDFKVKEEEKQSVFVSMLTTLIPVLIIAFLFIFFLSQAQGGGGGGRMMNFGKSKAKMYDNQKRRVRFSDVAGADEEKQELIEIVDFLKDNKKFKQMGSRIPKGVLLVGPPGTGKTLLARAVAGEAGAPFFSISGSDFVEMFVGVGASRVRDLFENAKKNAPCIIFIDEIDAVGRQRGAGVGGGHDEREQTLNQLLVEMDGFGENEGIIMIAATNRPDILDPALLRPGRFDRQIQVGRPDVKGREAILHVHSKNKPLDETVDLKAISQRTPGFSGADLENLLNEASLIAAREGKNKIDMRDIEEATDRVIAGPAKKSRVISDKERNIVAHHEAGHTVIGMVLDEAEVVHKVTIVPRGQAGGYAMMLPKQDRFLMTEPELLDKICGLLGGRVSEDINFHEVSTGASNDFERATQIARSMVTEYGMSKKLGPLQFSSSGGGQVFLGKDMQGEPNYSGQIAYEIDKEVQRIIKEQYERCKQILLDHEKELKLIAKTLLTEETLVAEQIRSLFYDGVLPEVDYDAARVVKDEDSDYSEGKYGKSYDDIREEQLEEGKEDMREDRKEDNDMNRERRHRQRDDRDNQTGHDQLRDGSNGDNDQHRGHSNNEEDTGHEQSPNIDKPYNPNDPNNPSNR

SEQ ID NO: 66 - protein, 456 aa, Staphylococcus aureus
MDSFSRKEIVIGRLKFITMSLIGILLFLVPIPVEQDGQKQTTLPVAFLAGVLKDVLGGVMPFLIVTIITLSGIITLICSTILKDKLKPDGLLHNAFNVRIGWLILRLLAVVFAWMTFLRIGSKVIYSDETGGLLFSSLLPTLVAVFLFAALFLPLLMEYGLLEMLGPIFRPVMRPLFTLPGRSTVDNLASFIGDGTVGVLITSRQYGEGYYSRREATVISTTFSVVSITFAIVVAETVHMQNQFFAFYLSVIVSCLVAEVIMPRIWPLNKIPDEYAKEVPESARTEALPEGKTALRHGFDTATEVGIKAPGIVDFFKSGLKTVVDMWFVILPVVMSIGTIATIIANYTPLFVILGKPFVPFLELMHIPEAAQASQTIIIGFADMFLPSILIEGVQNDITRFVIGALSISQLIYLSEVGGVILGSKIPVSIGKLFMIFLIRTIITLPIISLMAHLLL

SEQ ID NO: 67 - protein, 318 aa, Staphylococcus haemolyticus
MARHKIVLIGSGYVGSAFAHAIVAKGLVDEMAIIDIDEDKAKADVWDLNHATPFGDNFVNVHVGQYEDFKDADIVVICASAKLAKGETRLKLLEDNVDIFVPMIQRIVDSGFDGYFVLPSNPVDIMSYVVKRVSNFPKNKIIGSGTSLDTARFQFFLSREFDVAPNQVYAPIIGEHGDSQVAVWSHAQIAGEPVLDLLPSNTNLEAFKTSISNRTTQIGYDIYVRKGTTNFGISLSLVRIVEAILFNKNIIMNVSSYVEGEYGLSDVYIGVPTVINRNGADRIIELALSKEELSQLHHSADIILDYQRQADAIIDQMC

SEQ ID NO: 68 - protein, 446 aa, Staphylococcus aureus
MPKRTFTKEDIRKFAEEENVRYLRLQFTDILGTIKNVEVPVSQLEKVLDNEMMFDGSSIEGFVRIEESDMYLHPDLDTWVIFPWTAGQGKVARLICDVYKTDGTPFEGDPRANLKRVLKEMEDLGFTDFNLGPEPEFFLFKLDEKGEPTLELNDDGGYFDLAPTDLGENCRRDIVLELEDMGFDIEASHHEVAPGQHEIDFKYADAVTACDNIQTFKLVVKTIARKHNLHATFMPKPLFGVNGSGMHFNVSLFKGKENAFFDPNTEMGLTETAYQFTAGVLKNARGFTAVCNPLVNSYKRLVPGYEAPCYIAWSGKNRSPLIRVPSSRGLSTRIEVRSVDPAANPYMALAAILEAGLDGIKNKLKVPEPVNQNIYEMNREEREAVGIQDLPSTLYTALKAMRENEVIKKALGNHIYNQFINSKSIEWDYYRTQVSEWERDQYMKQY

SEQ ID NO: 69 - protein, 140 aa, Staphylococcus aureus
MAKQIIVTDSTSDLSHEYLKQHNIHVIPLSLTIDGKSYTDQADISSSEYIDHIENDADVKTSQPPIGRFIETYEQLAQDDVEIISIHLSSGLSGTYNTAVQASHMVDGNITVIDSKSISFGLGYQIKQIVELVLLQSKKI

SEQ ID NO: 70 - protein, 163 aa, Staphylococcus sp.
MIRCAKKEDLNAILAIYNDAIINTTAVYTYEPQTIDERVAWFETKQRKHEPIFVFEENGSVLGFATFGSFRPWPAYLYTIEHSIYVDASARGKGIASQLLHHLIVEAKAKGYRALVAGIDASNKASIQLHQKFAFKHAGTLTNVGFKFNQWLDLAFYELDLQD

SEQ ID NO: 71 - protein, 146 aa, Bacillus licheniformis
MKLHELKPAEGSRKTRNRVGRGIGSGNGKTAGKGHKGQNARSGGGVRPGFEGGQMPLFQRLPKRGFTNINRKEFAVVNLDKLNSFAEGTEVTPELLLETGVISKLKSGVKILGDGKLEKKLTVKANKFSASAKQAIEAAGGTAEVI

SEQ ID NO: 72 - protein, 178 aa, Staphylococcus aureus
MNNTLTIDQLQELLQIQKEFDDRIPTLNLGDSKIAYVVEFFEWFNTLETFKNWKKKPGKPLDVQLDELADMLAFGLSIANQVGVSSEEIKEAIESSFKDTEFHKMFNFKDKEFAQGAVVSTPQIIFKEFYPDQLAIVIAIDIAYNLYTIDQLIDAYKKKMKRNHERQDGTADAGKGYV

SEQ ID NO: 73 - protein, 166 aa, Staphylococcus aureus
MHKLAHISFGIVGMFVNTCMVAKYVIINWEMYSMKKVNNDTVFGILQLETLLGDINSIFSEIESEYKMSREEILILLTLWQKGSMTLKEMDRFVEVKPYKRTRTYNNLVELEWIYKERPVDDERTVIIHFNEKLQQEKVELLNFISDAIASRATAMQNSLNAIIAV

SEQ ID NO: 74 - protein, 944 aa, Staphylococcus epidermidis
MKGPSIVVKGARAHNLKGVDIELPKNKLIVMTGLSGSGKSSLAFDTIYAEGQRRYVESLSAYARQFLGQMDKPDVDTIEGLSPAISIDQKTTSKNPRSTVATVTEIYDYIRLLYARVGKPYCPYHGIEIESQTVQQMVDRILELEERTKIQLLAPVISHRKGSHEKLIEDIGKKGYVRLRVDDEIVDVNEVPQLDKNKNHTIEVVVDRLVVKDGIETRLADSIETALELAEGNLTVDVINGEELKFSENHACPICGFSIGELEPRMFSFNSPFGACPTCDGLGQKLKVDLDLVIPDKNKTLNEGAIEPWEPTSSDFYPTLLKRVCEVYKINMDKPYKKLTDRQKNILMNGSGEKEIEFTFTQRNGGTRKRKMVFEGVVPNIDRRYHESPSEYTREMMSKYMTELPCETCHGKRLSKEALSVYVGDYNIGEVVEYSIKNALYYFENLKLSDQDKSIADQILKEIISRLSFLNNVGLEYLTLDRSSGTLSGGEAQRIRLATQIGSRLTGVLYVLDEPSIGLHQRDNDRLINTLKEMRDLGNTLIVVEHDDDTMRAADYLVDVGPGAGNHGGEVVSSGTPNKVMKDKKSLTGQYLSGKKRIEVPEYRREITDRKIQIKGAKSNNLKNVNVDFPLSVLTVVTGVSGSGKSSLVNEILYKALAQKINKSKVKPGNFDEIKGIDQLDKIIDIDQSPIGRTPRSNPATYTGVFDDIRDVFAQTNEAKIRGYQKGRFSFNVKGGRCEACKGDGIIKIEMHFLPDVYVPCEVCDGKRYNRETLEVTYKGKNIADVLEMTVEEATHFFENIPKIKRKLQTLVDVGLGYITLGQQATTLSGGEAQRVKLASELHKRSTGRSIYILDEPTTGLHVDDISRLLKVLNRIVENGDTVVIIEHNLDVIKTADHIIDLGPEGGEGGGTIIATGTPEEIAQNKGSYTGQYLKPVLERDSVE

SEQ ID NO: 75 - protein, 281 aa, Staphylococcus aureus
MKKLIFLIVIALVLSACNSNSSHAKELNDLEKKYNAHIGVYALDTKSGKEVKFNSDKRFAYASTSKAINSAILLEQVPYNKLNKKVHINKDDIVAYSPILEKYVGKDITLKALIEASMTYSDNTANNKIIKEIGGIKKVKQRLKELGDKVTNPVRYEIELNYYSPKSKKDTSTPAAFGKTLNKLIANGKLSKENKKFLLDLMLNNKSGDTLIKDGVPKDYKVADKSGQAITYASRNDVAFVYPKGQSEPIVLVIFTNKDNKSDKPNDKLISETAKSVMKEF

SEQ ID NO: 76 - protein, 701 aa, Staphylococcus sp.
MNNNFFNSDFDSILRRMMQDMQNSNQTSNKKYYINGKEVSPEELAQFTQQGGNHSAEQSAQAFQQAALRQQGQQSGNGNYLEQIGRNLTQEARDGLLDPVIGRDKEIQETAEVLSRRTKNNPILVGEAGVGKTAIVEGLAQAIVEGNVPAAIKDKEIISIDISSLEAGTQYRGAFEENIQKLVEGVKSSQNAVLFFDEIHQIIGSGATGSDSGSKGLSDILKPALSRGEISIIGATTQDEYRNNIMKDAALTRRFNEVLVNEPSAKDTVEILKGIREKFETHHQVKLPDDVLKACVDLSIQYIPQRLLPDKAIDVLDITAAHLSAQSPAIDKVTTEKRISELEHDKRKAVSAEEYKKADDIQKEIKSLQDKLENSNGEHTAVATVHDISDTIQRLTGIPVSQMDANDIERLKNISSRLRSKIIGQDKAVEMVSRAIRRNRAGFDDGNRPIGSFLFVGPTGVGKTELAKQLAIDLFGNKEALIRLDMSEYSDTTAVSKMIGTTAGYVGYDDNSNTLTEKVRRNPYSVILFDEIEKANPQILTLLLQVMDDGNLTDGQGNVINFKNTIIICTSNAGFGNGNDTEEKDIMHEMKKFFRPEFLNRFNGIVEFLHLDKDALQDIVNLLLDDVQVTLDKKGITMDVSQDAKDWLIEEGYDEELGARPLRRIVEQQVRDKITDYYLDHTDVKHVDIDVEDNELVVKGK

SEQ ID NO: 77 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 78 - protein, 152 aa, Staphylococcus carnosus
MNFEGKLLGQDLKIGIVVSRFNDFITGRLLDGAKDVLVRHEVESEKIDVAFVPGAFEIPLAAKKLAETGKYDAVITLGCVIRGATSHYDYVCNEVAKGVSKVSDTTGLPVIFGVLTTETIEQAVERAGTKAGNKGAEAAMAAIEMANLLKSI

SEQ ID NO: 79 - protein, 639 aa, Staphylococcus haemolyticus
MDNDKKHVIPREQYRRKRHEYFHNEEREERLEREREQRERLAKKEQEQAKVNEERVKDNMRKARIEKLTQEEIHQQQHLAKLRSDNESDQELNDTNTHHLTLPEEQQLKNEHKENNDKVTKPTDEMEKQEKEDNNSASSKHDEIEPKYSRVEKNKGKQKQDNINKSEVNHLDKSEQTKKHKETKESSEDVLETNKSQKIEQKEQKASSNETSNKELNSYTKDKNNKVEDNQDLKKASSQNLAHSNKLEENEHLENEPKNNDTMDKVKDFLKLHWLKIVIVVAIILIVILISAIISTMNQNSSIEQSSNNDTKYTTTMKNAETAVKSVVTIENDTPKNITTQTIDKTNINSNNEVGSGVVYKAVDDTFFILTNTHIVGSNKRVNITYDDDKTATATVVGRDMWSDIAVLKATIKNKNMQPIKIGHSKHLKLGESILVVGNPLGNDFKNTVTKGIISGLNRAVPVDFDKDNKNDEWVNTFQIDASVNPGNSGGAVVNRVGELVGLVSLKINMPNIEGMGFAIPIDAAREIAEELEKKGEIQYPNTGIGIKNVSDLMPYERNLLKVPEDVQNGIVVEKLKENGLGKKSGLKIGDVVVELDSKSIQNNLQYRQIIFNHRQDLKTLSAKIYREGKSQEIRIKLK

SEQ ID NO: 80 - protein, 254 aa, Staphylococcus sp.
MMIITEKRHELILEELSHKDFLTLQELIDRTGCSASTIRRDLSKLQQLGKLQRVHGGAMLKENRMVEANLTEKLATNLDEKKMIAKIAANQINDNECLFIDAGSSTLELIKYIQAKDIIVVTNGLTHVEALLKKGIKTIMLGGQVKENTLATIGSSAMEILRRYCFDKAFIGMNGLDIELGLTTPDEQEALVKQTAMSLANQSFVLIDHSKFNKVYFARVPLLESTTIITSEKALNQESLKEYKQKYHFIGGTL

SEQ ID NO: 81 - protein, 411 aa, Staphylococcus haemolyticus
MTQGPIQVNSEIGRLKTVLLKRPGKELENLVPDHLSGLLFDDIPYLKVAQEEHDKFAQTLRDEGVEVVYLEKLAAEAIADKDVREQFIDDILAESQKTVLGHEAEIKTFFAKLSDQELIDKIMAGVRKEEIELKTTHLVEYMDDRYPFYLDPMPNLYFTRDPQASVGRGMTINRMYWRARRRESLFMTYILKYHPRFKDADVPVWLDRNSPFNIEGGDELILSKEALAIGISERTSAQAIERLARNIFKDESTTFKKVIAIEIPNSRTFMHLDTVFTMIDYDKFTVHSAIFKEENNMNLFTIEYDEAKDDIKITHSNKLRETLADVLGVEKIEFIPTGNGDVIDGAREQWNDGSNTLCIRPGVVVTYDRNYVSNQLLRDKGIKVLEITGSELVRGRGGPRCMSQPLFREDI

SEQ ID NO: 82 - protein, 226 aa, Staphylococcus sp.
MAKSYDYQSAFDIIGPVMMGPSSSHTAGAVKIGNSARAILGDIPKNIEIHYYESFAQTHQGHGTDVAIVGGAMGYSTFDNRIKSSLEIAADDGIKIDIVEEEGDSIGQHPNCAYIKSFRNDGRYIEIIGISIGGGTIKIKGIHINGLEVDLNHGLPILVVDGNMTKAQVNHFINDINDMKLDCKDEFIMIDEDKCLVVIPLNKAISESTLNTIKEKYSDVNVSYIN

SEQ ID NO: 83 - protein, 381 aa, Staphylococcus aureus
MTINLSETFANAKNEFINAVNNGEPQERQNELYGDMINQLFEETKLQAKAEAERVSSLPKSAQSLSANQRSFFMDINKNVNYKEEKLLPEETIDRIFEDLTTNHPLLADLGIKNAGLRLKFLKSETSGVAVWGKIYGEIKGQLDAAFSEETAIQNKLTAFVVLPKDLNDFGPAWIERFVRVQIEEAFAVALETAFLKGTGKDQPIGLNRQVQKGVSVTEGAYPEKEEQGTLTFANPRATVNELMQVFKYHSTNEKGKSVAVKGNVTMVVNPSDAFEVQAQYTHLNANGVYVTALPFNLNVIESTVQEAGKVLTYVKGLYDGYLAGGINVQKFKETLALDDMDLYTAKQFAYGKAKDNKVAAVWKLDLKGHKPALEGTEETL

SEQ ID NO: 84 - protein, 311 aa, Staphylococcus carnosus
MFHHVSVMLRETIDELNVKKDGVYVDCTLGGAGHALYLLNQLDENGHLIAIDQDLTAIENAKEVLKDHLDQVTFVHSNFRQINQILDDLDIDKVDGIYYDLGVSSPQLDVPERGFSYHQDARLDMRMDQTQELSAYEVVNEWPYEDLVRIFYRYGEEKFSKQIARRIEKTRENEPIETTLQLVDVIKEGIPAKARRKGGHPAKRVFQAIRIAVNDELKAFEDSLEQAIERVKVHGRISVITFHSLEDRLCKQIFQEYEKGPEVPRGLPIIPEEYTPKLKRVNRKPISASEEDLAENNRARSAKLRVAEILK

SEQ ID NO: 85 - protein, 1009 aa, Staphylococcus sp.
MKPLHLKLNNFGPFLKEEIDFSKIDNNELFLISGKTGSGKTMIFDAMTYALFGKASTEQREENDLRSHFADGKQPMSVTFEFQLNNRIYKVHRQGPYIKEGNTTKTNAKFDVFEMVDGKYEIRESKVISGTQFIIELLGVNADQFRQLFILPQGEFKRFLISNSREKQGILRTLFDSEKFEAIREILKEEVKKENAQIENRYQQIDLLWQEIESFDDDKIKGLLEVATQQIDKVIENIPLLQARSKEILAFVNESKETAIKDYEIIEKKTLENNILKDNINQLNKNKIDFVQLKEQQPEIEEIEAKLKLLQDITNLLNYIENREKIETKIANSKKDISETNNKILNLECDKRNIDKEKRMLEENGDLIESKISFIDKTRVLFNDINKYQQSYLNIERLRTEGEQLADELNNLIEGLEKVEDSIGNNESDYEKIIELNNAITNINNEINVIKENEKAKAELDKLLGSKQELENQINEETSTLKNLEIKLDRYDKSKLDLNDKESFISEIKSAVKIGDQCPICGNEIQDLGHHIDFDSIAKRQNEIKEIEANIHTMKSNIAVHNSEIKFVNEKISNINIKTQSDFSLEVLNKRLLENENALNNQRELNKFIEQMKEEKDNLTLQIHNKQLRLNKNESELKLCRNLITEFETLSKYNNITNFEVDYKKYVQDVNQHQEHSNQIEDKLIQLSQRKLIEQNNLNHYEKQLETYNNDLELNEQSIEMEMSRLNLTDNNDINEIIAWRGEQEELEQKRDTYKKRYHEFEMEIARLESLTKDKELLDSDKLKDEYELKKGKMNTLIDEYSAVHYQCQNNINKTQSIVSHINYLNQELKDQQEIFQLAEIVSGKNNKNLTLENFVLIYYLDQIIAQANLRLATMSDNRYQLIRREAVSHGLSGLEIDVFDLHSNKSRHISSLSGGETFQSSLALALGLSEIVQQQSGGISLESIFIDEGFGTLDQETLETALDTLLNLKSTGRMVGIISHVSELKNRIPLVLEVKSDQYQSSTRFKRN

SEQ ID NO: 86 - protein, 188 aa, Staphylococcus aureus
MVKIAIFASGSGSNFENIVEHVESGKLENIEVTALYTDHQNAFCIDRAKKHDIPVYINEPKQFDSKAAYEQHLVSLLNEDKVEWIILAGYMRLIGPDLLASFEGKILNIHPSLLPKYKGIDAIGQAYHSGDTITGSTVHYVDCGMDTGEIIEQRQCDIRPDDSKEQLEEKVKKLEYELYPSVIAKIVK

SEQ ID NO: 87 - protein, 223 aa, Staphylococcus sp.
MKFAVLVFPGSNCDRDMFNAAIKSGVEAEYVDYRETSLSGFDGVLIPGGFSFGDYLRSGAMASVAPVISEVKRLAAEGKPVLGVCNGFQILTEIGLLPGALLHNDSHLFISRNEELEIVNNQTAFTNLYEQGEKVIYPVAHGEGHYYCTDEIYQKLKANNQIILKYVNNPNGSYDDIAGIVNEKGNVCGMMPHPERALETLLGTDSGVKLFEAMVKSWREQHV

SEQ ID NO: 88 - protein, 1170 aa, Staphylococcus saprophyticus
MKSIITEYINEDKRYQELNEVFGKENVLVTGLSGAAKATIIAEKYLNSEQQLLVVTNNLYQADKLESDLLQFVEDSEIYKYPMQDIMTEEFSTQSPQFMSERVRTLTALAQEERGLFIVPLNGLKKWLTPVDMWKSHQLTLNVGDDIDIDEFLNKLVNMGYRRESVVSHIGEFSLRGGIVDIYPLIGKPVRIEFFDTEVDSIRDFDVESQRSEGNIEHVDITTASDYIITEDVLKHTKHKLKQAYEDTRPKIEKSVRNELKETYESFQLFEAEMFDHQVLRRLVAFMYEQPATIMDYFKEDAIIAVDEYNRIKETETTLVTEIDEFMQNLIESGKGFIDQSFLQYEGFENLLKPFAVTYFTLFTATMPVQLNEIIKFSCKPVQQFYGQYDIMRSEFQRFIQNDYTIVVLAETETKKERIQSMLNEMHIPTFIDTPSRRNEGGSAIITEGSLSEGFELPYMQLVVVTERELFKSKQKKKPKQHKTLTNAEKIKSYQDLKVGDYVVHVHHGVGRYLGVETLEVGGVHKDYIKLQYKGTDQLFVPVDQMDQVQKYVASEDKSPKLNKLGGTEWKKTKAKVQQSVEDMADELIELYKAREMSVGYKFGPDTAEQNDFEIDFPYELTPDQSKSIEEIKQDMEIERPMDRLLCGDVGYGKTEVAVRAAFKAVMEGKQVAFLVPTTILAQQHYETLIERMQDFPIEVQLISRFRTTKEVKETKEGLKSGFVDIVVGTHKLLGKDIQYKDLGLLIVDEEQRFGVRHKERIKSLKNNVDVLTLTATPIPRTLHMSMLGVRDLSVIETPPENRFPVQTYVLEQNTNFIKEALERELSRDGQVFYLYNKVQSIYEKREQLQMLMPDANIGVAHGQMNERDLEETMLSFINHEYDIIVTTTIIETGVDVPNANTLIIEDADRFGLSQLYQLRGRVGRSSRIGYAYFLHPTNKVLSETAEDRLQAIKEFTELGSGFKIAMRDLNIRGAGNLLGKQQHGFIDSVGFDLYSQMLEEAVNEKRGIKAEKQDAPEIEIELNIDAYLPAEYIPNEQSKIEIYKKLRKIESETQLMDVKDELIDRFNDYPIEVERLLEMMEIKVHALHAGVTLIKDVGKQVEVYLSEKGTTDINGETLFKQTQPLGRTMKVGVQEGKMKVTLNKTGNWLDSLKFLTKSLEESMVITDEI

SEQ ID NO: 89 - protein, 707 aa, Staphylococcus carnosus
MSKQRIYEYAKDLNLKSKDVIDELKKMNVEVSNHMQALEPNEIKELDKKFKPGSSKDKQDNKAAQNNHKKHQNNNKDKNNNNNGGNAKKNNKKNHNNKNQKNNKNNKNNKNQKQAPKQEAVKEMPEKITYTEGITVGELAEKMNIESSNIVKKLFLLGIMANINQSLDDETVELIADDYGIEIEKEVVIDEEDLDIYFDDEEEDPDAIERPAVVTIMGHVDHGKTTLLDSIRNTHVTEGEAGGITQHIGAYQIENDGKKITFLDTPGHAAFTTMRARGAQVTDITILVVAADDGVMPQTIEAINHAKEADVPIIVAVNKVDKPTANPDRVMQELTEYGLFPEDWGGDTIFVPLSALSGDGIDDLLEMIVLVSEVQELKANPEKRAVGTVIEAELDKSRGPAASLLVQNGTLNVGDAIVVGNTYGRVRAMVNDAGKRVKSAGPSTPVEITGINDVPQAGDRFVTFSDEKQARRIGEARHEESIIQQRQESKNVSLDNLFEQMKQGEMKDLNIIIKGDVQGSVEALAASLMKIDVEGVNVRIIHTAVGAINESDVTLANASNGIIIGFNVRPDSGAKRAAEQENVDMRLHRVIYNVIEEIEAAMKGMLDPEYEEKVIGQAEVRQTFKVSKVGTIAGCYVTDGKITRNAGVRVIRDGIVVFEGQLDTLKRFKDDVKEVAQGYECGITIEKFNDIKVDDIIEAYEMVEIQR

SEQ ID NO: 90 - protein, 450 aa, Staphylococcus aureus
MNNKRHSTNEQLSLDEINNTIKFDHRSSNKQKFLSFLGPGLLVAVGYMDPGNWITSMQGGAQYGYTLLFVILISSLSAMLLQSMTVRLGIATGMDLAQMTRHYLSRPIAIIFWIIAELAIIATDIAEVIGSAIALNLLFNIPLIVGALITVLDVFLLLFIMKYGFRKIEAIVGTFIFTVLFIFIFEVYISSPQLNAVLNGFIPHSEIITNNGILYIALGIIGATIMPHNLYLHSSIVQSRTYSRHNNEEKAQAIKFATIDSNIQLSIAFVVNCLLLVLGASLFFNSNADDLGGFYDLYHALKTEPVLGATMGAIMSTLFAVALLASGQNSTITGTLAGQIVMEGFLRLHIPNWLRRLITRSLAVIPVIVCLIIFKGNAAKIEQLLVFSQVFLSIALPFCLIPLQLATSNKDLMGPFYNKTWVNIISWTLIIILSILNVYLIVQTFQELQS

SEQ ID NO: 91 - protein, 272 aa, Staphylococcus epidermidis
MTRQWETLREYDEIKYEFFEGIAKVTINRPEVRNAFTPKTVAEMIDAFSRARDDQNVSVIVLTGEGDKAFCSGGDQKKRGHGGYVGEDDIPRLNVLDLQRLIRVIPKPVIAMVRGYAIGGGNVLNVVCDLTIAADNAIFGQTGPKVGSFDAGYGSGYLARIVGHKKAREIWYLCRQYNAQEALDMGLVNTVVPLEQVEDETVKWCKDIMQHSPTALRFLKAAMNADTDGLAGLQQMAGDATLLYYTTDEAKEGRDAFKEKRNPDFDQFPKFP

SEQ ID NO: 92 - protein, 398 aa, Staphylococcus carnosus
MSNNRRLFTSESVTEGHPDKIADQISDAILDELLKKDPDARVACETVVTTGMAMIVGEISTATYVDFPKVVRDTVEEIGYTRAKYGYDSQTMAVMSAIDEQSPDIAQAVDRALEYRNEISEEEIEATGAGDQGLMFGFATNETDTYMPTPIYFSHQLAKRLSDVRKDGTLKYLRPDGKVQVTVEYDENDKPVRIDTIVISTQHAEDVTLDQIQEDIRKHVIDPIVPASLLDDETKFFINPTGRFVIGGPQGDAGLTGRKIIVDTYGGFARHGGGCFSGKDPTKVDRSAAYAARYVAKNIVAAGLADKCEVQLAYAIGVAEPVSISIDTFGTGKVPESKLVEAVRDNFDLRPAGIIKMLDLKRPIYKQTAAYGHFGRTDVALPWERVDKVDVLKEAVQA

SEQ ID NO: 93 - protein, 591 aa, Staphylococcus sp.
MYYSYGNYEAFARPKKPENVENKSAYLIGSGLASLAAACFLIRDGQMEGSKIHILEELPKAGGSLDGENMPLKGYVVRGGREMENHFECLWDLFRSIPSLEIDHASVLDEFYWLNKEDPNYSRCRVIEKQGQRLVTDGDFTLTKTAIKEILDLCLTNEEDLDDVKITDVFSDDFFNSNFWIYWKTMFAFEPWHSAMEMRRYLMRFVHHIGGLADFSALKFTKYNQYESLVLPMVEYLKSHGVQFEYDVKVKDIKVDVTTSQKIAREILIDRNGNAESIKLTINDLVFVTNGSITESSTYGDNDTPAPPTDELGGSWTLWKNLARQSPEFGNPDKFCQNIPKKSWFVSATSTTNNKDIIDTIESICKRDPLAGKTVTGGIITINDSAWQISFTINRQQQFKDQPKNEISTWIYALYSDVNGDYIKKPITECSGNEICQEWLYHLGVPTDKIEDLAKHASNTIPVYMPYITSYFMTRAIGDRPLVVPHQSQNLAFIGNFAETERDTVFTTEYSVRTAMEAVYQLLNIDRGIPEVINSTFDLRVLMDAVYELNDHQDLREITKDSKMQKLALAGFLKKIKGTYIESLLKEHKLL

SEQ ID NO: 94 - protein, 87 aa, unidentified
  REGION 1..87: note = Eubacteria sp.
MPKSILDIKNSIDCHVGNRIVLKANGGRKKTIKRSGILKETYPSVFIVELDQDKHNFERVSYTYTDVLTENVQVSFEEDNHHESIAH

SEQ ID NO: 95 - protein, 234 aa, Staphylococcus sp.
MTNVLYQHGTLGTLMAGLLEGTATINELLEHGNLGIATLTGSDGEVIFLDGKAYHANEHKEFIELKGDEKVPYASITNFKASKTFPLQQLSQDDVFAQIKNEMLSENLFSAVKIYGTFKHMHVRMMPAQQPPYTRLIDSARRQPEEKRQDIRGAIVGFFTPELFHGVGSAGFHIHFADDERAYGGHVLDFEVDDVVVEIQNFETFQQHFPVNNETFVKAKIDYKDVAEEIREAE

SEQ ID NO: 96 - protein, 526 aa, Staphylococcus aureus
TITVNGTSQNILSNLTFKKNQQISYKDLENNVKSVLKSNRGITDVDLRLSKQAKFTVNFKNGTKKVIDLKAGIYTANLINTGDIKNININVETKKQAKDKEAKANNQVPYSINLNGTTTNIQSNLAFSNKPWTNYKNLTAKVKSVLKSDRGVSERDLKHAKKAYYTVYFKNGGKRVIHLNSNIYTANLVHAKDIKKIEVTVKTGSKANAERYVPYTIAVNGTSTPNLSDLKFKGDSRVSYSDITKKVKSVLKYDRGIGERELKYAKKATYTVHFKNGTKKVINLNSKISQLNLLFVKDIKKIDVDVKTGSKAKADSYVPYTIAVNGTSTPIASKLKLSNKQLIGYQDLNKKVKSVLKHDRGINDIELKFAKQAKYTVHFKNGKTQVVDLKSDIFTRNLFSVKDIKKIDIDVKQHTKSNKALNKVANIATKVKFPVTINGFSNVVSNEFAFLHPHKITTNDLNAKLRLALASDQGITKHDIGLSERTVYKVYFKDGSSKFVDLKAAKQDSKVFKATDIKKVDIEIKF

SEQ ID NO: 97 - protein, 223 aa, Staphylococcus epidermidis
MKFAVLVFPGSNCDRDMYNAAIKSGVDAAYVDYRETTLDGFDGVLIPGGFSFGDYLRSGAMASVAPIIKEVKRFAKEGKPVLGVCNGFQILTEIGLLPGALLHNDSHLFISRNETLKITNNQTPFTHLYVKNENVVYPVAHGEGHYYCTDEIYRELNDNNQIILKYTNNPNGSYEDIAGIVNKEGNVCGMMPHPERALETLLGTNSGVKLFESMVKSWREQNV

SEQ ID NO: 98 - protein, 341 aa, Staphylococcus sp.
MISFENDYLEGAHEKVLKRLVDTNLVQASGYGFDQFTAQAIEKIKDTIDCPNATIRFLVGGTQTNQVVINSMLESYEGVISADTGHVAVHEGGAIEYSGHKVITIPSKEGKVSASDVETYMETFKSDFKKDHMVFPGMVYISHPTEYGTLYSKSELEELCKVCKQYQLPLFMDGARLGYGLMSDQSDMTIKDIAKYCDVFYIGGTKIGALCGEAIVFTKNNEPKQFTTRIKHHGALLAKGRLTGIQFLELFTDNLYFNISRHAIEMANKMKDGFINKGYRLYFDSPTNQQFFILSNEKIAELEQKVKFAVWEKYDDQHRVVRFATSWATTEENLNKLLELI

SEQ ID NO: 99 - protein, 243 aa, Staphylococcus sp.
MSNYSTGELAKLCNVTTRTIQYYDRKGILKPQGFTEGKRRVYTEQQRQTLELILLLKDLGCALSDIDMLLKGEGTLKTLNTLLTMKQQEINQQVKQQQAVLDKIKNVQYYVNEASTSPITHLKDIEHVMSKSAEMKSIRRNIWISAGIIGIIQYSSILSSILMKNKWPFLIALPFMIGYGIGITIYYQRKVAYLCPNCQHIFSPSLWAVIKAKHTATTRRFECPNCHETHYCIEVPKTHMGTE

SEQ ID NO: 100 - protein, 1335 aa, Staphylococcus epidermidis
MAKKFNYKLPSMVALTLFGTAFTAHQANAAEQPQNQSNHKNVLDDQTALKQAEKAKSEVTQSTTNVSGTQTYQDPTQVQPKQDTQSTTYDASLDEMSTYNEISSNQKQQSLSTDDANQNQTNSVTKNQQEETNDLTQEDKTSTDTNQLQETQSVAKENEKDLGANANNEQQDKKMTASQPSENQAIETQTASNDNESQQKSQQVTSEQNETATPKVSNTNASGYNFDYDDEDDDSSTDHLEPISLNNVNATSKQTTSYKYKEPAQRVTTNTVKKETASNQATIDTKQFTPFSATAQPRTVYSVSSQKTSSLPKYTPKVNSSINNYIRKKNMKAPRIEEDYTSYFPKYGYRNGVGRPEGIVVHDTANDNSTIDGEIAFMKRNYTNAFVHAFVDGNRIIETAPTDYLSWGAGPYGNQRFINVEIVHTHDYDSFARSMNNYADYAATQLQYYNLKPDSAENDGRGTVWTHAAISNFLGGTDHADPHQYLRSHNYSYAELYDLIYEKYLIKTKQVAPWGTTSTKPSQPSKPSGGTNNKLTVSANRGVAQIKPTNNGLYTTVYDSKGHKTDQVQKTLSVTKTATLGNNKFYLVEDYNSGKKYGWVKQGDVVYNTAKAPVKVNQTYNVKAGSTLYTVPWGTPKQVASKVSGTGNQTFKATKQQQIDKATYLYGTVNGKSGWISKYYLTTASKPSNPTKPSTNNQLTVTNNSGVAQINAKNSGLYTTVYDTKGKTTNQIQRTLSVTKAATLGDKKFYLVGDYNTGTNYGWVKQDEVIYNTAKSPVKINQTYNVKPGVKLHTVPWGTYNQVAGTVSGKGDQTFKATKQQQIDKATYLYGTVNGKSGWISKYYLTAPSKVQALSTQSTPAPKQVKPSTQTVNQIAQVKANNSGIRASVYDKTAKSGTKYANRTFLINKQRTQGNNTYVLLQDGTSNTPLGWVNINDVTTQNIGKQTQSIGKYSVKPTNNGLYSIAWGTKNQQLLAPNTLANQAFNASKAVYVGKDLYLYGTVNNRTGWIAAKDLIQNSTDAQSTPYNYTFVINNSKSYFYMDPTKANRYSLKPYYEQTFTVIKQKNINGVKWYYGQLLDGKYVWIKSTDLVKEKIKYAYTGMTLNNAINIQSRLKYKPQVQNEPLKWSNANYSQIKNAMDTKRLANDSSLKYQFLRLDQPQYLSAQALNKLLKGKGVLENQGAAFSQAARKYGLNEIYLISHALVETGNGTSQLAKGGDVSKGKFTTKTGHKYHNVFGIGAFDNNALVDGIKYAKNAGWTSVSKAIIGGAKFIGNSYVKAGQNTLYKMRWNPANPGTHQYATDINWANVNAQVLKQFYDKIGEVGKYFEIPIYK

SEQ ID NO: 101 - protein, 120 aa, Staphylococcus epidermidis
MISKIDKNKVRLKRHARVRTKLSGTAEKPRLNVYRSNKHIYAQIIDDVKGVTLAQASSQDKDIANTSASKVDLATTVGQEIAKKANDKGIKEIVFDRGGYLYHGRVKALADAARENGLEF

SEQ ID NO: 102 - protein, 209 aa, Staphylococcus aureus
MTIKVLFVDDHEMVRIGISSYLSTQSDIEVVGEGASGKEAIAKAHELKPDLILMDLLMDDMDGVEATTQIKKDLPQIKVLMLTSFIEDKEVYRALDAGVDSYILKTTSAKDIADAVRKTSRGESVFEPEVLVKMRNRMKKRAELYEMLTEREMEILLLIAKGYSNQEIASASHITIKTVKTHVSNILSKLEVQDRTQAVIYAFQHNLIQ

SEQ ID NO: 103 - protein, 801 aa, Staphylococcus sp.
MAKQKLVMIGNGMAGIRTIEEILERANDLYDITVIGKEPYPNYNRIMLSNILQNKMTVEETIMNPYEWYEEHDIKLITNDPVVDVDRANQNVTTANGIEVAYDKLIFATGSKAFVIPVPGSTLPSVIGWRTIDDTEQMMNIAKTKKKAIVIGGGLLGLECARGLLDQGMEVTVLHLAEWLMEMQLDRKAGNMLKADLEKQGMKFEMQANTTEILGEDDVEGVKLADGREIPTDLVVMAVGIRPYTEVAKESGLDVNRGIVVNDVMQTSDSNVYAVGECAEHNGKVYGLVAPLYEQGKVLADHLTNKETNGYKGSTTFTSLKVSGCDLYSAGQIVENAEIKGIEIFNSVDNNYKKIFLKDGNVVGAVLYGDIDDGSRFYNMMKKGESTEDYTLVSLLTKGGEEASLSIADMADDETICGCNGVDKGTIVNAITENGFTTVEEVTAKTKAGNSCGKCKPQIAQILQHTLGDDFVAAKPAGICGCTDLTRDQIVTQIRAKGLKTSKEVRHVLNFKNKGGCPKCRPAINYYLNMVYPHDHEDERESRFANERYHANIQNDGTFSVIPQMRGGVTDADQLIRLGEVAKKYHVPLVKVTGSQRVGLYGVKKEELPNIWEDLGMRSASAYGKKTRSVKSCVGKEFCRFGTQYTTRLGIRLEKTFEYIDTPHKFKMGVSGCPRSCVESGVKDFGIISVENGFQIYIGGNGGTEVEKAEFLTTVETEDEVIKLCGALMQYYRETGIYAERTAPWLRRLGFENVKEVLLDPERQNELFERIMDAKKAVEAEPWEAITSNAQARKIFEVEKV

SEQ ID NO: 104 - protein, 234 aa, Staphylococcus sp.
MTLLYEGKAKRIFSTNQENELRVEYKDEVTAGNGAKKDTMAGKGRLNNQITSIIFKYLQENGIESHFIKQLSETEQLVKPVKIIPLEVVVRNIASGSITKRLGFENGEVFREPLVEFFYKNDALNDPLITDDHIKLLNIATDKDIETLKTKALEINQVLKQLMHAMNLKLVDFKIEFGKTETGQILLADEISPDTCRIWDKATNANFDKDVYRNNTGSLIETYQIFLNKLEDLK

SEQ ID NO: 105 - protein, 230 aa, Staphylococcus aureus
MYVDRKPSLYLEDFRHDFKNSLSKFENGDEAFDTLLGFVELDHIYSSALKEISTKLSILDDNFNHIYKHNPIHHMERRVKEMRSLIEKLNRKGLQISAETAKEHILDIAGIRVVCNYLDDIYLIEEMLLKQEDVQLIKRKDYIQHPKENGYRSLHIVVSIPVFLAERVEVLPVEIQIRTIGMDMWASLEHKIRYKNKAETEKYRDLLKECATEITEVENKLQQIHSEITE

SEQ ID NO: 106 - protein, 689 aa, Staphylococcus aureus
MKFKSLITTTLALGVIASTGANLDTNEASAAAKQIDKSSSSLHHGYSKIQIPYTITVNGTSQNILSSLTFNKNQQISYKDIENKVKSVLYFNRGISDIDLRLSKQAKYTVHFKNGTKRVVDLKAGIHTADLINTSDIKAISVNVDTKKQVKDKEAKANVQVPYTITVNGTSQNILSNLTFKKNQQISYKDLENNVKSVLKSNRGITDVDLRLSKQAKFTVNFKNGTKKVIDLKAGIYTANLINTGGIKNININVETKKQAKDKEAKVNNQVPYSINLNGTTTNIQSNLAFSNKPWTNYKNLTTKVKSVLKSDRGVSERDLKHAKKAYYTVYFKNGGKRVIHLNSNIYTANLVHAKDVKRIEVTVKTVSKVKAERYVPYTIAVNGASNPTLSDLKFTGDSRVSYSDIKKKVKSVLKHDRGIGERELKYAEKATYTVHFKNGTKKVINLNSNISQLNLLYVKDIKNIDIDVKTGAKAKVYSYVPYTIAVNGTTTPIASKLKLSNKQLIGYQDLNKKVKSVLKHDRGINDIELKFAKQAKYTIHFKNGKTQVVDLKSDIFTRNLFSVKDIKKIDINVKQQSKSNKALNKVTNKATKVKFPVTINGFSNLVSNEFAFLHPHKITTNDLNAKLRLALRSDQGITKHDIGLSERTVYKVYFKDGSSKLEDLKAAKQDSKVFKATDIKKVDIEIKF

SEQ ID NO: 107 - protein, 207 aa, Staphylococcus sp.
MNKVILVDDHYIVRQGLRFLLSTIENIEVLQDFADGETFLEYLKEHEHPDIVLLDLVMPGMNGIEITEYIKAHYPDIKVLVLTSYVDDEHVISAINKGADGYEMKDVEPQQLIETIRRVMNGEKMIHPKAQDVFETVSQKPHYTNKLSKREIEVLREMVKGKTNKEIAETLFVSEKTIKTHVSHIFSKLQVSDRTQAAIYAMENKLI

SEQ ID NO: 108 - protein, 268 aa, Staphylococcus aureus
MKFAVIGNPISHSLSPVMHRANFNSLGLDDTYEALNIPIEDFHLIKEIISKKELDGFNITIPHKERIIPYLDHVDEQAINAGAVNTVLIKDDKWIGYNTDGIGYVKGLHSVYPDLENAYILILGAGGASKGIAYELAKFVKPKLTVANRTMARFESWNLNINQISLADAEKYLAEFDIVINTTPAGMAGNNESIINLKHLSPNTLMSDIVYIPYKTPILEEAERKGNHIYNGLDMFVYQGAESFKIWTNKDADINSMKTAVLQQLKGE

SEQ ID NO: 109 - protein, 645 aa, Staphylococcus aureus
MEQINIQFPDGNKKAFDKGTTTEDIAQSISPGLRKKAVAGKFNGQLVDLTKPLETDGSIEIVTPGSEEALEVLRHSTAHLMAHAIKRLYGNVKFGVGPVIEGGFYYDFDIDQNISSDDFEQIEKTMKQIVNENMKIERKVVSRDEVKELFSNDEYKLELIDAIPEDENVTLYSQGDFTDLCRGVHVPSTAKIKEFKLLSTAGAYWRGDSNNKMLQRIYGTAFFDKKELKAHLQMLEERKERDHRKIGKELELFTNSQLVGAGLPLWLPNGATIRREIERYIVDKEVSMGYDHVYTPVLANVDLYKTSGHWDHYQEDMFPPMQLDETESMVLRPMNCPHHMMIYANKPHSYRELPIRIAELGTMHRYEASGAVSGLQRVRGMTLNDSHIFVRPDQIKEEFKRVVNMIIDVYKDFGFEDYSFRLSYRDPEDKEKYFDDDDMWNKAENMLKEAADELGLSYEEAIGEAAFYGPKLDVQVKTAMGKEETLSTAQLDFLLPERFDLTYIGQDGEHHRPVVIHRGVVSTMERFVAFLTEETKGAFPTWLAPKQVQIIPVNVDLHYDYARQLQDELKSQGVRVSIDDRNEKMGYKIREAQMQKIPYQIVVGDKEVENNQVNVRQYGSQDQETVEKDEFIWNLVDEIRLKKHR

SEQ ID NO: 110 - protein, 175 aa, Staphylococcus epidermidis
MSTIAKDQTQINEKIRAKELRLIGQDGEQIGVKSKREALEMAERVDLDLVVVAPNAKPPVARIMDYGKYKFEQQKKEKEMKKKQKVINVKELRLSPTIEEHDFQTKLKNGRKFLSKGDKCKVSIRFRGRAITHKEIGQRVLEKFADECKDIATVEQKPKMEGRQMFIMLAPINEK

SEQ ID NO: 111 - protein, 513 aa, Staphylococcus carnosus
MEMAKEQEMILVLDFGSQYNQLITRRIREMGVYSELHDHEISIEEIKKMNPKGIILSGGPNSVYEEGSYTIDPEIYNLGVPVLGICYGMQLTTKLLGGKVERANEREYGKAIIHAKADELFFGLPEEQTVWMSHSDKVIEIPEGFESIADSPSTPYAAIEDKERRIYGVQFHPEVRHTEYGNDILRNFVRRICDCTGEWTMENFIDLEIEKIREQVGDRKVLCAMSGGVDSSVVAVLLHKAIGDQLTCIFVDHGLLRKGEGDMVMEQFGEGFNMNIIRVNAKDRFMDKLKGVSDPEQKRKIIGNEFVYVFDDEASKLEGVDFLAQGTLYTDVIESGTKTAQTIKSHHNVGGLPEDMQFELIEPINTLFKDEVRALGIELGIPEHLVWRQPFPGPGLGIRVLGEITEEKLEIVRESDAILREVIREEGLEREIWQYFTVLPGMRSVGVMGDYRTYDYTIGIRAVTSIDGMTSDFARIDWEVLQKISSRIVNEVDHVNRVVYDVTSKPPSTIEWE

SEQ ID NO: 112 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 113 - protein, 233 aa, unidentified
  REGION 1..233: note = Bacilli sp.
MTHKYISTQMLIIFTALMIIANFYYIFFEKIGFLLVLLLGCVLVYVGYLYFHKIRGLLAFWIGALLIAFTLLSNKYTIIILFVFLLLLIVRYLIHKFKPKKVVATDEVMTSPSFIKQKWFGEQRTPVYVYKWEDVQIQHGIGDLHIDLTKAANIKENNTIVVRHILGKVQVILPVNYNINLHVAAFYGSTYVNEKSYKVENNNIHIEEMMKPDNYTVNIYVSTFIGDVEVIYR

SEQ ID NO: 114 - protein, 392 aa, Staphylococcus haemolyticus
MTTHEQIINYTIKPHGGTLINRVVEGEERDHLLEAAQSYKVITLNPWSISDLELIGIGGFSPLTGFMGVADYTKVVEDTHLENGLVWSIPITLPVTEEEADKLEIGDDIALYGEDGELYGTLKLEEKYTYDKKKEAQNVYGTTDEAHPGVKKVYDKGNVYLAGPIQLINRPKHDEFSDFHLDPAETRQLFHDLGWKTVVGFQTRNPVHRAHEYIQKSALEIVDGLLLNPLVGETKSDDIPANVRMESYQAILKNYFPKDRARLVIYPAAMRYAGPREAILHATVRKNYGCTHFIVGRDHAGVGDYYGTYEAQELISQFEDELDIQILKFEHAFYCEKCGNMATAKTCPHDASEHVHLSGTKVREKLRNGESLPTKFSRPEVAEVLIKGLKQN

SEQ ID NO: 115 - protein, 393 aa, Staphylococcus aureus
MQFDNIDSALMALKNGEPIIVVDDENRENEGDLVAVTEWMNDNTINFMAKEARGLICAPVSKDIAQRLDLVQMVDDNSDIFGTQFTVSIDHVDTTTGISAYERTLTAKKLIDPSSEAKDFNRPGHLFPLVAQDKGVLARNGHTEAAVDLAKLTGAKPAGVICEIMNDDGTMAKGQDLQKFKEKHQLKMITIDDLIEYRKKLEPEIEFKAKVKMPTDFGTFDMYGFKATYTDEEIVVLTKGAIRQHENVRLHSACLTGDIFHSQRCDCGAQLESSMKYINEHGGMIIYLPQEGRGIGLLNKLRAYELIEQGYDTVTANLALGFDEDLRDYHIAAQILKYFNIEHINLLSNNPSKFEGLKQYGIDIAERIEVIVPETVHNHDYMETKKIKMGHLI

SEQ ID NO: 116 - protein, 299 aa, Staphylococcus sp.
MFDSIRETIDYAVENNMSFADIMVKEEMELSGKSRDEVRAQMKQNLDVMRDAVIKGTTGDGVESVTGYTGHDAAKLRDYNETHHALSGYEMIDAVKGAIATNEVNAAMGIICATPTAGSSGTIPGALFKLEKTHDLTEEQMIDFLFTSALFGRVVANNASVAGATGGCQAEVGSASAMAAAAAVAIFGGSPEASGHAMALAISNLLGLVCDPVAGLVEIPCVMRNAIGSGNALISADLALAGIESRIPVDEVIEAMDKVGRNLPASLRETGLGGLAGTPTGEAIKRKIFGTAEDMVKNN

SEQ ID NO: 117 - protein, 208 aa, Staphylococcus aureus
MNYPNGKPYRKNSAIDGGKKTAAFSNIEYGGRGMSLEKDTEHSNTFYLKSDIAVIHKKPTPVQIVNVNYPKRSKAVINEAYFRTPSTTDYNGVYQGYYIDFEAKETKNKTSFPLNNIHDHQVEHMKNAYQQKGIVFLMIRFKTLDEVYLLPYSKFEVFWKRYKDNIKKSITVDEIRKNGYHIPYQYQPRLDYLKAVDKLILDESEDRV

SEQ ID NO: 118 - protein, 305 aa, unidentified
  REGION 1..305: note = Bacilli sp.
MDWIKDAIKELTEGIADGMMNFIFQFLYTLFYEPLKFFIWLLDKILIDPNSISSDYISNASVLVNSLITSIAICVFAFKMVQVMKQNAEGNAESPGYYVAQLLPSSLLVAVLPWLVDIMTKLSYSATRAFLDVGQKSYLETIKEWSSLKKGQDGFKEMLGSMGTMAGSQIMFILFILLILVFTVVFIFQFVSRIADLVIMKVVAPLVAVSLLADENNYIGVWWRELLAISIQLPLQVFTFFGGINLFFGAKLDVGTLLLGIGFFIITVKSPSFIRSMVYSTGSGRMATGMAGSGAKLLVRKMLSK

SEQ ID NO: 119 - protein, 869 aa, Staphylococcus aureus
MDINKMTYAVQSALQQAVELSQQHKLQNIEIEAILSAALNESESLYKSILERANIEVDQLNKAYEDKLNTYASVEGDNIQYGQYISQQANQLITKAESYMKEYEDEYISMEHILRSAMDIDQTTKHYINNKVEVIKEIIKKVRGGNHVTSQNPEVNYEALAKYGRDLVEEVRQGKMDPVIGRDEEIRNTIRILSRKTKNNPVLIGEPGVGKTAIVEGLAQRIVKKDVPESLLDKTVFELDLSALVAGAKYRGEFEERLKAVLKEVKESDGRIILFIDEIHMLVGAGKTDGAMDAGNMLKPMLARGELHCIGATTLNEYREYIEKDSALERRFQKVAVSEPDVEDTISILRGLKERYEVYHGVRIQDRALVAAAELSDRYITDRFLPDKAIDLVDQACATIRTEMGSNPTELDQVNRRVMQLEIEESALKNESDNASKQRLQELQEELANEKEKQAALQSRVESEKEKIANLQEKRAQLDESRQALEDAQTNNNLEKAAELQYGTIPQLEKELRELEDNFQDEQGEDSDRMIREVVTDEEIGDIVSQWTGIPVSKLVETEREKLLHLSDILHKRVVGQDKAVDLVSDAVVRARAGIKDPNRPIGSFLFLGPTGVGKTELAKSLAASLFDSEKHMIRIDMSEYMEKHAVSRLIGAPPGYIGHDEGGQLTEAVRRNPYSVILLDEVEKAHTDVFNVLLQILDEGRLTDSKGRSVDFKNTIIIMTSNIGSQVLLENVKETGEITESTEKAVMTSLNAYFKPEILNRMDDIVLFKPLSIDDMSMIVDKILTQLNIRLLEQRISIEVSDDAKAWLGQEAYEPQYGARPLKRFVQRQIETPLARMMIKEGFPEGTTIKVNLNSDNNLTFNVEKIHE

SEQ ID NO: 120 - protein, 250 aa, Staphylococcus aureus
MKYNNHDKIRDFIIIEAYMFRFKKKVKPEVDMTIKEFILLTYLFHQQENTLPFKKIVSDLCYKQSDLVQHIKVLVKHSYISKVRSKIDERNTYISISEEQREKIAERVTLFDQIIKQFNLADQSESQMIPKDSKEFLNLMMYTMYFKNIIKKHLTLSFVEFTILAIITSQNKNIVLLKDLIETIHHKYPQTVRALNNLKKQGYLIKERSTEDERKILIHMDDAQQDHAEQLLAQVNQLLADKDHLHLVFE

SEQ ID NO: 121 - protein, 67 aa, unidentified
  REGION 1..67: note = Eubacteria sp.
MRNRLKELRARDGLNQTQLAKQAGVSRQTISLIERNNFMPSVLTAIKIARIFNEKVETIFIIEEDEV

SEQ ID NO: 122 - protein, 262 aa, Staphylococcus sp.
MNWKLTKTLFIFVFILVNIVLVSIYVNKVNRSHINEVESNNEVNFQQEEIKVPASILNKSVKGIQLEQITGRSKDFSSKAKGDSDLTTSDGGKLLNANISQSVKVSDNNLKDLKDYVNKRVFKGSEYQLSEISSGSVKYEQTYDNFPILNNSKAMLNFNIEDNKATSYKQSMMDDIKPTDGADKKHQVIGVRKAIEALYYNRYLKKGDEVINARLGYYSVVNETNVQLLQPNWEIKVKHDGKDKTNTYYVEATNNSPKIINH

SEQ ID NO: 123 - protein, 305 aa, Staphylococcus sp.
MATKNEEILRKPDWLKIKLNTNENYTGLKKMMREKNLNTVCEEAKCPNIHECWGARRTATFMILGAVCTRACRFCAVKTGLPNELDLNEPERVAESVELMNLKHVVITAVARDDLRDAGSNVYAETVRKVRERNPFTTIEILPSDMGGDYDALETLMASRPDILNHNIETVRRLTPRVRARATYDRTLEFLRRSKELQPDIPTKSSIMVGLGETIEEIYETMDDLRANDVDILTIGQYLQPSRKHLKVQKYYTPLEFGKLRKVAMDKGFKHCQAGPLVRSSYHADEQVNEAAKEKQRQGEAQLNS

SEQ ID NO: 124 - protein, 88 aa, Staphylococcus saprophyticus
MAVKLRLTRLGSKRNPFYRIVAADARAPRDGRNIEQIGTYNPNNVNAAEVKIDEELALKWLKNGAKPTDTVHNILSREGILKKFDDQK

SEQ ID NO: 125 - protein, 440 aa, Staphylococcus sp.
MSNGKELQKNIGFFSAFAIVMGTVIGSGVFFKISNVTEVTGTAGMALFVWFLGGIITICAGLTAAELAAAIPETGGLTKYIEYTYGDFWGFLSGWAQSFIYFPANVAALSIVFATQLINLFHLSIGSLIPIAIASALSIVLINFLGSKAGGILQSVTLVIKLIPIIVIVIFGIFQSGDITFSLIPTTGNSGNGFFTAIGSGLLATMFAYDGWIHVGNVAGELKNPKRDLPLAISVGIGCIMAVYLLINATFLLTLPIELLAGNLNAASDTSKILFGENGGKIITIGILISVYGTINGYTMTGMRVPYAMAERKLLPFSHLFAKLTKSGAPWFGAIIQLIIAIIMMSMGAFDTITNMLIFVIWLFYCMSFVAVIILRKREPNMERPYKVPLYPIIPLIAILAGSFVLINTLFTQFILAIIGILITALGIPVYYYKKKQKAA

SEQ ID NO: 126 - protein, 220 aa, Staphylococcus carnosus
MTKGILGRKIGMTQVFGENGDLIPVTVVEAGQNVVLQKKTEEVDGYNAIQVGFEDKQAYKKDSKSNKYANKPAEGHAKKAGTAPKRFIREFKNINVDEYEVGQEVSVDTFEAGDIIDVTGVSKGKGFQGNIKRHGHARGPMAHGSHFHRAPGSVGMASDASKVFKGHKMPGRMGGNTVTVQNLEVVQIDAENNVILVKGNVPGPKKGLVEIKTSIKKGNK

SEQ ID NO: 127 - protein, 224 aa, Staphylococcus sp.
MINKNDIVADVVTDYPKAADIFRSVGIDFCCGGQVSIEAASLEKKNVDLNELLQRLNEVEQTNTPGSLNPKFLNVSSLIQYIQSAYHEPLREEFKNLTPYVTKLSKVHGPNHPYLVELKETYDTFKNGMLEHMQKEDDVDFPKLIKYEQGEVVDDINTVIDDLVSDHIATGQLLVKMSELTSSYEPPIEACGTWRLVYQRLKALEVLTHEHVHLENHVLFKKVS

SEQ ID NO: 128 - protein, 413 aa, Staphylococcus aureus
MSYSIVRVSKVKSGTNTTGIQKHVQRENNNYENEDIDHSKTYLNYDLVNANKQNFNNLIDEKIEQNYTGKRKIRTDAIKHIDGLITSDNDFFDNQTPEDTKQFFEYAKEFLEQEYGKDNLLYATVHMDEKTPHMHYGVVPITDDGRLSAKEVVGNKKALTAFQDRFNEHVKQRGYGLERGQSRQVTNAKHEQISQYKQKTEYHKQEYERESQKTDHIKQKNDKLMQEYQKSLNTLKKPINVPYEQETEKVGGLFSKEIQEAGNVVISQKDFNEFQKQIKAAQDISEDYEYIKSGRALDDKDKEIREKDDLLNKAVERIENADDNFNQLYENAKPLKENIEIALKLLKILLKELERVLGRNTFAERVNKLTEDEPKLNGLAGNLDKKMNPELYSEQEQQQEQQKNQKRDRGMHL

SEQ ID NO: 129 - protein, 652 aa, Staphylococcus sp.
MRVTELLTKDTIAMDLMANDKNGVIDELVNQLDKAGKLSDIASFKEAIHNRESQSTTGIGEGIAIPHAKVAAVKSPAIAFGKSKVGVDYQSLDMQPAHLFFMIAAPEGGAQTHLDALAKLSGILMDENVREKLLHASSPEEVLAIIDHADDEATKEEEAEAEAQQVATAEQSSKQSNEPYVLAVTACPTGIAHTYMARDALKKQADKMGIKIKVETNGSSGIKNHLTEQDIKNATGIIVAADVHVETDRFDGKNVVEVPVADGIKRPEELINKALDTSRKPFVARDGQRKGNSNDSQEKLSPGKAFYKHLMNGVSNMLPLVISGGILMAIVFLFGANSFDPKSSEYNAFAEQLWNIGSKSAFALIIPILSGFIARSIADKPGFASGLVGGMLAISGGSGFIGGIIAGFLAGYLTQGVKVMTRKLPQALEGLKPTLIYPLLTVTATGLLMIYVFNPPASWLNHLLLDGLNNLSGSNIVLLGLVIGAMMAIDMGGPFNKAAYVFATGALIEGNAAPITAAMIGGMIPPLAIATAMLIFRRKFTKEQRGSIIPNYVMGMSFITEGAIPFAAADPLRVIPSMMIGSGIGGAIALGLGSRITAPHGGIIVIVGTDGAHLLQTLIALVVGTLVSALIYGLIKPKLTETEIEASKSMDE

SEQ ID NO: 130 - protein, 371 aa, Staphylococcus epidermidis
MKLNTLQLENYRNYEQVTLDCHPEVNILIGENAQGKTNLLESIYTLALAKSHRTSNDKELIRFKSDYAKIEGELSYRHGTMPLTMFITKKGKQVKVNHLEQSRLTQYIGHLNVVLFAPEDLNIVKGSPQIRRRFIDMELGQISAVYLNDLAQYQRILKQKNNYLKQLQIGQKTDTTMLEVLNQQFVEYALKVTLRREHFIKELETLAQPIHAGITNDQETLTLDYVPSLKLSNYEANQSELIEEVLALLNDNLQREKERGVCLYGPHRDDLSFNVNGMDAQTYGSQGQQRTTALSIKLAEIELMNIEVGEYPILLLDDVLSELDDSRQTHLLSTIQHKVQTFVTTTSVEGIDHEIMNNAKLYRISQGEILK

SEQ ID NO: 131 - protein, 189 aa, Staphylococcus epidermidis
MSLINKEILPFTAQAYDPKKDEFKEVTQEDFKGSWNVVCFYPADFSFVCPTELEDLQNQYAKLQELGVNVYSVSTDTHFVHKAWHDHSDAISKLEYSMIGDPSQTITRNFDVLDEESGLAQRGTFIIDPDGVVQAAEINADGIGRDASTLVNKIKAAQYVRQHPGEVCPAKWEEGSESLQPGLDLVGKI

SEQ ID NO: 132 - protein, 234 aa, Staphylococcus sp.
MASVLYQHGTLGTLMAGLLKGTASINELLQHGDLGIATLTGSNGEVIFLDGKAYHANEHKEFVELKGDELTPYATVTKFVADTSYETKDKSSEAVFAEIKEKMLSENLFSAVKISGLFKKMHVRMMPAQEPPYTRLIDSARRQPEQTETYVKGAVVGFFTPELFHGIGSAGFHVHFANDDRNFGGHVLDFEVEDVKVEIQNIETFEQHFPIQDEDFINANIDYKDIADEIREAE

SEQ ID NO: 133 - protein, 49 aa, Staphylococcus epidermidis
MRVNITLACTECGDRNYITTKNKRNNPERIEMKKYCPRLNKYTLHRETK

SEQ ID NO: 134 - protein, 220 aa, Staphylococcus sp.
MEFKTIQSVKDPHYNEALKLYDTKLNLSLTEDKRIIKRSLENNKTENDYVFIVGLENDVVVSLATAHYEATTNSAFLIYLFAKDLPNHDELMTQTLHQIEHELNLLSNLVHSRSINFLMMEVPKEPADLTQDNGLIIEHRRQFLYEHQFERQTEIDYIHPTNDGHTKKVDLFLKKNIKLTKDIYGTSVKSNYILKYVFANGIAREIIYDLLEEMDLRKPF

SEQ ID NO: 135 - protein, 351 aa, Staphylococcus aureus
MKNKKRVLIASSLSCAILLLSAATTQANSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQKYDTIAIGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 136 - protein, 62 aa, unidentified
  REGION 1..62: note = Eubacteria sp.
MGKQCFVTGRKASTGNRRSHALNSTKRRWNANLQKVRILVDGKPKKVWVSARALKSGKVTRV

SEQ ID NO: 137 - protein, 283 aa, Staphylococcus aureus
MGEYIVTKTLNNNVVVCTNNDQEVILIGKGIGFNKKEGMALNDQTITIEKIYKLESEQQKAHYKSLVEIADDNVLQVIIDSLNFISNTAMNVDSKQLVISLTDHIIFAYKRLKQNQVISNPFVMETMQLYSDAYHIAKQVIDQLNAALDVHFPEDEIGFIALHIASNTEDLSMHEMTLINNVIKKGIDIIESDLVTTVDKESLQYQRFIRHVQFLIRRLRRKEYIHAQDDFVSMIKNHYPICYNTAYKILTMIQKQFDVNISESEIIYLTLHIHHFEERINQS

SEQ ID NO: 138 - protein, 885 aa, Staphylococcus sp.
MSNTESLNIGKKRGSLTIYIGYSPGVGKTFEMLSNAIELFQSNVDIKLGYIEPHQRDETNALAEQLPKIATNFTKHGSHHFQYLDVDRIIEESPTIVLIDELAHTNISRDRHEKRYMDIEEILNHGIDVHTTLNIQHIESLSSQIELMTGVHVKERVPDYFIMSADVLEVVDISPEQLIKRLKAGKVYKKDRLDVAFSNFFTYAHLSELRTLTLRTVADLMSDKEKVRHNHKTSLKPHIAVAISGSIYNEAVIKEAFHIAQKEHAKFTAIYIDVFEKSRQYKDSQKQVHQHLMLAKSLGAKVKVVYSQTVALGLDEWCKNQDVTKLIIGQHIRNKRRDFFNKPLIDHLMSFEHSYKIEIVPIKQIPVELKMNKSPYRPKGKRFAIDMLKMILIQIICVMMGLWIYQLDKHESSTIILMIFLIGIILLSIWTRSYIIGFLAAIINVFVFNYFFTEPRYTFEVYRFDYPITFIVSILTSILTSALLKQIKFQYAITKKQLYRTDLLFQFNDSIKQTYTVENLLINAGYQINQLLQQSITIYVINQSKVIKTIPLQNHIDNKTQQHEQALSWVIKNERQAGATTDTFPGINKWLIPIGTSPIKGILAIDYQSSQVINPYDASILESMLNELSLAVENVTLLKQTRESMLQAERQLTHSNFLRSISHDIRTPLTTIMGNLDILVSHSKDMSIIEKEQLLVHSFQESQYLYLLVTNILSLTKLQSSNVQIKLQPYLVSELVEEIDMILERRHLKKRITVSSSVDLQFIHIDSKLILQALFNLIENAVKHTSTDTKINLSIRYASDEQIEFAVIDEGPGISIEEQQKIFEPFYTGSNKYFKDNQKESMGLGLYLVQTILHKHQSNLQYKPNQPHGSIFYFNIDTDFNEGDI

SEQ ID NO: 139 - protein, 139 aa, unidentified
  REGION 1..139: note = Bacilli sp.
MTDALQQKIHIELLDLLDDVKFELTELNAQKGLYINGPANQLLKRGVHMAYVQGQKQAIDNIMTIVEQQLEDQHFLEHYDKFQNEVAHRNYDKTANFAELSDIPRQFDNFLDQFYQIKGQYFIITHINTLIGDFHSEAH

SEQ ID NO: 140 - protein, 1004 aa, Staphylococcus aureus
MLNRENKTAMTRKGMVSNRLNKFSIRKYTVGTASILVGTTLIFGIGSQEAKAAEVTNKEMKEDATSVNNDQVSKKVDTGQLNNDGNTSKVDTEQLKDENINKVANQKEVTRVENSVALEKNNNSQPSENGKLQTSDVKKTDDNNATSDDQVTTTVNSQQLNTDNTTANATMSSAPINVSKDDLKINSEEVRNVEEKRDNTDNANSSELIKPKSTAPKRLNTRMRMAAVQPSSTDSKNVNDLITSNTTLTVVDADKNNKIVPAQDYLALKSQIKVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNDTTKTTANIQYPDYVSRDNNSIGSAFTETVSHAGNAEDPGYYKQTVYVNPSEKSLTNAKLKVEAYHKDYPDNVGQINKDVTKIKIYQAPKDYVLNKGYDVNTNQLIDVTEQFKDKITYGANDSVNVDFGSINNSYVVMVDTKFEYTTSESPTLVQMATLTSDGNRSVSTGNALGFTNNQSGGAGQEVYKIGNYVWEDTNKNGVQDLGEVGVKGVTVVAYDNKTNKEVGRTITDDKGGYLIPNLPNGDYRVEFSNLPQGYEVTPSKQGNNEELDSNGVSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTTGNDTEKDSNGLTTTGVIKDADNMTLDSGFYKTPK

SEQ ID NO: 141 - protein, 415 aa, Staphylococcus aureus
MNVLVIGAGGREHALAYKLNQSNLVKQVFVIPGNEAMTPIAEVHTEISESDHQGILDFAKQQNVDWVVIGPEQPLIDGLADILRANGFKVFGPNKQAAQIEGSKLFAKKIMEKYNIPTADYKEVERKKDALTYIENCEFPVVVKKDGLAAGKGVIIADTIEAARSAIEIMYGDEEEGTVVFETFLEGEEFSLMTFVNGELAVPFDCIAQDHKRAFDHDEGPNTGGMGAYCPVPHISDDVLKLTNETIAQPIAKAMLNEGYQFFGVLYIGAILTKDGPKVIEFNARFGDPEAQVLLSRMESDLMQHIIDLDEGKRTEFKWKNEAIVGVMLASKGYPDAYEKGHKVSGFDLNENYFVSGLKKQDDTFVTSGGRVILAIGKGDNVHDAQRDAYEKVSQIQSDHLFYRHDIANKALQLK

SEQ ID NO: 142 - protein, 817 aa, Staphylococcus epidermidis
MLFGRLTERAQRVLAHAQEEAIRLNHSNIGTEHLLLGLMKEPEGIAAKVLVSFNITEDKVIEEVEKLIGHGQEQMGTLHYTPRAKKVIELSMDEARKLHHNFVGTEHILLGLIRENEGVAARVFANLDLNITKARAQVVKALGSPEMSNKNAQANKSNNTPTLDGLARDLTVIAKDGTLDPVVGRDKEITRVIEVLSRRTKNNPVLIGEPGVGKTAIAEGLAQAIVKNEVPETLKDKRVMSLDMGTVVAGTKYRGEFEERLKKVMEEIHQAGNVILFIDELHTLVGAGGAEGAIDASNILKPALARGELQCIGATTLDEYRKNIEKDAALERRFQPIQVDEPTVEDTIEILKGLRDRYEAHHRINISDEALEAAAKLSDRYVSDRFLPDKAIDLIDEASSKVRLKSHTTPSNLKEIEQEIDKVKNEKDAAVHAQEFENAANLRDKQSKLEKQYEDAKNEWKNAQGGLDTALSEENIAEVIAGWTGIPLTKINETESDRLLNLEDTLHKRVIGQNDAVNSISKAVRRARAGLKDPKRPIGSFIFLGPTGVGKTELARALAESMFGEDDAMIRVDMSEFMEKHAVSRLVGAPPGYVGHDDGGQLTEKVRRKPYSVILFDEIEKAHPDVFNILLQVLDDGHLTDTKGRTVDFRNTVIIMTSNVGAQELQDQRFAGFGGASEGSDYETVRKTMMKELKNSFRPEFLNRVDDIIVFHKLTKDELKEIVTMMVNKLTHRLSEQNINIVVTDKAKEKIAEEGYDPEYGARPLIRAIQKTVEDNLSELILDGNKIEGKEVTIDHDGKEFKYDIYEITAKKETTES

SEQ ID NO: 143 - protein, 451 aa, Staphylococcus carnosus
MGKYFGTDGVRGVANKELTPELAFKLGRYGGYVLAHNEDKKHPQVLVGRDTRVSGEMLESALIAGLISIGAEVMRLGVISTPGVAYLTKEMDAELGVMISASHNPVADNGIKFFGSDGFKLSDDQENEIEALLDEENPDLPRPVGNEIVHYSDYFEGAQKYLSYIKSTIDVNLDGLKIALDGANGSTSQLAPFLFGDLEADTEVIGCSPDGYNINEGVGSTHPEALAKTVVETESDFGLAFDGDGDRLIAVDEKGNIVDGDQIMFIIGQEMAKNQELNDNMIVSTVMSNLGFYKALEAEGIKSNKTKVGDRYVVEEMRKGNYNLGGEQSGHIVLMDYNTTGDGLLTGVQLAAVVKLTGKSLSELAEQMKKYPQSLINVRVTDKHGVTDNEDVKAEMDRVEADMNGEGRILVRPSGTEPLVRVMVEAATDEKAQSYAQQIADVVEEKMGLDN

SEQ ID NO: 144 - protein, 412 aa, Staphylococcus haemolyticus
MSYIQNQDKAVYEAIQNEYNRQNNNIELIASENFVSEAVMEAQGSVMTNKYAEGYPGRRYYGGCDYVDVTETIAIERAKALFGAEHVNVQPHSGSQANMAVYLVALEMGDTVLGMNLSHGGHLTHGSPVNFSGKFYNFVDYGVDKETEKIDYEVVRQLAHEHKPKLIVAGTSAYSRQLDFKKFKEIADEVGAKLMVDMAHIAGLVAAGLHPNPVEHADFVTTTTHKTLRGPRGGLILCKEEYKKDIDKTIFPGIQGGPLEHVIAAKAVAFGEALEQDFKVYQEQVIKNAKVLSQTLQEEGFRIVSGGTDNHLLSVDVKNSVNVTGKEAEATLDSIGITCNKNTIPFDQEKAFVTSGIRLGTPTATTRGFDEEAFKEVGRIISLALKNPNNDTKLKEARERVSRLTAKYPLYE

SEQ ID NO: 145 - protein, 150 aa, Staphylococcus sp.
MTPEAIIEDRRFQETLDKIRKEEGYDFAAIAFYESNKPSSPIKWHYVSGNKNNRFKLIILRKGRGLAGTVMKTGKRMIIANVGLALGPEEKIDYPILLSESLTAVLAVPLWYKNQVYGVLLFGQRDGRSLPKIFDNDDIQRKFGIFNDDK

SEQ ID NO: 146 - protein, 191 aa, Staphylococcus sp.
MNAKEQLVDNLMKTSSQLFKFHGEVAMQLFLNDELKLPSIVEICVERKRLSDIVKVIPQSYALLYIDKQDQAIAKEDLSLSKIAKVYVQYDNTTIMSIFVYDVVNDEWIFRLDPNIRIPKSNIYFHSLNWDVDYIKPEIVLMYDLMQHHQYHHYSNYKRVIDALSYYQFFILKFVVGEQRIKDAIQRTINN

SEQ ID NO: 147 - protein, 215 aa, Staphylococcus sp.
MQQSDIISAAKKYMESIHQNDFTGHDIAHVYRVTALAKSIAENEGVNDTLVIELACLLHDTVDEKVVDANKQYVELKSFLSSLSLSTEDQEHILFIINNMSYRNGKNDHVTLSLEGQIVRDADRLDAIGAIGVARTFQFAGHFGEPMWTEQMSLDKINDDLVEQLPPSAIKHFFEKLLKLESLMHTDTAKMIAKERHDFMMMYLKQFLTEWNYHD

SEQ ID NO: 148 - protein, 585 aa, Staphylococcus aureus
MAKLLIMSIVSFCFIFLLLLFFRYILKRYFNYMLNYKVWYLTLLAGLIPFIPIKFSLFKFNNVNNQAPTVESKSHDLNHNINTTKPIQEFATDIHKFNWDSIDNISTVIWIVLVIILSFKFLKALLYLKYLKKQSLYLNENEKNKIDTILFNHQYKKNIVIRKAETIQSPITFWYGKYIILIPSSYFKSVIDKRLKYIILHEYAHAKNRDTLHLIIFNIFSIIMSYNPLVHIVKRKIIHDNEVEADRFVLNNINKNEFKTYAESIMDSVLNVPFFNKNILSHSFNGKKSLLKRRLINIKEANLKKQSKLILIFICIFTFLLMVIQSQFLMGQSITDYNYKKPLHNDYQILDKSKIFGSNSGSFVMYSMKKDKYYIYNEKESRKRYSPNSTYKIYLAMFGLDRHIINDENSRMSWNHKHYPFDAWNKEQDLNTAMQNSVNWYFERISDQIPKNYTATQLKQLNYGNKNLGSYKSYWMEDSLKISNLEQVIVFKNMMEQNNHFSKKAKNQLSSSLLIKKNEKYELYGKTGTGIVNGKYNNGWFVGYVITNHDKYYFATHLSDGKPSGKNAELISEKILKEMGVLNGQ

SEQ ID NO: 149 - protein, 494 aa, Staphylococcus aureus
MFNYSGLNEECGVFGIWNHPEAAQLTYMGLHSLQHRGQEGAGIVVSDQNELKGERGLGLLTEAIKDDQMERLKGYQHAIGHVRYATSGNKGIENIQPLLYHFYDMSVGICHNGNLINAKSLRQNLEKQGAIFHSSSDTEVIMHLIRRSKAPTFEEALKESLRKIKGGFTFAILTKDALYGAVDPNAIRPLVVGKMKDGTYILASETCAIDVLGAEFVQDIHAGEYVVINDKGITVKSYTHHTTTAISAMEYIYFARPDSTIAGKNVHAVRKASGKMLAQESPVKADMVIGVPNSSLSAASGYAEEIGLPYEMGLVKNQYVARTFIQPTQELREQGVRVKLSAVKDIVDGKNIILVDDSIVRGTTIRRIVKMLKDSGANKVHVRIASPEFMFPSFYGIDVSTTAELISASKSPEEIKDYIGADSLAYLSVDGLIESIGLDYDAPYSGLCVESFTGDYPAGLYDYEANYKAHLSHRQKQYISKNKHYFDSEGNLNV

SEQ ID NO: 150 - protein, 234 aa, Staphylococcus sp.
MLKYEHIAKQLNAFIHQSNFKPGDKLPSVTQLKERYQVSKSTIIKALGLLEQDGLIYQAQGSGIYVRNIADANRINVFKTNGFSKSLGEHRMTSKVLVFKEIATPPKSVQDELQLNADDTVYYLERLRFVDDDVLCIEYSYYHKEIVKYLNDDIAKGSIFDYLESNMKLRIGFSDIFFNVDQLTSSEASLLQLSAGEPCLRYHQTFYTMTGKPFDSSDIVFHYRHAQFYIPSKK

SEQ ID NO: 151 - protein, 492 aa, Staphylococcus sp.
MKKAILSVSNKTGIVEFAKALTQLNYELYSTGGTKRILDEANVPVRSVSDLTHFPEIMDGRVKTLHPAVHGGILADRNKPQHLNELSEQHIDLIDMVVVNLYPFQQTVANPDVTMDEAIENIDIGGPTMLRAAAKNYKHVTTIVHPADYQEVLTRLRNDSLDESYRQSLMIKVFEHTAEYDEAIVRFFKGDKETLRYGENPQQSAYFVRTSNAKHTIAGAKQLHGKQLSYNNIKDADATLALVKKFDTPAAVAVKHMNPCGVGIGDTLEQAFHHAYEADSQSIFGGIVALNRAVTPELAEQLHSIFLEVIIAPKFTDEALNILKQKKNVRLLEIDMTIDSNEEEFVSVSGGYLVQDKDNYVVPKEEMKVVTEVAPTDEQWEAMLLGWKVVPSVKSNAIILSNNKQTVGIGAGQMNRVGAAKIALERAIEINDHVALVSDGFFPMGDTVELAAQHGIKAIIQPGGSIKDQDSIDMANKHGIAMVVTGTRHFKH

SEQ ID NO: 152 - protein, 61 aa, Staphylococcus saprophyticus
MAKTSMVAKQQKKQKFQVREYTRCERCGRPHSVYRKFKLCRICFRELAYKGQIPGVRKASW

SEQ ID NO: 153 - protein, 178 aa, Staphylococcus sp.
MITVLFGGSRPNGNTAQLTKFALQDLEYQWIDVTQHQFKPIRDVRHTAETITSYDDDYLPILDKILASDTIIFASPVYWYSISAPLKAFIEHWSETLQDKRYPNFKAQMAEKDFRVILVGGDCPKIKAKPAITQMKYSLDFLGATLNGYIIGTAEKPGDIMKDNYALARATEWNSILQ

SEQ ID NO: 154 - protein, 154 aa, unidentified
  REGION 1..154: note = Eubacteria sp.
MDRQSFTDLIQTKFKMVRIEAGYTQDTMAQTIGLSKKTLVQIEKERVLPNWTTCISICALFRDSEVLNSTFGCDPLEIVQTISRNHCAYPNHAPTSDIYWNNIETRNGYILQSNKVSNIYRVLNPDNQPIFGTSKMREAETYFNRNAKEELVHI

SEQ ID NO: 155 - protein, 333 aa, Staphylococcus sp.
MALLKNFFIGLSNNSFLNNAAKKVGPRLGANKVVAGNTIPELINTIEYLNDKNIAVTVDNLGEFVGTVEESNHAKEQILTIMDALHQHGVKAHMSVKLSQLGAEFDLELAYQNLREILLKANTYNNMHINIDTEKYASLQQIVQVLDRLKGEFRNVGTVIQAYLYDSHELVDKYQDLRLRLVKGAYKENESIAFQSKEDVDANYIKIIEQRLLNARNFTSIATHDHRIINHVKQFMKENHIEKDRMEFQMLYGFRSELAEEIANEGYNFTIYVPYGDDWFAYFMRRLAERPQNLSLAAKEFVKPAGLKRVGIIAALGATVMLCLSTIKKLCRK

SEQ ID NO: 156 - protein, 221 aa, Staphylococcus carnosus
MNWSEVFHDITNRHDFKEMHDFLDKEYSTKTVYPDKENIYQAFDLTPFDKVKVVIIGQDPYHGPKQAHGLAFSVQPEGKFPPSLRNMYKELEDDIGCKRTVSHLQDWAREGVLLLNTVLTVRKGEAASHRNIGWEVFTDEIIKAVSEYRDHVVFILWGKPAQQKERLIDTSKHDVIKSVHPSPLSAYRGFFGSKPYSRANTYLEAHGETPIDWCGEEAQRG

SEQ ID NO: 157 - protein, 426 aa, Staphylococcus sp.
MKKLNIALLGLGTVGSGVVKIIEENRQQIQDTLNKDIVIKHILVRDKSKKRPLNISQYHLTEDVNEILNDDSLDIIVEVMGGIEPTVDWLRTALKNKKHVITANKDLLAVHLKLLEDLAEENGVALKFEASVAGGIPIVNAINNGLNANNISKFMGILNGTSNFILSKMTKEQTTFEEALDEAKRLGFAEADPTDDVEGVDAARKVVITSYLSFNQVIKLNDVKRRGISGVTLTDINVADQLGYKIKLIGKGIYENGKVNASVEPTLIDKKHQLAAVEDEYNAIYVIGDAVGDTMFYGKGAGSLATGSAVVSDLLNVALFFESDLHTLPPHFELKTDKTREMMDSDAEINIKEKSNFFVVVNHVKGSIENFENELKAILPFHRSLRVANYDNQAYAAVIVGLESSPEELITKHGYEVDKVYPVEGV

SEQ ID NO: 158 - protein, 296 aa, Staphylococcus sp.
MNKISKYIAIASLSVAVTVSAPQTTNSTAFAKSSAEVQQTQQASIPASQKANLGNQNIMAVAWYQNSAEAKALYLQGYNSAKTQLDKEIKKNKGKHKLAIALDLDETVLDNSPYQGYASIHNKPFPEGWHEWVQAAKAKPVYGAKEFLKYADKKGVDIYYISDRDKEKDLKATQKNLKQQGIPQAKKSHILLKGKDDKSKESRRQIVQKDHKLVMLFGDNLLDFTDPKEATAESREALIEKHKDDFGKKYIIFPNPMYGSWEATIYNNNYKASDKAKDKLRKNAIKQFDPKTGEIK

SEQ ID NO: 159 - protein, 353 aa, Staphylococcus sp.
MRRWQGLVEEFKAHLPVNENTPKLTLNEGNTPLIHCENMSEILGIDLYVKYEGANPTGSFKDRGMVMAVTKAKEQGKKIVICASTGNTSASAAAYAARAGLKAIVVIPEGKIALGKLSQAVMYGAEIVSIEGNFDEALEIVKEIAKSGEIELVNSVNPFRIEGQKTGSFEIVQQLDGEAPDILAIPVGNAGNITAYWKGFKEYHEAKGSQLPKMFGFQAEGASPIVQNKVIKNPETIATAIRIGNPASWDKATNALKESNGLIDSVTDDEILEAYQLMTTKEGVFSEPASNASIAGLIKLHRQGKLPQGKKVVAILTGNGLKDPDTAISLLDNPIKPLPNDKDSIIDYIKGAL

SEQ ID NO: 160 - protein, 202 aa, Staphylococcus sp.
MCFDINNFDIKKLNFRKVKNAIHLSLSGDKYQFLEDGKTINNTYFLAVYDNAINIFEDLFSPSDSINLVHVVYVYNEPYKRTNVIKKFTSLTIDELYYFKEYKKEKDDNILCEEFSYKCSIKDIKYKKLLKAISNQDFKGLVPNINQNDNYSEIYIINNTKNLIYHLYDDRGLWLAFNNNEDYARYSEKYNDLILEFDNEDI

SEQ ID NO: 161 - protein, 100 aa, unidentified
  REGION 1..100: note = Eubacteria sp.
MHFTQREQDKLMIVVAAEVARRRKARGLKLNHPEALALISDELLEGARDGKTVAELMSYGRQILNKEDVMDGVEHMITDIEIEATFPDGTKLITVHHPIV

SEQ ID NO: 162 - protein, 544 aa, Staphylococcus sp.
MKGIELLNNPFLNKGTAFTNEERKQLGLEGLLPANVRTLEQQAEQCYEQFKAKQTDFEKRLFLMAIFNRNRTLFYKLTSEHLVEFMPIIYDPVIAQSIEQYNENFSRPQDAVFLSVDRQDQIEQELRNVANGRDIRLIVVTDAEGILGIGDWGVDGVDIAIGKLIVYTAAAGINPAQVLPVSLDVGTNNEQLLNDELYLGNRHKRVDDETYYTFVDKFVSAVRKEYPNALLHWEDFGRGHAKNILDKYEDTLPTFNDDIQGTGIVTLAGVLGALNISKVDYTNQTFLVYGGGTAGMGITNILKDELIKQDVSEDKANQHFYIMDKQGLLFDDMDDLTEAQQVFAKNRNEFSNTVNWKNLKEVIENIKPTVLIGTSTQTGAFTEDAVKEMLKHTERPMIFPLSNPTKLAEATAQDLLKWTNGKALIGTGIPYNDIEYNGVNYSIGQANNALMYPGLGLGLIASKAKKVNQQILSAASHALGGMVNPDEPGAAVLPPVEKIHQCSRAIAKAVAQSVIDQGLNAETIDDIDEAIEAEIWYPEYKSYI

SEQ ID NO: 163 - protein, 359 aa, Staphylococcus sp.
MRIAILGATNIKHMSLLSHYLNHIDLNINEVDIIYTDKYDIEENIQGINNYYKYKVDIKEDWTFIKKAIAYYRFRPYAMKILKENHYDFVIVWGSYTGHLFKSFLEKHYKNKFILNIRDYFFENNKLIKYRMKKIVDASRLTTLSSEGFLKFLPKSEKYRIIYSYNMSIIRESNVTDEFKKRWPINIGFIGNVRFNEINQKLIKELANDSRFHMQYFGTGSEKLEVFARENFINNITFSGGFDLEETPKYLNEIDILNNLFGNKNIALDTALSIRMYYALFLNKPIITTDDTFTATEANKFGLGFSVNPENLKGIGDELMDWYNNLNVMDINHKREAYRNDVIENNKQFYQEIGRIFNE

SEQ ID NO: 164 - protein, 681 aa, Staphylococcus aureus
MRKKLFGQLQRIGKALMLPVAILPAAGLLLAIGTAMQGESLQHYLPFIQNGGVQTVAKLMTGAGGIIFDNLPMIFALGVAIGLAGGDGVAAIAAFVGYIIMNKTMGDFLQVTPKNIGDPASGYASILGIPTLQTGVFGGIIIGALAAWCYNKFYNINLPSYLGFFAGKRFVPIMMATTSFILAFPMALIWPTIQSGLNAFSTGLLDSNTGVAVFLFGFIKRLLIPFGLHHIFHAPFWFEFGSWKNAAGEIIHGDQRIFIEQIREGAHLTAGKFMQGEFPVMMFGLPAAALAIYHTAKPENKKVVAGLMGSAALTSFLTGITEPLEFSFLFVAPLLFFIHAVLDGLSFLTLYLLDLHLGYTFSGGFIDYFLLGILPNKTQWWLVIPVGLVYAVIYYFVFRFLIVKLKYKTPGREDKQSQAATASATELPYAVLEAMGGKANIKHLDACITRLRVEVNDKSKVDVPGLKDLGASGVLEVGNNMQAIFGPKSDQIKHEMQQIMNGQVVENPTTMEDDKDETVVVAEDKSATSELSHIVHAPLTGEVTPLSEVPDQVFSEKMMGDGIAIKPSQGEVRAPFNGKVQMIFPTKHAIGLVSDSGLELLIHIGLDTVKLNGEGFTLHVEEGQEVKQGDLLINFDLDYIRNHAKSDITPIIVTQGNITNLDFKQGEHGNISFGDQLFEAK

SEQ ID NO: 165 - protein, 315 aa, Staphylococcus sp.
MALTIRYQTTYEPFKVVDDIKEIPKDATIVWYDFDEPNEQENEWFKAHFSFNDLEVDDAINGMPRAKYKSYKDYQYLVFHSIMGSNYSPIALNIFIQDNVLVTYHHQTLESLNKVVYKYMNTLDAELDCADVVILILDMMVDKYFNFVYALEDSVYHFEDRHVDDRFNKMVMDSVFKLRSDLIKVKRVLFPMQELIDTMKQNGDLIIDNKHSLYIQHIDDHLIKQRNIIRTAQEMTNEIRENFESYTSFRMNSIMQVLTLVSVIFSPLTFIAGIYGMNFVNMPALHLHYGYYICLAVMFVIAVVLIIFFRRKKWF

SEQ ID NO: 166 - protein, 288 aa, Staphylococcus sp.
MNEQWLEHLPLKDIKEISPVSGGDVNEAYRVETDTDTFFLLVQRGRKESFYAAEIAGLNEFERAGITAPRVIASGEVNGDAYLVMTYLEEGASGSQRQLGQLVAQLHSQQQEEGKFGFSLPYEGGDISFDNHWQDDWCTIFVDKRMDHLKDELLNRGLWDANDIKVYDKVRRQIVAELEKHQSKPSLLHGDLWGGNYMFLQDGRPALFDPAPLYGDREFDIGITTVFGGFTSEFYDAYNKHYPLAKGASYRLEFYRLYLLMVHLLKFGEMYRDSVTHSMDKILQDTTD

SEQ ID NO: 167 - protein, 306 aa, Staphylococcus sp.
MIYTVTFNPSIDYVIFTNDFKIDGLNRATATYKFAGGKGINVSRVLKTLDVESTALGFAGGFPGKFIIDTLNNSAIQSNFIEVDEDTRINVKLKTGQETEINAPGPHITSTQFEQLLQQIKNTTSEDIVIVAGSVPSSIPSDAYAQIAQITAQTGAKLVVDAEKELAESVLPYHPLFIKPNKDELEVMFNTTVNSDADVIKYGRLLVDKGAQSVIVSLGGDGAIYIDKEISIKAVNPQGKVVNTVGSGDSTVAGMVAGIASGLSIEKAFQQAVACGTATAFDEDLATRDAIEKIKSQVTICVLDGE

SEQ ID NO: 168 - protein, 442 aa, Staphylococcus aureus
MAMYHFQNKFVSKVNGQSATAKSAYNSASRIKDFKENEFKDYSNKQCDYSEILLPNNADDKFKDREYLWNKVHDVENRKNSQVAREIIIGLPNEFDPNSNIELAKEFAESLSNEGMIVDLNIHKINEENPHAHLLCTLRGLDKNNEFEPKRKGNDYIRDWNTKEKHNEWRKRWENVQNKHLEKNGFSVRVSADSYKNQNIDLEPTKKEGWKARKFEDETGKKSSISKHNESIKKRNQQKIDKMFNEVDDMKSHKLNAFSYMNKSDSTTLKNMAKDLKIYVTPINMYKENERLYDLKQKTSLITDDEDRLNKIEDIEDRQKKLESINEVFEKQAGIFFDKNYPDQSLNYSDDEKIFITRTILNDRDVLPANNELEDIVKEKRIKEAQISLNTVLGNRDISLESIEKESNFFADKLSNILEKNNLSFDDVLENKHEGMEDSLKI

SEQ ID NO: 169 - protein, 126 aa, Staphylococcus sp.
MKIILKNNSDFPIYEQIKQQVKQNILKGHVAPGEHLPSMRELAKDLQVSLITTKRAYEDLEKDGFVTTIRGKGTFVKEQDSSILKEKQFFTIENLVKELVNEAQAIEMSLEELQDILTFIYEEESS

SEQ ID NO: 170 - protein, 342 aa, Staphylococcus saprophyticus
MSKAYQQAGVDINAGYEAVERMTSHVQRTMRKEVLGGLGGFGATFDLSQLNMKAPLLVSGTDGVGTKLKLAIDNDKHDTIGIDAVAMCVNDILTTGAEPLYFLDYIATNKVVPEVIEQIVKGVSDGCEETNTALIGGETAEMGEMYHEGEYDLAGFAVGAVEKDDYIDGSSVEPGQAIIGLESSGIHSNGYSLVRKLIQQSGIKLSEPFNQEQTFLDAFLKPTRLYVKPVLAVKSSIKIYAMTHITGGGFYENIPRALPEGITAKIDVTQFPTPAIFDWLQEQGNISTDEMYNIFNMGIGFTLVVDNNQVESTLEILNGQNIKAYKIGETVKGNEPIQLTGV

SEQ ID NO: 171 - protein, 327 aa, Staphylococcus sp.
MKKWQFVGTTALGATLLLGACGGGNGGSGNSDLKGEAKGDGSSTVAPIVEKLNEKWAQDHSDAKISAGQAGTGAGFQKFIAGDIDFADASRPIKDEEKQKLQDKNIKYKEFKIAQDGVTVAVNKENDFVDELDKQQLKAIYSGKAKTWKDVNSKWPDKKINAVSPNSSHGTYDFFENEVMNKEDIKAEKNADTNAIVSSVTKNKEGIGYFGYNFYVQNKDKLKEVKIKDENGKATEPTKKTIQDNSYALSRPLFIYVNEKALKDNKVMSEFIKFVLEDKGKAAEEGGYVAAPEKTYKSQLDDLKAFIDKNQKSDDKKSDDKKSEDKK

SEQ ID NO: 172 - protein, 342 aa, Staphylococcus aureus
MSKAYEQSGVNIHAGYEAVERMSSHVKRTMRKEVIGGLGGFGATFDLSQLNMTAPVLVSGTDGVGTKLKLAIDYGKHDSIGIDAVAMCVNDILTTGAEPLYFLDYIATNKVVPEVIEQIVKGISDACVETNTALIGGETAEMGEMYHEGEYDVAGFAVGAVEKDDYVDGSEVKEGQVVIGLASSGIHSNGYSLVRKLINESDIDLASNFDNRPFIDVFLEPTKLYVKPVLALKKEVSIKAMNHITGGGFYENIPRALPAGYAARIDTTSFPTPKIFDWLQQQGNIDTNEMYNIFNMGIGYTVIVDEKDVSRALKILAEQNVEAYQIGHIVKNESTAIELLGV

SEQ ID NO: 173 - protein, 224 aa, Staphylococcus aureus
MVQCLVVDDDPRILNYIASHLQIEHIDAYTQPSGEAALKLLEKQRVDIAVVDIMMDGMDGFQLCNTLKNDYDIPVIMLTARDALSDKERAFISGTDDYVTKPFEVKELIFRIRAVLRRYNINSNSEMTIGNLTLNQSYLELQVSNKTMTLPNKEFQLLFMLAARPKQIFTREQIIEKIWGYDYEGDERTVDVHIKRLRQRLKKLNATLTIETVRGQGYKVENHV

SEQ ID NO: 174 - protein, 397 aa, Staphylococcus aureus
MKFNKVKLVIHACVLLFIIISIGLVFHRLQTKTNSIEPIHKETKLSDNAKYLVDRNKGKGEPSKLKEVYNSKDPKYKKIDRYLQNSLFNGSVAVYENGKLKMSKGYGYQDFEKGIKNTPNTMFLIGSAQKFSTGLLLKQLEEEHKININDPVSKYIPWFKTSKPIPLKDLMLHQSGLYKYKSSKDYKNLDQAVRAIQKRGIDPKKYKKHMYNDGNYLVLAKVIEEVTGKSYAENYYTKIGDPLKLQHSAFYDEKSFRKYFAKGYSYNSTGLSFLKPNVLEQYYGAGNIYMTPTDMGKLITQIQQYKLFSPKITNPLLHEFGTKQYPDEYRYGFYVKPTLNRLNGGFFGQVFTVYYNDKYVVVLALNVKGNNEVRIKHIYNDILKQNKPYNSKGVIVQ

SEQ ID NO: 175 - protein, 383 aa, Staphylococcus aureus
MNELEFVTKHRRHLHQHPELSLHEFETTAYIKAFLDSLNIKYDCPLETGVIAYLEGNGSHTIAYRADIDALPILEENDVPYRSQSDHVMHACGHDGHTTTLMLFVQRCKDMQDAGQLPQNVVFIFQPAEETGGGANRLIKAGAFDKYPIEAVFGIHVNPFADEGIAVIRDEEITASATEYRFFLTGLSSHVADKEQGHSCGEALQHVLTQISQIQQFHLNGLKRNIVHIGHFKAGEAINTVPSNGYLEGTIRTYDIDDLTIVKNQMHKIAESVKLLFNVDCEVKFAEGYPPTINSPKLRTQIEDALIKADLNVYDKPTPFLFGEDFSFYGQQLAPAYFVFIGTRNEDKGFVTGLHTSYLNFDEKVLINVVNFYENLLNNYKEV

SEQ ID NO: 176 - protein, 307 aa, Staphylococcus sp.
MKPKVLLAGGTGYIGKYLSEVIENDAELFAISKYPDNKKTDDVEMTWIQCDIFHYEQVVAAMNQIDIAVFFIDPTKNSAKITQSSARDLTLIAADNFGRAAAINQVKKVIYIPGSRYDNETIERLGAYGTTVETTNLVFKRSLVNVELQVSKYDDVRSTMKVVLPKGWTLKNVVNHFIAWMGYTKGTFVKTEKSHDQFKIYIKNKVRPLAVFKIVETADGIITLILLSGSLVKKYTVNQGKLEFRLIKESSVVYIHLYDYIPRLFWPIYYFIQAPMQKMMIHGFEVDCRIKDFQSRLKSGENMKYTK

SEQ ID NO: 177 - protein, 418 aa, unidentified
  REGION 1..418: note = Staphylococcaceae sp.
MIDNFMQVLKLIKEKRTNNVVKKSDWDKGDLYKTLVHDKLPKQLKVHIKEDKYSVVGKVATGNYSKVPWISIYDENITKETKDGYYLVYLFHPEGEGIYLSLNQGWSKISDMFPRDKNAAKQRALTLSSELNKYITSNEFNTGRFYYAENKDSSYDLKNDYPSGYSHGSIRFKYYDLNEGFTEEDMLEDLKKFLELFNELASKVTKTSYDSLVNSIDEIQEDSEIEEIRTAQKDKTLKEVEAPKGIIPKYKKGVSKTTKNDSEIEKSNKENKLTGKVGEKLALNYFNELIDNKIDEDKKEQFRNILNDNPGSQHGHGYDLVAFDPTNTDKAVEKFIEIKTSTSSSIEEPFFMSLNEMFAMKEYKQKYLILRIFNVSGKEPQFYFIDPYANYSEFKDVDDLIDKVFNVEAIQYKVFGEK

SEQ ID NO: 178 - protein, 571 aa, Staphylococcus sp.
MSFKMTQNQYTSLYGPTVGDSIRLGDTNLFAQIEKDYAVYGEEATFGGGKSIRDGMAQNPRVTRDDVNVADLVISNAVIIDYDKVVKADIGIKNGYIFAIGNAGNPDIMDNVDIIIGSTTDIIAAEGKIVTAGGIDTHVHFINPEQAEVALESGITTHIGGGTGASEGSKATTVTPGPWHIHRMLEAAEGLPINVGFTGKGQATNPTALIEQINAGAIGLKVHEDWGATPSALSHALDVADEFDVQIALHADTLNEAGFMEDTMAAVKDRVLHMYHTEGAGGGHAPDLIKSAAFPNILPSSTNPTLPYTHNTVDEHLDMVMITHHLNAAIPEDIAFADSRIRKETIAAEDVLQDMGVFSMISSDSQAMGRVGEVITRTWQVAHRMKEQRGPLDGDYEHNDNNRIKRYIAKYTINPAITHGISEYVGSIEPGKLADIVLWDPIFFGVKPELVVKGGLINSAVNGDANGSIPTSEPMKYRKMYGQYGGNLTSTSMTFVSKTAYENGINRALNLKRMVRPVKNIRQLSKADMKNNSATPKLDVDPQTYEVYVDGEKITSNAATELPLTQRYFLF

SEQ ID NO: 179 - protein, 87 aa, Staphylococcus sp.
MKTIELHITLQPQVLDTQGQTLTRAVHDLGYAQVNDIRVGKVLYMTVDEVSDEKVHNIITTLSEKLFANTVIEEYSYKVLEDEKENA

SEQ ID NO: 180 - protein, 221 aa, Staphylococcus aureus
MALVVKDIVKNFGEGLSETKVLKGINFEVEQGEFVILNGASGSGKTTLLTILGGLLSQTSGTVLYNDAPLFDKQHRPSDLRLEDIGFIFQSSHLVPYLKVIEQLTLVGQEAGMTKQQSSTRAIQLLKNIGLEDRLNVYPHQLSGGEKQRVAIMRAFMNNPKIILADEPTASLDADRATKVVEMIRQQIKEQQMIGIMITHDRRLFEYADRVIELEDGKITD

SEQ ID NO: 181 - protein, 166 aa, Staphylococcus epidermidis
MFKKLFGKAKEVDKNIKIYAPLTGEYVKIEDIPDPVFAQKMMGEGFGINPTEGEVVSPIEGKVDNVFPTKHAVGLKAENGLELLVHIGLDTVQLDGKGFEVLVESGDDIKIGDPLIRFDLEYINNNAKSIISPIIITNSDQTESIHIEDVQAVVKGETQVIDVTVS

SEQ ID NO: 182 - protein, 433 aa, Staphylococcus aureus
MRMIDIIEKKRDGHTLTTEEINFFIDGYVKGDIPDYQASSLAMAIYFQDMNDDERAALTMAMVNSGDMIDLSDIKGVKVDKHSTGGVGDTTTLVLAPLVAAVDVPVAKMSGRGLGHTGGTIDKLEAIDGFHVEIDEATFVKLVNENKVAVVGQSGNLTPADKKLYALRDVTGTVNSIPLIASSIMSKKIAAGADAIVLDVKTGSGAFMKTLEDAEALAHAMVRIGNNVGRNTMAIISDMNQPLGRAIGNALELQEAIDTLKGQGPKDLTELVLTLGSQMVVLANKAETLEEARALLIEAINSGAALEKFKTFIKNQGGDETVIDHPERLPQAQYQIEYKAKKSGYVTELVSNDIGVASMMLGAGRLTKEDDIDLAVGIVLNKKIGDKVEEGESLLTIHSNRQDVDDVVKKLDSSITIADHVVSPTLIHKIIIE

SEQ ID NO: 183 - protein, 609 aa, Staphylococcus epidermidis
MGKVIGIDLGTTNSCVSILEGDEPKVIQNPEGARTTPSVVAFKNGETQVGEVAKRQAITNPNTVQSIKRHMGTDYKVDIEGKSYTPQELSAMILQNLKSTAENYLGDTVDKAVITVPAYFNDGERQATKDAGKIAGLEVERIINEPTAAALAYGLDKTETDQKVLVFDLGGGTFDVSILELGDGVFEVLSTAGDNKLGGDDFDQVIIDYLVSEFKKENGVDLSQDKMALQRLKDAAEKAKKDLSGVSQTQISLPFISAGENGPLHLEISLTRSKFEELADSLIKKTMEPTRQALKDAGLSTSEIDEVILVGGSTRIPAVQEAVKKEIGKEPHKGVNPDEVVAMGAAIQAGVITGDVKDVVLLDVTPLSLGIEIMGGRMNTLIERNTTIPTSKSQVYSTAADNQPAVDIHVLQGERPMASDNKTLGRFQLTDIPPAPRGVPQIEVTFDIDKNGIVNVTAKDLGTNKEQNITIQSSSSLSDEEIDRMVKDAEENAEADKKRREEVDLRNEADSLVFQVEKTVKDLGENISDEDKKNAEEKKDALKTALEGEDIDDIKAKKEELEKVIQELSAKVYEQAQQAQQQGQEEQGSQDSTVEDADFKEVKDDEDKK

SEQ ID NO: 184 - protein, 252 aa, Staphylococcus aureus
MFKVVICDDERIIREGLKQIIPWGDYHFNTIYTAKDGVEALSLIQQHQPELVITDIRMPRKNGVDLLNDIAHLDCNVIILSSYDDFEYMKAGIQHHVLDYLLKPVDHAQLEVILGRLVRTLLEQQSQNGRSLAPCHDAFQPLLKVEYDDYYVNQIVDQIKQSYQTKVTVSDLIQHIDVSESYAMRTFKDHVGITIVDYLNRYRILQSLQLLDRHYKHYEIADKVGFSEYKMFSYHFKKYLQMAPSDYCKQAK

SEQ ID NO: 185 - protein, 295 aa, Staphylococcus saprophyticus
MDTIIEEYLKFIQLEKGLSSNTIGAYRRDLKKYNTFLELQKISHIDFIDRASIQQCLGYLHDNGASAKSLARFISTIRSFHQFALREKYAAKDPTVLIETPKYDRKLPDVLEINEVLALLETPNLAKINGYRDRTMLELLYATGMRVSELIQIELEDVNLIMGFVKVFGKGNKERIVPLGDTVIEYLKTYIETIRPQLLKKTVTNTLFLNMHGKPLSRQAIWKMIKQNAIKANITKSLTPHTLRHSFATHLLENGADLRAVQEMLGHSDISTTQLYTHVSKSQIRKMYNEFHPRA

SEQ ID NO: 186 - protein, 476 aa, Staphylococcus sp.
MNESGDNKLSKSSLIGLVIGSMIGGGAFNIMSDMGGKAGGLAIIIGWIITAIGMISLAFVFQNLTNERPELDGGIYSYAQAGFGDFVGFISAWGYWFSAFLGNVAYATLLMSAVGNFFPIFKGGNTLPSIIVASLLLWGVHFLILKGVETAAFINSIVTVAKLIPILLVIICMIIAFNFDTFKTGFFGMTSEGVLPFSWASTMSQVKSTMLVTVWVFIGIEGAVIFSSRAKNKKDVGSATVIGLISVLIIYFLLTVLAQGVILQNHISQLDSPSMAQVLASIVGGWGSTLVNIGLIISVLGAWLGWTLLAGELPFIVAKDGLFPKWFAKENKNGAPVNALLITNILVQLFLISMLFTQSAYQFAFSLASSAILYPYMFSAFYQVKYTLEHRQQATTKQWTIGIIASIYAIWLIYAAGINYLLLTMLLYIPALLVYSIVQKNNQTRLIKSDYILFMIIIVLAVIGLIKLLMGTINVF

SEQ ID NO: 187 - protein, 231 aa, Staphylococcus carnosus
MAKKGKKYLDAASKVDRNEHYSVEDAISLAKETSVANFDATVEVAFRLVIDTRKNDQQIRGAVVLPHGTGKTQRVLVFAKGDKASEAEAAGADYVGESEYATKIQQGWFDFDVVVATPDMMGEVGKLGRVLGPKGLMPNPKTGTVTMDVKKAVEEIKAGKVEYRAEKAGIIHASIGKVSFSTEDLVENFNTLQDVIAKAKPASAKGTYFKSVAVTTTMGPGVKVDTSNFKL

SEQ ID NO: 188 - protein, 382 aa, Staphylococcus sp.
MSDKYYRSAYMNVDLNAVASNFKVFSTLHPNKTVMAVVKANAYGLGSVKVARHLMENGATFFAVATLDEAIELRMHGITAKILVLGVLPAKDIDKAIQHRVALTVPSKQWLKEAIKNISGEQEKKLWLHIKLDTGMGRLGIKDTKTYQEVIEIIQQYEQLVFEGVFTHFACADEPGDMTTEQYHRFKDMVNEAIKPEYIHCQNSAGSLLMDCQFCNAIRPGISLYGYYPSEYVQQKVKVHLKPSVQLIANVVQTKTLQAGESVSYGATYTATDPTTIALLPIGYADGYLRIMQGSFVNVNGHQCEVIGRVCMDQTIVKVPDQVKAGDSVILIDNHRESPQSVEVAAEKQHTINYEVLCNLSRRLPRIYHDGDQRFVTNELLK

SEQ ID NO: 189 - protein, 87 aa, Staphylococcus aureus
MSERNDRKVYVGKVVSDKMDKTITVLVETYKTHKLYGKRVKYSKKYKTHDENNSAKLGDIVKIQETRPLSATKRFRIVEIVEESVII

SEQ ID NO: 190 - protein, 339 aa, Staphylococcus sp.
MIVEVEHQRQGVDLIKIDNDETKIIFTNYGARVVSWKYHDNNIVLGNVVEADEFYHSNPFKFGASIGRYSGRIDNAKFKLQGKEYQLEKNNGEHHLHGGCHGLDNKLFDYEIRNEIAQIKVIFKTVLKSADDHFPGDIDVTITHIYDADHQWSIEYEAVASEDTLFSPTNHVYFNLNRDNNVVDNHRISSNQLDMYVLDERNIVTGDILDLHEVFEDNKIKLSDIFTSQHAQLSQQMTRFGGLDHPFTVGEHKMYVENHEFMLEVDTDMPHVVFFTFNQPDEWDSPFNIYKPHSGFTLETQFLPNDINLYGEKAPSILKAGEVFNSKTSFRIEEKVEND

SEQ ID NO: 191 - protein, 433 aa, Staphylococcus sp.
MRMIDIIEKKRDGHTLTTEEINFFIDGYVKGDIPDYQASSLAMAIYFQDMNDDERAALTMAMVNSGDMIDLSDIKGVKVDKHSTGGVGDTTTLVLAPLVAAVDVPVAKMSGRGLGHTGGTIDKLEAIDGFHVEIDEATFVKLVNENKVAVVGQSGNLTPADKKLYALRDVTGTVNSIPLIASSIMSKKIAAGADAIVLDVKTGSGAFMKTLEDAEALAHAMVRIGNNVGRNTMAIISDMNQPLGRAIGNALELQEAIDTLKGQGPKDLTELVLTLGSQMVVLANKAETLEEARALLIEAIDSGAALEKFKTFIKNQGGDETVIDHPERLPQAQYQIEYKAKKSGYVTELVSNDIGVASMMLGAGRLTKEDDIDLAVGIVLNKKIGDKVEEGESLLTIHSNRQDVDDVVKKLDSSITIADYVVSPTLIHKIITE

SEQ ID NO: 192 - protein, 185 aa, unidentified
  REGION 1..185: note = Bacilli sp.
MANIGYARVSTRDQSLDLQIDALSNFGCEKIFSEKVSGRKVNRTELDKCLDYLREGDTLVIYKLDRLGRTTKQLIDLAQWLENNNIELQIIDMNINTKDPMGKMFFTMMSAFAELEANLLSERTKKGLESARARGNMGGRKAMPQEKKEYIKHLYDTKEYTGQEIAKKTNVSRDTVYRILREFKK

SEQ ID NO: 193 - protein, 140 aa, Staphylococcus sp.
MKISTKGRYGLTLMISLAKKEGQGCISLKSIAEENNLSDLYLEQLVGPLRNAGLIRSVRGAKGGYQLRVPAEEISAGDIIRLLEGPITFVESIESEPPAQKQLWIRMRDAVRDVLDNTTLKYLAEYVDTSEDLDGYMFYI

SEQ ID NO: 194 - protein, 358 aa, Staphylococcus sp.
MLFKEAQAFIENMYKECHYETQIINKRLHDIELEIKETGTYTHTEEELIYGAKMAWRNSNRCIGRLFWDSLNVIDARDVTDEASFLSSINYHIAQATNEGKLKPYITIYAPKDGPKIFNNQLIRYAGYDNCGDPAEKEVTRLANHLGWKGKGTNFDVLPLIYQLPNESVKYYEYPTSLIKEVPIEHDHYPKLRKLNLKWYAVPIISNMDLKIGGIVYPTAPFNGWYMVTEIGVRNFIDDYRYNLLEKVADAFEFDTLKNNSFNKDRALVELNYAVYHSFKKEGVSIVDHLTAAKQFELFERNEAQQGRQVTGKWSWLAPPLSPTLTSNYHHGYDNTVKDPNFFYKKKKSNANQCPFHH

SEQ ID NO: 195 - protein, 252 aa, Staphylococcus sp.
MTILSVQHVSKTYGKKHTFQALKDINFDIQKGEFVAIMGPSGSGKTTLLNVLSSIDQISSGSVIANGQELNKLNQKALAKFRKESLGFIFQDYSILPTLTVKENIMLPLSVQKMSKATMEENYKAITTALGIYDLGNKYPSELSGGQQQRTAAARAFVHKPQIIFADEPTGALDSKSANDLLQRLEEMNKSFDTTIVMVTHDPVAASFAERVIMLKDGQIHTQLYQEGRSKQAFYEDIVHLQSVLGGVSNDI

SEQ ID NO: 196 - protein, 145 aa, Staphylococcus carnosus
MRQTFMANESNIERKWYVIDAAGQTLGRLSSEVASILRGKHKATYTPHVDSGDYVIIINAGQIEMTGKKASDKVYYRHSNYPGGIKSITAGELREKNPERLLEISIKGMLPSSRLGEKQGKKLFVYGGAEHPHAAQQPENYELRG

SEQ ID NO: 197 - protein, 175 aa, Staphylococcus sp.
MGLVRKFFMPNSYVQSIFQIDLDKLVDKGVKGIITDLDNTLVGWDVKEPTERVKAWFKEANEKGITITIVSNNNESRVASFSQHLDIDFIFKARKPMGKAFDKAITKMNIRPDQTVVIGDQMLTDVFGGNRRGLYTIMVVPVKRTDGFITKFNRLIERRLLRHFSKKGYITWEEN

SEQ ID NO: 198 - protein, 641 aa, Staphylococcus hyicus
MKETKHQHTFSIRKSAYGAASVMVASCIFVIGGGVAEANDSTTQTTTPLEVAQTSQQETHTHQTPVTSLHTATPEHVDDSKEATPLPEKAESPKTEVTVQPSSHTQEVPALHKKTQQQPAYKDKTVPESTIASKSVESNKATENEMSPVEHHASNVEKREDRLETNETTPPSVDREFSHKIINNTHVNPKTDGQTNVNVDTKTIDTVSPKDDRIDTAQPKQVDVPKENTTAQNKFTSQASDKKPTVKAAPEAVQNPENPKNKDPFVFVHGFTGFVGEVAAKGENHWGGTKANLRNHLRKAGYETYEASVSALASNHERAVELYYYLKGGRVDYGAAHSEKYGHERYGKTYEGVLKDWKPGHPVHFIGHSMGGQTIRLLEHYLRFGDKAEIAYQQQHGGIISELFKGGQDNMVTSITTIATPHNGTHASDDIGNTPTIRNILYSFAQMSSHLGTIDFGMDHWGFKRKDGESLTDYNKRIAESKIWDSEDTGLYDLTREGAEKINQKTELNPNIYYKTYTGVATHETQLGKHIADLGMEFTKILTGNYIGSVDDILWRPNDGLVSEISSQHPSDEKNISVDENSELHKGTWQVMPTMKGWDHSDFIGNDALDTKHSAIELTNFYHSISDYLMRIEKAESTKNA

SEQ ID NO: 199 - protein, 573 aa, Staphylococcus carnosus
MAKQIKGIAASDGVAIAKAYLLVEPDLSFDNESVTDTDAEVAKFNGALNKSKVELTKIRNNAEKQLGADKAAIFDAHLLVLEDPELIQPIEDKIKNESVNAAQALTDVSNQFITIFESMDNEYMAERAADIRDVSKRVLAHILGVELPNPSIVDESVVIIGNDLTPSDTAQLNKEYVQGFVTNIGGRTSHSAIMSRSLEIPAVVGTKSITEEVEAGDTIVVDGMTGDVLINPSDEVIAEYQEKRENFFKDKQELQKLRDAESVTADGHHVELAANIGTPNDLPGVIENGAEGIGLYRTEFLYMGRDQMPTEEEQFEAYKAVLEAMKGKRVVVRTLDIGGDKELPYLDLPEEMNPFLGYRAIRLCLDQPEIFRPQLRALLRASVFGKLNIMFPMVATIQEFRDAKALLEEERANLKNEGYEVADDIELGIMVEIPSTAALADIFAKEVDFFSIGTNDLIQYTMAADRMSERVSYLYQPYNPAILRLVKQVIEASHAEGKWTGMCGEMAGDQTAIPLLLGLGLDEFSMSATSILKARRLIRSLNESEMKELSERAVQCATSEEVVDLVEEYTKNA

SEQ ID NO: 200 - protein, 290 aa, Staphylococcus sp.
MSNVLTEIDSQYPYMTKNEKKIAKFILNSPQKVIKMRSQDLASLLDISTSSVIRFSKKITDGGFHDLKINISKYVPKASSIYNVELMNNESTESLRTKLHTRTTRALNHANNELNDKTIDQICHCLKRSETIFIYGFGASFVVATDLYQKLSRIGLNIQLVQETHIFATLLATHNSNDSVILITNNGTQSEMQSMVKVIDDYHIPIITITSTRDNPVAQASNIVLTYGKTDENEMHMGATTSLFAQMFTIDILYYRYVALNYHASLDFITQSKMALDNYRKHLSNINFKH

SEQ ID NO: 201 - protein, 416 aa, Staphylococcus sp.
MNFVTLTSDEFNAFTTKHFSHYTQSAIHYNHRVDLKGDVHLVGVKDDNGQVIAGCLLTEARTLKFFKYFYTHRGPVMDYTNQSLVAFFFKALTSYLKKQNCLYVLVDPYLIENLRNADGEIVKSYDNRAFVRTMDTLGYKHQGFPVGYDSMSQIRWLSVLDLKDKTEDQLLKEMDYQTRRNIKKTYDIGVKTKTLTIDETQTFFDLFHMAEEKHGFKFRELPYFEEMQKLYDDHAMLKLAYIDLNEYLKTLQLKQQQLTAELSGVEEALEESPNSKKNKTKRTQLEQQLNSNKRKIDNTIEQIEQDGAVLNLASALFIYNEHEVYYLSSGSNPKYNAYMGAYHLQWEMIKFAKAHHIDRYNFYGITGDFSESAEDYGVQQFKKGFNAHVEEYIGDFIKPIKPILYKLQTYLKHKRR

SEQ ID NO: 202 - protein, 450 aa, Staphylococcus aureus
MKINTTGGQIHGITQDGLDIFLGIPYAEPPVHDNRFKHSTLKTQWSEPIDATEIQPIPPQPDNKLEDFFSSQSATFTEHEDCLYLNIWKQHNDQSKKPVIIYFYGGSFENGHGTAELYQPAHLVQNNDIIVITCNYRLGALGYLDWSYFNKGFHSNNGLSDQINVIKWVHQFIESFGGDANNITLMGQSAGSMSIWTLLKIPDIEPYFHKVVLLSGALRLDTLESARNKAQHFQKLMLDYLDTDDVKSLSTDDILMLMAKLKQSRGPSKGLDLIYAPIKTDYIQNNYPTTKPIFACYTKDEGDIYITSEQKKLSPQRFIDIMELNDIPLKYEDVQTAKQQSLAITHCYFKQPMKQFLQQLNIQDSNAQLWLAEFAWHDTSSAHYRSAYHILDMVFWFGNLQILAAHQYPTTAHLKFLSRQMQNDLANFAKSGKMPWPMYHNERRYYRTYQ

SEQ ID NO: 203 - protein, 261 aa, Staphylococcus sp.
MRGENTMTPVFELKNVNYYYDHKKVLENINIKINKGEFLAIVGPNGAGKSTLLKLILGLLPLQSGEIFVEGIDFKNKKTSIKLSYVSQKANAFNSGFPASVKEVVLSGLTKTKRLFQTFNSKDNEKVIKVLERLNISDLIHKNIAELSGGQQQRVMIARALISEPAVLVLDEPTNGIDAKHVSEFYNTLDQLKQEGITIILVTHDIGVVADTATEVACLNKHLHFHGTTDEFKSLDEVEISKIYGHPVRFVDHQHNRECCN

SEQ ID NO: 204 - protein, 117 aa, Staphylococcus carnosus
MEAKAVARTIRIAPRKVRLVLDLIRGKNVGEAIAILKLTNKASSPVVEKLLMSALANAEHNYDMNTDELVVKEAYANEGPTLKRFRPRAQGRASAINKRTSHITIVVSDGKEEAKEA

SEQ ID NO: 205 - protein, 212 aa, Staphylococcus carnosus
MFQSKDLNVYFICGTQDIPEGRTIQEVLKEALEGGITLYQFREKGNGAKTGQDKVALAKELQALCKSYNVPFIVNDDVALAEEIDADGIHVGQDDEAVDDFNNRFEGKIIGLSIGNLEELNASDLTYVDYIGVGPIFATPSKDDASEPVGPKMIETLRKEVGDLPIVAIGGISLDNVQEVAKTSADGVSVISAIARSPHVTETVHKFLQYFK

SEQ ID NO: 206 - protein, 187 aa, Staphylococcus aureus
MSHLALKDLFSGFFVIDDEEEVEVPDKQQQVNEAPAKEQSQQTTKQNAIKSVPQKSASRYTTTSEERNNRMSNYSKNNSRNVVTMNNATPNNASQESSKMCLFEPRVFSDTQDIADELKNRRATLVNLQRIDKVSAKRIIDFLSGTVYAIGGDIQRVGTDIFLCTPDNVEVAGSITDHIENVEHSFD

SEQ ID NO: 207 - protein, 480 aa, Staphylococcus sp.
MNYKQSAEDILNAIGGEENLDAMAHCATRLRLVLNDESLVNEEALNNMDVVKGTFSTGGQYQIIIGSGTVNKVFSELEKLTGKEASTTSEVKAQSAKNMNPVQRFVKMLSDIFVPIIPAIVAGGLLMGLNNILTAKDLFFSGKSLIDVYSQFAGLAEMINVFANAPFTLLPILIGFSAAKRFGGNPFLGAALGMILVHPSLMSAYDFPKAVEAGKAIPYWDVFGLHINQVGYQGQVLPMLVAAYILASIEKGLRKVIPTVLDNLLTPLLSIFITAFLTFSFVGPITRQLGYWLSDGLTWLYEFGGAIGGLIFGLLYAPIVITGMHHSFIAVETTLIADATKTGGSFIFPIATMSNVAQGGAAIAAFFIIKQNKKLKGVASAAGISALLGITEPAMFGVNLKLRYPFIGAIVGSGIGSAYIAFFKVKAIALGTAGLPGFISINPVHAGWLHYFVGMTISFIVAITVTLILSKRKANKEVVE

SEQ ID NO: 208 - protein, 690 aa, Staphylococcus aureus
MLRGQEERKYSIRKYSIGVVSVLAATMFVVSSHEAQASEKTPTNAAVQKETLNQPGEQGNAITSHQMQSGKQLDDMHKENGKSGTVTEGKDTLQLSKYQSTQNSKTIRTQNDNQVKQDSERQGSKQSHQNNATNNTERQNDQVQNTHHAERNGSQSTTSQSNDVDKSQPSIPAQKVLPNHDKAAPTSTTPPSNDKTAPKSTKAQDATTDKHPNQQDTHQPAHQIIDAKQDDTVRQSEQKPQVGDLSKHIDGQNSPEKPTDKNTDNKQLIKDALQAPKTRSTTNAAADAKKVRPLKANQVQPLNKYPVVFVHGFLGLVGDNAPALYPNYWGGNKFKVIEELRKQGYNVHQASVSAFGSNYDRAVELYYYIKGGRVDYGAAHAAKYGHERYGKTYKGIMPNWEPGKKVHLVGHSMGGQTIRLMEEFLRNGNKEEIAYHKAHGGEISPLFTGGHNNMVASITTLATPHNGSQAADKFGNTEAVRKIMFALNRFMGNKYSNIDLGLTQWGFKQLPNESYIDYIKRVSKSKIWTSDDNAAYDLTLDGSAKLNNMTSMNPNITYTTYTGVSSHTGPLGYENPDLGTFFLMDTTSRIIGHDAREEWRKNDGVVPVISSLHPSNQPFVNVTNDEPATRRGIWQVKPIIQGWDHVDFIGVDFLDFKRKGAELANFYTGIINDLLRVEATESKGTQLKAS

SEQ ID NO: 209 - protein, 601 aa, Staphylococcus aureus
MCGIVGYIGYDNAKELLLKGLEKLEYRGYDSAGIAVVNDDNTTVFKEKGRIAELRKVADSSDFDGPVGIGHTRWATHGVPNHENSHPHQSSNGRFTLVHNGVIENYEELKGEYLQGVSFISETDTEVIVQLVEYFSNQGLSTEEAFTKVVSLLHGSYALGLLDAEDKDTIYVAKNKSPLLLGVGEGFNVIASDALAMLQVTSEYKEIHDHEIVIVKKDEVIIKDADGNVVERDSYIAEIDASDAEKGVYAHYMLKEIHEQPAVMRRIIQEYQDAEGNLKIDQDIINDVKEADRIYVIAAGTSYHAGLVGKEFLEKWAGVPTEVHVASEFVYNMPLLSEKPLFVYISQSGETADSRAVLVETNKLGHKSLTITNVAGSTLSREADHTLLLHAGPEIAVASTKAYTAQIAVLSILSQIVAKEHGREADIDLLRELAKVTTAIEAIVDDAPIMEQIATDFLETTRNAFFIGRTIDYNVSLEGALKLKEISYIQAEGFAGGELKHGTIALIEEGTPVVGLATQEKVNLSIRGNVKEVVARGAHPCIISMEGLEKEGDTYVIPHVHELLTPLVSVVALQLISYYAALHRDLDVDKPRNLAKSVTVE

SEQ ID NO: 210 - protein, 690 aa, Staphylococcus aureus
MLRGQEERKYSIRKYSIGVVSVLAATMFVVSSHEAQASEKTSTNAAAQKETLNQPGEQGNAITSHQMQSGKQLDDMHKENGKSGTVTEGKDTLQSSKHQSTQNSKTIRTQNDNQVKQDSERQGSKQSHQNNATNNTERQNDQVQNTHHAERNGSQSTTSQSNDVDKSQPSIPAQKVIPNHDKAAPTSTTPPSNDKTAPKSTKAQDATTDKHPNQQDTHQPAHQIIDAKQDDTVRQSEQKPQVGDLSKHIDGQNSPEKPTDKNTDNKQLIKDALQAPKTRSTTNAAADAKKVRPLKANQVQPLNKYPVVFVHGFLGLVGDNAPALYPNYWGGNKFKVIEELRKQGYNVHQASVSAFGSNYDRAVELYYYIKGGRVDYGAAHAAKYGHERYGKTYKGIMPNWEPGKKVHLVGHSMGGQTIRLMEEFLRNGNKEEIAYHKAHGGEISPLFTGGHNNMVASITTLATPHNGSQAADKFGNTEAVRKIMFALNRFMGNKYSNIDLGLTQWGFKQLPNESYIDYIKRVSKSKIWTSDDNAAYDLTLDGSAKLNNMTSMNPNITYTTYTGVSSHTGPLGYENPDLGTFFLMATTSRIIGHDAREEWRKNDGVVPVISSLHPSNQPFVNVTNDEPATRRGIWQVKPIIQGWDHVDFIGVDFLDFKRKGAELANFYTGIINDLLRVEATESKGTQLKAS

SEQ ID NO: 211 - protein, 475 aa, Staphylococcus aureus
MRDYTKQYINGEWVESNSNETIEVINPATEEVIGKVAKGNKADVDKAVEAADDVYLEFRHTSVKERQALLDKIVKEYENRKDDIVQAITDELGAPLSLSERVHYQMGLNHFVAARDALDNYEFEERRGDDLVVKEAIGVSGLITPWNFPTNQTSLKLAAAFAAGSPVVLKPSEETPFAAVILAEIFDKVGVPKGVFNLVNGDGAGVGNPLSEHPKVRMMSFTGSGPTGSKIMEKAAKDFKKVSLELGGKSPYIVLDDVDIKEAAKATTGKVVNNTGQVCTAGTRVLVPNKIKDAFLAELKEQFSQVRVGNPREDGTQVGPIISKKQFDQVQNYINKGIEEGAELFYGGPGKPEGLEKGYFARPTIFINVDNQMTIAQEEIFGPVMSVITYNDLDEAIQIANDTKYGLAGYVIGKDKETLHKVARSIEAGTVEINEAGRKPDLPFGGYKQSGLGREWGDYGIEEFLEVKSIAGYFK

SEQ ID NO: 212 - protein, 302 aa, Staphylococcus epidermidis
MSVFIDKNTKVMVQGITGSTALFHTKQMLDYGTQIVAGVTPGKGGQVVEGVPVYNTVEEAKNETGANVSVVYVPAPFAADSIIEAADADLDMVICITEHIPVVDMVKVKRYLQGRKTRLVGPNCPGVITADECKIGIMPGYIHKKGHVGVVSRSGTLTYEAVHQLTEEGIGQTTAVGIGGDPVNGTNFIDVLKAFNEDSETKAVVMIGEIGGTAEEEAAQWIKENMNKPVVGFIGGQTAPPGKRMGHAGAIISGGKGTASEKIKTLNDCGVETADTPSEIGTTLIDAAKKAGIYEELLTIKK

SEQ ID NO: 213 - protein, 585 aa, Staphylococcus epidermidis
MRKTKIVCTIGPASESEEMLEKLMNAGMNVARLNFSHGSHEEHKARIDTIRKVAKRLNKTIGLLLDTKGPEIRTHNMKDGLIVLEKGKEVIVSMNEVEGTPEKFSVTYENLINDVNIGSYILLDDGLVELQVKEINKDKGEVKCDILNTGELKNKKGVNLPGVKVNLPGITDKDADDIRFGIKENVDFIAASFVRRPSDVLDIRQILEEEKAEITIFPKIENQEGIDNIEEILEVSDGLMVARGDMGVEIPPESVPMVQKDLIRKCNKLGKPVITATQMLDSMQRNPRATRAEASDVANAIYDGTDAVMLSGETAAGQYPEEAVKTMRNIAVSAEAAQDYKKLLSDRTKLVETSLVNAIGVSVAHTALNLNVKAIVAATESGSTARTISKYRPHSDIIAVTPSEKTARQCAIVWGVNPVVKEGRKTTDALLNNAVATAVETGRVSNGDLIIITAGVPTGEKGTTNMMKIHLVGDEIAKGQGVGRGSVVGHAIVADSASDLEGKDLSDKVIITNSVDETLVPYVEKAIGLITEENGITSPSAIIGLEKGIPTVVGVEQATKEIKNDMLVTLDASQGKVFEGYANVL

SEQ ID NO: 214 - protein, 356 aa, Staphylococcus sp.
MTKIAFTGGGTVGHVSVNLSLIPTALSQGYEVLYIGSKNGIEREMIESQLPEIKYYPISSGKLRRYISLENAKDVFKVLKGILDARKVLKKEKPDLLFSKGGFVSVPVVIAAKSLNIPTIIHESDLTPGLANKIALKFAKKIYTTFEETLNYLPKEKADFIGATIREDLKNGNAHNGYQLTGFNENKKVLLVMGGSLGSKKLNSIIRENLDALLQQYQVIHLTGKGLKDAQVKKSGYIQYEFVKEDLTDLLAITDTVISRAGSNAIYEFLTLRIPMLLVPLGLDQSRGDQIDNANHFADKGYAKTIDEEQLTAQILLQELNKMEQERTRIINNMKSYEQSYTKEALFDKMIKDALN

SEQ ID NO: 215 - protein, 132 aa, Staphylococcus carnosus
MTMSDPIADMLTRVRNANMVRHEKIELPASNIKKQIAEILKSEGFIKNVEFVEDDKQGVLRLFLKYGQNNERVITGLKRISKPGLRVYAKADEVPKVLNGLGIALVSTSEGIVTDREARKRNVGGEVLAYVW

SEQ ID NO: 216 - protein, 381 aa, Staphylococcus sp.
MFQLLAVCPMGLEAVVAREIQELGYETNVENGRIFFEGDASAIVKANLWLRTADRIKIVVGRFNATTFDELFEQTKALPWESIIDKEGNFPVQGRSVKSTLHSVPDCQAITKKAIVERLRRAYNEKGWLNESGAKYPVEVAILKDNVLLTIDTSGSGLNRRGYRLAQGEAPIKETLAASLIRLANWKGDTPLIDPFCGSGTIAIEACLIAQNIAPGFNREFVSEQWNIMPANIYDDYRDEADKMADYDKEIEVYASDIDPEMVEIAKRNAEEVGLSDIIKFSVKDVNTLTIDTEEPVALIGNPPYGERIGDREEVEEMYRYIGKLMKQHPFLSTYILTSNKEFEYLVDRKATKRRKLFNGYIECTYYQYWGKKTERKTIEN

SEQ ID NO: 217 - protein, 1049 aa, Homo sapiens
MDWSFFRVVAMLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 218 - protein, 428 aa, Staphylococcus haemolyticus
MLDIKLFREQADTVKSKIELRGDDPKVVDEVLELDNERRQLIGKTEEMKARRNKVSEEIAEKKRNKENADDVIKEMRELGDEIKENDAKLNEVDNKVRDILIRIPNLIAEDVPQGDSDEENVEVKKWGTPREFDFEPKAHWDLVEELKMADFERAAKISGARFVYLTKDGALLERALMNYMLTKHTTQHGYTEMMTPQLVNADTMFGTGQLPKFEEDLFKVEKEGLYTIPTAEVPLTNFYRDEIIQPGVLPEKFTAQTACFRSEAGSAGRDTRGLIRLHQFDKVEMVRIEKPEDSWDALEDMTQNAEAILEELGLPYRRVILCTGDIGFSASKTYDLEVWLPSYDNYKEISSCSNCTDFQARRANIRFKRDAASKPELAHTLNGSGLAVGRTFAAIVENYQNADGSITIPEALVPFMGGKTEIRPVND

SEQ ID NO: 219 - protein, 463 aa, Staphylococcus aureus
MEKPSREAFEGNNKLLIGIVLSVITFWLFAQSLVNVVPILEDSFNTDIGTVNIAVSITALFSGMFVVGAGGLADKYGRIKLTNIGIILNILGSLLIIISNIPLLLIIGRLIQGLSAACIMPATLSIIKSYYIGKDRQRALSYWSIGSWGGSGVCSFFGGAVATLLGWRWIFILSIIISLIALFLIKGTPETKSKSISLNKFDIKGLVLLVIMLLSLNILITKGSELGVSSLLFITLLAITIGSFSLFIVLEKRATNPLIDFKLFKNKAYTGATASNFLLNGVAGTLIVANTFVQRGLGYSSLQAGSLSITYLVMVLIMIRVGEKLLQTLGCKKPMLIGTGVLIVGECLISLTFLPEILYVICCIIGYLFFGLGLGIYATPSTDTAIANAPLEKVGVAAGIYKMASALGGAFGVALSGAVYAIVSNMTNIYTGAMIALWLNAGMGILSFVIILLLVPKQNDTQL

SEQ ID NO: 220 - protein, 370 aa, Staphylococcus epidermidis
MAPKLQAQFDAVKVLNETQSKFEMVQILDEDGNVVNEDLVPDLTDEQLVELMERMVWTRILDQRSISLNRQGRLGFYAPTAGQEASQLASQYALESEDFILPGYRDVPQIIWHGLPLTDAFLFSRGHFKGNQFPEGVNALSPQIIIGAQYIQTAGVAFGLKKRGKNAVAITYTGDGGSSQGDFYEGINFASAYKAPAIFVIQNNNYAISTPRSKQTAAETLAQKAISVGIPGIQVDGMDALAVYQATLEARERAVAGEGPTVIETLTYRYGPHTMAGDDPTRYRTSDEDAEWEKKDPLVRFRKYLEAKGLWNEDKENEVVERAKSEIKAAIKEADNTEKQTVTSLMDIMYEEMPQNLAEQYEIYKEKESK

SEQ ID NO: 221 - protein, 122 aa, Staphylococcus epidermidis
MGYRKLGRTSDQRKAMLRDLATSLIVSERIETTEARAKEVRSVVEKLITLGKKGDLASRRNAAKTLRNVEILNEDDSTQTALQKLFGEIAERYSERQGGYTRILKVGPRRGDGAESVIIELV

SEQ ID NO: 222 - protein, 448 aa, Staphylococcus aureus
MSHRYIPLTEKDKQEMLQTIGAKSIGELFGDVPSDILLNRDLNIAEGEAETTLLRRLNRIASKNITKETHTSFLGAGVYDHYAPSVVDAMISRSEFYTAYTPYQPEISQGELQAIFEFQTLICELTDMDVANSSMYDGMTSFAEACILAFSQTKKNKIVVSKGLHYQALQVLHTYAKTRKEFEVVEIDLDGTVTDLKKLEAAVDDETAAVAVQYPNFYGSIEDLKKIHSFIEDKKALFIVYANPLALGLLTPPGSFGADIVVGDTQPFGIPAQFGGPHCGYFATTKKLMRKVPGRLVGQTQDDEGNRGFVLTLQAREQHIRRDKATSNICSNQALNALASSIAMSALGKQGIYDIAVQNIEHANYAKQQFIKKGFEVLDGTSFNEFVVKFDKPIQQVNEELVKYNIIGGFDLGVVSDDFKNHMLIAVTELRTKDEIDTFVEKAGELND

SEQ ID NO: 223 - protein, 290 aa, Staphylococcus sp.
MKLEHITKKYGSNVVLNDIDFDFGDSRIVGLIGKNGVGKTTVMKVMNGNIIKFDGKVDIDNADNIGFLIEHPKLYDNKSGLYNLKLFAQVLGKGFDKAYTDKIIDAFGMRPYIKKKVKKYSMGMKQKLAIAVSLMNKPKFLILDEPTNGMDPDGSIDVLTTIKSLVNELDMRILISSHKLEDIELICDRAVFLRDGHFVQDVNMEEGVASDTTIVTVDHKDFDRTEKYLAEHFQLQNVDKADGHLMINAQKNYQVILKALSELDIYPKYIETRKSSLRDTYFNINQRGDK

SEQ ID NO: 224 - protein, 486 aa, Staphylococcus aureus
MSNNFKDDFEKNRQSIDTNSHQDHTEDVEKDQSELEHQDTIENTEQQFPPRNAQRRKRRRDLATNHNKQVHNESQTSEDNVQNEAGTIDDRQVESSHSTESQEPSHQDSTPQHEEEYYNKNAFAMDKSHPEPIEDNDKHETIKDAENNTEHSTVSDKSIAEQSQQPKPYFATGANQANTSKDKHDDVTVKQDKDESKDHHSGKKGAAIGAGTAGVAGAAGAMGVSKAKKHSNDAQNKSNSDKSNNSTEDKASQDKSKDHHNGKKGAAIGAGTAGLAGGAASKSASAASKPHASNNASQNHDEHDNHDRDKERKKGGMAKVLLPLIAAVLIIGALAIFGGMALNNHNNGTKENKIANTNKNNADESKDKDTSKDASKDKSKSTDSDKSKEDQDKATKDESDNDQNNANQANNQAQNNQNQQQANQNQQQQQQRQGGGQRHTVNGQENLYRIAIQYYGSGSPENVEKIRRANGLSGNNIRNGQQIVIP

SEQ ID NO: 225 - protein, 205 aa, Staphylococcus sp.
MPFTPNEIKNKEFSRVKNGLEPTEVANFLEQLSTEIERLKEDKKQLEKVIEERDTNIKSYQDVHQSVSDALIQAQKAGEETKQAAEKQAEAIIAKAEAQANQMVGDAVEKARRLAFQTEDMKRQSKVFRSRFRMLVEAQLDLLKNEDWDYLLNYDLDAEQVTLENIHHLHENDLKPDEVAANAQNNASNTSDNNQQSNDSETTKK

SEQ ID NO: 226 - protein, 546 aa, Staphylococcus sp.
MKQRIGAYLIDAIHRAGVDKIFGVPGDFNLAFLDDIISNPNVDWVGNTNELNASYAADGYARLNGLAALVTTFGVGELSAVNGIAGSYAERIPVIAITGAPTRAVEQAGKYVHHSLGEGTFDDYRKMFAHITVAQGYITPENAATEIPRLINTAIAERRPVHLHLPIDVAISEIEIPTPFEVTAAKDTDASTYIELLVSKLHQSKQPIIITGHEINSFHLHQELEDFVNQTQIPVAQLSLGKGAFNEENPYYMGIYDGKIAEDKIRDYVDNSDLILNIGAKLTDSATAGFSYQFNIDDVVMLNHHNIKIDDVTNDEISLSSLLQQLTDISYTNNASFPAYHRPTSPDYTVGTEPLTQQTYFKMMQNFLKPNDVIIADQGTSFFGAYDLALYKSNTFIGQPLWGSIGYTLPATLGSQLADKDRRNLLLIGDGSLQLTVQAISTMIRQHIKPVLFVINNDGYTVERLIHGMYEPYNEIHMWDYKALPAVFGGKNVEIHDVESSKDLQEVFNTINGHPDVMHFVEVKMSVEDAPKKLIDIAKAFSQQNK

SEQ ID NO: 227 - protein, 588 aa, Staphylococcus aureus
MSKRTTYCGLVTEAFLGQEITLKGWVNNRRDLGGLIFVDLRDREGIVQVVFNPAFSEEALKIAETVRSEYVVEVQGTVTKRDPETVNPKIKTGQVEVQVTNIKVINKSETPPFSINEENVNVDENIRLKYRYLDLRRQELAQTFKMRHQITRSIRQYLDDEGFFDIETPVLTKSTPEGARDYLVPSRVHDGEFYALPQSPQLFKQLLMISGFDKYYQIVKCFRDEDLRADRQPEFTQVDIEMSFVDQEDVMQMGEEMLKKVVKEVKGVEINGAFPRMTYKEAMRRYGSDKPDTRFEMELIDVSQLGRDMDFKVFKDTVENDGEIKAIVAKGAAEQYTRKDMDALTEFVNIYGAKGLAWVKVVEDGLTGPIGRFFEAENVETLLTLTGAEAGDLVMFVADKPNVVAQSLGALRVKLAKELGLIDETKLNFLWVTDWPLLEYDEDAKRYVAAHHPFTSPKEADIAKLGTAPEEAEANAYDIVLNGYELGGGSIRIHDGELQEKMFEVLGFTKEQAQEQFGFLLDAFKYGAPPHGGIALGLDRLVMLLTNRTNLRDTIAFPKTASATCLLTNAPGEVSDKQLEELSLRIRH

SEQ ID NO: 228 - protein, 124 aa, Staphylococcus sp.
MTLEISNDYGKIDISNEVIASVVGGKAVECYGIVGMASRQQVRDGIAEILGHENYAKGIKVTENNGVVDIDMYIIVSYGVKISEVANNVQSTVKYTLEKSLNVSVNSINIYVQGVRVNNTGKKA

SEQ ID NO: 229 - protein, 1183 aa, Staphylococcus epidermidis
MAGQVVQYGRHRKRRNYARISEVLELPNLIEIQTKSYDWFLKEGLLEMFRDISPIEDFTGNLSLEFVDYRLGEPKYDLEESKNRDATYAAPLRVKVRLIIKETGEVKEQEVFMGDFPLMTDTGTFVINGAERVIVSQLVRSPSVYFNEKIDKNGRENYDATIIPNRGAWLEYETDAKDVVYVRIDRTRKLPLTVLLRALGFSTDQEIVDLLGDSEYLRNTLEKDGTENTEQALLEIYERLRPGEPPTVENAKSLLYSRFFDPKRYDLASVGRYKANKKLHLKHRLFNQKLAEPIVNSETGEIVVDEGTVLDRRKLDEIMDVLETNANSEVFELEGSVIDEPVEIQSIKVYVPNDEEGRTTTVIGNALPDSEVKCITPADIVASMSYFFNLLNGIGYTDDIDHLGNRRLRSVGELLQNQFRIGLSRMERVVRERMSIQDTDSITPQQLINIRPVIASIKEFFGSSQLSQFMDQANPLAELTHKRRLSALGPGGLTRERAQMEVRDVHYSHYGRMCPIETPEGPNIGLINSLSSYARVNEFGFIETPYRKVDLDTNSITDQIDYLTADEEDSYVVAQANSRLDENGRFLDDEVVCRFRGNNTVMAKEKMDYMDVSPKQVVSAATACIPFLENDDSNRALMGANMQRQAVPLMNPEAPFVGTGMEHVAARDSGAAITAKHRGRVEHVESNEILVRRLVEENGTEHEGELDRYPLAKFKRSNSGTCYNQRPIVSIGDVVEYNEILADGPSMELGEMALGRNVVVGFMTWDGYNYEDAVIMSERLVKDDVYTSIHIEEYESEARDTKLGPEEITRDIPNVSESALKNLDDRGIVYVGAEVKDGDILVGKVTPKGVTELTAEERLLHAIFGEKAREVRDTSLRVPHGAGGIVLDVKVFNREEGDDTLSPGVNQLVRVYIVQKRKIHVGDKMCGRHGNKGVISKIVPEEDMPYLPDGRPIDIMLNPLGVPSRMNIGQVLELHLGMAAKNLGIHVASPVFDGANDDDVWSTIEEAGMARDGKTVLYDGRTGEPFDNRISVGVMYMLKLAHMVDDKLHARSTGPYSLVTQQPLGGKAQFGGQRFGEMEVWALEAYGAAYTLQEILTYKSDDTVGRVKTYESIVKGENISRPSVPESFRVLMKELQSLGLDVKVMDEHDNEIEMADVDDEDAAERKVDLQQKSAPESQKETTD

SEQ ID NO: 230 - protein, 470 aa, Staphylococcus aureus
MEEHYYVSIDIGSSSVKTIVGEKFHNGINVIGTGQTYTSGIKNGLIDDFDIARQAIKDTIKKASIASGVDIKEVFLKLPIIGTEVYDESNEIDFYEDTEINGSHIEKVLEGIREKNDVQETEVINVFPIRFIVDKENEVSDPKELIARHSLKVEAGVIAIQKSILINMIKCVEACGVDVLDVYSDAYNYGSILTATEKELGACVIDIGEDVTQVAFYERGELVDADSIEMAGRDITDDIAQGLNTSYETAEKVKHQYGHAFYDSASDQDIFTVEQVDSDETVQYTQKDLSDFIEARVEEIFFEVFDVLQDLGLTKVNGGFIVTGGSANLLGVKELLSDMVSEKVRIHTPSQMGIRKPEFSSAISTISSSIAFDELLDYVTINYHDSEETEEDVIDVKDKDNESKLGGFDWFKRKTNKKDTHENEVESTDEEIYQSEDNHQEHKQNHEHVQDKDKDKEESKFKKLMKSLFE

SEQ ID NO: 231 - protein, 319 aa, unidentified
  REGION 1..319: note = Staphylococcaceae sp.
MSKQFFTVEENYKERFYQLPKVFFTNPNYKDLSNDAKIAYAILRDRLQLSIKNNWIDTEGNIYFIYTVADLEVILNCGNKKITKIKKELENVDLLIQKRQGLNKPNLLYLLKPAITKNDIYEIDKAENEVEALQDKEVSKGHVQKCQKDTSRNVKRTRLEMSKGHTNDTDFIDTDFIDTESNDMNNMNDTNQHSNHSNHFSNIHDKESLKYIELQELPELIKSYINNFSYEEVKSIKSVILKAKKSFNNKYDTFYMLEDIDEELLLVLKRFKGYLVKKQEKVANMEGYLMRSIIAELEEMHSTIMRRKNMENNPLSLFN

SEQ ID NO: 232 - protein, 282 aa, Staphylococcus aureus
MEKIFLNGEFVSPSEAKVSYNDRGYVFGDGIYEYIRVYNGKLFTVTEHYERFLRSANEIGLDLNYSVEELIELSRKLVDMNQIETGAIYIQATRGVAERNHSFPTPEVEPAIVAYTKSYDRPYDHLENGVNGVTVEDIRWLRCDIKSLNLLGNVLAKEYAVKYNAVEAIQHRGETVTEGSSSNAYAIKDGVIYTHPINNYILNGITRIVIKKIAEDYNIPFKEETFTVDFLKNADEVIVSSTSAEVTPVIKLDGEPVNDGKVGPITRQLQEGFEKYIESHSI

SEQ ID NO: 233 - protein, 917 aa, Staphylococcus aureus
MDYKETLLMPKTDFPMRGGLPNKEPQIQEKWDAEDQYHKALEKNKGNETFILHDGPPYANGNLHMGHALNKILKDFIVRYKTMQGFYAPYVPGWDTHGLPIEQALTKKGVDRKKMSTAEFREKCKEFALEQIELQKKDFRRLGVRGDFNDPYITLKPEYEAAQIRIFGEMADKGLIYKGKKPVYWSPSSESSLAEAEIEYHDKRSASIYVAFDVKDDKGVVDADAKFIIWTTTPWTIPSNVAITVHPELKYGQYNVDGEKYIIAEALSDAVAEALDWDKASIKLEKEYTGKELEYVVAQHPFLDRESLVINGDHVTTDAGTGCVHTAPGHGEDDYIVGQKYELPVISPIDDKGVFTEEGGQFEGMFYDKANKAVTDLLTEKGALLKLDFITHSYPHDWRTKKPVIFRATPQWFASISKVRQDILDAIENTNFKVNWGKTRIYNMVRDRGEWVISRQRVWGVPLPVFYAENGEIIMTKETVNHVADLFAEHGSNIWFEREAKDLLPEGFTHPGSPNGTFTKETDIMDVWFDSGSSHRGVLETRPELSFPADMYLEGSDQYRGWFNSSITTSVATRGVSPYKFLLSHGFVMDGEGKKMSKSLGNVIVPDQVVKQKGADIARLWVSSTDYLADVRISDEILKQTSDVYRKIRNTLRFMLGNINDFNPDTDSITESELLEVDRYLLNRLREFTASTINNYENFDYLNIYQEVQNFINVELSNFYLDYGKDILYIEQRDSHIRRSMQTVLYQILVDMTKLLAPILVHTAEEVWSHTPHVKEESVHLADMPKVVEVDQALLDKWRTFMNLRDDVNRALETARNEKVIGKSLEAKVTIASNDKFNASEFLTSFDALHQLFIVSQVKVVDKLDDQATAYEHGDIVIEHADGEKCERCWNYSEDLGAVDELTHLCPRCQQVVKSLV

SEQ ID NO: 234 - protein, 140 aa, Staphylococcus aureus
MAKKVDKVVKLQIPAGKANPAPPVGPALGQAGVNIMGFCKEFNARTQDQAGLIIPVEISVYEDRSFTFITKTPPAPVLLKKAAGIEKGSGEPNKAKVATVTKDQVREIANSKMQDLNAADEEAAMRIIEGTARSMGIVVE

SEQ ID NO: 235 - protein, 555 aa, Staphylococcus aureus
MTHLSDLDIANQSTLQPIKDIAASVGISEDALEPYGHYKAKIDINKITPRENKGKVVLVTAMSPTPAGEGKSTVTVGLADAFHELNKNVMVALREPALGPTFGIKGGATGGGYAQVLPMEDINLHFNGDFHAITTANNALSAFIDNHIHQGNELGIDQRRIEWKRVLDMNDRALRHVNVGLGGPTNGVPREDGFNITVASEIMAILCLSRSIKDLKDKISRITIGYTRDRKPVTVADLKVEGALAMILKDAIKPNLVQSIEGTPTLVHGGPFANIAHGCNSILATETARDLADIVVTEAGFGSDLGAEKFMDIKVREAGFDPAAVVVVATIRALKMHGGVAKDNLKEENVEAVKAGIVNLERHVNNIKKFGVEPVVAINAFIHDTDAEVEYVKSWAKENNVRIALTEVWKKGGKGGVDLANEVLEVIDQPNSFKPLYELELPLEQKIEKIVTEIYGGSKVTFSSKAQKQLKQFKENGWDNYPVCMAKTQYSFSDDQTLLGAPSGFEITIRELEAKTGAGFIVALTGAIMTMPGLPKKPAALNMDVTDDGHAIGLF

SEQ ID NO: 236 - protein, 548 aa, Staphylococcus aureus
MISKINGKLFADMIIQGAQNLSNNADLVDSLNVYPVPDGDTGTNMNLTMTSGREEVENNLSKNIGELGKTFSKGLLMGARGNSGVILSQLFRGFCKNIESESEINSKLLAESFQAGVETAYKAVMKPVEGTILTVAKDAAQAAIEKANNTEDCIELMEYIIVKANESLENTPNLLAVLKEVGVVDSGGKGLLCVYEGFLKALKGEKVEAKVAKIDKDEFVHDEHDFHGVINTEDIIYGYCTEMMVRFGKNKKAFDEQEFRQDMSQFGDSLLVINDEEIVKVHVHTEYPGKVFNYGQQYGELIKLKVENMREQHREVIRKEQHTAKPKMETVETAIITISMGEGISEIFKSMGATHIISGGQTMNPSTEDIVKVIEQSKCKRAIILPNNKNILMASEQAASIVDAEAVVIPTKSIPQGISALFQCDVDATLEENKAQMADSVNNVKSGSLTYAVRDTKIDGVEIKKDAFMGLIEDKIVSSQSDQLTTVTELLNEMLAEDSEILTVIIGQDAEQAVTDNMINWIEEQYPDVEVEVHEGGQPIYQYFFSVE

SEQ ID NO: 237 - protein, 428 aa, Staphylococcus epidermidis
MLDIRLFRNEPEKVKSKIELRGDDPKVVDQVLELDEQRRELISKTEEMKAKRNKVSEEIAQKKRNKEDADDVIAEMRHLGDEIKDIDNQLNEVDNKIRDILIRIPNLINEDVPQGASDEENVEVKKWGTPRDFEFEPKAHWDLVEELKMADFERAAKVSGARFVYLTKDGALLERALMNYMLTKHTTQHGYTEMMTPQLVNADTMFGTGQLPKFEEDLFKVEKEGLYTIPTAEVPLTNFYRDEIIQPGVLPELFTAQTACFRSEAGSAGRDTRGLIRLHQFDKVEMVRIVQPEDSWDALEEMTQNAEAILEELGLPYRRVILCTGDIGFSASKTYDLEVWLPSYNDYKEISSCSNCTDFQARRANIRFKRDAASKPELVHTLNGSGLAVGRTFAAIVENYQNEDGTLTIPEALVPFMGGKTKIEKPIK

SEQ ID NO: 238 - protein, 323 aa, Staphylococcus sp.
MIAVNWNTQEDMTNMFWRQNISQMWVETEFKVSKDIASWKTLSEAEQDTFKKALAGLTGLDTHQADDGMPLVMLHTTDLRKKAVYSFMAMMEQIHAKSYSHIFTTLLPSSETNYLLDEWVLEEPHLKYKSDKIVANYHKLWGKEASIYDQYMARVTSVFLETFLFFSGFYYPLYLAGQGKMTTSGEIIRKILLDESIHGVFTGLDAQHLRNELSESEKQKADQEMYKLLNDLYLNEESYTKMLYDDLGITEDVLNYVKYNGNKALSNLGFEPYFEEREFNPIIENALDTTTKNHDFFSVKGDGYVLALNVEALQDDDFVFDNK

SEQ ID NO: 239 - protein, 363 aa, Staphylococcus sp.
MFTYFKSAFKNAKPQLLITLIYALIAFAVIAVVYLLANFQLAKYVQTIAIYSQFGQKPPVDAYLKVIAVLLIAAVVSLFVLVQIFIGITNVMKRAMSHEKVKFTDLFIAFKKGNYLKSVLIGLVSIAMIIVLSLLTSLLYKLFSPVSEMIMNSVQSSYADSTHLVGIAITTQSIIIIVVLLIKAIITWLLLIPIFNFMTSFVESTNDKVKTHLANGFKAMKNGQKTFFKFFIGILLLNLVIILFKTPVGYLISFNTQSLSQSVAENIIRVYTVITIILFVVIHAVILMGIVQYYLKRGQKITKDKVKTADKNKKVVTEPKNTKVENDKVTTSVETKTEKAQDSLNDNTTKTMTSDKPEDNQPK

SEQ ID NO: 240 - protein, 663 aa, Staphylococcus aureus
MNKQNNYSDDSIQVLEGLEAVRKRPGMYIGSTDKRGLHHLVYEIVDNSVDEVLNGYGNEIDVTINKDGSISIEDNGRGMPTGIHKSGKPTVEVIFTVLHAGGKFGQGGYKTSGGLHGVGASVVNALSEWLEVEIHRDGNIYHQSFKNGGSPSSGLVKKGKTKKTGTKVTFKPDDTIFKASTSFNFDVLSERLQESAFLLKNLKITLNDLRSGKERQEHYHYEEGIKEFVSYVNEGKEVLHDVATFSGEANGIEVDVAFQYNDQYSESILSFVNNVRTKDGGTHEVGFKTAMTRVFNDYARRINELKTKDKNLDGNDIREGLTAVVSVRIPEELLQFEGQTKSKLGTSEARSAVDSVVADKLPFYLEEKGQLSKSLVKKAIKAQQAREAARKAREDARSGKKNKRKDTLLSGKLTPAQSKNTEKNELYLVEGDSAGGSAKLGRDRKFQAILPLRGKVINTEKARLEDIFKNEEINTIIHTIGAGVGTDFKIEDSNYNRVIIMTDADTDGAHIQVLLLTFFFKYMKPLVQAGRVFIALPPLYKLEKGKGKTKRVEYAWTDEELNKLQKELGKGFTLQRYKGLGEMNPEQLWETTMNPETRTLIRVQVEDEVRSSKRVTTLMGDKVQPRREWIEKHVEFGMQEDQSILDNSEVQVLENDQFDEEEI

SEQ ID NO: 241 - protein, 328 aa, Staphylococcus aureus
MADLLNVLKDKLSGKNVKIVLPEGEDERVLTAATQLQATDYVTPIVLGDETKVQSLAQKLNLDISNIELINPATSELKAELVQSFVERRKGKTTEEQAQELLNNVNYFGTMLVYAGKADGLVSGAAHSTGDTVRPALQIIKTKPGVSRTSGIFFMIKGDEQYIFGDCAINPELDSQGLAEIAVESAKSALSFGMDPKVAMLSFSTKGSAKSDDVTKVQEAVKLAQQKAEEEKLEAIIDGEFQFDAAIVPGVAEKKAPGAKLQGDANVFVFPSLEAGNIGYKIAQRLGGYDAVGPVLQGLNSPVNDLSRGCSIEDVYNLSFITAAQALQ

SEQ ID NO: 242 - protein, 610 aa, Staphylococcus sp.
MSKIIGIDLGTTNSCVTVLEGDEPKVIQNPEGSRTTPSVVAFKNGETQVGEVAKRQAITNPNTVQSIKRHMGTDYKVDIEGKSYTPQEISAMILQNLKNTAESYLGEKVDKAVITVPAYFNDAERQATKDAGKIAGLEVERIINEPTAAALAYGLDKTDKDEKVLVFDLGGGTFDVSILELGDGVFEVLSTAGDNKLGGDDFDQVIIDYLVAEFKKENGVDLSQDKMALQRLKDAAEKAKKDLSGVSQTQISLPFISAGENGPLHLEVNLTRSKFEELSDSLIRRTMEPTRQAMKDAGLTNSDIDEVILVGGSTRIPAVQEAVKKEIGKEPNKGVNPDEVVAMGAAIQGGVITGDVKDVVLLDVTPLSLGIEILGGRMNTLIERNTTIPTSKSQIYSTAVDNQPSVDVHVLQGERPMAADNKTLGRFQLTDIPPAERGKPQIEVTFDIDKNGIVNVTAKDLGTNKEQRITIQSSSSLSDEEIDRMVKDAEVNAEADKKRREEVDLRNEADSLVFQVEKTLTDLGENIGEEDKKSAEEKKDALKTALEGQDIEDIKSKKEELEKVIQELSAKVYEQAAQQQQQAQGANAGQNNDSTVEDAEFKEVKDDDKK

SEQ ID NO: 243 - protein, 84 aa, unidentified
  REGION 1..84: note = Eubacteria sp.
MKQGIHPEYHQVIFLDTTTNFKFLSGSTKTSSEMMEWEDGKEYPVIRLDISSDSHPFYTGRQKFAAADGRVERFNKKFGLKSNN

SEQ ID NO: 244 - protein, 414 aa, Staphylococcus aureus
MTENNNLVTSTQGIIKEALHKLGFDEGMYDLIKEPLRMLQVRIPVRMDDGTVKTFTGYRAQHNDAVGPTKGGVRFHPDVDEEEVKALSMWMTLKCGIVNLPYGGGKGGIVCDPRQMSIHEVERLSRGYVRAISQLVGPNKDIPAPDVFTNSQIMAWMMDEYSALDKFNSPGFITGKPIVLGGSHGRDRSTALGVVIAIEQAAKRRNMQIEGAKVVIQGFGNAGSFLAKFLYDLGAKIVGISDAYGALHDPNGLDIDYLLDRRDSFGTVTNLFEETISNKELFELDCDILVPAAISNQITEDNAHDIKASIVVEAANGPTTPEATRILTERGILLVPDVLASAGGVTVSYFEWVQNNQGYYWSEEEVNEKLREKLEAAFDTIYELSQNRKIDMRLAAYIIGIKRTAEAARYRGWA

SEQ ID NO: 245 - protein, 716 aa, Staphylococcus sp.
MTENKGSSQPKKNGNNGGKSNSKKNRNVKRTIIKIIGFMIIAFFVVLLLGILLFAYYAWKAPAFTEAKLQDPIPAKIYDKNGELVKTLDNGQRHEHVNLKDVPKSMKDAVLATEDNRFYEHGALDYKRLFGAIGKNLTGGFGSEGASTLTQQVVKDAFLSQHKSIGRKAQEAYLSYRLEQEYSKDDIFQVYLNKIYYSDGVTGIKAAAKYYFNKDLKDLNLAEEAYLAGLPQVPNNYNIYDHPKAAEDRKNTVLYLMHYHKRITDKQWEDAKKIDLKANLVNRTAEERQNIDTNQDSEYNSYVNFVKSELMNNKAFKDENLGNVLQSGIKIYTNMDKDVQKTLQNDVDNGSFYKNKDQQVGATILDSKTGGLVAISGGRDFKDVVNRNQATDPHPTGSSLKPFLAYGPAIENMKWATNHAIQDESSYQVDGSTFRNYDTKSHGTVSIYDALRQSFNIPALKAWQSVKQNAGNDAPKKFAAKLGLNYEGDIGPSEVLGGSASEFSPTQLASAFAAIANGGTYNNAHSIQKVVTRDGETIEYDHTSHKAMSDYTAYMLAEMLKGTFKPYGSAYGHGVSGVNMGAKTGTGTYGAETYSQYNLPDNAAKDVWINGFTPQYTMSVWMGFSKVKQYGENSFVGHSQQEYPQFLYENVMSKISSRDGEDFKRPSSVSGSIPSINVSGSQDNNTTNRSTHGGSDTSANSSGTAQSNNNTRSQQS

SEQ ID NO: 246 - protein, 305 aa, Staphylococcus sp.
METYEFNITDKEQTGMRVDKLLPELNSDWSRNQIQDWIKAGLVVANDKVVKSNYKVKLNDHIVVTEKEVVEADILPENLNLDIYYEDDDVAVVYKPKGMVVHPSPGHYTNTLVNGLMYQIKDLSGINGEIRPGIVHRIDMDTSGLLMVAKNDIAHRGLVEQLMDKSVKRKYIALVHGNIPHDYGTIDAPIGRNKNDRQSMAVVDDGKEAVTHFNVLEHFKDYTLVECQLETGRTHQIRVHMKYIGFPLVGDPKYGPKKTLDIGGQALHAGLIGFEHPVTGEYIERHAELAQDFEDLLDTIRKRDA

SEQ ID NO: 247 - protein, 121 aa, Staphylococcus sp.
MKASRILFGIGVGVAAGFVVALQGRDDKSVKNNTIDRTAPTGSKSELQREFETIKQSFNDILNYGVQIKNESAEFGSSIGGEIKSLLGNFKSDINPNIERLQSHIENLQNRGEDIGNEISK

SEQ ID NO: 248 - protein, 178 aa, Staphylococcus epidermidis
MSRVGKKIIDIPSDVTVTFDGSHVTVKGPKGELERTLNERMTFKQEENTVEVVRPSDSKEDRTDHGTTRALLNNMVLGVSQGYEKTLELVGVGYRAQMQGKDLVLNVGYSHPVEIKAEEGITFAVEKNTTVKVSGVSKEQVGAIASNIRSVRPPEPYKGKGIRYQGEYVRRKEGKTGK

SEQ ID NO: 249 - protein, 876 aa, Staphylococcus sp.
MNKLVLIDGNSLSFRAFYALPLLSNKAGIHTNAVYGFAMLLEKILKEEKPNHFLVAFDAGKTTFRHEKYSEYKGGRQKTPPELSEQFPYIRQLLDAYHIKRYELDNYEADDIIGTLSKEADKAGFQTIIITGDRDLTQLATDNVTIYYTKKGVTDVDHYTPDFIAEKYNGLTPNQIIDMKGLMGDTSDNIPGVAGVGEKTAIKLLNQFDTVEGVYEHLDEISGKKLKEKLQNSKEDALMSKELATINVDSPIEVKLEDTLMTHQDEQQEKIELFKKLEFKQLLADIDQSASGEDAIEKTFEIETSFDNVDFTSLKEATIHFELDGGNYLRNNILKFSLFTGEKHIVINADDIINYAELVSWLENPNTKKVVYDAKKTYVASHRLGIDIQNISFDIMLASYIIDPSRTISDVQSVVSLYGQSFVKDDVSIYGKGKKFKVPEDDVLNPYVASITDAIYFAKPNMDKQLEEYNQVELLADLELPLAKILSEMEEIGIYTDVHDLEEMEKEIQEKLDVLIRNIHDAAGEDFNINSPKQLGVVLFETLQLPVIKKTKTGYSTAVDVLEQLQGEHPIIDYILEYRQLSKLQSTYVEGLQKVISDDQRIHTRFNQTLAQTGRLSSVDPNLQNIPVRLEEGRKIRKAFKPTSKDSVILSADYSQIELRVLAHITQDESMKEAFINGDDIHTATAMKVFGVEADQVDSLMRRQAKAVNFGIVYGISDYGLSQSLGITRKKAKAFIDDYLASFPGVKQYMSDIVKDAKALGFVETLLHRRRYIPDITSRNFNLRGFAERTAMNTPIQGSAADIIKLAMVKFAQKMKETTYQAKLLLQVHDELIFEVPKSEVDSFSEFVEEIMENALQLDVPLKVDSSYGATWYDAK

SEQ ID NO: 250 - protein, 662 aa, Staphylococcus saprophyticus
MNFPWDQLLVQGNWMITMGQIGAPFAVIAVIAVISYFKLWKYLYREWFTSIDHKKIGAMYLICAVLMFVRGGIDALMMRTQLAIPDNTFLEGNHYNEVFSTHGVIMIIFMAMPFIFGLWNVVVPLQIGARDVAFPVLNNVSFWLFFSGMILFNLSFIVGGSPAAGWTNYAPLAGEFSPGPGVNYYLIAIQISGIGTLMTGINFFVTILRCKTPTMKFMEMPMFTVTTFITALIVILAFPVLTVTLALFTTDRIFDTAFFSVANGGMPMLWANFFWVWGHPEVYIVILPAFGIYSEIIPTFARKRLFGHQSMVWATAGIAFLSFLVWVHHFFTMGNGALINSFFSISTMLIGIPTGVKIFNWLFTLYKGRVTFESPMLFALGFIPNFLIGGVTGVMLSMAAADYQYHNTYFLVAHFHYTLVAGVVFACLGALIFWWPKMTGFKLNETLNKWCFWLFMIGFNVCFIPQFILGLDGMPRRLYTYMPEDGWWLLNAISTVGALLMALGFMFLVASIVYSFFKAPREATGDNWDGLGRTLEWATAGAMPPKYNFAITPDWNDYDTFVDMKEHGRHYLDNHNYTDIHMPNNTPVGFWMGILFTIGGFFLIFETLVPAIICLIGIFATMVYRSFQIDHGYHIPASEVAETEKRLRDARIKEREAVSHES

SEQ ID NO: 251 - protein, 166 aa, Staphylococcus epidermidis
MAVDNNKAKQAYDNQTGVNEQERKEQQQAQNNQPQFENKLTFSDEVVEKIAGIAAREVKGILDMKGGFTDSFTNAFSNGNNVTTGVSVEVGEKQAAVDLKVILEYGESAPKIFRKVTDLVKEQVKYITGLEVVEVNMQVDDVMTKKEWQQKNEKDNKENNEREGLK

SEQ ID NO: 252 - protein, 322 aa, Staphylococcus epidermidis
MKKIAVLTSGGDSPGMNAAVRAVTRTAIYNNIEVYGVYQGYQGLLDDDIHKLELGSVGDTIQRGGTFLFSARCPQFKEEDVRKKAIENLRKRGIEGLVVIGGDGSYRGAQRISEECKEIQTIGIPGTIDNDINGTDFTIGFDTALNTIIESVDKIRDTASSHARTFIVEVMGRDCGDLALWAGLSVGAETIVLPEVNTDIKDVAEKIEQGIKRGKKHSIVMVAEGCMSGQECADELTKYINIDTRVSVLGHIQRGGSPSGADRVLASRLGGYAVELLKQGETAKGVGIRNNQLTSTPFDEIFAESNRKFNSQMYELAKELSI

SEQ ID NO: 253 - protein, 820 aa, Staphylococcus saprophyticus
MLFGRLTERAQRVLAHAQEEAIRLNHSNIGTEHLLLGLMKEPEGIAAKVLETFEITEEKVVEEVEKLIGHGQEQMGALHYTPRAKKVIELSMDEARKLHHNFVGTEHILLGLIRENEGVAARVFANLDLNITKARAQVVKALGSPEMSNKNAQANKSNNTPTLDSLARDLTVIAKDGTLDPVIGRDKEITRVIEVLSRRTKNNPVLIGEPGVGKTAIAEGLAQAIVNNEVPETLKGKRVMSLDMGTVVAGTKYRGEFEERLKKVMEEIHQAGNVILFIDELHTLVGAGGAEGAIDASNILKPALARGELQCIGATTLDEYRKNIEKDAALERRFQPVQVDEPTVEDTIEILKGLRDRYEAHHRINISDEALVSAAKLSHRYVSDRFLPDKAIDLIDEASSKVRLKSHTTPPNLKEIEQQIEQVKNEKDAAVHAQEFENAANLRDKQTKLEKQYEEANTNWKNTQNGDNTSLSEEDIGEVIAGWTGIPLTKINETESDRLLNLEDTLHNRVIGQKDAVTSISKAVRRARAGLKDPKRPIGSFIFLGPTGVGKTELARALAESMFGEDDAMIRVDMSEFMEKHAVSRLVGAPPGYVGHDDGGQLTEKVRRKPYSVILFDEIEKAHPDVFNILLQVLDDGHLTDTKGRRVDFRNTVIIMTSNVGAQELQDQRFAGFGGATEGQDYESIRKTMMKELKNAFRPEFLNRVDDIIVFHKLSKDELKQIVTMMVNKLTNRLSEQDINISVTEAAKEKIAEEGYDPEYGARPLIRAIQKTVEDNLSELILDGNQIEGKEVKIDHDGKEFKYDITERQDSAKDKETSES

SEQ ID NO: 254 - protein, 94 aa, Staphylococcus saprophyticus
MLKLNLQFFSSKKGLGSTKNGRDSESKRLGAKRADGQYVTGGSILFRQRGTKIYPGENVGRGGDDTLFAKIDGVVKFERKGRDKKQVSVYAAAE

SEQ ID NO: 255 - protein, 178 aa, Staphylococcus carnosus
MSRVGKKIIEIPSDVTVDIKGNVITVKGPKGELTRTFDDSMSYKLEDNTLEVVRPSDSQKDRTVHGTTRALINNMVQGVSKGFEKTLELIGVGYRAQLQGSNLVLNVGYSHPVEFKPEDGITFTVEKNTTVKVEGISKELVGATASNIRAVRPPEPYKGKGIRYQGEYVRRKEGKTGK

SEQ ID NO: 256 - protein, 310 aa, Staphylococcus epidermidis
MAQKPVDYVTQIIGNTPVVKLRNVVDDDAADIYVKLEYQNPGGSVKDRIALAMIEKAEREGKIKPGDTIVEPTSGNTGIGLAFVCAAKGYKAVFTMPETMSQERRNLLKAYGAELVLTPGSEAMKGAIKKAKELKEEHGYFEPQQFENPANPEIHELTTGPELVEQFEGRQIDAFLAGVGTGGTLSGVGKVLKKEYPNVEIVAIEPEASPVLSGGEPGPHKLQGLGAGFVPDTLNTEVYDSIIKVGNDTAMDMARRVAREEGILAGISSGAAIYAAIQKAKELGKGKTVVTVLPSNGERYLSTPLYSFDN

SEQ ID NO: 257 - protein, 388 aa, Staphylococcus aureus
MKRNFPKLIALSLILSLSVTPIANAESNSNIKAKDKKHVQVNVEDKSIPTEVRNLAQKDYLSYVTSLDKIYNKEKASYTLGEPFKIYKFNKKSDGNYYFPVLNTEGNIDYIVTISPKVTKYSSSSSKYTINVSPFLSKVLNQYKDQQITILTNSKGYYVVTQNHKAKLVLKTPRLEDKKLKKTESIPTGNNVTQLKQKASVTMPTSQFKSNNYTYNEQYVNKLENFKIRETQGNNGWCAGYTMSALLNATYNTNKYHAEAVMRFLHPNLQGQRFQFTGLTPREMIYFGQTQGRSPQLLNRMTTYNEVDNLTKNNKGIAVLGSRVESRNGMHAGHAMAVVGNAKLDNGQEVIIIWNPWDNGFMTQDAKNNVIPVSNGDHYQWYSSIYGY

SEQ ID NO: 258 - protein, 154 aa, Staphylococcus sp.
MDQQSKEQLNIREMTKEDVPQVFDIERRSFNDSSWTIDAFYHEIEQNNFAKYFVLEFEQQIIGYLGLWIVIDQAQITTVAIDDNYRGYGLGQMLLKYGINYASHTCDVMSLEVRVNNKVAQHVYENLGFQYGGKRKNYYGEGEDAMVMWVNLND

SEQ ID NO: 259 - protein, 292 aa, Staphylococcus sp.
MPNKILLVDGMALLFRHFYATSLHKQFMYNSQGVPTNGIQGFVRHIFSAIHEIRPTHVAVCWDMGQSTFRNDMFDGYKQNRSAPPEELIPQFDYVKEISEQFGFVNIGVKNYEADDVIGTLAQQYSTDNDVYIITGDKDLLQCINDNVEVWLIKKGFNIYNRYTLHRFNEEYALEPQQLIDIKAFMGDTADGYAGVKGIGEKTAIKLIQQYQSVENVVENIDALSAGQRNKINDNLDELYLSKRLAEIHTQVPIDSEALFEKMSFATTLNHILSICNEHELHVSGKYISSHF

SEQ ID NO: 260 - protein, 224 aa, Staphylococcus aureus
MINKNDIVADIVIDYPKAADIFRSVGIDFCCGGQVSIEAASLEKKNVDLNELLQRLNDVEQTNTPGSLNPKFLNVSSLIQYIQAAYHEPLREEFKNLTPYVTKLSKVHGPNHPYLVELKETYDTFKSGMLEHMQKEDDVDFPKLIKYEQGEVVNDINTVIDDLVSDHIATGQLLVKMSDLTSSYEPPIEACGTWRLVYQRLKALEVLTHEHVHLENHVLFKKVS

SEQ ID NO: 261 - protein, 372 aa, Staphylococcus aureus
MKIGIPREIKNNENRVGLSPSGVHALVESGHTVLVETNAGSGSFFEDVDYKEAGAEIVAEQAKVWDVDMVIKVKEPLESEYPYFKEGLVLFTYLHLANEEKLTQALIDRKVISIAYETVQLPDRSLPLLSPMSEVAGRMSAQVGAEFLQKLNGGMGILLGGVPGVPKGKVTIIGGGQAGTNAAKIALGLGADVTILDVNPKRLQQLDDLFGGRVHTIMSNPLNIELYVKQSDLVIGAVLIPGAKAPRLVTEDMIKQMKNGSVIIDIAIDQGGIFETTDKITTHDDPTYIKHGVVHYAVANMPGAVPRTSTLALNNATLPYALMLANKGYREAFKSNQPLSLGLNTYKGHVTNKGVAEAFEMEYKSVEEALQL

SEQ ID NO: 262 - protein, 625 aa, Staphylococcus aureus
MVQEYDVIVIGAGHAGVEAGLASARRGAKTLMLTINLDNIAFMPCNPSVGGPAKGIVVREIDALGGQMAKTIDKTHIQMRMLNTGKGPAVRALRAQADKVLYQQEMKRVIEDEENLHIMQGMVDELIIEDNEVKGVRTNIGTEYLSKAVIITTGTFLRGEIILGNMKYSSGPNHQLPSITLSDNLRELGFDIVRFKTGTPPRVNSKTIDYSKTEIQPGDDVGRAFSFETTEYILDQLPCWLTYTNAETHKVIDDNLHLSAMYSGMIKGTGPRYCPSIEDKFVRFNDKPRHQLFLEPEGRNTNEVYVQGLSTSLPEHVQRQMLETIPGLEKADMMRAGYAIEYDAIVPTQLWPTLETKMIKNLYTAGQINGTSGYEEAAGQGLMAGINAAGKVLNTGEKILSRSDAYIGVLIDDLVTKGTNEPYRLLTSRAEYRLLLRHDNADLRLTDMGYELGMISEERYARFNEKRQQIDAEIKRLSDIRIKPNEHTQAIIEQHGGSRLKDGILAIDLLRRPEMTYDIILEILEEEHQLNADVEEQVEIQTKYEGYINKSLQQVEKVKRMEEKKIPEDLDYSKIDSLATEAREKLSEVKPLNIAQASRISGVNPADISILLIYLEQGKLQRVSY

SEQ ID NO: 263 - protein, 428 aa, Staphylococcus aureus
MRYTKSEEAMKVAETLMPGGVNSPVRAFKSVDTPAIFMDHGKGSKIYDIDGNEYIDYVLSWGPLILGHRDPQVISHLHEAIDKGTSFGASTLLENKLAQLVIDRVPSIEKVRMVSSGTEATLDTLRLARGYTGRNKIVKFEGCYHGHSDSLLIKAGSGVATLGLPDSPGVPEGIAKNTITVPYNDLDALKIAFEKFGDDIAGVIVEPVAGNMGVVPPIEGFLQGLRNITTEYGALLIFDEVMTGFRVGYHCAQGYFGVTPDLTCLGKVIGGGLPVGAFGGKKEIMDHIAPLGNIYQAGTLSGNPLAMTSGYETLSQLTPETYEYFNMLGDILEDGLKRVFAKHNVPITVNRAGSMIGYFLNEGPVTNFEQANKSDLKLFAEMYREMAKEGVFLPPSQFEGTFLSTAHTKEDIEKTIQAFDTALSRIVK

SEQ ID NO: 264 - protein, 436 aa, Staphylococcus aureus
MKNKYISKLLVGAATITLATMISNGEAKASENTQQTSTKHQTTQNNYVTDQQKAFYQVLHLKGITEEQRNQYIKTLREHPERAQEVFSESLKDSKNPDRRVAQQNAFYNVLKNDNLTEQEKNNYIAQIKENPDRSQQVWVESVQSSKAKERQNIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLDALVAQKDAEKKVAPKVEAPQIQSPQIEKPKAESPKVEVPQIQSPKVEVPQSKLLGYYQSLKDSFNYGYKYLTDTYKSYKEKYDTAKYYYNTYYKYKGAIDQTVLTVLGSGSKSYIQPLKVDDKNGYLAKSYAQVRNYVTESINTGKVLYTFYQNPTLVKTAIKAQETASSIKNTLSNLLSFWK

SEQ ID NO: 265 - protein, 228 aa, Staphylococcus aureus
MPKLILCRHGQSEWNAKNLFTGWEDVNLSEQGINEATRAGEKVRENNIAIDVAFTSLLTRALDTTHYILTESKQQWIPVYKSWRLNERHYGGLQGLNKDDARKEFGEEQVHIWRRSYDVKPPAETEEQREAYLADRRYNHLDKRMMPYSESLKDTLVRVIPFWTDHISQYLLDGQTVLVSAHGNSIRALIKYLEDVSDEDIINYEIKTGAPLVYELTDDLEVIDKYYL

SEQ ID NO: 266 - protein, 420 aa, Staphylococcus aureus
MFKFNEDEENLKCSFCGKDQDQVKKLVAGSGVYICNECIELCSEIVEEELAQNTSEAITELPTPKEIMDHLNEYVIGQEKAKKSLAVAVYNHYKRIQQLGPKEDDVELQKSNIALIGPTGSGKTLLAQTLAKTLNVPFAIADATSLTEAGYVGDDVENILLRLIQAADFDIDKAEKGIIYVDEIDKIARKSENTSITRDVSGEGVQQALLKILEGTTASVPPQGGRKHPNQEMIQIDTTNILFILGGAFDGIEDVIKRRLGEKVIGFSSNEADKYDEQALLAQIRPEDLQAYGLIPEFIGRVPIVANLETLDVTALKNILTQPKNALVKQYTKMLELDDVDLEFTEEALSAISEKAIERKTGARGLRSIIEETLIDIMFDVPSNENVTKVVITAQTINEETEPELYDAEGNLINNSKTSA

SEQ ID NO: 267 - protein, 351 aa, Staphylococcus aureus
MTKISKIIDELNNQQADAAWITTPLNVYYFTGYRSEPHERLFALLIKKDGKQVLFCPKMEVEEVKASPFTGEIVGYLDTENPFSLYPQTINKLLIESEHLTVARQKQLISGFNVNSFGDVDLTIKQLRNIKSEDEISKIRKAAELADKCIEIGVSYLKEGVTECEVVNHIEQTIKQYGVNEMSFDTMVLFGDHAASPHGTPGDRRLKSNEYVLFDLGVIYEHYCSDMTRTIKFGEPSKEAQEIYNIVLEAETSAIQAIKPGIPLKDIDHIARNIISEKGYGEYFPHRLGHGLGLQEHEYQDVSSTNSNLLEAGMVITIEPGIYVPGVAGVRIEDDILVTNEGYEVLTHYEK

SEQ ID NO: 268 - protein, 688 aa, Staphylococcus aureus
MFKKLFGQLQRIGKALMLPVAILPAAGILLAFGNAMHNEQLVEIAPWLKNDIIVMISSVMEAAGQVVFDNLPLLFAVGTALGLAGGDGVAALAALVGYLIMNATMGKVLHITIDDIFSYAKGAKELSQAAKEPAHALVLGIPTLQTGVFGGIIMGALAAWCYNKFYNITLPPFLGFFAGKRFVPIVTSVVAIATGVLLSFAWPPIQDGLNSLSNFLLNKNLTLTTFIFGIIERSLIPFGLHHIFYSPFWFEFGSYTNHAGELVRGDQRIWMAQLKDGVPFTAGAFTTGKYPFMMFGLPAAAFAIYKNARPERKKVVGGLMLSAGLTAFLTGITEPLEFSFLFVAPVLYGIHVLLAGTSFLVMHLLGVKIGMTFSGGFIDYILYGLLNWDRSHALLVIPVGIVYAIVYYFLFDFAIRKFKLKTPGREDEETEIRNSSVAKLPFDVLDAMGGKENIKHLDACITRLRVEVVDKSKVDVAGIKALGASGVLEVGNNMQAIFGPKSDQIKHDMAKIMSGEITKPSETTVTEEMSDEPVHVEALGTTDIYAPGIGQIIPLSEVPDQVFAGKMMGDGVGFIPEKGEIVAPFDGTVKTIFPTKHAIGLESESGVEVLIHIGIDTVKLNGEGFESLINVDEKVTQGQPLMKVNLAYLKAHAPSIVTPMIITNLENKELVIEDVQDADPGKLIMTVK

SEQ ID NO: 269 - protein, 243 aa, Staphylococcus aureus
MSKQKKSEIVNRFRKRFDTKMTELGFTYQNIDLYQQAFSHSSFINDFNMNRLDHNERLEFLGDAVLELTVSRYLFDKHPNLPEGNLTKMRATIVCEPSLVIFANKIGLNEMILLGKGEEKTGGRTRPSLISDAFEAFIGALYLDQGLDIVWKFAEKVIFPHVEQNELLGVVDFKTQFQEYVHQQNKGDVTYNLIKEEGPAHHRLFTSEVILQGEAIAEGKGKTKKESEQRAAKSAYKQLKQIK

SEQ ID NO: 270 - protein, 335 aa, Staphylococcus sp.
MTHNIHDNISQWMKSNEETPIVMSSRIRLARNLENHVHPLMYATENDGFRVINEVQDALPNFELMRLDQMDQQSKMKMVAKHLISPELIKQPAAAVLVNDDESLSVMINEEDHIRIQAMGTDTTLQALYNQASSIDDELDRSLDISYDEQLGYLTTCPTNIGTGMRASVMLHLPGLSIMKRMTRIAQTINRFGYTIRGIYGEGSQVYGHTYQVSNQLTLGKSELEIIETLTEVVNQIIHEEKQIRQKLDTYNQLETQDRVFRSLGILQNCRMITMEEASYRLSEVKLGIDLNYIELQNFKFNELMVAIQSPFLLDEEDDKSVKEKRADILREHIK

SEQ ID NO: 271 - protein, 276 aa, Staphylococcus sp.
MALKKVLTIAGSDTSAGAGMQADLKTFQELDTYGMVALTAIVTMDKDTWSHDVTPLPMDVFEKQLETALSIGPDAIKTGMLGTEEIIKRAGEVYEASNAQYFVVDPVMVCKGEDEVLNPGNTEAMIKYLLPKATVVTPNLFEAGQLSGLGKLNSIEDMKKAATIIFDKGAQHVIIKGGKALDQDKSYDLYYDGQTFYQLTTDMFQQSYNHGAGCTFAAATTAYLANGKSPKEAVISAKAFVASAIKNGWKMNDFVGPVDHGAYNRIEHIDVEVTEV

SEQ ID NO: 272 - protein, 334 aa, Staphylococcus sp.
MTETFKAFVIDKDESGKVTPTFKQLSTTDLPKGDVLIKVHYSGINYKDALATQDHNAVVKSYPMIPGIDLAGTIVESEAPGFEKGEQVIVTSYDLGVSHYGGFSEYARVKSEWIVKLPDTLTLEESMIYGTAGYTAGLAIERLEKVGMNIEDGPVLVRGASGGVGTLAVLMLNELGYKVIASTGKQDVSNQLLELGAKEVIDRLPVEDDHKKPLASSTWQACVDPVGGEGINYVTKRLNHSGSIAVIGMTAGNTYTNSVFPHILRGVNILGIDSVFTAMKLRQRVWRRLAKDLKPENLHEIKQVITFDELPEQLNKVIKHENKGRIVIDFGVDK

SEQ ID NO: 273 - protein, 236 aa, Staphylococcus sp.
MTKGTPHIQPNGVKIAKTVLMPGDPLRAKYIADNFLENVEQFNDVRNMFGYTGTYKGKEVSVMGSGMGIPSIGIYSYELYNFFDVDTIIRIGSCGALQENVNLYDVIIAQAASTNSNYVDQYNIPGHFAPIADFELVTKAKNVADQIGATTHVGNVLSSDTFYNADPTFNDAWKKMGILGIEMESAGLYLNAIHAGKKALGIFTVSDHILRDEATTPEERQNSFTQMMEIALEIAE

SEQ ID NO: 274 - protein, 380 aa, Staphylococcus sp.
MEIYADYAATTPVKPEVVDAMMTIYNSHYGNPSSIHAKGRDARKYLDESRRQIAQLLGADTHEIIFTSGATESNNTAIKGIVKANEQLGNHIITSKIEHHSVLHVFEQLEREGFDVTYLDVDDTGAIDLDQLEETITDKTILVSIMFVNNEVGTVQQIYDIQDIIAETNAYFHVDAVQAIGHLDVKFDEFEIDAMSITAHKFGGPKGVGALLVKDHVTLDYPQLGGEQELKRRAGTENLAQIVGMAKALQLAEKNRDDNNIHLMNLKEQFLVKLQERAIPFELNGSMTDATGHIVNLYFPFVEVETMLTLLDMAQIYVSSGSACTAGSTQPSHVLDAMFEDEERSNHSIRFSFNELTTENEINAIVAEIHKIYFKFKEES

SEQ ID NO: 275 - protein, 342 aa, Staphylococcus sp.
MFDDKILLITGGTGSFGNAVMKRFLDSNIKEIRIFSRDEKKQDDIRKKYNNSKLKFYIGDVRDSQSVETAMRDVDYVFHAAALKQVPSCEFFPVEAVKTNIIGTENVLQSAIHQNVKKVICLSTDKAAYPINAMGISKAMMEKVFVAKSRNIRSEQTLICGTRYGNVMASRGSVIPLFIDKIKAGEPLTITDPDMTRFLMSLEDAVELVVHAFKHAETGDIMVQKAPSSTVGDLATALLELFEADNAIEIIGTRHGEKKAETLLTREEYAQCEDMGDYFRVPADSRDLNYSNYVETGNEKITQSYEYNSDNTHILTVEEIKEKLLTLEYVRNELNDYKASMR

SEQ ID NO: 276 - protein, 211 aa, Staphylococcus sp.
MIEFLHEHGGQLMSKTLEHFYISIVALLLAIIVAVPIGILLSKTKRTANIILTVAGVLQTIPTLAVLAIMIPIFGVGKTPAIVALFIYVLLPILNNTVLGVQNIDSNIKEAGKSMGMTQFQLMKDVELPLALPLILGGIRLSSVYVISWATLASYVGAGGLGDFIFNGLNLYDPLMIVTATVLVTALALGVDALLALVEKWVVPKGLKVSG

SEQ ID NO: 277 - protein, 164 aa, Staphylococcus sp.
MIEIKTLTNNDFNEYKRLVSTVNEEFTQDSHYSQTMTDTLIHDILNQGSPKCIVFGCYENETLIATAALEQIRYVGKEHKSLIKYNFVTNNDKSINSELINFIINYARQNNYESLLTSIVSNNIGAKVFYSALGFDILGFEKNAIKIGNTYFDEHWLFYDLINK

SEQ ID NO: 278 - protein, 404 aa, Staphylococcus sp.
MIPYGDFTFFLIALIALLPVIILGFLGKRSYIYNGVVTAFMIVLIFSSDKHNLFDQKYLSVQLISFIIYVVWQVLLIMFYYHSKPKNNSFSKFVTVMVLSILPLALVKVLQSTWLGGHQIHFHESKLIEFVGFLGISYVTFKSVQLIMEIRDGSIKEIKVWKLIQFISFFPTISSGPIDRYKRFVKDDKKVPTGNEYRELVLKAIHMIMLGFLYKYIVAYFINTYAIMPLQLDLHGFVNLWLYMYAYSLYLFFDFAGYSLFAIAFSYLFGIKTPPNFDKPFKAKNIKDFWNRWHMTLSFWFRDCIYMRSLFYMSRKKLLKSQFAMSNVAFLINFFIMGIWHGIEVYYIVYGLYHAALFIGYGYYERWRKKHPPRWQNGFTTALSIVITFHFVTFGFLIFSGKLI

SEQ ID NO: 279 - protein, 328 aa, Staphylococcus sp.
MKKCIKTLFLSIILVVMGGWYHSAHALDSLSKSPENWMSKLDESKHLTEINMPGSHDSGSFTLTDPVKSVWAKTQDKDYLTQMKSGVRFFDIRGRASADNMISVHHGMVYLHHELGKFLDDAKYYLSAYPNETIVMSMKKDYDSDSKVTKTFEEIFREYYYNNPQYQNLFYTGSNANPTLKETKGKIVLFNRMGGTYIKSGYGADTSGIQWADNATFETKINNGSLNLQVQDEYKDYYDKKVEAVKNLLAKAKTDSNKDNVYVNFLSVASGGSAFNSTYNYASHINPEIAKTIKANGKARTGWLIVDYAGYPWPGYDDIVSEIIDSNK

SEQ ID NO: 280 - protein, 211 aa, Staphylococcus sp.
MNQWDQFLTPYKQAVDELKVKLKGMRKQYEVGEQASPIEFVTGRVKPIASIIDKANKRQIPFDRLREEMYDIAGLRMMCQFVEDIDVVVNILRQRKDFKVIEERDYIRNTKESGYRSYHVIIEYPIETLQGQKFILAEIQIRTLAMNFWATIEHTLRYKYDGAYPDEIQHRLERAAEAAYLLDEEMSEIKDEIQEAQKYYTQKRSKKHEND

SEQ ID NO: 281 - protein, 390 aa, Staphylococcus aureus
MKIATLNKGKETKCFNGYPLIEEEDIYSQDHLKEGDIFQIVTDKSQYVATAYVGRQHKGLGWVLTYDKAQEVNTAFFVKLFNTALAERDYYFNIDGTNAFRLFNAEGDGVGGLTIDNYDGHLLIQWYSKGIYKFKYAILEAVRKVFDYKSIYEKVRFKDSEYSGGFVEGDAPEFPIVIEENFTFYNVDLEDGLMTGIFLDQKEVRKKLRDQYAKERHVLNLFSYTGAFSVIAASEASSTTSVDLANRSRSLTEENFGLNAIDPKSQYIYVMDTFDFYKYAARHGHSYDTIVIDPPSFARNKKRTFSVQKDYDKLITGALNILSSEGTLLLCTNASVYPLKQFKNTIKKTLEESGVDYELTEVMGLPKDFKTHPHYKPSKYLKAVFVNIRH

SEQ ID NO: 282 - protein, 693 aa, Staphylococcus carnosus
MARDFSLEKTRNIGIMAHIDAGKTTTTERILYYTGRIHKIGETHEGASQMDWMEQEQDRGITITSAATTAQWHDYRVNIIDTPGHVDFTVEVERSLRVLDGAVTVLDAQSGVEPQTETVWRQATTYGVPRIVFVNKMDKLGANFEYSVSTLHDRLQANAQPIQLPIGAEDEFEAIIDLVTMKCYQYNGDFGEEVVEIEIPDDYKDKAAEARESLIEAVAEANEDLMEKYLEGEEITIDELKAAIRQATLDIEFYPVLCGTAFKNKGVQLMLDAVIDYLPSPLDVKPIVGHHADNPDEEVIAPADDDADFAALAFKVMTDPYVGKLTFFRVYSGTLNSGSYVKNSTKDKRERVGRLLQMHANTRKEISTVYSGDIAAAVGLKETGTGDTLCGEKNDIILESMEFPEPVIHLSVEPKSKADQDKMTQALVKLQEEDPTFHAHTDEETGQVIIGGMGELHLDILVDRMKKEFNVEANVGAPMVSYRETFKTPAKVQGKFSRQSGGRGQYGDVHIEFTPNEVGAGFEFENAIVGGVVPREYIPSVEAGLKDAMENGVVAGYPLIDVKAKLYDGSYHDVDSSEMAFKIAASLALKEAAKVADPVILEPMMKVEILMPEEYMGDIMGDVTSRRGRVDGMEARGNAQMVRAFVPLSEMFGYATSLRSNTQGRGTYTMYFDHYEEVPKSIAEDIIKKNKGE

SEQ ID NO: 283 - protein, 555 aa, Staphylococcus carnosus
MTHLSDLDIANQATIKPISEIAEKIGIPEDALEQYGHYKAKIDINKLDDKGDRGKVVLVTAMSPTPAGEGKSTVTVGLADAFHELGENVMMALREPALGPVFGIKGGATGGGYAQVLPMEDINLHFNGDFHAITTANNALAAFIDNHIHQGNELGIDQRRIEWKRVLDMNDRELRKVVVGLGGPTQGVPREDGFNITVASEIMTILCLSTGLKDLKASIANITIGYTRDRKPVTVADLKVEGALAMILKDAIKPNLVQSIEGTPALIHGGPFANIAHGCNSIIATETARKLADIVVTEAGFGSDLGAEKFMNIKARKAGFEPSAAVVVATIRALKMHGGVAKDDLKEENVQAVRDGLANLERHIENIRSFGVEPVVALNAFVSDTEAEEQVVEDWAKEHGVRIALTEVWEKGGKGGVELAKQVQEVLNEKHDFKHTYDLDLPIEEKIEKVVTNIYGGNKVTFTSGALKQLKQIKENGWDNYPVCMAKTQYSFTDDKDRLGAPDDFEITIRELQPKTGAGFIVALTGAIMTMPGLPKKPAALNMDVTEDGHAKGLF

SEQ ID NO: 284 - protein, 595 aa, Staphylococcus carnosus
MEAYKEKIKEKLSVVPFEPGCYLMKDRNNQVIYVGKAKRLRNRLRSYFTGAHDAKTTRLVGEIRNFEFIVTDSETESLLLELNLIKQYQPRYNILLKDDKSYPFIKITKEKYPRLLVTRTVKKGSGKYFGPYPNAYAAVETKKLLDRIYPFRKCDKMPNKLCLYYHIGQCLGPCVYDVDQKEYDDMTQEVSDFLNGEDKTIIKNLESRMQAASENLEFEQAKEYRDLIQNIHNLTKKQNIMSADNTARDVFGYYISKGWMCIQVFFVRNGNMIQRDTTMIPLQQTPEEEFYTFIGQFYRLNQHLLPKEVHVPKNLDKKMIESVVDTKILQPVRGQKKDLVNMANHNAEVSLQNKFELIARDESRTVKAIEELGEQMGIQTPIRIEAFDNSNIQGVDAVSAMVTFVDGKPDKKGYRKYKIKTVEGPDDYKSMQEVIRRRYTRVLNDGLPLPDLIIVDGGKGQMSVAIDVLENELGLDIPVAGLRKNDKHRTSELLYGPAAEVVPLKKNSQAFYLLQRIQDEVHRFAITFHRKTRQKHSFTSALDEIEGIGPKRKTTLLRTFGSIKKMREATLEDLKEAGLPEKVAESLQKELNKED

SEQ ID NO: 285 - protein, 437 aa, Staphylococcus aureus
MTHYHFVGIKGSGMSSLAQIMHDLGHEVQGSDIENYVFTEVALRNKGIKILPFDANNIKEDMVVIQGNAFASSHEEKARAHQMKLDVVSYNDFLGQIIDQYTSVAVTGAHGKTSTTGLLSHVMNGDKKTSFLIGDGTGMGLPESDYFAFEACEYRRHFLSYKPDYAIMTNIDFDHPDYFKDINDVFDAFQEMAHNVKKGIIAWGDDEHLRKIEADVPIYYYGFKDSDDIYAQNIQITDKGTAFDVYVDGEFYDHFLSPQYGDHTVLNALAVIAISYLEKLDVTNIKEALETFGGVKRRFNETTIANQVIVDDYAHHPREISATIETARKKYPHKEVVAVFQPHTFSRTQAFLNEFAESLSKADRVFLCEIFGSIRENTGALTIQDLIDKIEGASLINEDSINVLEQFDNAVVLFKGAGDIQKLQNAYLDKLGMKNAF

SEQ ID NO: 286 - protein, 244 aa, Staphylococcus aureus
MNEKNIKHSQNFITSKHNIDKIMTNIRLNEHDNIFEIGSGKGHFTLELVQRCNFVTAIEIDHKLCKTTENKLVDHDNFQVLNKDILQFKFPKNQSYKIFGNIPYNISTDIIRKIVFDSIADEIYLIVEYGFAKRLLNTKRSLALLLMAEVDISILSMVPREYFHPKPKVNSSLIRLNRKKSRISHKDKQKYNYFVMKWVNKEYKKIFTKNQFNNSLKHAGIDDLNNISFEQFLSLFNSYKLFNK

SEQ ID NO: 287 - protein, 358 aa, Staphylococcus aureus
MSQAVKVERRETLKQKPNTSQLGFGKYFTDYMLSYDYDADKGWHDLKIVPYGPIEISPAAQGVHYGQSVFEGLKAYKRDGEVALFRPEENFKRLNNSLARLEMPQVDEAELLEGLKQLVDIERDWIPEGEGQSLYIRPFVFATEGALGVGASHQYKLLIILSPSGAYYGGETLKPTKIYVEDEYVRAVRGGVGFAKVAGNYAASLLAQTNANKLGYDQVLWLDGVEQKYIEEVGSMNIFFVENGKVITPELNGSILPGITRKSIIELAKNLGYEVEERRVSIDELFESYDKGELTEVFGSGTAAVISPVGTLRYEDREIVINNNETGEITQKLYDVYTGIQNGTLEDKNGWRVVVPKY

SEQ ID NO: 288 - protein, 431 aa, Bacillus licheniformis
MFKTISNFMRVKDIRNKIIFTLLMLIVFRLGSFIPVPHVNTEVLKAQDQMSVFGILNTFGGGALFNFSILAMGIMPYITASIIIQLLQMDVVPKFTEWSKQGEVGRRKLAQFTRYFTIVLGFIQALGMSYGFNNMAGGALITDPGVGTYLLIAIVLTAGTAFLMWLGEQITSHGVGNGISIIIFAGIVAGIPQTINQIYAQQFVDAGDQLFLQIIKVVVILVAILAIVVGVIFIQQAVRKISIQYAKGSGRSPVPGGQSTHLPLKVNPAGVIPVIFAVAFITTPRTVATFFGSNDVTNWIQKTFDYTHPVGMGVYAALIIAFTYFYAFVQVNPEQMADNLKKQGGYIPGVRPGKMTQDRITSILYRLTFVGSIFLAVIAILPVLFVNIAGLPSSAQIGGTSLLIVIGVALETMKQLESQLVKRNYRGFMKH

SEQ ID NO: 289 - protein, 701 aa, Staphylococcus aureus
MNNGFFNSDFDSIFRRMMKDMQGSNQVGNKKYYINGKEVSPEELAQLTQQGGNHSAEQSAQAFQQAAQRQQGQQGGNGNYLEQIGRNLTQEARDGLLDPVIGRDKEIQETAEVLSRRTKNNPILVGEAGVGKTAIVEGLAQAIVEGNVPAAIKDKEIISVDISSLEAGTQYRGAFEENIQKLIEGVKSSQNAVLFFDEIHQIIGSGATGSDSGSKGLSDILKPALSRGEISIIGATTQDEYRNNILKDSALTRRFNEVLVNEPSAKDTVEILKGIREKFEEHHQVKLPDDVLKACVDLSIQYIPQRLLPDKAIDVLDITAAHLSAQSPAVDKVETEKRISELENDKRKAVSAEEYKKADDIQNEIKSLQDKLENSNGEHTAVATVHDISDTIQRLTGIPVSQMDDNDIERLKNISNRLRSKIIGQDQAVEMVSRAIRRNRAGFDDGNRPIGSFLFVGPTGVGKTELAKQLAIDLFGNKDALIRLDMSEYSDTTAVSKMIGTTAGYVGYDDNSNTLTEKVRRNPYSVILFDEIEKANPQILTLLLQVMDDGNLTDGQGNVINFKNTIIICTSNAGFGNGNDAEEKDIMHEMKKFFRPEFLNRFNGIVEFLHLDKDALQDIVNLLLDDVQVTLDKKGITMDVSQDAKDWLIEEGYDEELGARPLRRIVEQQVRDKITDYYLDHTDVKHVDIDVEDNELVVKGK

SEQ ID NO: 290 - protein, 154 aa, Staphylococcus aureus
MVDVAFVCLGNICRSPMAEAIMRQRLKDRNIHDIKVHSRGTGSWNLGEPPHEGTQKILNKHNIPFDGMISELFEATDDFDYIVAMDQSNVDNIKFINPNLKGQLFKLLEFSNMEESDVPDPYYTNNFEGVYDMVLSSCDNLIDYIVKDANLKEG

SEQ ID NO: 291 - protein, 349 aa, Staphylococcus saprophyticus
MDNDRQKALDTVIKNMEKSFGKGAVMKLGDNKARKVSSVSSGSVTLDNALGVGGYPKGRIVEIYGPESSGKTTVALHAIAEVQKNGGVAAFIDAEHALDPVYAEALGVDIQNLYLSQPDHGEQGLEIAEAFVRSGAVDIVVVDSVAALTPKAEIEGEMGDTHVGLQARLMSQALRKLSGAISKSNTTAVFINQIREKVGVMFGNPEVTPGGRALKFYSSVRLEVRRAEQLKQGQEIVGNRTKIKVVKNKVAPPFRVAEVDIMYGKGISKEGELIDLGAEHEIVNKSGAWYSYNGERMGQGKENVKLYLKENPAIREEIDQKLREKLGIFDGDVEEKEAESPATLFDEEE

SEQ ID NO: 292 - protein, 37 aa, Staphylococcus saprophyticus
MKVRPSVKPICEKCKVIKRKGKVMVICSNPKHKQRQG

SEQ ID NO: 293 - protein, 499 aa, Staphylococcus haemolyticus
MEKYIMSIDQGTTSSRAILFDKEGDIKGVAQREFKQYFPKSGWVEHDANEIWTSVLAVMTEVLNENEINADQIEGIGITNQRETTVIWDKNTGRPIYHAIVWQSRQTQSICHELKEQGHEETFRNKTGLLLDPYFAGTKVKWILDNVDGAREKAENGDLLFGTIDTWLVWKLSGGEAHITDYSNASRTLMYNIYDLQWDDELLDLLNVPKQLLPEVKESSEIYAHTKDYHFFGQEVPISGIAGDQQAALFGQACFERGDVKNTYGTGGFMLMNTGEEPVKSESGLLTTIAYGLDGKVNYALEGSIFVSGSAIQWLRDGLRIINSAPQSENYATRVDSTDNVYFVPAFVGLGTPYWDSEARGAIFGLSRGTEKEHFIRATLESLCYQTRDVMEAMSKDSKIEVNNLRVDGGAVKNNFIMQFQADIVNTAVERPEIQETTALGAAYLAGLAVGFWDSKDEIANRWQLETEFTPQMSEEDRTKLYKGWKKAVEATQVFKLED

SEQ ID NO: 294 - protein, 379 aa, Staphylococcus aureus
MAKRDYYEVLGISKDASKDEIKKAYRKLSKKYHPDINKEEGADEKFKEISEAYEVLSDDNKRASYDQFGHDGPQGFGGQGFNGSDFGGFSGFGGGGFEDIFSSFFGGGRQRDPNAPQKGDDLQYTMTLTFEEAVFGTTKEISIRKDVTCETCHGDGAKPGTSKKTCSYCNGAGHVAVEQNTILGRVRTEQVCPKCNGSGQEFEEACPTCHGKGTENKTVKLEVKVPEGVDNEQQIRLAGEGSPGVNGGPAGDLYVVFRVKPSETFKRDGDDIYYKLNVSFPQAALGDEIKIPTLNNEVMLTIPAGTQTGKQFRLKEKGIKNVHGYGYGDLYVDIKVVTPTKLTDRQKELMKEFAQLNGEEINEQPSNFKDRAKRFFKGE

SEQ ID NO: 295 - protein, 394 aa, Staphylococcus aureus
MNSIIELTDYYSSNNYAPLKLVISKGKGVKVWDTDGKQYIDCISGFSVANQGHCHPTIVKAMTEQASKLSIISRVLYSDNLGKWEEKICHLAKKDKVLPLNSGTEAVEAAIKIARKWGSEVKGITDGQVEIIAMNNNFHGRTLGSLSLSNHDAYKAGFHPLLQGTTTVDFGDIEQLTQAISPNTAAIILEPIQGEGGVNIPPKGYIQAVRQLCDKHQILLIADEIQVGLGRTGKWFAMEWEQVVPDIYILGKALGGGLYPVSAVLANNDVMRVLTPGTHGSTFGGNPLAIAISTAALDVLKDEQLVERSERLGSFLLKALLQLKHPSIKEIRGRGLFIGIELNTDAAPFVDQLIQRGILCKDTHRTIIRLSPPLVIDKEEIHQIVAAFQDVFKN

SEQ ID NO: 296 - protein, 601 aa, Staphylococcus epidermidis
MCGIVGYIGYDNAKELLLKGLEKLEYRGYDSAGIAVVNDDGTKLFKEKGRIAELRKVADNSDEDGTLGIGHTRWATHGVPNYENSHPHQSTSGRFTLVHNGVIENYEELKAEYLSDVTFSSETDTEVIVQLVDYFSRQGLATEDAFTKVVKLLHGSYALGLLDDNDKDTIYVAKNKSPLLVGVGEGFNVIASDALAMLQTTNQYKEIHDHEIVIVKRDTVEIKDLEGHIQQRDTYTAEIDAADAEKGVYDHYMLKEIHEQPAVMRRIIQEYQDEKGNLKIDSEIINDVADADRIYIVAAGTSYHAGLVGKEFIEKWAGVPTEVHVASEFVYNMPLLSEKPLFIYISQSGETADSRAVLVETNKLGHKSLTITNVAGSTLSREADHTLLLHAGPEIAVASTKAYTAQIAVLSILSQIVAKNHGRETDVDLLRELAKVTTAIETIVDDAPKMEQIATDFLKTTRNAFFIGRTIDYNVSLEGALKLKEISYIQAEGFAGGELKHGTIALIEDGTPVIGLATQENVNLSIRGNMKEVVARGAYPCMISMEGLNKEGDTYVIPQVHELLTPLVSVVTMQLISYYAALQRDLDVDKPRNLAKSVTVE

SEQ ID NO: 297 - protein, 401 aa, Staphylococcus epidermidis
MKDKIVLAYSGGLDTSVAVQWLIDKGYDVVACCLDVGEGKDLDVVYQKALDMGAVECHIIDATKEFSDDYVSYAIKGNLMYENAYPLVSALSRPLIAKKLVEIAEKTNSIGIAHGCTGKGNDQVRFEVAIKALNPKLKAFAPVREWAWSREEEIDYAIKHNIPVSINYDSPYSIDQNLWGRANECGILEDPYAAPPEDAFDLTTPLEETPDNADEIILTFKQGIPVQVDGKDYQLDDLILYLNQLAGKHGIGRIDHVENRMVGIKSREIYETPGAEVILKAHKALETITLTKDVAHFKPVIEKQFSEQIYNGLWFSPLTDSLKLFIDSTQQYVEGDVRIKLFKGNAIVNGRQSPYTLYDEKLATYTKEDAFNQESAVGFIDIYGLPTQVNALLHGGYSNEQ

SEQ ID NO: 298 - protein, 490 aa, Staphylococcus aureus
MTSKSSPLIFERSREGRYAYSLPKSDIKTNSVESLLDDKFIRKNKAEFPEVAELDLVRHYTELSNKNFGVDNGFYPLGSCTMKYNPKINEKVARIPGFSESHPLQDEDQVQGSLEIIYSLQEELKEITGMDEVTLQPAAGAHGEWTALMIFKAYHENNGEGHRDEVIVPDSAHGTNPASASFAGFKSVTVKSNERGEVDIDDLKRVVNENTAAIMLTNPNTLGIFEKNIMEIREIVHNAGGLLYYDGANLNAIMDKVRPGDMGFDAVHLNLHKTFTGPHGGGGPGSGPVGVVKELASYLPKPMVIKDGDKFKYDNDIKNSIGRVKPFYGNFGIYLRAYTYIRTMGATGLKEVSEAAVLNANYIKARLSKHFEIPYKQYCKHEFVLSGVRQKEFGVRTLDMAKRLLDFGVHPPTIYFPLNVEEGMMIEPTETESKETLDYFIDTLISIAEEAKNDPDKVLEAPHTTVIDRLDEATAARKPILKFENLKQEK

SEQ ID NO: 299 - protein, 220 aa, Staphylococcus aureus
MKEKFDLVKLLNILKKNIKLLLILPAICLVVSAALTFFVMPDKYTASTQILVNMKKSSSDLAFQNVQSSLQSVNTYTEIIKSPRILDKVSREFDGQYSTAELNSFLKVTNQTNSQIITVSVTTGNKSESDKIVNRISKVFAHDMPKIMSVDNVTILSSAHDNAVKVSPIVSVNLVISIIVGIVLAILIIFLKELLDKRIKTEEDVESQLGLPILGSIQKF

SEQ ID NO: 300 - protein, 290 aa, Staphylococcus aureus
MKKKTLLPLFLGIMVFLAGCDYSKPEKRSGFFYNTFVDPMKNVLDWLGNNLLNDNYGLAIIILVLVIRIILLPFMLSNYKNSHMMRQKMKVAKPEVEKIQEKVKRARTQEEKMAANQELMQVYKKYDMNPIKSMLGCLPMLIQLPIIMGLYFVLKDQLVDGLFKYPHFLWFDLGRPDIWITIIAGVLYFIQAYVSSKTMPDEQRQMGYMMMVISPIMIIWISLSSASALGLYWSVSAAFLVVQTHFANIYYEKVAKKEVQPFIEAYEREHNGGSNKKGKNTQVVSKKKKK

SEQ ID NO: 301 - protein, 465 aa, Staphylococcus aureus
MAKKAPDVGDYKYGFHDDDVSIFRSERGLTENIVREISNMKNEPEWMLDFRLKSLKLFYKMPMPQWGGDLSELNFDDITYYVKPSEQAERSWDEVPEEIKRTFDKLGIPEAEQKYLAGVSAQYESEVVYHNMEKELEEKGIIFKDTDSALQENEELFKKYFASVVPAADNKFAALNSAVWSGGSFIYVPKNIKLDTPLQAYFRINSENMGQFERTLIIADEGASVHYIEGCTAPVYTTSSLHSAVVEIIVHKDAHVRYTTIQNWANNVYNLVTKRTFVYENGNMEWVDGNLGSKLTMKYPNCVLLGEGAKGSTLSIAFAGKGQVQDAGAKMIHKAPNTSSTIVSKSISKNGGKVIYRGIVHFGRKAKGARSNIECDTLILDNESTSDTIPYNEVFNDQISLEHEAKVSKVSEEQLFYLMSRGISEEEATEMIVMGFIEPFTKELPMEYAVEMNRLIKFEMEGSIG

SEQ ID NO: 302 - protein, 417 aa, Staphylococcus epidermidis
MSKLILAVNAGSSSLKFQLIKMPEEKLVTKGVIERIGLSDSIFTIHVNGEKLTDIRDIHNHEEAVNIMLDSFKEHEMIKDITDIQGTGHRVVHGGETFPKSVVVTDEVESQIEELSELAPLHNPANLMGIRAFRKLLPEIPHVAVFDTSFHQTMPEQAYLYSLPYHYYEDYGIRKYGFHGTSHKYVSRRAAQIVGRPIEDLRIISCHIGNGASIAAIDGGESIDTSMGFTPLAGVTMGTRSGNLDPALIPFIMEKTGKTADEVLEILNKESGLLGLTGTSSDLRDLTEEAKHGRQRSRVALDLFASKIHKYIGSYAARMHGVDVIVFTAGIGENSHIIRGKVLEGLEFMGVYWDPKKNESLHGEEGYINYPHSPVKVLVVPTDEEVMISRDVIKYGKLNDNTPKKEEFDTNESIEVN

SEQ ID NO: 303 - protein, 49 aa, Staphylococcus epidermidis
MRVNVTLACTECGDRNYISTKNKRNNPERVEMKKYCSRDNKHTLHRETK

SEQ ID NO: 304 - protein, 149 aa, Staphylococcus aureus
MERTFLMIKPDAVQRNLIGEVISRIERKGLKLVGGKLMQVPMELAETHYGEHQGKPFYNDLISFITSAPVFAMVVEGEDAVNVSRHIIGSTNPSEASPGSIRGDLGLTVGRNIIHGSDSLDSAEREINLWFNENEITSYASPRDAWLYE

SEQ ID NO: 305 - protein, 368 aa, Staphylococcus aureus
MSDNTVKIKKQTIDPTLTLEDVKKQLIEKGKKEGHLSHEEIAEKLQNFDIDSDQMDDFFDQLNDNDISLVNEKDSSDTDEKLNPSDLSAPPGVKINDPVRMYLKEIGRVNLLSAQEEIELAKRIEQGDEVAKSRLAEANLRLVVSIAKRYVGRGMLFLDLIQEGNMGLIKAVEKFDFNKGFKFSTYATWWIRQAITRAIADQARTIRIPVHMVETINKLIRVQRQLLQDLGRDPAPEEIGEEMDLPAEKVREVLKIAQEPVSLETPIGEEDDSHLGDFIEDQEAQSPSDHAAYELLKEQLEDVLDTLTDREENVLRLRFGLDDGRTRTLEEVGKVFGVTRERIRQIEAKALRKLRHPSRSKRLKDFMD

SEQ ID NO: 306 - protein, 401 aa, Staphylococcus aureus
MKEKIVLAYSGGLDTSVAVQWLIDKGYDVVACCLDVGEGKDLDIVYKKALDMGAVECHIIDATKEFSDEYVSYAIKGNLMYENAYPLVSALSRPLIAKKLVEIAEKTNSVGIAHGCTGKGNDQVRFEVAIKALNPSLKAFAPVREWAWSREEEIDYAIKHNIPVSINHDSPYSIDQNLWGRANECGILEDPYAAPPEDAFDLTNALEETPDAADEIILTFDKGIPVQIDGKTYELDDLILTLNALAGKHGIGRIDHVENRLVGIKSREIYEAPAAEVILKAHKALETITLTKDVAHFKPIIEKQFAEQLYNGLWFSPLTDSLKLFIDSTQQYVSGDVRIKLFKGNAIVNGRKSPYTLYDEKLATYTKEDAFNQDAAVGFIDIYGLPTQVNSMLHGGYSNEQ

SEQ ID NO: 307 - protein, 396 aa, Staphylococcus epidermidis
MAKKIVSDLDLKGKVVLERADFNVPIKDGEITNDNRIVQALPTIEYIIEQGGKLVLFSHLGKVKEESDKEGLSLKPVAENLSKKLGKEVIFVPETRGEKLETAIENLNEGDVLLVENTRFEDLDGKKESKNDPELGKYWASLGDVFVNDAFGTAHREHASNVGISTHLETAAGYLMEKEIKFIGGVVNDPQKPVVAILGGAKVSDKINVIKNLVNIADKILIGGGMAYTFIKAQGKEIGLSLLEEDKIDFAKDLLENNGDQIVLPVDCKIAKEFSNDAKITEVSINEIPSDQEAMDIGPKTVELFNKELQGAHTVVWNGPMGVFEFSNFAKGTIGVCESIAKLEDATTIIGGGDSAAAAISLGFEDDFTHISTGGGASLEYLEGKELPGIKAINDK

SEQ ID NO: 308 - protein, 470 aa, Staphylococcus haemolyticus
MSNGRVTQVMGPVIDVRFEHNEVPEINNALIIEVPKEDGTFELTLEVALQLGDDVVRTIAMDSTDGVQRGMEVQNTGKDISVPVGEVTLGRVFNVLGDTIDLEDKLDGSVRRDPIHRQSPNFDELSTEVEILETGIKVVDLLAPYIKGGKIGLFGGAGVGKTVLIQELINNIAQEHGGISVFAGVGERTREGNDLYYEMRDSGVIKKTAMVFGQMNEPPGARMRVALSALTMAEYFRDEQGQDVLLFIDNIFRFTQAGSEVSALLGRMPSAVGYQPTLATEMGQLQERITSTNKGSVTSIQAVFVPADDYTDPAPATAFAHLDATTNLERKLTEMGIYPAVDPLASTSRALEPAIVGQEHYEVARDVQSTLQKYRELQDIIAILGMDELSEEDKLTVERARRIQFFLSQNFHVAEQFTGQKGSYVPVKTTVADFKDILDGKYDHIPEDAFRLVGSMEDVIAKAKDMGVEV

SEQ ID NO: 309 - protein, 325 aa, Staphylococcus aureus
MKMNKLVKSSVATSMALLLLSGTANAEGKITPVSVKKVDDKVTLYKTTATADSDKFKISQILTFNFIKDKSYDKDTLVLKATGNINSGFVKPNPNDYDFSKLYWGAKYNVSISSQSNDSVNVVDYAPKNQNEEFQVQNTLGYTFGGDISISNGLSGGLNGNTAFSETINYKQESYRTTLSRNTNYKNVGWGVEAHKIMNNGWGPYGRDSFHPTYGNELFLAGRQSSAYAGQNFIAQHQMPLLSRSNFNPEFLSVLSHRQDGAKKSKITVTYQREMDLYQIRWNGFYWAGANYKNFKTRTFKSTYEIDWENHKVKLLDTKETENNK

SEQ ID NO: 310 - protein, 115 aa, Staphylococcus sp.
MSKINDINDLVNATFQVKKFFRDTKKKFNLNYEEIYILNHILRSESNEISSKEIAKCSEFKPYYLTKALQKLKDLKLLSKKRSLQDERTVIVYVTDTQKANIQKLISELEEYIKN

SEQ ID NO: 311 - protein, 116 aa, Staphylococcus sp.
MSNKVQRFIEAERELSQLKHWLKTTHKISIEEFVVLFKVYEAEKISGKELRDTLHFEMLWDTSKIDVIIRKIYKKELISKLRSETDERQVFYFYSTSQKKLLDKITKEIEVLSVTN

SEQ ID NO: 312 - protein, 124 aa, unidentified
  REGION 1..124: note = Eubacteria sp.
MAITKINDCFELLSMVTYADKLKSLIKKEFSISFEEFAVLTYISENKEKEYYLKDIINHLNYKQPQVVKAVKILSQEDYFDKKRNEHDERTVLILVNAQQRKKIESLLSRVNKRITEANNEIEL

SEQ ID NO: 313 - protein, 675 aa, Staphylococcus carnosus
MWKKFFGQLQRIGKALMLPVAILPAAGLLLALGNAFQGDALQSLMPFIKAEGFQNVAKMMEGAGGIIFDNLAIIFALGVAIGLASGDGVAAIAAFVGFIVLNKTMGMFLGVTPEKAADAATGFANVLGIPTLQTGVFGGIIIGALAAWCYNKFYNISLPSYLGFFAGKRFVPIMMATCSFILAFPMAIIWPSIQGGLNAFSEGLLASNTGLAVFLFGFIKRLLIPFGLHHIFHAPFWFEFGSYKNAAGQIIHGDQRIFIEQIRDNVPLTAGKFMQGEFPVMMFGLPAAALAIYQTAKKENKKVVAGLMLSGALTSFLTGITEPLEFSFLFVAPLLFFIHAVLDGLSFLILYLLDLHLGYTFSGGFIDFFLLGILPNKTQWWLVIPVGLVYAAIYYIIFRFLIVKFNFKTPGREDKEVKSSNVAASELPFKVLDAMGGKANIKHLDACITRLRVEVNDKAKVDVQELKDLGASGVLEVGNNMQAIFGPKSDQIKHDMQQIMDGKITSPEETTVTEEGDKETAEIAAAGGGVVYAPIKGEVVDISEVPDKVFSEKMMGDGIAIKPETGEVVAPFDGVVKMVFPTKHAIGLESKDGIELLIHFGLETVKLEGKGFDILVKENDNIVLGQPLMKVDLDYIKEHADSTITPIVVTNLNGRTMEVLQHGEVKQGDKVILVK

SEQ ID NO: 314 - protein, 366 aa, Staphylococcus aureus
MSKFKSLLLLFGTLILLSGCSNIEIFNAKGPVASSQKFLILYSIVFMLVICFVVLGMFAIFIYKYSYNKNAESGKMHHNAIIETIWFVIPIIIVAALAIPTVKTLYDYEKPPKSEKDPMVVYAVSAGYKWFFAYPDEHIETVNTLTIPKDRPVVFKLQAMDTMTSFWIPQLGGQKYAMTGMTMNWTLEASQTGTFRGRNSNFNGEGFSRQTFKVNAVSQKDYDKWVKEVKGKKTLDQDTFDKQLLPSTPNKALEFNGTHMAFVDPAADPEYIFYAYKRFNFELKDPNFTSEENMFKDVSEKPLIPARKAQITNANYKRHGMKLMILGNDEPYNNEFKKDESKNAKEMKKISKDAQDQDNDDHGGGH

SEQ ID NO: 315 - protein, 204 aa, Staphylococcus aureus
MANPIKIGIGGPVGAGKTQLIEKVVKRLSKEMSIGVITNDIYTKEDEKILVNSGVLPESRIIGVETGGCPHTAIREDASMNFAAIDELLERHDDIELIFIESGGDNLAATFSPELVDFSIYIIDVAQGEKIPRKGGQGMIKSDFFVINKTDLAPYVGASLEQMAEDTKVFRDKRPFTFTNLKTDEGLDEVIDWIKRDTLLKGLS

SEQ ID NO: 316 - protein, 411 aa, Staphylococcus aureus
MTDGPIKVNSEIGALKTVLLKRPGKELENLVPDYLDGLLFDDIPYLEVAQKEHDHFAQVLREEGVEVLYLEKLAAESIENPQVRSEFIDDVLAESKKTILGHEEEIKTLFATLSNQELVDKIMSGVRKEEINPKCTHLVEYMDDKYPFYLDPMPNLYFTRDPQASIGHGITINRMFWRARRRESIFIQYIVKHHPRFKDANIPIWLDRDCPFNIEGGDELVLSKDVLAIGVSERTSAQAIEKLARRIFENPQATFKKVVAIEIPTSRTFMHLDTVFTMIDYDKFTMHSAILKAEGNMNIFIIEYDDVNKDIAIKQSSHLKDTLEDVLGIDDIQFIPTGNGDVIDGAREQWNDGSNTLCIRPGVVVTYDRNYVSNDLLRQKGIKVIEISGSELVRGRGGPRCMSQPLFREDI

SEQ ID NO: 317 - protein, 443 aa, Staphylococcus aureus
MTHIQLDFSKTLEFFGEHELKQQQEIVKSIHKTIHEGTGAGSDFLGWVDLPVDYDKEEFSRIVEASKRIKENSDVLVVIGIGGSYLGARAAIEMLTSSFRNSNEYPEIVFVGNHLSSTYTKELVDYLADKDFSVNVISKSGTTTEPAVAFRLFKQLVEERYGKEEAQKRIFATTDKEKGALKQLATNEGYETFIVPDDVGGRYSVLTAVGLLPIATAGINIEAMMIGAAKAREELSSDKLEENIAYQYATIRNILYAKGYTTEMLINYEPSMQYFNEWWKQLFGESEGKDFKGIYPSSANYTTDLHSLGQYVQEGRRFLFETVVKVNHPKYDITIEKDSDDLDGLNYLAGKTIDEVNTKAFEGTLLAHTDGGVPNMIVNIPQLDEETFGYVVYFFELACAMSGYQLGVNPFNQPGVEAYKQNMFALLGKPGFEDLKKELEERL

SEQ ID NO: 318 - protein, 586 aa, Staphylococcus haemolyticus
MRKTKIVCTIGPASESEEMLEKLMKAGMNVARLNFSHGSHEEHKARIDTIRKVADRLGKTIGILLDTKGPEIRTHDMKDGLIMLEKGKEVIVSMSQVEGTPEKFSVTYEDLINDVQVGSYILLDDGLVELQVKDIDKTKGEVKCDILNTGELKNKKGVNLPGVKVNLPGITDKDADDILFGIKEDVDYIAASFVRRPSDVLDIREILERENNHNITIFPKIENQEGIDNIEEILEVSDGLMVARGDMGVEIPPESVPIVQKDLIRKCNKLGKPVITATQMLDSMQRNPRATRAEASDVANAIYDGTDAVMLSGETAAGLYPEEAVKTMRNIAVSAEAAQDYKKLLSDRTKLVETSLVNAIGVSVAHTALNLNVKAIVAATESGSTAVTISKYRPHSDIIAVTPSEHTARQLALVWGAYPVIKKGRKTTDDLLNNAVATAVETGRVTNGDLIIITAGVPTGEKGTTNMMKLHLVGDEIAKGQGVGRGSVVGKTVVANSASDLEGVDLSESVIVTNSVDETLVPYIEQAVGLITEENGITSPSAIIGLEKSIPTIIGVENATKELKDGILVTVDAAQGKIFEGYANVL

SEQ ID NO: 319 - protein, 394 aa, Staphylococcus epidermidis
MLEFEQGFNHLATLKVIGVGGGGNNAVNRMIDHGMNNVEFIAINTDGQALNLSKAESKIQIGEKLTRGLGAGANPEIGKKAAEESREQIEDAIQGADMVFVTAGMGGGTGTGAAPVVAKIAKEMGALTVGVVTRPFGFEGRKRQTQAAAGVESMKAAVDTLIVIPNDRLLDIVDKSTPMMEAFKEADNVLRQGVQGISDLIAVSGEVNLDFADVKTIMSNQGSALMGIGVSSGENRAVEAAKKAISSPLLETSIVGAQGVLMNITGGESLSLFEAQEAADIVQDAADEDVNMIFGTVINPELQDEIVVTVIATGFEDKPSSQGRKATSTGFGSSVNSSSNHQSGASAKEDSFSAHTSHSQSSESVSERSHTTKDDDIPSFIRNREERRSRRTRR

SEQ ID NO: 320 - protein, 978 aa, Staphylococcus sp.
MIIKSLEIYGYGQFVQRKIEFNKNFTEIFGENEAGKSTIQAFIHSILFGFPTKKSKEPRLEPRLGNQYGGKLVLILDDGLEIEVERIKGSAQGDVKVYLPNGAVRDDAWLQKKLNYISKKTYQGIFSFDVLGLQDIHRNLNEKQLQDYLLQAGALGSTEFTSMREVINRKKDELYKKSGKNPIINQQIEQLKQLESQIREEEAKLETYHRLVDDRDKSSRRLENLKHNLNQLSKMHEEKQKEVALHDHSQEWKSLEQQLNIEPITFPEKGVDRYEKARAHKQSLERDIGLRNERLAQLKEEATQLEPVKQSDIDAFISLNQQENEIKNKEFELTAIEKDIANKQRDKDELQANIGWSETHHDVDSSEAMKSYVSEQIKNKQEQAAYIKQLERSLEENKIEDNAVHSELDSVEEKIVPEETFEKKKEYSQQVIELNEKENLYSKLKERFEIEQQEKQKRQKLLRTTFILLTLVGIGLTVFSFISNNMLFGIIFAVLTLVFVIGIIMSKSKEVYYSEAITDEIEVIKAQLAILDENYDLDFDLDEQYRIRDHWQQALKNKDILEEKRQYIEGRLNDAKGRHDELQSTVENVKDELYLSSKISNDLIVDSISTMANIKALDQHISDLNQQRQQLVQELDTFYNHAEAVTKSQFVYFNKLSLFHDVQQWLKSAEDTNEKWRINAENTKLVTNELNHLNAQLEENNKEITALFDFINVGTEEDFYQHHEDYQTYTSNLSRFNDLTKYLENQNYSYELSSSLSEKTTAQLEEEDHLLATQVDEYNEQYLEMQAQVSDLSAQINHMETDTTLANLRHEYHSLKNQLNDIAKDWASLSYLQSLVDEHIKQIKDKRLPQVINEAVEILKHLTDGRYTMINYNEDSITVKHVNGQLYDPVELSQSTKELLYVALRISLIKVLRPYYPFPLIVDDAFVHFDKKRTEKMLNYLRSLSEHYQVLYFTCVKDNIVPSKEVITLNKIEEGGKR

SEQ ID NO: 321 - protein, 296 aa, Staphylococcus aureus
MNKEQLEKMKNGKGFIAALDQSGGSTPKALKEYGVNEDQYSNEDEMFQLVHDMRTRVVTSPSFSPDKILGAILFEQTMDREVEGKYTADYLADKGVVPFLKVDKGLAEEQNGVQLMKPIDNLDSLLDRANERHIFGTKMRSNILELNEQGIKDVVEQQFEVAKQIIAKGLVPIIEPEVNINAKDKAEIEKVLKAELKKGLDSLNADQLVMLKLTIPTEPNLYKELAEHPNVVRVVVLSGGYSREKANELLKDNDELIASFSRALASDLRADQSKEEFDKALGDAVESIYDASVNKN

SEQ ID NO: 322 - protein, 498 aa, Staphylococcus aureus
MAKSNSKDIVLIGAGVLSTTFGSMLKEIEPDWNIHVYERLDRPAIESSNERNNAGTGHAALCELNYTVLQPDGSIDIEKAKVINEEFEISKQFWGHLVKSGSIENPRGFINPLPHISYVRGKNNVKFLKDRYEAMKAFPMFDNIEYTEDIEVMKKWIPLMMKGREDNPGIMAASKIDEGTDVNFGELTRKMAKSIEAHPNATVQFNHEVVDFEQLSNGQWEVTVKNRLTGEKFKQVTDYVFIGAGGGAIPLLQKTGIPESKHLGGFPISGQFLACTNPQVIEQHDAKVYGKEPPGTPPMTVPHLDTRYIDGQRTLLFGPFANVGPKFLKNGSNLDLFKSVKTYNITTLLAAAVKNLPLIKYSFDQVIMTKEGCMNHLRTFYPEARNEDWQLYTAGKRVQVIKDTPEHGKGFIQFGTEVVNSQDHTVIALLGESPGASTSVSVALEVLERNFPEYKTEWAPKIKKMIPSYGESLIEDEKLMRKIRKQTSKDLELGYYEN

SEQ ID NO: 323 - protein, 567 aa, Staphylococcus aureus
MKQSKVFIPTMRDVPSEAEAQSHRLLLKSGLIKQSTSGIYSYLPLATRVLNNITAIVRQEMERIDSVEILMPALQQAELWEESGRWGAYGPELMRLQDRHGRQFALGPTHEELVTSIVRNELKSYKQLPMTLFQIQSKFRDEKRPRFGLLRGREFIMKDAYSFHADEASLDQTYQDMYQAYSRIFERVGINARPVVADSGAIGGSHTHEFMALSAIGEDTIVYSKESDYAANIEKAEVVYEPNHKHTTVQPLEKIETPNVKTAQELADFLGRPVDEIVKTMIFKVDGEYIMVLVRGHHEINDIKLKSYFGTDNIELATQDEIVNLVGANPGSLGPVIDKEIKIYADNFVQDLNNLVVGANEDGYHLINVNVGRDFNVDEYGDFRFILEGEKLSDGSGVAHFAEGIEVGQVFKLGTKYSESMNATFLDNQGKAQSLIMGCYGIGISRTLSAIVEQNHDDNGIVWPKSVTPFDLHLISINPKKDDQRELADALYAEFNTKFDVLYDDRQERAGVKFNDADLIGLPLRIVVGKRASEGIVEVKERLTGDSEEVHIDDLMTVITNKYDNLK

SEQ ID NO: 324 - protein, 1229 aa, Staphylococcus sp.
MGKFGLNFFKPTEKFNGNWSILESKSREWEKMYRERWSHDKEVRTTHGVNCTGSCSWKVFVKNGVITWENQQTDYPSCGPDMPEYEPRGCPRGASFSWYEYSPLRIKYPYIRGKLWDLWTEALEENNGNRVAAWASIVENEDKAKQYKQARGMGGHVRSNWKDVTDIIAAQLLYTIKKYGPDRIAGFTPIPAMSMISYAAGARFINLLGGEMLSFYDWYADLPPASPQIWGEQTDVPESSDWYNASYIIMWGSNVPLTRTPDAHFMTEVRYKGTKVISVAPDYAENVKFADNWLAPNPGSDAAIAQAMTHVILQEHYVNQPNERFINYAKQYTDMPFIIMLDEDENGYKAGRFLRASDLGQTTEQSEWKPVIHDTISDSLVVPNGTMGQRWEEGKKWNLKLETEDGSKINPTLSMTEGGYELETIQFPYFDSDGDGIFNRPIPTRQVTLANGDKVRIATIFDLMASQYGVRRFDHKLESNGYDDAESKYTPAWQEAISGVKQSVVIQVAKEFAQNAIDTEGRSMIIMGAGINHWFNSDTIYRSILNLVMLCGCQGVNGGGWAHYVGQEKCRPIEGWSTVAFAKDWQGPPRLQNGTSWFYFATDQWKYEESNVDRLKSPLAKTEELKHQHPADYNVLAARLGWLPSYPQFNKNSLLFAEEAKDEGIDSNEAILQRAIDEVKSKQTQFAIEDPDLKKNHPKSLFIWRSNLISSSAKGQEYFMKHLLGTKSGLLATPNEDEKPEEITWREETTGKLDLVVSLDFRMTATPLYSDIVLPAATWYEKHDLSSTDMHPYVHPFNPAIDPLWESRSDWDIYKTLAKAFSEMAKDYLPGTFKDVVTTPLSHDTKQEISTPYGVVKDWSKGEIEAVPGRTMPNFAIVERDYTKIYDKYVTLGPVLEKGKVGAHGVSFGVSEQYEELKSMLGTWSDTNDDSVRANRPRIDTARNVADAILNISSATNGKLSQKSYEDLEEQTGMPLKDISSERAAEKISFLNITSQPREVIPTAVFPGSNKQGRRYSPFTTNVERLVPFRTLTGRQSYYVDHEVFQQFGESLPVYKPTLPPMVFGNRDKKIKGGTDALVLRYLTPHGKWNIHSMYQDNKHMLTLFRGGPTVWISNEDAEKHDIQDNDWLEVYNRNGVVTARAVISHRMPKGTMFMYHAQDKHIQTPGSEITDTRGGSHNAPTRIHLKPTQLVGGYAQISYHFNYYGPIGNQRDLYVAVRKMKEVNWLED

SEQ ID NO: 325 - protein, 533 aa, Staphylococcus sp.
MLQVTDVSLRFGDRKLFEDVNIKFTEGNCYGLIGANGAGKSTFLKILSGELDSQTGHVSLGKNERLAVLKQDHYAYEDERVLDVVIKGHERLYEVMKEKDEIYMKPDFSDEDGIRAAELEGEFAEMNGWNAEADAANLLSGLGIDPTLHDKKMAELENNQKIKVLLAQSLFGEPDVLLLDEPTNGLDIPAISWLEDFLINFDNTVIVVSHDRHFLNNVCTHIADLDFGKIKVYVGNYDFWYQSSQLAQKMAQEQNKKKEEKMKELQDFIARFSANASKSKQATSRKKQLEKIELDDIQPSSRRYPFVKFTPEREIGNDLLIVQNLSKTIDGEKVLDNVSFTMNPNDKAILIGDSEIAKTTLLKILAGEMEPDEGSFKWGVTTSLSYFPKDNSEFFEGVNMNLVDWLRQYAPEDEQTETFLRGFLGRMLFSGEEVKKKASVLSGGEKVRCMLSKMMLSSANVLLLDEPTNHLDLESITAVNDGLKSFKGSIIFTSYDFEFINTIANRVIDLNKQGGVSKEIPYEEYLQEIGVLK

SEQ ID NO: 326 - protein, 565 aa, Staphylococcus aureus
MKQLHPNEVGVYALGGLGEIGKNTYAVEYKDEIVIIDAGIKFPDDNLLGIDYVIPDYTYLVQNQDKIVGLFITHGHEDHIGGVPFLLKQLNIPIYGGPLALGLIRNKLEEHHLLRTAKLNEINEDSVIKSKHLTISFYLTTHSIPETYGVIVDTPEGKVVHTGDFKFDFTPVGKPANIAKMAQLGEEGVLCLLSDSTNSLVPDFTLSEREVGQNVDKIFRNCKGRIIFATFASNIYRVQQAVEAAIKNNRKIVTFGRSMENNIKIGMELGYIKAPPETFIEPNKINTVPKHELLILCTGSQGEPMAALSRIANGTHKQIKIIPEDTVVFSSSPIPGNTKSINRTINSLYKAGADVIHSKISNIHTSGHGSQGDQQLMLRLIKPKYFLPIHGEYRMLKAHGETGVECGVEEDNVFIFDIGDVLALTHDSARKAGRIPSGNVLVDGSGIGDIGNVVIRDRKLLSEEGLVIVVVSIDFNTNKLLSGPDIISRGFVYMRESGQLIYDAQRKIKTDVISKLNQNKDIQWHQIKSSIIETLQPYLFEKTARKPMILPVIMKVNEQKESNNK

SEQ ID NO: 327 - protein, 80 aa, Staphylococcus saprophyticus
MAGGPRRGGRRRKKVCYFTANGITHIDYKDTELLKRFISERGKILPRRVTGTSAKYQRMLTLAIKRSRHMALLPYVKEEQ

SEQ ID NO: 328 - protein, 310 aa, Staphylococcus epidermidis
MAKIVVALGGNALGKSPQEQLELVKNTAKSLVGLITKGHEIVISHGNGPQVGSINLGLNYAAEHDQGPAFPFAECGAMSQAYIGYQLQESLQNELHSMGIDKQVVTLVTQVEVDEGDPAFNSPSKPIGLFYTKEEANRIQQEKGYQFVEDAGRGYRRVVPSPQPISIIELESIKTLVENDTLVIAAGGGGIPVIREQHDSFKGIDAVIDKDKTSALLGADIHCDQLIILTAIDYVYINYHTDQQQALKTTNIDTLKTYIEEEQFAKGSMLPKIESAISFIENNPNGSVLITSLNQLDAALEGKIGTLITK

SEQ ID NO: 329 - protein, 102 aa, Staphylococcus carnosus
MAKQKIRIRLKAYDHRVIDQSAEKIVETAKRSGAEVSGPIPLPTEKSVYTIIRAVHKYKDSREQFEQRTHKRLIDIVNPTPKTVDALMGLNLPSGVDIEIKL

SEQ ID NO: 330 - protein, 207 aa, Staphylococcus sp.
MANYDVLKLDGTKSGSIELSDAVFGIEPNNSVLFEAINLQRASLRQGTHAVKNRSAVSGGGRKPWKQKGTGRARQGTIRAPQWRGGGIVFGPTPRSYAYKMPKKMRRLALRSALSFKVQENGLTVVDAFNFEAPKTKEFKNVLSTLEQPKKVLVVTENEDVNVELSARNIPGVQVTTAQGLNVLDITNADSLVITEAAAKKVEEVLG

SEQ ID NO: 331 - protein, 309 aa, Staphylococcus sp.
MAKTYIFGHKNPDTDAISSAIIMAEFEQLRGNSGAKAYRLGDVSAETQFALDTFNVPAPELLTDDLDGQDVILVDHNEFQQSSDTIASATIKHVIDHHRIANFETAGPLCYRAEPVGCTATILYKMFRERGFEIKPEIAGLMLSAIISDSLLFKSPTCTQQDVKAAEELKDIAKVDIQKYGLDMLKAGASTTDKSVEFLLNMDAKSFTMGDYVTRIAQVNAVDLDEVLNRKEDLEKEMLAVSAQEKYDLFVLVVTDIINSDSKILVVGAEKDKVGEAFNVQLEDDMAFLSGVVSRKKQIVPQITEALTK

SEQ ID NO: 332 - protein, 186 aa, Staphylococcus aureus
MRHGQTLFNFKGLIQGFGDSPLTELGIAQAQKARSYYETKGINFDLYASSTQERASDTLENVAPNQSYQRFKGLKEWHFGLFEGESVYLFDNLYKPEDLFGDRIVPFKGEARQQVEDRIVKTLHDIMSQTKNNALVVSHGTIMGVFLRYCLKLDEALKHNIGNCNILKFEYDNGTFKFVELIDPNL

SEQ ID NO: 333 - protein, 1149 aa, Staphylococcus aureus
MINRDNKKAITKKGMISNRLNKFSIRKYTVGTASILVGTTLIFGLGNQEAKAAENTSTENAKQDEASASDNKEVVSETENNSTQKNDLTNPIKKETNTDSHQEAKEAPTTSSTQQQQNNATTSTETKPQNIEKENVKPSTDKTATEDTSVILEEKKAPNNTNNDVTTKPSTSEIQTTPTTPQESTNIENSQPQPTPSKVDNQVTDAINPKEPVNVSKEELKNNPEKLKELVRNDSNTDRSTKPVATAPTSVAPKRVNAKIRFAVAQPAAVASNNVNDLITVTKQMITEGIKDDGVIQAHDGEHIIYTSDFKIDNAVKAGDTMTVKYDKHTIPSDITDDFTPVDITDPSGEVIAKGTFDLNTKTITYKFTDYVDRYENVNAKLELNSYIDKKEVPNETNLNLTFATADKETSKNVKVEYQKPIVKDESNIQSIFSHLDTTKHEVEQTIYVNPLKLNAKNTNVTIKSGGVADNGDYYTGDGSTIIDSNTEIKVYKVASGQQLPQSNKIYDYSQYEDVTNSVTINKNYGTNMANINFGDIDSAYIVKVVSKYTPGAEDDLAVQQGVRMTTTNKYNYSSYAGYTNTILSTTDSGGGDGTVKPEEKLYKIGDYVWEDVDKDGVQGTDSKEKPMANVLVTLTYPDGTTKSVRTDANGHYEFGGLKDGETYTVKFETPAGYLPTKENGTTDGEKDSNGSSVTVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTAEDKDSNGLTTTGVIKDADNWTLDSGFYKTPKYSLGDYVWYDSNKDGKQDSTEKGIKDVTVTLQNEKGEVIGTTKTDENGKYRFDNLDSGKYKVIFEKPAGLTQTGTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSATKDHHNKAKALPETGSENNGSNNATLFGGLFAALGSLLLFGRRKKQNK

SEQ ID NO: 334 - protein, 901 aa, Staphylococcus epidermidis
MASNIKEQAKKQFELNGQSYTYYDLQTLEEKGLAKISKLPYSIRVLLESVLRQEDDFVITDDHIKALSKFGNAGNEGEVPFKPSRVILQDFTGVPAVVDLASLRKAMNDVGGDINKINPEVPVDLVIDHSVQVDSYANPEALERNMKLEFERNYERYQFLNWATKAFDNYNAVPPATGIVHQVNLEYLANVVHVRDVDGEKTAFPDTLVGTDSHTTMINGIGVLGWGVGGIEAEAGMLGQPSYFPIPEVIGVRLTHSLPQGSTATDLALRVTEELRKKGVVGKFVEFFGPGVQHLPLADRATIANMAPEYGATCGFFPVDEESLKYMKLTGRDEEHIELVKEYLQQNHMFFDVEKEDPEYTDVIDLDLSTVEASLSGPKRPQDLIFLSDMKKEFEKSVTAPAGNQGHGLDQSEFDKKAEINFNDGSKATMKTGDIAIAAITSCTNTSNPYVMLGAGLVAKKAVEKGLKVPEFVKTSLAPGSKVVTGYLRDSGLQQYLDDLGFNLVGYGCTTCIGNSGPLLPEIEKAVADEDLLVTSVLSGNRNFEGRIHPLVKANYLASPQLVVAYALAGTVDIDLQNEPIGKGKDGKDVYLQDIWPSIQEVSDTVDKVVTPELFLEEYKNVYHNNEMWNEIDVTDEPLYDFDPNSTYIQNPTFFQGLSKEPGKIEPLKSLRVMGKFGDSVTTDHISPAGAIGKDTPAGKYLLDHDVAIRNFNSYGSRRGNHEVMVRGTFANIRIKNQLAPGTEGGFTTYWPTGEIMPIYDAAMKYKEDGTGLVVLAGNDYGMGSSRDWAAKGTNLLGVKTVIAQSYERIHRSNLVMMGVLPLQFQQGESAEALGLDGKEEISVDINEDVQPHDLVNVTAKKENGEIINFKAIVRFDSLVELDYYRHGGILQMVLRNKLAQ

SEQ ID NO: 335 - protein, 229 aa, Staphylococcus aureus
MAKKGKKYQEAASKVDRTQHYSVEEAIKLAKETSIANFDASVEVAFRLGIDTRKNDQQIRGAVVLPNGTGKSQSVLVFAKGDKIAEAEAAGADYVGEAEYVQKIQQGWFDFDVVVATPDMMGEVGKLGRVLGPKGLMPNPKTGTVTMDVKKAVEEIKAGKVEYRAEKAGIVHASIGKVSFTDEQLIENFNTLQDVLAKAKPSSAKGTYFKSVAVTTTMGPGVKIDTASL

SEQ ID NO: 336 - protein, 388 aa, Staphylococcus aureus
MNIHEYQGKEIFRSMGVAVPEGRVAFTAEEAVEKAKEFNSDVYVVKAQIHAGGRGKAGGVKIAKSLSEVETYAKELLGKTLVTHQTGPEGKEIKRLYIEEGCAIQKEYYVGFVIDRATDQVTLMASEEGGTEIEEVAAKTPEKIFKETIDPVIGLSPFQARRIAFNINIPKESVNKAAKFLLALYNVFIEKDCSIVEINPLVTTADGDVLVLDAKINFDDNALFRHKDVVELRDLEEEDPKEIEASKHDLSYIALDGDIGCMVNGAGLAMATMDTINHFGGNPANFLDAGGSATREKVTEAFKIILGDENVKGIFVNIFGGIMKCDVIAEGIVEAVKEVDLTLPLVVRLEGTNVELGKKILKDSGLAIEPAATMAEGAQKIVKLVKEA

SEQ ID NO: 337 - protein, 1183 aa, Staphylococcus aureus
MAGQVVQYGRHRKRRNYARISEVLELPNLIEIQTKSYEWFLREGLIEMFRDISPIEDFTGNLSLEFVDYRLGEPKYDLEESKNRDATYAAPLRVKVRLIIKETGEVKEQEVFMGDFPLMTDTGTFVINGAERVILSQLVRSPSVYFNEKIDKNGRENYDATIIPNRGAWLEYETDAKDVVYVRIDRTRKLPLTVLLRALGFSSDQEIVDLLGDNEYLRNTLEKDGTENTEQALLEIYERLRPGEPPTVENAKSLLYSRFFDPKRYDLASVGRYKTNKKLHLKHRLFNQKLAEPIVNTETGEIVVEEGTVLDRRKIDEIMDVLESNANSEVFELHGSVIDEPVEIQSIKVYVPNDDEGRTTTVIGNAFPDSEVKCITPADIIASMSYFFNLLSGIGYTDDIDHLGNRRLRSVGELLQNQFRIGLSRMERVVRERMSIQDTESITPQQLINIRPVIASIKEFFGSSQLSQFMDQANPLAELTNKRRLSALGPGGLTRERAQMEVRDVHYSHYGRMCPIETPEGPNIGLINSLSSYARVNEFGFIETPYRKVDLDTHAITDQIDYLTADEEDSYVVAQANSKLDENGRFMDDEVVCRFRGNNTVMAKEKMDYMDVSPKQVVSAATACIPFLENDDSNRALMGANMQRQAVPLMNPEAPFVGTGMEHVAARDSGAAITAKHRGRVEHVESNEILVRRLVEENGVEHEGELDRYPLAKFKRSNSGTCYNQRPIVAAGDVVEFNEILADGPSMELGEMALGRNVVVGFMTWDGYNYEDAVIMSERLVKDDVYTSIHIEEYESEARDTKLGPEEITRDIPNVSESALKNLDDRGIVYIGAEVKDGDILVGKVTPKGVTELTAEERLLHAIFGEKAREVRDTSLRVPHGAGGIVLDVKVFNREEGDDTLSPGVNQLVRVYIVQKRKIHVGDKMCGRHGNKGVISKIVPEEDMPYLPDGRPIDIMLNPLGVPSRMNIGQVLELHLGMAAKNLGIHVASPVFDGANDDDVWSTIEEAGMARDGKTVLYDGRTGEPFDNRISVGVMYMLKLAHMVDDKLHARSTGPYSLVTQQPLGGKAQFGGQRFGEMEVWALEAYGAAYTLQEILTYKSDDTVGRVKTYEAIVKGENISRPSVPESFRVLMKELQSLGLDVKVMDEQDNEIEMTDVDDDDVVERKVDLQQNDAPETQKEVTD

SEQ ID NO: 338 - protein, 438 aa, Staphylococcus aureus
MPKIVVVGAVAGGATCASQIRRLDKESDIIIFEKDRDMSFANCALPYVIGEVVEDRKYALVYTPEKFYDRKQITVKTYHEVIAINDERQTVTVLNRKTNEQFEESYDKLILSPGASANSLGFESDITFTLRNLEDTDAIDQFIKANQVDKVLVIGAGYVSLEVLENLYERGLHPTLIHRSDKINKLMDADMNQPILDELDKREIPYRLNEEIDAINGNEITFKSGKVEHYDMIIEGVGTHPNSKLIESSNIKLDRKGFIPVNDKFETNVPNIYAIGDIATSHYRHVDLPASVPLAWGAHRAASIVAEQIAGNDTIEFKGFLGNNIVKFFDYTFASVGVKPNELKQFDYKMVEVTQGAHANYYPGNSPLHLRVYYDTSNRQILRAAAVGKEGADKRIDVLSMAMMNQLTVDELTEFEVAYAPPYSHPKDLINMIGYKAK

SEQ ID NO: 339 - protein, 467 aa, Staphylococcus aureus
MDTAGIRLTPKEIVSKLNEYIVGQNDAKRKVAIALRNRYRRSLLDEESKQEISPKNILMIGPTGVGKTEIARRMAKVVGAPFIKVEATKFTEVGYVGRDVESMVRDLVDVSVRLVKAQKKSLVQDEATAKANEKLVKLLVPSMKKKASQTNNPLESLFGGAIPNFGQNNEDEEEPPTEEIKTKRSEIKRQLEEGKLEKEKVRIKVEQDPGALGMLGTNQNQQMQEMMNQLMPKKKVEREVAVETARKILADSYADELIDQESANQEALELAEQMGIIFIDEIDKVATNNHNSGQDVSRQGVQRDILPILEGSVIQTKYGTVNTEHMLFIGAGAFHVSKPSDLIPELQGRFPIRVELDSLSVEDFVRILTEPKLSLIKQYEALLQTEEVTVNFTDEAITRLAEIAYQVNQDTDNIGARRLHTILEKMLEDLSFEAPSMPNAVVDITPQYVDDKLKSISTNKDLSAFIL

SEQ ID NO: 340 - protein, 199 aa, Staphylococcus aureus
MAFKLPNLPYAYDALEPYIDQRTMEFHHDKHHNTYVTKLNATVEGTELEHQSLADMIANLDKVPEAMRMSVRNNGGGHFNHSLFWEILSPNSEEKGGVIDDIKAQWGTLDEFKNEFANKATTLFGSGWTWLVVNNGKLEIVTTPNQDNPLTEGKTPILLFDVWEHAYYLKYQNKRPDYMTAFWNIVNWKKVDELYQAAK

SEQ ID NO: 341 - protein, 188 aa, Staphylococcus aureus
MKGLIIIGSAQVNSHTSALARYLTEHFKTHDIEAEIFDLAEKPLNQLDFSGTTPSIDEIKQNMKDLKEKAMAADFLILGTPNYHGSYSGILKNALDHLNMDYFKMKPVGLIGNSGGIVSSEPLSHLRVIVRSLLGIAVPTQIATHDSDFAKNEDGSYYLNDSEFQLRARLFVDQIVSFVNNSPYEHLK

SEQ ID NO: 342 - protein, 326 aa, Staphylococcus sp.
MEDLEKVLITGGAGFIGSHLVDDLQQDYDVYVLDNYRTGKRENIKSLADDHVFELDIREYDAVEQIMKTYQFDYVIHLAALVSVAESVEKPILSQEINVVATLKLLETIKKYNSHIKRFIFASSAAVYGDLPDLPKSDQSLILPLSPYAIDKYYGERTTLNYCSLYNIPTAVVKFFNVFGPRQDPKSQYSGVISKMFDSFEHNKPFTFFGDGLQTRDFVYVYDVVQSVRLIMEHKDAVGHGYNIGTGTFTNLLEVYRIIGELYGKSVEHDFKEARKGDIKHSYADISNLKALGFVPKYTVETGLKDYFNFEVDNIEEVTAKEVEMS

SEQ ID NO: 343 - protein, 670 aa, Staphylococcus aureus
MKKIKIVPLILIVVVVGFGIYFYASKDKEINNTIDAIEDKNFKQVYKDSSYISKSDNGEVEMTERPIKIYNSLGVKDINIQDRKIKKVSKNKKRVDAQYKIKTNYGNIDRNVQFNFVKEDGMWKLDWDHSVIIPGMQKDQSIHIENLKSERGKILDRNNVELANTGTHMRLGIVPKNVSKKDYKAIAKELSISEDYINNKWIKIGYKMIPSFHFKTVKKMDEYLSDFAKKFHLTTNETESRNYPLGKATSHLLGYVGPINSEELKQKEYKGYKDDAVIGKKGLEKLYDKKLQHEDGYRVTIVRVDDNSNTIAHTLIEKKKKDGKDIQLTIDAKVQKSIYNNMKNDYGSGTAIHPQTGELLALVSTPSYDVYPFMYGMSNEEYNKLTEDKKEPLLNKFQITTSPGSTQKILTAMIGLNNKTLDDKTSYKIDGKGWQKDKSWGGYNVTRYEVVNGNIDLKQAIESSDNIFFARVALELGSKKFEKGMKKLGVGEDIPSDYPFYNAQISNKNLDNEILLADSGYGQGEILINPVQILSIYSALENNGNINAPHLLKDTKNKVWKKNIISKENINLLNDGMQQVVNKTHKEDIYRSYANLIGKSGTAELKMKQGETGRQIGWFISYDKDNPNMMMAINVKDVQDKGMASYNAKISGKVYDELYENGNKKYDIDE

SEQ ID NO: 344 - protein, 655 aa, Staphylococcus sp.
MNRQSTKKALLIPFVIMIITAIVLMGVWFIFNSLIALIASIVLVVMIIVSIVLFRQALMKMDSYVDGLSAQISTTNNKAIKHLPIGIIVLDENDHIEWVNQFMTDHMEANVISESVNEVFPNILKQLDRVKSVEIEYNQYHFQVRYSENDHCLYFFDITEQVQTNELYENSKPIIATLFLDNYDEITQNMNDTQRSEINSMVTRVISRWATEYNIFFKRYSSDQFVAYLNQKILADLEESKFDILSQLREKSVGYRAQLTLSIGVGEGTENLIDLGELSQSGLDLALGRGGDQVAIKSINGNVRFYGGKTDPMEKRTRVRARVISHALKDILAEGDKVIIMGHKRPDLDAIGAAIGVSRFAMMNNLEAYIVLNETDIDPTLRRVMNEIDKKPELRERFITSDDAWDMMTSKTTVVIVDTHKPELVLDENVLNKANRKVVIDHHRRGESFISNPLLIYMEPYASSTAELVTELLEYQPTEQRLTRLESTVMYAGIIVDTRNFTLRTGSRTFDAASYLRAHGADTILTQHFLKDDVDTYINRSELIRTVKVEDNGIAIAHGSDDKIYHPVTVAQAADELLSLEGIEASYVVARREDNLIGISARSLGSVNVQLTMEALGGGGHLTNAATQLKGVTVEEAIAQLQQAITEQLSRSEDA

SEQ ID NO: 345 - protein, 122 aa, Staphylococcus saprophyticus
MIQQETRLKVADNSGAREVLTIKVLGGSGRKTANIGDVIVVSVKNATPGGVVKKGEVVKAVVVRTKSGVRRNDGSYIKFDENACVIIRDDKGPRGTRIFGPVARELREGNFMKIVSLAPEVL

SEQ ID NO: 346 - protein, 192 aa, Staphylococcus haemolyticus
MIRFEIHGDNLTITDAIRNYIEDKIGKLERYFNDVPNAVAHVKVKTYQNSTTKIEVTIPLKNVTLRAEERHDDLYAGIDLVTSKLERQVRKYKTRVNRKHKTHGEPEAFVAEVQEAPPENVDDVNAEPTNDSEIEIIRSKQFSLKPMDSEEAVLQMELLGHDFYIFTDRETDGTSIVYKRKDGKYGLIETTE

SEQ ID NO: 347 - protein, 130 aa, unidentified
  REGION 1..130: note = Bacilli sp.
MINKVIYSGRIATDLKLRKSKNEKSYVFFNIAVPRQFDNDRTDFINCVVWGKQAESLVDYQSKGNLIIIEGSTETYKKDNINMSRCLVEKTIFLESKKENKKHDVSEDDAISPAEDSGNDKSISDDDLPF

SEQ ID NO: 348 - protein, 284 aa, Staphylococcus sp.
MSFYVVLIIIIVALIGILVLNQRYSNSKIDTEVYARKQLIKKNKALSAENAELRSQMLSSNNDVGHHAYKNAKRELRKILDSYLENGKLKYYDIIVTSNLATKHPFFEYARSFDFIIVSDIGLINVDVKSWGEKTFYHFDVPDEHDTEMSNSNIEKVVGHYISQQYHDQFNSSRKSIYTFTETVQPNRVIYDFYDYDPYQLAANNAKALKDHIEQNFNFKVQSTGVIYFSDGTVNIIQGSEERDKYVDTVSTKSSLRRIISEAIELSKHPLNKEQVNQITAIFK

SEQ ID NO: 349 - protein, 644 aa, Staphylococcus aureus
MVTALSDVNNTDNYGAGQIQVLEGLEAVRKRPGMYIGSTSERGLHHLVWEIVDNSIDEALAGYANQIEVVIEKDNWIKVTDNGRGIPVDIQEKMGRPAVEVILTVLHAGGKFGGGGYKVSGGLHGVGSSVVNALSQDLEVYVHRNETIYHQAYKKGVPQFDLKEVGTTDKTGTVIRFKADGEIFTETTVYNYETLQQRIRELAFLNKGIQITLRDERDEENVREDSYHYEGGIKSYVELLNENKEPIHDEPIYIHQSKDDIEVEIAIQYNSGYATNLLTYANNIHTYEGGTHEDGFKRALTRVLNSYGLSSKIMKEEKDRLSGEDTREGMTAIISIKHGDPQFEGQTKTKLGNSEVRQVVDKLFSEHFERFLYENPQVARTVVEKGIMAARARVAAKKAREVTRRKSALDVASLPGKLADCSSKSPEECEIFLVEGDSAGGSTKSGRDSRTQAILPLRGKILNVEKARLDRILNNNEIRQMITAFGTGIGGDFDLAKARYHKIVIMTDADVDGAHIRTLLLTFFYRFMRPLIEAGYVYIAQPPLYKLTQGKQKYYVYNDRELDKLKSELNPTPKWSIARYKGLGEMNADQLWETTMNPEHRALLQVKLEDAIEADQTFEMLMGDVVENRRQFIEDNAVYANLDF

SEQ ID NO: 350 - protein, 345 aa, Staphylococcus aureus
MVHNKNNTILKMIKGEETSHTPVWFMRQAGRSQPEYRKLKEKYSLFDITHQPELCAYVTHLPVDNYHTDAAILYKDIMTPLKPIGVDVEIKSGIGPVIHNPIKTIQDVEKLSQIDPERDVPYVLDTIKLLTEEKLNVPLIGFTGAPFTLASYMIEGGPSKNYNFTKAMMYRDEATWFALMNHLVDVSVKYVTAQVEAGAELIQIFDSWVGALNVEDYRRYIKPHMIRLISEVKEKHDVPVILFGVGASHLINEWNDLPIDVLGLDWRTSINQAQQLGVTKTLQGNLDPSILLAPWNVIEERLKPILDQGMENGKHIFNLGHGVFPEVQPETLRKVSEFVHTYTQR

SEQ ID NO: 351 - protein, 436 aa, Staphylococcus saprophyticus
MTKPIVAIVGRPNVGKSTIFNRVVGERVSIVEDTPGVTRDRIYSSGEWLTHDFNIIDTGGIEIGDAPFQTQIRAQAEVAIDEADVIIFMVNVREGLTQSDEMVAQMLYKSKKPVVLAVNKVDNPEMRNEIYDFYSLGFGDPYPISGSHGLGLGDLLDAVVKHFKEEEPDPYDDDTIRLSIIGRPNVGKSSLVNAILGEDRVIVSNVAGTTRDAVDTEYSYDDQDYVLIDTAGMRKKGKVYENTEKYSVLRALKAIERSNVILIVIDAEQGIIEQDKRVAGYAHEEGKAIVIVVNKWDTVDKETNTMKKFKDEVRKEFQFLDYAEIAFVSAKEKQRLRTLFPYIKEASENHKKRVQSSTLNEVVTDAISMNPTPTDKGRRLNVFYATQVAIEPPTFIVFVNDVELMHFSYKRYLENQIRAAFGFEGTPVHIIARKRN

SEQ ID NO: 352 - protein, 233 aa, Staphylococcus aureus
MARKVVVVDDEKPIADILEFNLKKEGYDVYCAYDGNDAVDLIYEEEPDIVLLDIMLPGRDGMEVCREVRKKYEMPIIMLTAKDSEIDKVLGLELGADDYVTKPFSTRELIARVKANLRRHYSQPAQDTGNVTNEITIKDIVIYPDAYSIKKRGEDIELTHREFELFHYLSKHMGQVMTREHLLQTVWGYDYFGDVRTVDVTIRRLREKIEDDPSHPEYIVTRRGVGYFLQQHE

SEQ ID NO: 353 - protein, 205 aa, Staphylococcus sp.
MFSKKKDKFMVQLEEMVFNLDRAAIEFGKMDFNTHLDLKAYSDNIKTYESHGDELVHQVITDLNQTFITPIEREDILSLCDAIDDVLDAIEETAAMFEMYSIEYTDEYMAEFVDNIQKAVAEMKLAVGLLVDKKLSHMRIHSINIKEFETNCDGILRQSIKHIFNSETDPITLIKIKDIYESMEEIADKCQIVANNFETIIMKNS

SEQ ID NO: 354 - protein, 319 aa, Staphylococcus aureus
MKTFGKKVVLIGDGSVGSSYAFAMVTQGVADEFVIIDIAKDKVKADVQDLNHGTVHSPSPVDVKAGEYEDCKDADLVVITAGAPQKPGETRLQLVEKNTKIMKSIVKSVMDSGFDGYFLIAANPVDILTRFVKEYTGLPAERVIGSGTVLDSARLQYLISQELGVAPSSVDASIIGEHGDTELAVWSQANVAGISVYDTLKEQTGSEAKAEEIYVNTRDAAYEIIQAKGSTYYGIALALMRISKAILNNENNVLNVSIQLDGQYGGHKGVYLGVPTLVNQHGAVKIYEMPLSAEEQALFDKSVKILEDTFDSIKYLLED

SEQ ID NO: 355 - protein, 579 aa, Staphylococcus sp.
MAKIKANEALVKALQAWDIDHLYGIPGDSIDAVVDSLRTVRDQFKFYHVRHEEVASLAAAGYTKLTGKIGVALSIGGPGLIHLLNGMYDAKMDNVPQLILSGQTNSTALGTKAFQETNLQKLCEDVAVYNHQIEKGDNVFEIVNEAIRTAYEQKGVAVVICPNDLLTEKIKDTTNKPVDTSRPTVVSPKYKDIKKAVKLINKSKKPVMLIGVGAKHAKDELREFIEMAKIPVIHSLPAKTILPDDHPYSIGNLGKIGTKTSYQTMQEADLLIMVGTNYPYVDYLPKKNIKAIQIDTNPKNIGHRFNINVGIVGDSKIALHQLTENIKHVAERPFLNKTLERKAVWDKWMEQDKNNNSKPLRPERLMASINKFIKDDAVISADVGTATVWSTRYLNLGVNNKFIISSWLGTMGCGLPGAMASKIAYPNRQAIAIAGDGAFQMVMQDFATAVQYDLPLTVFVLNNKQLAFIKYEQQAAGELEYAVDFSDMDHAKFAEAAGGKGYTIKSASEVDAIVEEALAQDVPTIVDVYVDPNAAPLPGKIVNEEALGYGKWAFRSITEDKHLDLDQIPPISVAAKRFL

SEQ ID NO: 356 - protein, 422 aa, Staphylococcus aureus
MPEVKVPELAESITEGTIAEWLKNVGDSVEKGEAILELETDKVNVEVVSEEAGVLSEQLASEGDTVEVGQAIAIIGEGSGNASKENSNDNTPQQNEETNNKKEETTNNSVDKAEVNQANDDNQQRINATPSARRYARENGVNLAEVSPKTNDVVRKEDIDKKQQAPASTQTTQQAPAKEEKKYNQYPTKPVIREKMSRRKKTAAKKLLEVSNNTAMLTTFNEVDMTNVMELRKRKKEQFMKDHDGTKLGFMSFFTKASVAALKKYPEVNAEIDGDDMITKQYYDIGVAVSTDDGLLVPFVRDCDKKNFAEIEAEIANLAVKAREKKLGLDDMVNGSFTITNGGIFGSMMSTPIINGNQAAILGMHSIITRPIAIDQDTIENRPMMYIALSYDHRIIDGKEAVGFLKTIKELIENPEDLLLES

SEQ ID NO: 357 - protein, 309 aa, Staphylococcus aureus
MIKNKILTATLAVGLIAPLANPFIEISKAENKIEDIGQGAEIIKRTQDITSKRLAITQNIQFDFVKDKKYNKDALVVKMQGFISSRTTYSDLKKYPYIKRMIWPFQYNISLKTKDSNVDLINYLPKNKIDSADVSQKLGYNIGGNFQSAPSIGGSGSFNYSKTISYNQKNYVTEVESQNSKGVKWGVKANSFVTPNGQVSAYDQYLFAQDPTGPAARDYFVPDNQLPPLIQSGFNPSFITTLSHEKGKGDKSEFEITYGRNMDTTYAYVTRHRLAVDRKHDAFKNRNVTVKYEVNWKTHEVKIKSITPK

SEQ ID NO: 358 - protein, 397 aa, Staphylococcus aureus
MLNNKRLFTSESVTEGHPDKIADQVSDAILDAILKDDPNARVACETTVTTGMALIAGEISTTTYVDIPKVVRETIKEIGYTRAKYGYDYETMAILTAIDEQSPDIAQGVDKALEYRDKDSEEEIEATGAGDQGLMFGYATNETETYMPLAIYLSHQLAKRLSDVRKDGTLNYLRPDGKVQVTVEYDENDNPVRIDTIVVSTQHAEDVTLEQIQEDIKAHVIYPTVPENLINEQTKFYINPTGRFVIGGPQGDAGLTGRKIIVDTYGGIARHGGGCFSGKDPTKVDRSAAYAARYVAKNIVAAGLADQCEVQLAYAIGVAEPVSIAIDTFGTGKVSEGQLVEAVRKHFDLRPAGIIKMLDLKQPIYKQTAAYGHFGRTDVLFPWEKLDKVEELKDAVK

SEQ ID NO: 359 - protein, 505 aa, Staphylococcus aureus
MAKKPTALIILDGFANRESEHGNAVKLANKPNFDRYYNKYPTTQIEASGLDVGLPEGQMGNSEVGHMNIGAGRIVYQSLTRINKSIEDGDFFENDVLNNAIAHVNSHDSALHIFGLLSDGGVHSHYKHLFALLELAKKQGVEKVYVHAFLDGRDVDQKSALKYIEETEAKFNELGIGQFASVSGRYYAMDRDKRWEREEKAYNAIRNFDAPTYATAKEGVEASYNEGLTDEFVVPFIVENQNDGVNDGDTVIFYNFRPDRAAQLSEIFANRAFEGFKVEQVKDLFYATFTKYNDNIDAAIVFEKVDLNNTIGEIAQNNNLTQLRIAETEKYPHVTYFMSGGRNEEFKGERRRLIDSPKVATYDLKPEMSAYEVKDALLEELNKGDLDLIILNFANPDMVGHSGMLEPTIKAIEAVDECLGEVVDKILDMDGYAIITADHGNSDQVLTDDDQPMTTHTTNPVPVIVTKEGVTLRETGRLGDLAPTLLDLLNVEQPEDMTGESLIKH

SEQ ID NO: 360 - protein, 434 aa, Staphylococcus aureus
MPIITDVYAREVLDSRGNPTVEVEVLTESGAFGRALVPSGASTGEHEAVELRDGDKSRYLGKGVTKAVENVNEIIAPEIIEGEFSVLDQVSIDKMMIALDGTPNKGKLGANAILGVSIAVARAAADLLGQPLYKYLGGFNGKQLPVPMMNIVNGGSHSDAPIAFQEFMILPVGATTFKESLRWGTEIFHNLKSILSKRGLETAVGDEGGFAPKFEGTEDAVKTIIQAIEAAGYKPGEEVFLGFDCASSEFYENGVYDYSKFEGEHGAKRTAAEQVDYLEQLVDKYPIITIEDGMDENDWDGWKQLTERIGDRVQLVGDDLFVTNTEILAKGIENGIGNSILIKVNQIGTLTETFDAIEMAQKAGYTAVVSHRSGETEDTTIADIAVATNAGQIKTGSLSRTDRIAKYNQLLRIEDELFETAKYDGIKSFYNLDK

SEQ ID NO: 361 - protein, 602 aa, Staphylococcus sp.
MSQQLSREEQERKYPEYTWDLTTIFKDDEAFEAAFKEVENELGKEEQFKGHIGDSAETLYNALELEDTLGTKLEKVYVYAHLKQDQDTTNDKYTGMESRAHQLIIKFSSAWSFLVPEILQIDEDKIQSFVNSYDKLQKFAFDLKLINEKRPHILDAETEKLLTEAQDALSTPSNVYGMFSNADLVFEDAIDKDENAHPLTQGTFIKYLESDDRKLRESAFRNVYKAYGAHNNTLGATLAGEVKKNVFNARTHNYKTAREKALSNNHIPENVYDNLVKTVHKYLPLLHRYTELRKELLGLDDLKMYDLYTPLIKDIKFEMPYEEAKEWMLKALEPMGEEYLNVVKEGLNNRWVDVYENKGKRSGGYSSGAHLTNPFILLNWSNTISDLYTLVHEFGHSAHSYFSRKFQPSNSSDYTIFVAEVASTCNEALLSDYMDKHLDDEKRLLLLNQELERFRATLFRQTMFAEFEHKIHAIEEAGEPLTPTRMNEEYAKLNKLYFGDSVETDEDISKEWSRIPHFYMNYYVYQYATGYSAAQSLSHQILTEGKPAVDRYINEFLKKGSSNYPIEILKNAGVDMTTPEPIEQACEVFEQKLNAFEKLMKA

SEQ ID NO: 362 - protein, 499 aa, Staphylococcus epidermidis
MAMSDKKDVVLIGAGVLSTTFGSMLKTIAPDWDIHLYERLDRPGIESSNERNNAGTGHAALCELNYTVQQPDGSIDIEKAKEINEQFEISKQFWGHLVKSGEIQNPKEFINPLPHISFVRGKNNVKFLKDRYEAMKQFPMFDNIEYTEDIEEMRKWIPLMMKGREDKGYMAASKIDEGTDVNYGELTRKMAQNLKNSPNVEVQYKHEVVDFERLSNGKWSVKIKNLNNGQVFEHQTDYVFIGAGGGAIPLLQKTGIPESKHLGGFPISGQFIACTNPQVIEQHDAKVYGKEPPGTPPMTVPHLDTRYIDGERTLLFGPFANVGPKFLKHGSNLDLFKSIKPYNITTLLASAVKNLPLIKYSFDQVIMTKEGCMNHLRTFYPEARDEDWQVYTAGKRVQVIKDTEENGKGFIQFGTEVVNSEDHSVIALLGESPGASTSVSVALEVLEKNFPEYAKDWEPKIKKMIPSYGESLIDDVQLMRKIRKQTSKDLELGFYNKAK

SEQ ID NO: 363 - protein, 253 aa, unidentified
  REGION 1..253: note = Eubacteria sp.
MRTPIIAGNWKMNKTVQEAKDFVNALPTLPDSKEVESVICAPAIQLDALTTAVKEGKAQGLEIGAQNTYFEDNGAFTGETSPVALADLGVKYVVIGHSERRELFHETDEEINKKAHAIFKHGMTPIICVGETDEERESGKANDVVGEQVKKAVAGLSEDQLKSVVIAYEPIWAIGTGKSSTSEDANEMCAFVRQTIADLSSKEVSEATRIQYGGSVKPNNIKEYMAQTDIDGALVGGASLKVEDFVQLLEGAK

SEQ ID NO: 364 - protein, 1257 aa, Staphylococcus aureus
MAKKFNYKLPSMVALTLVGSAVTAHQVQAAETTQDQTTNKNVLDSNKVKATTEQAKAEVKNPTQNISGTQVYQDPAIVQPKAANKTGNAQVNQKVDTTQVNGDTRATQSTTSNNAKPVTKSTNTTAPKTNNNVTSAGYSLVDDEDDNSENQINPELIKSAAKPAALETQYKAAAPKATPVAPKAKTEATPKVTTFSASAQPRSAAAAPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQSTTTPTTPSKPSTPSKPSTPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLYGTVNGKSGWISKAYLAVPAAPKKAVAQPKTAVKAYAVTKPQTTQTVSKIAQVKPNNTGIRASVYEKTAKNGAKYADRTFYVTKERAHGNETYVLLNNTSHNIPLGWFNVKDLNVQNLGKEVKTTQKYTVNRSNNGLSMVPWGTKNQVILTGNNIAQGTFNATKQVSVGKDVYLYGTINNRTGWVNSKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQIGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 365 - protein, 693 aa, Staphylococcus sp.
MAREFSLEKTRNIGIMAHIDAGKTTTTERILYYTGRIHKIGETHEGASQMDWMEQEQDRGITITSAATTAAWEGHRVNIIDTPGHVDFTVEVERSLRVLDGAVTVLDAQSGVEPQTETVWRQATTYGVPRIVFVNKMDKLGANFEYSVSTLHDRLQANAAPIQLPIGAEDEFEAIIDLVEMKCFKYTNDLGTEIEEIEIPEDHLDRAEEARASLIEAVAETSDELMEKYLGDEEISVSELKEAIRQATTNVEFYPVLCGTAFKNKGVQLMLDAVIDYLPSPLDVKPIIGHRASNPEEEVIAKADDSAEFAALAFKVMTDPYVGKLTFFRVYSGTMTSGSYVKNSTKGKRERVGRLLQMHANSRQEIDTVYSGDIAAAVGLKDTGTGDTLCGEKNDIILESMEFPEPVIHLSVEPKSKADQDKMTQALVKLQEEDPTFHAHTDEETGQVIIGGMGELHLDILVDRMKKEFNVECNVGAPMVSYRETFKSSAQVQGKFSRQSGGRGQYGDVHIEFTPNETGAGFEFENAIVGGVVPREYIPSVEAGLKDAMENGVLAGYPLIDVKAKLYDGSYHDVDSSEMAFKIAASLALKEAAKKCDPVILEPMMKVTIEMPEEYMGDIMGDVTSRRGRVDGMEPRGNAQVVNAYVPLSEMFGYATSLRSNTQGRGTYTMYFDHYAEVPKSIAEDIIKKNKGE

SEQ ID NO: 366 - protein, 585 aa, Staphylococcus aureus
MRKTKIVCTIGPASESEEMIEKLINAGMNVARLNFSHGSHEEHKGRIDTIRKVAKRLDKIVAILLDTKGPEIRTHNMKDGIIELERGNEVIVSMNEVEGTPEKFSVTYENLINDVQVGSYILLDDGLIELQVKDIDHAKKEVKCDILNSGELKNKKGVNLPGVRVSLPGITEKDAEDIRFGIKENVDFIAASFVRRPSDVLEIREILEEQKANISVFPKIENQEGIDNIAEILEVSDGLMVARGDMGVEIPPEKVPMVQKDLIRQCNKLGKPVITATQMLDSMQRNPRATRAEASDVANAIYDGTDAVMLSGETAAGLYPEEAVKTMRNIAVSAEAAQDYKKLLSDRTKLVETSLVNAIGISVAHTALNLNVKAIVAATESGSTARTISKYRPHSDIIAVTPSEETARQCSIVWGVQPVVKKGRKSTDALLNNAVATAVETGRVSNGDLIIITAGVPTGETGTTNMMKIHLVGDEIANGQGIGRGSVVGTTLVAETVKDLEGKDLSDKVIVTNSIDETFVPYVEKALGLITEENGITSPSAIVGLEKGIPTVVGVEKAVKNISNNMLVTIDAAQGKIFEGYANVL

SEQ ID NO: 367 - protein, 377 aa, Staphylococcus sp.
MMEFTIKRDYFITQLNDTLKAISPRTTLPILTGIKIDAKEHEVILTGSDSEISIEITIPKTVDGEDIVNISETGSVVLPGRFFVDIIKKLPGKDVKLSTNEQFQTLITSGHSEFNLSGLDPDQYPLLPQVSRDDAIQLSVKVLKNVIAQTNFAVSTSETRPVLTGVNWLIQENELICTATDSHRLAVRKLQLEDVSENKNVIIPGKALAELNKIMSDNEEDIDIFFASNQVLFKVGNVNFISRLLEGHYPDTTRLFPENYEIKLSIDNGEFYHAIDRASLLAREGGNNVIKLSTGDDVVELSSTSPEIGTVKEEVDANDVEGGSLKISFNSKYMMDALKAIDNDEVEVEFFGTMKPFILKPKGDDSVTQLILPIRTY

SEQ ID NO: 368 - protein, 224 aa, Staphylococcus aureus
MINKNDIVADVVTDYPKAADIFRSVGIDFCCGGQVSIEAAALEKKNVDLNELLQRLNDVEQTNTPGSLNPKFLNVSSLIQYIQSAYHEPLREEFKNLTPYVTKLSKVHGPNHPYLVELKETYDTFKNGMLEHMQKEDDVDFPKLIKYEQGEVVDDINTVIDDLVSDHIATGELLVKMSELTSSYEPPIEACGTWRLVYQRLKALEVLTHEHVHLENHVLFKKVS

SEQ ID NO: 369 - protein, 678 aa, Staphylococcus saprophyticus
MFKKLFGQLQRIGKALMLPVAILPAAGLLLAIGTAMQGESLQHYLPFIQNGGIQSVAEMMTGAGGIIFDNLPMIFAMGVAIGLASGDGVAAIAAFVGYLVMNKTMGAFLHVSPDNVNDAASGYASVLGIPTLQTGVFGGIIIGALAAWCYNKFYNISLPSYLGFFAGKRFVPIMMATTSFILAFPMAWIWPSIQTGLNAFSEGLLDSNTGLAVFLFGFIKRLLIPFGLHHIFHAPFWFEFGAWKNAAGEMIHGDQRIFIEQIREGSKLTAGKFMQGEFPVMMFGLPAAALAIYHTAKPENKKVVAGLMGSAALTSFLTGITEPLEFSFLFVAPVLFFVHAILDGLSFLILYLLNVHLGYTFSGGFIDYVLLGVLPNKTQWWLVIPVGVVYAFIYYFVFRFLILKFKYKTPGREDKQAQFTNSSASELPFNVLKAMGGEENIKHLDACITRLRVEVKEKGKVDVAGLKALGASGVLEVGNNMQAIFGPKSDQIKHDMSLIMKGEITKPQETTVTEEESEEVVHIERASEVNIYAPGNGQVIPLSEVPDQVFAQKMMGDGVGFIPADGKIVAPFDGTVKTIFPTKHAIGLESDQGLELLIHIGIDTVKLNGEGFESFVETDDRVHKGQVLMQIDLDYITAHAPSTVTPLIITNLEDRQLSVEDVKDVTAEQLIIKVIDDK

SEQ ID NO: 370 - protein, 502 aa, Staphylococcus haemolyticus
MAIKAEEISALLRSQIENYESEMSVTDVGTVLQIGDGIALIHGLNDCMAGELVEFSNGVLGLAQNLEESNVGVVILGPYTEITEGDEVRRTGRIMEVPVGEELIGRVVNPLGQPIDGQGPINTTKTRPVEQKATGVMARKSVDEPLQTGIKAIDALVPIGRGQRELIIGDRQTGKTTIGIDTILNQKGLDTICIYVAIGQKDSTVRANVEKLRQAGALDYTIVVSASASEPSPLLYIAPYSGVTMGEEFMFNGKHVLIVYDDLTKQAAAYRELSLLLRRPPGREAYPGDVFYLHSRLLERAAKLNDDLGGGSITALPIIETQAGDISAYVPTNVISITDGQIFLQSDLFFSGVRPAINAGQSVSRVGGSAQIKAMKKVAGTLRLDLASYRELESFAQFGSDLDEFTARKLERGKRTVEVLKQDQNKPLPVENQVLIIYALTKGYLDDIPVEDITRFEDELNSWTKSNGSDLLNEIRETGGLPSDDKFEATINEFKKSFSKSE

SEQ ID NO: 371 - protein, 336 aa, Staphylococcus aureus
MTEIQKPYDLKGRSLLKESDFTKAEFEGLIDFAITLKEYKKNGIKHHYLSGKNIALLFEKNSTRTRAAFTVASIDLGAHPEFLGKNDIQLGKKESVEDTAKVLGRMFDGIEFRGFSQQAVEDLAKFSGVPVWNGLTDDWHPTQMLADFMTIKENFGYLEGINLTYVGDGRNNIAHSLMVAGAMLGVNVRICTPKSLNPKEAYVDIAKEKASQYGGSVMITDNIAEAVENTDAIYTDVWVSMGEESEFEQRINLLKDYQVNQQMFDLTGKDSTIFLHCLPAFHDTNTLYGQEIYEKYGLAEMEVTDQIFRSEHSKVFDQAENRMHTIKAVMAATLGS

SEQ ID NO: 372 - protein, 329 aa, Staphylococcus epidermidis
MFSIGFIIIAVIIVVALLILFSFVPVGLWISALAAGVHVGIGTLVGMRLRRVSPRKVIAPLIKAHKAGLNLTTNQLESHYLAGGNVDRVVDANIAAQRADIDLPFERGAAIDLAGRDVLEAVQMSVNPKVIETPFIAGVAMNGIEVKAKARITVRANIARLVGGAGEETIIARVGEGIVSTIGSSEHHTEVLENPDNISKTVLSKGLDSGTAFEILSIDIADVDISKNIGADLQTEQALADKNIAQAKAEERRAMAVASEQEMKARVQEMRAKVVEAESEVPLAMAEALRSGNIGVKDYYNLKNIEADTGMRNAINKRTDQNDDESPQQ

SEQ ID NO: 373 - protein, 536 aa, Staphylococcus aureus
MTKFIFVTGGVVSSLGKGITASSLGRLLKDRGLNVTIQKFDPYLNVDPGTMSPYQHGEVFVTDDGAETDLDLGHYERFIDINLNKFSNVTAGKVYSHVLKKERRGDYLGGTVQVIPHITNEIKERLLLAGESTNADVVITEIGGTTGDIESLPFIEAIRQIRSDLGRENVMYVHCTLLPYIKAAGEMKTKPTQHSVKELRGLGIQPDLIVVRTEYEMTQDLKDKIALFCDINKESVIECRDADSLYEIPLQLSQQNMDDIVIKRLQLNAKYETQLDEWKQLLDIVNNLDGKITIGLVGKYVSLQDAYLSVVESLKHAGYPFAKDIDIRWIDSSEVTDENAAEYLADVDGILVPGGFGFRASEGKISAIKYARENNVPFFGICLGMQLATVEFSRNVLGLEGAHSAELDPATPYPIIDLLPEQKDIEDLGGTLRLGLYSCSIKEGTLAQDVYGKAEIEERHRHRYEFNNDYREQLEANGMVISGTSPDGRLVEMVEIPTNDFFIACQFHPEFLSRPNRPHPIFKSFIEASLKYQQNK

SEQ ID NO: 374 - protein, 869 aa, Staphylococcus aureus
MDINKMTYAVQSALQQAVELSQQHKLQNIEIEAILSAALNESESLYKSILERANIEVDQLNKAYEDKLNTYASVEGDNIQYGQYISQQANQLITKAESYMKEYEDEYISMEHILRSAMDIDQTTKHYINNKVEVIKEIIKKVRGGNHVTSQNPEVNYEALAKYGRDLVEEVRQGKMDPVIGRDEEIRNTIRILSRKTKNNPVLIGEPGVGKTAIVEGLAQRIVKKDVPESLLDKTVFELDLSALVAGAKYRGEFEERLKAVLKEVKESDGRIILFIDEIHMLVGAGKTDGAMDAGNMLKPMLARGELHCIGATTLNEYREYIEKDSALERRFQKVAVSEPDVEDTISILRGLKERYEVYHGVRIQDRALVAAAELSDRYITDRFLPDKAIDLVDQACATIRTEMGSNPTELDQVNRRVMQLEIEESALKNESDNASKQRLQELQEELANEKEKQAALQSRVESEKEKIANLQEKRAQLDESRQALEDAQTNNNLEKAAELQYGTIPQLEKELRELEDNFQDEQGEDTDRMIREVVTDEEIGDIVSQWTGIPVSKLVETEREKLLHLSDILHKRVVGQDKAVDLVSDAVVRARAGIKDPNRPIGSFLFLGPTGVGKTELAKSLAASLFDSEKHMIRIDMSEYMEKHAVSRLIGAPPGYIGHDEGGQLTEAVRRNPYSVILLDEVEKAHTDVFNVLLQILDEGRLTDSKGRSVDFKNTIIIMTSNIGSQVLLENVKETGEITESTEKAVMTSLNAYFKPEILNRMDDIVLFKPLSIDDMSMIVDKILTQLNIRLLEQRISIEVSDDAKAWLGQEAYEPQYGARPLKRFVQRQIETPLARMMIKEGFPEGTTIKVNLNSDNNLTFNVEKIHE

SEQ ID NO: 375 - protein, 519 aa, Staphylococcus aureus
MNLLSLLLILLGIILGVVGGYVVARNLLLQKQSQARQTAEDIVNQAHKEADNIKKEKLLEAKEENQILREQTEAELRERRSELQRQETRLLQKEENLERKSDLLDKKDEILEQKESKIEEKQQQVDAKESSVQTLIMKHEQELERISGLTQEEAINEQLQRVEEELSQDIAVLVKEKEKEAKEKVDKTAKELLATAVQRLAADHTSESTVSVVNLPNDEMKGRIIGREGRNIRTLETLTGIDLIIDDTPEAVILSGFDPIRREIARTALVNLVSDGRIHPGRIEDMVEKARKEVDYIIREAGEQATFEVNAHNMHPDLVKIVGRLNYRTSYGQNVLKHSIEVAHLASMLAAELGEDETLAKRAGLLHDVGKAIDHEVEGSHVEIGVELAKKYGENETVINAIHSHHGDVEPTSIISILVAAADALSAARPGARKETLENYIRRLERLETLSESYDGVEKAFAIQAGREIRVIVSPEEIDDLKSYRLARDIKNQIEDELQYPGHIKVTVVRETRAVEYAK

SEQ ID NO: 376 - protein, 507 aa, Staphylococcus aureus
MLNADLKQQLKQLLELMEGNVEFVASLGSDEKSKELKELLTEISDMSPRLSLSEKSLKRTPSFSVNRPGEETGVTFAGIPLGHEFNSLVLAILQVSGRAPKEKQSIIDQIKNLEGSFHFETFISLTCQKCPDVVQALNLMSVINPNITHSMIDGAVFREESENIMAVPAVFLNGEEFGNGRMTIQDILSKLGSTADASEFENKEPYDVLIVGGGPASGSAAIYTARKGLRTGIVADRIGGQVNDTAGIENFITVKETTGSEFSSNLAAHIDQYDIDAMTGIRATDIEKTDEAIKVTLENGAVLESKTVIIATGAGWRKLNIPGEEQLINKGVAFCPHCDGPLFENKDVAVIGGGNSGVEAAIDLAGIVNHVTLFEFASELKADNVLQDRLRSLSNVDIKTNAKTTEVVGEDHVTGIRYEDMSTGEEHLLNLDGIFVQIGLLPNTSWLKDAVELNERGEIVIDCNNNTNVPGIFAAGDVTDQKNKQIIISMGAGANAALNAFDYIIRN

SEQ ID NO: 377 - protein, 330 aa, Staphylococcus aureus
MTKIMFFGTRDYEKEMALNWGKKNNVEVTTSKELLSSATVDQLKDYDGVTTMQFGKLENDVYPKLESYGIKQIAQRTAGFDMYDLDLAKKHNIVISNVPSYSPETIAEYSVSIALQLVRRFPDIERRVQTHDFTWQAEIMSKPVKNMTVAIIGTGRIGAATAKIYAGFGATITAYDAYPNKDLDFLTYKDSVKEAIKDADIISLHVPANKESYHLFDKAMFDHVKKGAILVNAARGAVINTPDLIAAVNDGTLLGAAIDTYENEAAYFTNDWTNKDIDDKTLLELIEHERILVTPHIAFFSDEAVQNLVEGGLNAALSVINTGTCETRLN

SEQ ID NO: 378 - protein, 485 aa, Staphylococcus aureus
MSIRYESVENLLTLIKDKKIKPSDVVKDIYDAIEETDPTIKSFLALDKENAIKKAQELDELQAKDQMDGKLFGIPMGIKDNIITNGLETTCASKMLEGFVPIYESTVMEKLHNENAVLIGKLNMDEFAMGGSTETSYFKKTVNPFDHKAVPGGSSGGSAAAVAAGLVPFSLGSDTGGSIRQPAAYCGVVGMKPTYGRVSRFGLVAFASSLDQIGPLTRNVKDNAIVLEAISGADVNDSTSAPVDDVDFTSEIGKDIKGLKVALPKEYLGEGVADDVKEAVQNAVETLKSLGAVVEEVSLPNTKFGIPSYYVIASSEASSNLSRFDGIRYGYHSKEAHSLEELYKMSRSEGFGKEVKRRIFLGTFALSSGYYDAYYKKSQKVRTLIKNDFDKVFENYDVVVGPTAPTTAFNLGEEIDDPLTMYANDLLTTPVNLAGLPGISVPCGQSNGRPIGLQFIGKPFDEKTLYRVAYQYETQYNLHDVYEKL

SEQ ID NO: 379 - protein, 502 aa, Staphylococcus aureus
MAIKAEEISALLRSQIENYESEMSVTDVGTVLQIGDGIALIHGLNDVMAGELVEFHNGVLGLAQNLEESNVGVVILGPYTGITEGDEVKRTGRIMEVPVGEELIGRVVNPLGQPIDGQGPINTTKTRPVEKKATGVMDRKSVDEPLQTGIKAIDTLVPIGRGQRELIIGDRQTGKTTIAIDTILNQKDQGTICIYVAIGQKDSTVRANVEKLRQAGALDYTIVVAASASEPSPLLYIAPYSGVTMGEEFMFNGKHVLIVYDDLTKQAAAYRELSLLLRRPPGREAYPGDVFYLHSRLLERAAKLNDDLGGGSITALPIIETQAGDISAYVPTNVISITDGQIFLQSDLFFSGVRPAINAGQSVSRVGGSAQIKAMKKVAGTLRLDLASYRELESFAQFGSDLDEFTASKLERGKRTVEVLKQDQNKPLPVEHQVLIIYALTKGYLDDIPVVDITRFEDELNHWAESNATELLNEIRETGGLPDAEKFDTAINEFKKSFSKSE

SEQ ID NO: 380 - protein, 498 aa, Staphylococcus aureus
MEKYILSIDQGTTSSRAILFNQKGEIAGVAQREFKQYFPQSGWVEHDANEIWTSVLAVMTEVINENDVRADQIAGIGITNQRETTVVWDKHTGRPIYHAIVWQSRQTQSICSELKQQGYEQTFRDKTGLLLDPYFAGTKVKWILDNVEGAREKAENGGLLFGTIDTWLVWKLSGKAAHITDYSNASRTLMFNIHDLEWDDELLELLTVPKNMLPEVKPSSEVYGKTIDYHFYGQEVPIAGVAGDQQAALFGQACFERGDVKNTYGTGGFMLMNTGDKAVKSESGLLTTIAYGIDGKVNYALEGSIFVSGSAIQWLRDGLRMINSAPQSESYATRVDSTEGVYVVPAFVGLGTPYWDSEARGAIFGLTRGTEKEHFIRATLESLCYQTRDVMEAMSKDSGIDVQSLRVDGGAVKNNFIMQFQADIVNTSVERPEIQETTALGAAYLAGLAVGFWESKDDIAKNWKLEEKFDPKMDEGEREKLYRGWKKAVEATQVFKTE

SEQ ID NO: 381 - protein, 315 aa, Staphylococcus aureus
MLKNKILTTTLSVSLLAPLANPLLENAKAANDTEDIGKGSDIEIIKRTEDKTSNKWGVTQNIQFDFVKDKKYNKDALILKMQGFISSRTTYYNYKKTNHVKAMRWPFQYNIGLKTNDKYVSLINYLPKNKIESTNVSQTLGYNIGGNFQSAPSLGGNGSFNYSKSISYTQQNYVSEVEQQNSKSVLWGVKANSFATESGQKSAFDSDLFVGYKPHSKDPRDYFVPDSELPPLVQSGFNPSFIATVSHEKGSSDTSEFEITYGRNMDVTHAIKRSTHYGNSYLDGHRVHNAFVNRNYTVKYEVNWKTHEIKVKGQN

SEQ ID NO: 382 - protein, 506 aa, Staphylococcus aureus
MQNFKELGISDNTVQSLESMGFKEPTPIQKDSIPYALQGIDILGQAQTGTGKTGAFGIPLIEKVVWKQGVQSLILAPTRELAMQVAEQLREFSRGQGVQVVTVFGGMPIERQIKALKKGPQIVVGTPGRVIDHLNRRTLKTDGIHTLILDEADEMMNMGFIDDMRFIMDKIPAVQRQTMLFSATMPKAIQALVQQFMKSPKIIKTMNNEMSDPQIEEFYTIVKELEKFDTFTNFLDVHQPELAIVFGRTKRRVDELTSALISKGYKAEGLHGDITQAKRLEVLKKFKNDQINILVATDVAARGLDISGVSHVYNFDIPQDTESYTHRIGRTGRAGKEGIAVTFVNPIEMDYIRQIEDANGRKMSALRPPHRKEVLQAREDDIKEKVENWMSKESESRLKRISTELLNEYNDVDLVAALLQELVEANDEVEVQLTFEKPLSRKGRNGKPSGSRNRNSKRGNPKFDSKSKRSKGYSSKKKSTKKFDRKEKSSGGSRPMKGRTFADHQK

SEQ ID NO: 383 - protein, 419 aa, Staphylococcus aureus
MAQEVIKIRGGRTLNGEVNISGAKNSAVAIIPATLLAQGHVKLEGLPQISDVKTLVSLLEDLNIKASLNGTELEVDTTEIQNAALPNNKVESLRASYYMMGAMLGRFKKCVIGLPGGCPLGPRPIDQHIKGFKALGAEIDESSTTSMKIEAKELKGAHIFLDMVSVGATINIMLAAVYATGQTVIENAAKEPEVVDVANFLTSMGANIKGAGTSTIKINGVKELHGSEYQVIPDRIEAGTYMCIAAACGENVILNNIVPKHVETLTAKFSELGVNVDVRDERIRINNNAPYQFVDIKTLVYPGFATDLQQPITPLLFMANGPSFVTDTIYPERFKHVEELKRMGANIEVDEGTATIKPSTLHGAEVYASDLRAGACLIIAGLIAEGVTTIYNVKHIYRGYTDIVEHLKALGADIWTETV

SEQ ID NO: 384 - protein, 26 aa, Staphylococcus aureus
MAQDIISTISDLVKWIIDTVNKFTKK

SEQ ID NO: 385 - protein, 4 aa, synthetic construct
  REGION 1..4: note = peptide
HMLK

SEQ ID NO: 386 - protein, 6 aa, synthetic construct
  REGION 1..6: note = peptide
HMHMHM

SEQ ID NO: 387 - protein, 10 aa, synthetic construct
  REGION 1..10: note = decapeptide
DEHGTAVMLK